SEQUENCE LISTING (168 sequences)

SEQ ID NO: 1 - protein, 132 aa, Artificial Sequence
  synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 2 - protein, 105 aa, Artificial Sequence
  synthetic construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 3 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEWFTYFYWEVWGNGTQVTVSS

SEQ ID NO: 4 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEWFTYFYWEVWGKGTQVTVSS

SEQ ID NO: 5 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNELFTYFYWEVWGNGTQVTVSS

SEQ ID NO: 6 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNELFTYFYWEVWGKGTQVTVSS

SEQ ID NO: 7 - protein, 132 aa, Artificial Sequence
  synthetic construct
QVQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNELFTYFYWEVWGKGTQVTVSS

SEQ ID NO: 8 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSS

SEQ ID NO: 9 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLKLKSVTAADTAVYYCARTLHGRRIYGVVAFNELFTYFYWEVWGKGTQVTVSS

SEQ ID NO: 10 - protein, 105 aa, Artificial Sequence
  synthetic construct
SDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVL

SEQ ID NO: 11 - protein, 112 aa, Artificial Sequence
  synthetic construct
SYVLTQPPSVSVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVL

SEQ ID NO: 12 - protein, 5 aa, Artificial Sequence
  synthetic construct
DSYWS

SEQ ID NO: 13 - protein, 16 aa, Artificial Sequence
  synthetic construct
YVHKSGDTNYSPSLKS

SEQ ID NO: 14 - protein, 24 aa, Artificial Sequence
  synthetic construct
TLHGRRIYGIVAFNEWFTYFYMDV

SEQ ID NO: 15 - protein, 11 aa, Artificial Sequence
  synthetic construct
GEKSLGSRAVQ

SEQ ID NO: 16 - protein, 7 aa, Artificial Sequence
  synthetic construct
NNQDRPS

SEQ ID NO: 17 - protein, 12 aa, Artificial Sequence
  synthetic construct
HIWDSRVPTKWV

SEQ ID NO: 18 - protein, 5 aa, Artificial Sequence
  synthetic construct
SSYWS

SEQ ID NO: 19 - protein, 16 aa, Artificial Sequence
  synthetic construct
YTHHSGDTNYAPSLKS

SEQ ID NO: 20 - protein, 24 aa, Artificial Sequence
  synthetic construct
TLHGRRIYGVVAFNEWFTYFYWEV

SEQ ID NO: 21 - protein, 24 aa, Artificial Sequence
  synthetic construct
TLHGRRIYGVVAFNELFTYFYWEV

SEQ ID NO: 22 - protein, 24 aa, Artificial Sequence
  synthetic construct
TLHGRRIYGVVAFNEFFTYFYWEV

SEQ ID NO: 23 - protein, 11 aa, Artificial Sequence
  synthetic construct
GGESIGSRAVQ

SEQ ID NO: 24 - protein, 7 aa, Artificial Sequence
  synthetic construct
NNQDRPP

SEQ ID NO: 25 - protein, 12 aa, Artificial Sequence
  synthetic construct
HIWDSRRPTNWV

SEQ ID NO: 26 - protein, 5 aa, Artificial Sequence
  synthetic construct
  MISC_FEATURE (1)..(1): Xaa can be Asp or Ser
XSYWS

SEQ ID NO: 27 - protein, 16 aa, Artificial Sequence
  synthetic construct
  MISC_FEATURE (2)..(2): Xaa can be Val or Thr
  MISC_FEATURE (4)..(4): Xaa can be Lys or His
  MISC_FEATURE (11)..(11): Xaa can be Ser or Ala
YXHXSGDTNYXPSLKS

SEQ ID NO: 28 - protein, 24 aa, Artificial Sequence
  synthetic construct
  MISC_FEATURE (7)..(7): Xaa can be Ile or Cys
  MISC_FEATURE (10)..(10): Xaa can be Ile or Val
  MISC_FEATURE (14)..(14): Xaa can be Asn or Gly
  MISC_FEATURE (16)..(16): Xaa can be Trp, Cys, Phe or Leu
  MISC_FEATURE (22)..(22): Xaa can be Met or Trp
  MISC_FEATURE (23)..(23): Xaa can be Asp or Glu
TLHGRRXYGXVAFXEXFTYFYXXV

SEQ ID NO: 29 - protein, 11 aa, Artificial Sequence
  synthetic construct
  MISC_FEATURE (2)..(2): Xaa can be Glu or Gly
  MISC_FEATURE (3)..(3): Xaa can be Lys or Glu
  MISC_FEATURE (5)..(5): Xaa can be Leu or Ile
GXXSXGSRAVQ

SEQ ID NO: 30 - protein, 7 aa, Artificial Sequence
  synthetic construct
  MISC_FEATURE (7)..(7): Xaa can be Ser or Pro
NNQDRPX

SEQ ID NO: 31 - protein, 12 aa, Artificial Sequence
  synthetic construct
  MISC_FEATURE (7)..(7): Xaa can be Val or Arg
  MISC_FEATURE (10)..(10): Xaa can be Lys or Asn
HIWDSRXPTXWV

SEQ ID NO: 32 - protein, 462 aa, Artificial Sequence
  synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 211 aa, Artificial Sequence
  synthetic construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 462 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEWFTYFYWEVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 35 - protein, 462 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEWFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 36 - protein, 462 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNELFTYFYWEVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 37 - protein, 462 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNELFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 38 - protein, 462 aa, Artificial Sequence
  synthetic construct
QVQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNELFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 39 - protein, 462 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 40 - protein, 462 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLKLKSVTAADTAVYYCARTLHGRRIYGVVAFNELFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 41 - protein, 211 aa, Artificial Sequence
  synthetic construct
SDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 218 aa, Artificial Sequence
  synthetic construct
SYVLTQPPSVSVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 43 - protein, 330 aa, Artificial Sequence [sequence omitted]

SEQ ID NO: 44 - protein, 330 aa, Artificial Sequence
  synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 45 - protein, 132 aa, Artificial Sequence; identical to SEQ ID NO: 1
  synthetic construct

SEQ ID NO: 46 - protein, 105 aa, Artificial Sequence
  synthetic construct
SDISVAPGETARISCGGESLGSRAVQWYQHRAGQAPSLIIYNNQDRPSGIPERFSGSPDIAFGTTATLTITSVEAGDEADYYCHIWDSRRPTNWVFGGGTTLTVL

SEQ ID NO: 47 - protein, 132 aa, Artificial Sequence
  synthetic construct
QMQLQESGPGLVKPGETLSLTCSVSGASISDSYWSWLRETPGKGLEWIGYVHKSGDTNYAPSLKSRVNLGLDTSKNQVSLSLVAATAADSGKYYCARTLHGRRIYGVVAFNEWFTYFYWDVWGNGTQVTVSS

SEQ ID NO: 48 - protein, 105 aa, Artificial Sequence; identical to SEQ ID NO: 10
  synthetic construct

SEQ ID NO: 49 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLVAATAADSGKYYCARTLHGRRIYGVVAFNEWFTYFYWEVWGNGTQVTVSS

SEQ ID NO: 50 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLVAATAADSGKYYCARTLHGRRIYGVVAFNEWFTYFYWEVWGKGTQVTVSS

SEQ ID NO: 51 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLVAATAADSGKYYCARTLHGRRIYGVVAFGEWFTYFYWEVWGNGTQVTVSS

SEQ ID NO: 52 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLVAATAADSGKYYCARTLHGRRCYGVVAFNECFTYFYWEVWGNGTQVTVSS

SEQ ID NO: 53 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLVAATAADSGKYYCARTLHGRRIYGVVAFNELFTYFYWEVWGNGTQVTVSS

SEQ ID NO: 54 - protein, 132 aa, Artificial Sequence; identical to SEQ ID NO: 3
  synthetic construct

SEQ ID NO: 55 - protein, 105 aa, Artificial Sequence; identical to SEQ ID NO: 10
  synthetic construct

SEQ ID NO: 56 - protein, 99 aa, Artificial Sequence
  synthetic construct
SYVLTQPPSVSVAPGKTARITCGGNNIGSKSVHWYQQKPGQAPVLVIYYDSDRPSGIPERFSGSNSNNNGNTATLTISRVEAGDEADYYCQVWDSSSDH

SEQ ID NO: 57 - protein, 97 aa, Artificial Sequence
  synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 58 - protein, 853 aa, Artificial Sequence
  synthetic construct
MRVKEMRKLWQHWWKGGILLLGMLMICSAKDNLWVTVYYGVPVWKEATTTLFCASDAKAYKAEVHNVWATHACVPTDPNPQEIVLENVTENFNMWKNNMVEQMHEDIISLWDQSLKPCVKLTPLCVTLNCIDLNNSTNNNNSSGVKTGIDKGEIKNCSFNTTTSVKDKEKKEYALFYNLDVVQIGNDNTSYRLTSCNTSVITQACPKVTFEPIPIHYCTPAGYAILKCNGKKFNGTGPCTNVSTVQCTHGIKPVVSTQLLLNGSLAEEDIVIRSENLTNNAKTIIVQLKDPVDINCTRPNNNTRKSIHIGPGRAFYATGDIIGDIRQAHCNLSRAQWNDTLSKIVTKLREQFENKTIKFQPPSGGDPEIVFHSFNCGGEFFYCNTTQLFNSTWTNNTEGTSNTTGNDTITLPCRIKQIVNMWQEVGKAMYAPPIKGKIKCSSNITGLLLTRDGGNNEMNTTEIFRPGGGDMRDNWRSELYKYKVVRIEPLGIAPTRAKRRVVQREKRAVGTLGAMFLGFLGAAGSTMGAASVALTVQARQLLSGIVQQQNNLLRAIEAQQHMLQLTVWGIKQLQARVLAVERYLGDQQLLGIWGCSGKLICTTTVPWNTSWSNKSLDDIWTNMTWMEWKREIDNYTSLIYTLIEESQRQQEKNEQELLELDKWDSLWNWFTISKWLWYIKIFIMIVAGLVGLRIVFAVLSIVNKVRQGYSPVSFQTRLPAQRGPDRPEEIEEEGGDRERERSGRLVTGLLEIIWDDLRSLCLFSYRRLRDLLLIVARIVELLGRRGWEALKYWWNLLQYWSQELKSSAVSLLNATAVAVAEGTDRIIEIAQRFFRGFLHIPRRIRQGLERALL

SEQ ID NO: 59 - protein, 24 aa, Artificial Sequence
  synthetic construct
TLHGRRIYGVVAFGEWFTYFYWEV

SEQ ID NO: 60 - protein, 24 aa, Artificial Sequence
  synthetic construct
TLHGRRCYGVVAFNECFTYFYWEV

SEQ ID NO: 61 - protein, 16 aa, Artificial Sequence
  synthetic construct
YVHKSGDTNYAPSLKS

SEQ ID NO: 62 - protein, 24 aa, Artificial Sequence
  synthetic construct
TLHGRRIYGVVAFNEWFTYFYWDV

SEQ ID NO: 63 - protein, 11 aa, Artificial Sequence
  synthetic construct
GGESLGSRAVQ

SEQ ID NO: 64 - protein, 24 aa, Artificial Sequence
  synthetic construct
  MISC_FEATURE (10)..(10): Xaa is Ile or Val
  MISC_FEATURE (14)..(14): Xaa is Asn or Gly
  MISC_FEATURE (16)..(16): Xaa is Trp, Phe or Leu
  MISC_FEATURE (22)..(22): Xaa is Met or Trp
  MISC_FEATURE (23)..(23): Xaa is Asp or Glu
TLHGRRIYGXVAFXEXFTYFYXXV

SEQ ID NO: 65 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWPTWGKGTQVTVSS

SEQ ID NO: 66 - protein, 105 aa, Artificial Sequence
  synthetic construct
SDISVAPGETVRITCGGESIGSRAVQWYQHRPGQAPRLIIYNNQDRPPGIPERFSGSPDIDFGTTATLTISNVEAGDEATYYCHIWDSRRPTNWELGPGTTLTVL

SEQ ID NO: 67 - protein, 24 aa, Artificial Sequence
  synthetic construct
TLHGRRIYGVVAFNEYYTYFYWPT

SEQ ID NO: 68 - protein, 12 aa, Artificial Sequence
  synthetic construct
HIWDSRRPTNWE

SEQ ID NO: 69 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVTIGLDPSKNQVSLSLTSVTAADTAVYYCARTLHGRRIYGVVAFNEYYTYFYWPTWGKGTQVTVSS

SEQ ID NO: 70 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVTISLDPSKNQVSLSLTSVTAADTAVYYCARTLHGRRIYGVVAFNEYYTYFYWPTWGKGTQVTVSS

SEQ ID NO: 71 - protein, 24 aa, Artificial Sequence
  synthetic construct
  MISC_FEATURE (7)..(7): Xaa is Ile or Cys
  MISC_FEATURE (10)..(10): Xaa is Ile or Val
  MISC_FEATURE (14)..(14): Xaa is Asn or Gly
  MISC_FEATURE (16)..(16): Xaa is Trp, Cys, Phe, Leu or Tyr
  MISC_FEATURE (17)..(17): Xaa is Phe or Tyr
  MISC_FEATURE (22)..(22): Xaa is Met or Trp
  MISC_FEATURE (23)..(23): Xaa is Asp, Pro, or Glu
  MISC_FEATURE (24)..(24): Xaa is Val or Thr
TLHGRRXYGXVAFXEXXTYFYXXX

SEQ ID NO: 72 - protein, 24 aa, Artificial Sequence
  synthetic construct
  MISC_FEATURE (10)..(10): Xaa is Ile or Val
  MISC_FEATURE (14)..(14): Xaa is Asn or Gly
  MISC_FEATURE (16)..(16): Xaa is Trp, Phe, Leu or Tyr
  MISC_FEATURE (17)..(17): Xaa is Phe or Tyr
  MISC_FEATURE (22)..(22): Xaa is Met or Trp
  MISC_FEATURE (23)..(23): Xaa is Asp, Pro or Glu
  MISC_FEATURE (24)..(24): Xaa is Val or Thr
TLHGRRIYGXVAFXEXXTYFYXXX

SEQ ID NO: 73 - protein, 12 aa, Artificial Sequence
  synthetic construct
  MISC_FEATURE (2)..(2): Xaa is Ile or Val
  MISC_FEATURE (7)..(7): Xaa is Val or Arg
  MISC_FEATURE (10)..(10): Xaa is Lys, Gln, or Asn
  MISC_FEATURE (12)..(12): Xaa is Val or Glu
HXWDSRXPTXWX

SEQ ID NO: 74 - protein, 30 aa, Artificial Sequence
  synthetic construct
QMQLQESGPGLVKPSETLSLTCSVSGASIS

SEQ ID NO: 75 - protein, 30 aa, Artificial Sequence
  synthetic construct
QMQLQESGPGLVKPGETLSLTCSVSGASIS

SEQ ID NO: 76 - protein, 30 aa, Artificial Sequence
  synthetic construct
QVQLQESGPGLVKPGETLSLTCSVSGASIS

SEQ ID NO: 77 - protein, 30 aa, Artificial Sequence
  synthetic construct
  MISC_FEATURE (2)..(2): Xaa is Met or Val
  MISC_FEATURE (15)..(15): Xaa is Ser or Gly
QXQLQESGPGLVKPXETLSLTCSVSGASIS

SEQ ID NO: 78 - protein, 14 aa, Artificial Sequence
  synthetic construct
WIRRSPGKGLEWIG

SEQ ID NO: 79 - protein, 14 aa, Artificial Sequence
  synthetic construct
WLRETPGKGLEWIG

SEQ ID NO: 80 - protein, 14 aa, Artificial Sequence
  synthetic construct
  MISC_FEATURE (2)..(2): Xaa is Ile or Leu
  MISC_FEATURE (4)..(4): Xaa is Arg or Glu
  MISC_FEATURE (5)..(5): Xaa is Ser or Thr
  MISC_FEATURE (10)..(10): Xaa is Ile, Leu, or Val
WXRXXPGKGXEWIG

SEQ ID NO: 81 - protein, 32 aa, Artificial Sequence
  synthetic construct
RVNLSLDTSKNQVSLSLVAATAADSGKYYCAR

SEQ ID NO: 82 - protein, 32 aa, Artificial Sequence
  synthetic construct
RVNLGLDTSKNQVSLSLVAATAADSGKYYCAR

SEQ ID NO: 83 - protein, 32 aa, Artificial Sequence
  synthetic construct
RVHLGLHPSKNQVSLSLTSVTAADTAVYYCAR

SEQ ID NO: 84 - protein, 32 aa, Artificial Sequence
  synthetic construct
RVHLGLHPSKNQVSLKLKSVTAADTAVYYCAR

SEQ ID NO: 85 - protein, 32 aa, Artificial Sequence
  synthetic construct
RVHLGLHPSKNQVSLSLVAATAADSGKYYCAR

SEQ ID NO: 86 - protein, 32 aa, Artificial Sequence
  synthetic construct
RVTIGLDPSKNQVSLSLTSVTAADTAVYYCAR

SEQ ID NO: 87 - protein, 32 aa, Artificial Sequence
  synthetic construct
RVTISLDPSKNQVSLSLTSVTAADTAVYYCAR

SEQ ID NO: 88 - protein, 32 aa, Artificial Sequence
  synthetic construct
  MISC_FEATURE (3)..(3): Xaa is Asn, His, or Thr
  MISC_FEATURE (4)..(4): Xaa is Leu or Ile
  MISC_FEATURE (5)..(5): Xaa is Ser or Gly
  MISC_FEATURE (7)..(7): Xaa is Asp or His
  MISC_FEATURE (8)..(8): Xaa is Thr or Pro
  MISC_FEATURE (9)..(9): Xaa is Ser or Glu
  MISC_FEATURE (16)..(16): Xaa is Ser or Lyslu
  MISC_FEATURE (18)..(18): Xaa is Val, Thr, or Lys
  MISC_FEATURE (19)..(19): Xaa is Ala or Ser
  MISC_FEATURE (20)..(20): Xaa is Ala or Val
  MISC_FEATURE (25)..(25): Xaa is Ser or Thr
  MISC_FEATURE (26)..(26): Xaa is Gly or Ala
  MISC_FEATURE (27)..(27): Xaa is Lys or Val
RVXXXLXXXKNQVSLXLXXXTAADXXXYYCAR

SEQ ID NO: 89 - protein, 15 aa, Artificial Sequence
  synthetic construct
YMDVWGNGTQVTVSS

SEQ ID NO: 90 - protein, 15 aa, Artificial Sequence
  synthetic construct
YWDVWGNGTQVTVSS

SEQ ID NO: 91 - protein, 15 aa, Artificial Sequence
  synthetic construct
YWEVWGNGTQVTVSS

SEQ ID NO: 92 - protein, 15 aa, Artificial Sequence
  synthetic construct
YWEVWGKGTQVTVSS

SEQ ID NO: 93 - protein, 15 aa, Artificial Sequence
  synthetic construct
YWPTWGKGTQVTVSS

SEQ ID NO: 94 - protein, 15 aa, Artificial Sequence
  synthetic construct
  MISC_FEATURE (2)..(2): Xaa is Met or Trp
  MISC_FEATURE (3)..(3): Xaa is Asp, Glu, or Pro
  MISC_FEATURE (4)..(4): Xaa is Val or Thr
  MISC_FEATURE (7)..(7): Xaa is Asn or Lys
  MISC_FEATURE (9)..(9): Xaa is Ile or Thr
  MISC_FEATURE (10)..(10): Xaa is Gln or Lys
YXXXWGXGXXVTVSS

SEQ ID NO: 95 - protein, 15 aa, Artificial Sequence
  synthetic construct
SDISVAPGETARISC

SEQ ID NO: 96 - protein, 15 aa, Artificial Sequence
  synthetic construct
SDISVAPGETVRISC

SEQ ID NO: 97 - protein, 22 aa, Artificial Sequence
  synthetic construct
SYVLTQPPSVSVAPGETVRISC

SEQ ID NO: 98 - protein, 15 aa, Artificial Sequence
  synthetic construct
SDISVAPGETVRITC

SEQ ID NO: 99 - protein, 15 aa, Artificial Sequence
  synthetic construct
  MISC_FEATURE (1)..(1): Xaa is Ser or Pro
  MISC_FEATURE (2)..(2): Xaa is Asp or Ser
  MISC_FEATURE (3)..(3): Xaa is Ile, Leu, or Val
  MISC_FEATURE (11)..(11): Xaa is Ala or Val
  MISC_FEATURE (14)..(14): Xaa is Ser or Thr
XXXSVAPGETXRIXC

SEQ ID NO: 100 - protein, 15 aa, Artificial Sequence
  synthetic construct
WYQHRAGQAPSLIIY

SEQ ID NO: 101 - protein, 15 aa, Artificial Sequence
  synthetic construct
WYQHRAGQAPKLIIY

SEQ ID NO: 102 - protein, 15 aa, Artificial Sequence
  synthetic construct
WYQHRPGQAPRLIIY

SEQ ID NO: 103 - protein, 15 aa, Artificial Sequence
  synthetic construct
  MISC_FEATURE (4)..(4): Xaa is Gln or His
  MISC_FEATURE (6)..(6): Xaa is Ala or Pro
  MISC_FEATURE (11)..(11): Xaa is Ser, Lys, Arg, or Pro
WYQXRXGQAPXLIIY

SEQ ID NO: 104 - protein, 35 aa, Artificial Sequence
  synthetic construct
GIPERFSGSPDSPFGTTATLTITSVEAGDEADYYC

SEQ ID NO: 105 - protein, 35 aa, Artificial Sequence
  synthetic construct
GIPERFSGSPDIDFGTTATLTITNVEAGDEATYYC

SEQ ID NO: 106 - protein, 35 aa, Artificial Sequence
  synthetic construct
GIPERFSGSPDIAFGTTATLTITSVEAGDEADYYC

SEQ ID NO: 107 - protein, 35 aa, Artificial Sequence
  synthetic construct
GIPERFSGSPDIDFGTTATLTISNVEAGDEATYYC

SEQ ID NO: 108 - protein, 35 aa, Artificial Sequence
  synthetic construct
  MISC_FEATURE (12)..(12): Xaa is Ser, Leu, or Ile
  MISC_FEATURE (13)..(13): Xaa is Pro, Ala, or Asp
  MISC_FEATURE (23)..(23): Xaa is Thr or Ser
  MISC_FEATURE (24)..(24): Xaa is Ser, Arg, or Asn
  MISC_FEATURE (32)..(32): Xaa is Asp or Thr
GIPERFSGSPDXXFGTTATLTIXXVEAGDEAXYYC

SEQ ID NO: 109 - protein, 10 aa, Artificial Sequence
  synthetic construct
FGGGTTLTVL

SEQ ID NO: 110 - protein, 10 aa, Artificial Sequence
  synthetic construct
LGPGTTLTVL

SEQ ID NO: 111 - protein, 10 aa, Artificial Sequence
  synthetic construct
  MISC_FEATURE (1)..(1): Xaa is Phe or Leu
  MISC_FEATURE (2)..(2): Xaa is Glu or Gly
  MISC_FEATURE (3)..(3): Xaa is Gly or Pro
XXXGTTLTVL

SEQ ID NO: 112 - protein, 132 aa, Artificial Sequence
  synthetic construct
QMQLQESGPGLVKPGETLSLTCSVSGASISSSYWSWLRETPGKGIEWIGYTHHSGDTNYAPSLKSRVHLGLHPEKNQVSLKLTSVTAADTAVYYCARTLHGRRIYGVVAGNEYYTYFYWPTWGKGTQVTVSS

SEQ ID NO: 113 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPEKNQVSLKLTSVTAADTAVYYCARTLHGRRIYGVVAGGEYYTYFYWPTWGKGTQVTVSS

SEQ ID NO: 114 - protein, 132 aa, Artificial Sequence
  synthetic construct
QMQLQESGPGLVKPGETLSLTCSVSGASISSSYWSWLRETPGKGIEWIGYTHHSGDTNYAPSLKSRVHLGLHPSKNQVSLKLTSVTAADTAVYYCARTLHGRRIYGVVAGNEFYTYFYWPTWGKGTQVTVSS

SEQ ID NO: 115 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPEKNQVSLSLTSVTAADTAVYYCARTLHGRRIYGVVAGNEYYTYFYWPTWGKGIQVTVSS

SEQ ID NO: 116 - protein, 132 aa, Artificial Sequence
  synthetic construct
QMQLQESGPGLVKPGETLSLTCSVSGASISSSYWSWLRETPGKGIEWIGYTHHSGDTNYAPSLKSRVHLGLHPSKNQVSLSLTSVTAADTAVYYCARTLHGRRIYGVVAGNEFYTYFYWPTWGKGTKVTVSS

SEQ ID NO: 117 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAGGEFYTYFYWPTWGKGTQVTVSS

SEQ ID NO: 118 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLKLTSVTAADTAVYYCARTLHGRRIYGVVAGNEYYTYFYWPTWGKGIQVTVSS

SEQ ID NO: 119 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVEGGEYYTYFYWPTWGKGTQVTVSS

SEQ ID NO: 120 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPEKNQVSLSLTSVTAADTAVYYCARTLHGRRIYGVVAGNEFYTYFYWPTWGKGTQVTVSS

SEQ ID NO: 121 - protein, 132 aa, Artificial Sequence
  synthetic construct
QMQLQESGPGLVKPGETLSLTCSVSGASISSSYWSWLRETPGKGIEWIGYTHHSGDTNYAPSLKSRVHLGLHPSKNQVSLSLTSVTAADTAVYYCARTLHGRRIYGVVEGGEYYTYFYWPTWGKGTKVTVSS

SEQ ID NO: 122 - protein, 132 aa, Artificial Sequence
  synthetic construct
QMQLQESGPGLVKPGETLSLTCSVSGASISSSYWSWLRETPGKGVEWIGYTHHSGDTNYAPSLKSRVHLGLHPEKNQVSLSLTSVTAADTAVYYCARTLHGRRIYGVVAGNEFYTYFYWPTWGKGIQVTVSS

SEQ ID NO: 123 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAGNEFYTYFYWPTWGKGIQVTVSS

SEQ ID NO: 124 - protein, 132 aa, Artificial Sequence
  synthetic construct
QMQLQESGPGLVKPGETLSLTCSVSGASISSSYWSWLRETPGKGIEWIGYTHHSGDTNYAPSLKSRVHLGLHPSKNQVSLSLTSVTAADTAVYYCARTLHGRRIYGVVAGGEYYTYFYWPTWGKGTQVTVSS

SEQ ID NO: 125 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAGGEYYTYFYWPTWGKGIQVTVSS

SEQ ID NO: 126 - protein, 132 aa, Artificial Sequence
  synthetic construct
QMQLQESGPGLVKPGETLSLTCSVSGASISSSYWSWLRETPGKGLEWIGYTHHSGDTNYAPSLKSRVHLGLHPSKNQVSLKLTSVTAADTAVYYCARTLHGRRIYGVVAGNEFYTYFYWPTWGKGTKVTVSS

SEQ ID NO: 127 - protein, 105 aa, Artificial Sequence
  synthetic construct
SDISVAPGETVRISCGGESIGSRAVQWYQHRAGQAPRLIIYNNQDRPPGIPERFSGSPDIDFGTTATLTITNVEAGDEATYYCHIWDSRRPTNWELGPGTTLTVL

SEQ ID NO: 128 - protein, 105 aa, Artificial Sequence
  synthetic construct
SDLSVAPGETVRITCGGESIGSRAVQWYQHRPGQAPRLIIYNNQDRPPGIPERFSGSPDIDFGTTATLTISNVEAGDEATYYCHIWDSRRPTNWVFEPGTTLTVL

SEQ ID NO: 129 - protein, 105 aa, Artificial Sequence
  synthetic construct
SDLSVAPGETVRISCGGESIGSRAVQWYQHRAGQAPRLIIYNNQDRPPGIPERFSGSPDIDFGTTATLTITNVEAGDEATYYCHIWDSRRPTNWVFEPGTTLTVL

SEQ ID NO: 130 - protein, 105 aa, Artificial Sequence
  synthetic construct
SDLSVAPGETVRITCGGESIGSRAVQWYQHRPGQAPKLIIYNNQDRPPGIPERFSGSPDIDFGTTATLTISRVEAGDEATYYCHVWDSRRPTNWELGPGTTLTVL

SEQ ID NO: 131 - protein, 105 aa, Artificial Sequence
  synthetic construct
SDLSVAPGETVRISCGGESIGSRAVQWYQHRAGQAPKLIIYNNQDRPPGIPERFSGSPDIDFGTTATLTITRVEAGDEATYYCHVWDSRRPTNWELGPGTTLTVL

SEQ ID NO: 132 - protein, 105 aa, Artificial Sequence
  synthetic construct
SDISVAPGETVRITCGGESIGSRAVQWYQHRPGQAPRLIIYNNQDRPPGIPERFSGSPDLDFGTTATLTISNVEAGDEATYYCHVWDSRRPTNWELGPGTTLTVL

SEQ ID NO: 133 - protein, 105 aa, Artificial Sequence
  synthetic construct
SDISVAPGETVRISCGGESIGSRAVQWYQHRAGQAPRLIIYNNQDRPPGIPERFSGSPDLDFGTTATLTITNVEAGDEATYYCHVWDSRRPTNWELGPGTTLTVL

SEQ ID NO: 134 - protein, 105 aa, Artificial Sequence
  synthetic construct
SDISVAPGETVRITCGGESIGSRAVQWYQHRPGQAPRLIIYNNQDRPPGIPERFSGSPDIDFGTTATLTISNVEAGDEATYYCHVWDSRRPTQWVLEGGTTLTVL

SEQ ID NO: 135 - protein, 105 aa, Artificial Sequence
  synthetic construct
SDISVAPGETVRISCGGESIGSRAVQWYQHRAGQAPRLIIYNNQDRPPGIPERFSGSPDIDFGTTATLTITNVEAGDEATYYCHVWDSRRPTQWVLEGGTTLTVL

SEQ ID NO: 136 - protein, 105 aa, Artificial Sequence
  synthetic construct
SDLSVAPGETVRITCGGESIGSRAVQWYQHRPGQAPRLIIYNNQDRGPGIPERFSGSPDIDFGTTATLTISNVEAGDEATYYCHIWDSRRPTNWELGGGTTLTVL

SEQ ID NO: 137 - protein, 105 aa, Artificial Sequence
  synthetic construct
SDLSVAPGETVRISCGGESIGSRAVQWYQHRAGQAPRLIIYNNQDRGPGIPERFSGSPDIDFGTTATLTITNVEAGDEATYYCHIWDSRRPTNWELGGGTTLTVL

SEQ ID NO: 138 - protein, 105 aa, Artificial Sequence
  synthetic construct
SDISVAPGETVRITCGGESIGSRAVQWYQQRPGQAPRLIIYNNQDRPPGIPERFSGSPDIDFGTTATLTISNVEAGDEATYYCHVWDSRRPTNWELGPGTTLTVL

SEQ ID NO: 139 - protein, 105 aa, Artificial Sequence
  synthetic construct
SDISVAPGETVRISCGGESIGSRAVQWYQQRAGQAPRLIIYNNQDRPPGIPERFSGSPDIDFGTTATLTITNVEAGDEATYYCHVWDSRRPTNWELGPGTTLTVL

SEQ ID NO: 140 - protein, 24 aa, Artificial Sequence
  synthetic construct
TLHGRRIYGVVAGNEYYTYFYWPT

SEQ ID NO: 141 - protein, 24 aa, Artificial Sequence
  synthetic construct
TLHGRRIYGVVAGGEYYTYFYWPT

SEQ ID NO: 142 - protein, 24 aa, Artificial Sequence
  synthetic construct
TLHGRRIYGVVAGNEFYTYFYWPT

SEQ ID NO: 143 - protein, 24 aa, Artificial Sequence
  synthetic construct
TLHGRRIYGVVAGGEFYTYFYWPT

SEQ ID NO: 144 - protein, 24 aa, Artificial Sequence
  synthetic construct
TLHGRRIYGVVEGGEYYTYFYWPT

SEQ ID NO: 145 - protein, 7 aa, Artificial Sequence
  synthetic construct
NNQDRGP

SEQ ID NO: 146 - protein, 12 aa, Artificial Sequence
  synthetic construct
HVWDSRRPTNWE

SEQ ID NO: 147 - protein, 12 aa, Artificial Sequence
  synthetic construct
HVWDSRRPTQWV

SEQ ID NO: 148 - protein, 14 aa, Artificial Sequence
  synthetic construct
WLRETPGKGIEWIG

SEQ ID NO: 149 - protein, 14 aa, Artificial Sequence
  synthetic construct
WLRETPGKGVEWIG

SEQ ID NO: 150 - protein, 32 aa, Artificial Sequence
  synthetic construct
RVHLGLHPEKNQVSLKLTSVTAADTAVYYCAR

SEQ ID NO: 151 - protein, 32 aa, Artificial Sequence
  synthetic construct
RVHLGLHPSKNQVSLKLTSVTAADTAVYYCAR

SEQ ID NO: 152 - protein, 32 aa, Artificial Sequence
  synthetic construct
RVHLGLHPEKNQVSLSLTSVTAADTAVYYCAR

SEQ ID NO: 153 - protein, 15 aa, Artificial Sequence
  synthetic construct
YWPTWGKGIQVTVSS

SEQ ID NO: 154 - protein, 15 aa, Artificial Sequence
  synthetic construct
YWPTWGKGTKVTVSS

SEQ ID NO: 155 - protein, 15 aa, Artificial Sequence
  synthetic construct
SDLSVAPGETVRITC

SEQ ID NO: 156 - protein, 15 aa, Artificial Sequence
  synthetic construct
SDLSVAPGETVRISC

SEQ ID NO: 157 - protein, 15 aa, Artificial Sequence
  synthetic construct
WYQHRAGQAPRLIIY

SEQ ID NO: 158 - protein, 15 aa, Artificial Sequence
  synthetic construct
WYQHRPGQAPKLIIY

SEQ ID NO: 159 - protein, 15 aa, Artificial Sequence
  synthetic construct
WYQQRPGQAPRLIIY

SEQ ID NO: 160 - protein, 15 aa, Artificial Sequence
  synthetic construct
WYQQRAGQAPRLIIY

SEQ ID NO: 161 - protein, 35 aa, Artificial Sequence
  synthetic construct
GIPERFSGSPDIDFGTTATLTISRVEAGDEATYYC

SEQ ID NO: 162 - protein, 35 aa, Artificial Sequence
  synthetic construct
GIPERFSGSPDIDFGTTATLTITRVEAGDEATYYC

SEQ ID NO: 163 - protein, 35 aa, Artificial Sequence
  synthetic construct
GIPERFSGSPDLDFGTTATLTISNVEAGDEATYYC

SEQ ID NO: 164 - protein, 35 aa, Artificial Sequence
  synthetic construct
GIPERFSGSPDLDFGTTATLTITNVEAGDEATYYC

SEQ ID NO: 165 - protein, 10 aa, Artificial Sequence
  synthetic construct
FEPGTTLTVL

SEQ ID NO: 166 - protein, 10 aa, Artificial Sequence
  synthetic construct
LEGGTTLTVL

SEQ ID NO: 167 - protein, 10 aa, Artificial Sequence
  synthetic construct
LGGGTTLTVL

SEQ ID NO: 168 - protein, 7 aa, Artificial Sequence
  synthetic construct
  MISC_FEATURE (6)..(6): Xaa can be Pro or Gly
  MISC_FEATURE (7)..(7): Xaa can be Ser or Pro
NNQDRXX

(19) United States
(12) Patent Application Publication
(10) Pub. No.: US 2021/0292396 A1
(43) Pub. Date: Sep. 23, 2021

(54) ENGINERRED ANTIBODIES TO HIV ENV

(71) Applicants: International AIDS Vaccine Initiative, New York, NY (US); The Scripps Research Institute, La Jolla, CA (US)

(72) Inventors: Devin SOK, San Diego, CA (US); Joseph JARDINE, La Jolla, CA (US)

(21) Appl. No.: 17/263,490

(22) PCT Filed: Jul. 26, 2019
(86) PCT No.: PCT/US2019/043578

§ 371 Date: Jan. 26, 2021

Related U.S. Application Data
(60) Provisional application No. 62/816,721, filed on Mar. 11, 2019.
(60) Provisional application No. 62/711,312, filed on Jul. 27, 2018.

(51) Int. Cl. C07K 16/10; A61P 31/18; A61K 45/06; A61K 39/42

(52) U.S. Cl. CPC C07K 16/1063; A61P 31/18; A61K 45/06; A61K 39/42; C07K 2317/33; C07K 2317/567; C07K 2317/76; C07K 2317/92; C07K 2317/34; C07K 2317/565

(57) ABSTRACT

The present disclosure relates to anti-HIV Env antibodies and their use in the treatment or prevention of HIV/AIDS.

### CROSS REFERENCE TO RELATED APPLICATIONS

This application claims the benefit of U.S. Prov. Appl. No. 62/711,312, filed Jul. 27, 2018 and U.S. Prov. Appl. No. 62/816,721, filed Mar. 11, 2019, each of which is herein incorporated by reference in its entirety.

### GOVERNMENT INTEREST

The invention was made with government support under Grant No. AID-OAA-A-16-00032 awarded by the USAID.

### FIELD OF THE INVENTION

The field of the invention generally relates to anti-HIV Env antibodies and their use in the treatment or prevention of HIV/AIDS.

### BACKGROUND

In 2015, there were approximately 2.1 million new human immunodeficiency virus (HIV) infections, over 36.7 million people living with HIV, and 1.1 million acquired immune deficiency syndrome (AIDS) related deaths. unaids.org/en/resources/fact-sheet (accessed on Jun. 29, 2017) While great progress has been made in the treatment of HIV/AIDS, all individuals living with HIV will have to be treated with anti-retroviral therapy (ART) for the rest of their lives since drug therapy is unable to clear latent viral reservoirs that exist in resting CD4+ T cells at a frequency of about 1/10⁶ cells. See, Eriksson, S. 2013. PLoS Pathog 9:e1003174.

HIV isolates can be classified into different groups and clades based on genotype and geographic location. For example, the population episensus (i.e., epitope based consensus sequence) antigen is central to the B clade epidemic in the United States while the population episensus antigen is central to the HIV C clade epidemic in South Africa. Broadly neutralizing anti-Env antibodies, for example PGT-121 can neutralize more than one HIV isolate. U.S. Pat. No. 9,464,131.

Until a vaccine is discovered, many agree that a single product or approach will not completely halt new HIV infections. Accordingly, the use of HIV broadly neutralizing antibodies (bnAbs) has the potential to complement existing prevention methods by addressing important shortfalls or gaps in current product profiles. To achieve the goal of making bnAbs affordable and feasible products for widespread use in HIV prevention efforts, maximizing the potency of current and future bnAb candidates represents a promising and inadequately explored opportunity.

Thus, there remains a need for the development of broadly neutralizing antibodies that can be used in the treatment and prevention of HIV.

### BRIEF SUMMARY

In one aspect, provided herein are enhanced engineered anti-HIV Env antibodies that were derived from the PGT121 parent antibody using directed-evolution and yeast display.

In one aspect, provided herein are pharmaceutical compositions comprising the enhanced engineered anti-HIV Env antibodies disclosed herein.

In one aspect, provided herein are isolated polynucleotides encoding the enhanced engineered anti-HIV Env antibodies disclosed herein.

In one aspect, provided herein are methods of producing the enhanced engineered anti-HIV Env antibodies disclosed herein.

In one aspect, provided herein are methods of neutralizing an HIV virus, comprising contacting the virus with the enhanced engineered anti-HIV Env antibodies disclosed herein.

In one aspect, provided herein are methods of reducing the likelihood of HIV infection in a subject exposed to HIV comprising administering to the subject the enhanced engineered anti-HIV Env antibodies disclosed herein.

In one aspect, provided herein are methods of treating HIV/AIDS comprising administering to a subject in need thereof the enhanced engineered anti-HIV Env antibodies disclosed herein.

In one aspect, provided herein are methods of reducing viral load comprising administering to a subject in need thereof the enhanced engineered anti-HIV Env antibodies disclosed herein.

In one aspect, provided herein are methods of producing an engineered variant of PGT121.

In some embodiments, the disclosure provides:
[1] An antibody or antigen binding fragment thereof capable of binding HIV gp120, wherein the antibody or antigen binding fragment thereof comprises a VH and a VL, wherein the VH comprises a VH CDR1, VH CDR2, and VH CDR3, and the VL comprises a VL CDR1, VL CDR2, and VL CDR3, wherein
   a) the VH CDR1 comprises the sequence of X1SYWS, wherein X1 is D or S (SEQ ID NO: 26);
   b) the VH CDR2 comprises the sequence of YX1HX2SGDTNYX3PSLKS wherein X1 is V or T, X2 is K or H, and X3 is S or A (SEQ ID NO: 27);
   c) the VH CDR3 comprises the sequence of
      TLHGRRX1YGX2VAFX3EX4X5TYFYX6X7X8 wherein X1 is I or C, X2 is I or V, X3 is N or G, X4 is W, C, F, L or Y, X5 is F or Y, X6 is M or W, X7 is D, P or E, and X8 is V or T (SEQ ID NO: 71) or
      TLHGRRIYGX1VAFX2EX3X4TYFYX5X6X7 wherein X1 is I or V, X2 is N or G, X3 is W, F, L or Y, X4 is F or Y, X5 is M or W, X6 is D, P or E, and X7 is V or T (SEQ ID NO: 72);
   d) the VL CDR1 comprises the sequence of GX1X2SX3GSRAVQ wherein X1 is E or G, X2 is K or E, and X3 is L or I (SEQ ID NO: 29);
   e) the VL CDR2 comprises the sequence of NNQDRX1X2 wherein X1 is P or G, and X2 is S or P (SEQ ID NO: 168); and
   f) the VL CDR3 comprises the sequence of HX1WDSRX2PTX3WX4 wherein X1 is I or V, X2 is V or R, X3 is K, Q or N, and X4 is V or E (SEQ ID NO: 73);
   wherein at least one of the VH CDR1, VH CDR2, VH CDR3, VL CDR1, VL CDR2, and VL CDR3 comprises a different amino acid sequence than the corresponding CDR of the PGT-121 antibody;
[2] An antibody or antigen binding fragment thereof capable of binding HIV gp120, wherein the antibody or antigen binding fragment thereof comprises a VH and a VL, wherein the VH comprises a VH CDR1, VH CDR2, and VH CDR3, and the VL comprises a VL CDR1, VL CDR2, and VL CDR3, wherein
   a) the VH CDR1 comprises the sequence of X1SYWS, wherein X1 is D or S (SEQ ID NO: 26);
   b) the VH CDR2 comprises the sequence of YX1HX2SGDTNYX3PSLKS wherein X1 is V or T, X2 is K or H, and X3 is S or A (SEQ ID NO: 27);
   c) the VH CDR3 comprises the sequence of
      TLHGRRX1YGX2VAFX3EX4FTYFYX5X6V wherein X1 is I or C, X2 is I or V, X3 is N or G, X4 is W, C, F or L, X5 is M or W, and X6 is D or E (SEQ ID NO: 28) or TLHGRRIYGX1VAFX2EX3FTYFYX4X5V wherein X1 is I or V, X2 is N or G, X3 is W, F or L, X4 is M or W, and X5 is D or E (SEQ ID NO: 64);
   d) the VL CDR1 comprises the sequence of GX1X2SX3GSRAVQ wherein X1 is E or G, X2 is K or E, and X3 is L or I (SEQ ID NO: 29);
   e) the VL CDR2 comprises the sequence of NNQDRPX1 wherein X1 is S or P (SEQ ID NO: 30); and
   f) the VL CDR3 comprises the sequence of HIWDSRX1PTX2WV wherein X1 is V or R, and X2 is K or N (SEQ ID NO: 31);
   wherein at least one of the VH CDR1, VH CDR2, VH CDR3, VL CDR1, VL CDR2, and VL CDR3 comprises a different amino acid sequence than the corresponding CDR of the PGT-121 antibody;
[3] the antibody or antigen binding fragment thereof of [1] or [2], wherein the VH CDR1 comprises the sequence of DSYWS (SEQ ID NO: 12);
[4] the antibody or antigen binding fragment thereof of [1] or [2], wherein the VH CDR1 comprises the sequence of SSYWS (SEQ ID NO: 18);
[5] the antibody or antigen binding fragment thereof of any one of [1] to [4], wherein the VH CDR2 comprises the sequence of YVHKSGDTNYAPSLKS (SEQ ID NO: 13);
[6] the antibody or antigen binding fragment thereof of any one of [1] to [4], wherein the VH CDR2 comprises the sequence of YTHHSGDTNYAPSLKS (SEQ ID NO: 19);
[7] the antibody or antigen binding fragment thereof of any one of [1] to [4], wherein the VH CDR2 comprises the sequence of YVHKSGDTNYAPSLKS (SEQ ID NO: 61);
[8] the antibody or antigen binding fragment thereof of any one of [1] to [7], wherein the VH CDR3 comprises the sequence of TLHGRRIYGIVAFNEWFTYFYMDV (SEQ ID NO: 14);
[9] the antibody or antigen binding fragment thereof of any one of [1] to [7], wherein the VH CDR3 comprises the sequence of TLHGRRIYGVVAFNEWFTYFYWEV (SEQ ID NO: 20);
[10] the antibody or antigen binding fragment thereof of any one of [1] to [7], wherein the VH CDR3 comprises the sequence of TLHGRRIYGVVAFNELFTYFYWEV (SEQ ID NO: 21);
[11] the antibody or antigen binding fragment thereof of any one of [1] to [7], wherein the VH CDR3 comprises the sequence of TLHGRRIYGVVAFNEFFTYFYWEV (SEQ ID NO: 22);
[12] the antibody or antigen binding fragment thereof of any one of [1] to [7], wherein the VH CDR3 comprises the sequence of TLHGRRIYGVVAFGEWFTYFYWEV (SEQ ID NO: 59);
[13] the antibody or antigen binding fragment thereof of any one of [1] to [7], wherein the VH CDR3 comprises the sequence of TLHGRRCYGVVAFNECFTYFYWEV (SEQ ID NO: 60);
[14] the antibody or antigen binding fragment thereof of any one of [1] to [7], wherein the VH CDR3 comprises the sequence of TLHGRRIYGVVAFNEWFTYFYWDV (SEQ ID NO: 62);
[15] the antibody or antigen binding fragment thereof of any one of [1] to [7], wherein the VH CDR3 comprises the sequence of TLHGRRIYGVVAFNEYYTYFYWPT (SEQ ID NO: 67);
[16] the antibody or antigen binding fragment thereof of any one of [1] to [7], wherein the VH CDR3 comprises the sequence of TLHGRRIYGVVAGNEYYTYFYWPT (SEQ ID NO: 140);
[17] the antibody or antigen binding fragment thereof of any one of [1] to [7], wherein the VH CDR3 comprises the sequence of TLHGRRIYGVVAGGEYYTYFYWPT (SEQ ID NO: 141);
[18] the antibody or antigen binding fragment thereof of any one of [1] to [7], wherein the VH CDR3 comprises the sequence of TLHGRRIYGVVAGNEFYTYFYWPT (SEQ ID NO: 142);
[19] the antibody or antigen binding fragment thereof of any one of [1] to [7], wherein the VH CDR3 comprises the sequence of TLHGRRIYGVVAGGEFYTYFYWPT (SEQ ID NO: 143);
[20] the antibody or antigen binding fragment thereof of any one of [1] to [7], wherein the VH CDR3 comprises the sequence of TLHGRRIYGVVEGGEYYTYFYWPT (SEQ ID NO: 144);
[21] the antibody or antigen binding fragment thereof of any one of [1] to [20], wherein the VL CDR1 comprises the sequence of GEKSLGSRAVQ (SEQ ID NO: 15);
[22] the antibody or antigen binding fragment thereof of any one of [1] to [20], wherein the VL CDR1 comprises the sequence of GGESIGSRAVQ (SEQ ID NO: 23);
[23] the antibody or antigen binding fragment thereof of any one of [1] to [20], wherein the VL CDR1 comprises the sequence of GGESLGSRAVQ (SEQ ID NO: 63);
[24] the antibody or antigen binding fragment thereof of any one of [1] to [23], wherein the VL CDR2 comprises the sequence of NNQDRPS (SEQ ID NO: 16);
[25] the antibody or antigen binding fragment thereof of any one of [1] to [23], wherein the VL CDR2 comprises the sequence of NNQDRPP (SEQ ID NO: 24);
[26] the antibody or antigen binding fragment thereof of any one of [1] to [23], wherein the VL CDR2 comprises the sequence of NNQDRGP (SEQ ID NO: 145);
[27] the antibody or antigen binding fragment thereof of any one of [1] to [26], wherein the VL CDR3 comprises the sequence of HIWDSRVPTKWV (SEQ ID NO: 17);
[28] the antibody or antigen binding fragment thereof of any one of [1] to [26], wherein the VL CDR3 comprises the sequence of HIWDSRRPTNWV (SEQ ID NO: 25);
[29] the antibody or antigen binding fragment thereof of any one of [1] to [26], wherein the VL CDR3 comprises the sequence of HIWDSRRPTNWE (SEQ ID NO: 68);
[30] the antibody or antigen binding fragment thereof of any one of [1] to [26], wherein the VL CDR3 comprises the sequence of HVWDSRRPTNWE (SEQ ID NO: 146);
[31] the antibody or antigen binding fragment thereof of any one of [1] to [26], wherein the VL CDR3 comprises the sequence of HVWDSRRPTQWV (SEQ ID NO: 147);
[32] the antibody or antigen binding fragment thereof of [1] or [2], wherein
   a) the VH CDR1 comprises the sequence of DSYWS (SEQ ID NO: 12);
   b) the VH CDR2 comprises the sequence of YVHKSGDTNYSPSLKS (SEQ ID NO: 13); and
   c) the VH CDR3 comprises the sequence of TLHGRRIYGIVAFNEWFTYFYMDV (SEQ ID NO: 14);
[33] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO:19); and
   c) the VH CDR3 comprises the sequence of TLHGRRIYGVVAFNEWFTYFYWEV (SEQ ID NO: 20);
[34] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO: 19); and
   c) the VH CDR3 comprises the sequence of TLHGRRIYGVVAFNELFTYFYWEV (SEQ ID NO: 21;
[35] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO: 19); and
   c) the VH CDR3 comprises the sequence of TLHGRRIYGVVAFNEFFTYFYWEV (SEQ ID NO: 22);
[36] the antibody or antigen binding fragment thereof of [1] or [2], wherein
   a) the VH CDR1 comprises the sequence of SEQ ID NO: 12;
   b) the VH CDR2 comprises the sequence of SEQ ID NO: 61; and
   c) the VH CDR3 comprises the sequence of SEQ ID NO: 62;
[37] the antibody or antigen binding fragment thereof of [1] or [2], wherein
   a) the VH CDR1 comprises the sequence of SEQ ID NO: 18;
   b) the VH CDR2 comprises the sequence of SEQ ID NO: 19; and
   c) the VH CDR3 comprises the sequence of SEQ ID NO: 59;
[38] the antibody or antigen binding fragment thereof of [1] or [2], wherein
   a) the VH CDR1 comprises the sequence of SEQ ID NO: 18;
   b) the VH CDR2 comprises the sequence of SEQ ID NO: 19; and
   c) the VH CDR3 comprises the sequence of SEQ ID NO: 60;
[39] the antibody or antigen binding fragment thereof of [1] or [2], wherein
   a) the VH CDR1 comprises the sequence of SEQ ID NO: 18;
   b) the VH CDR2 comprises the sequence of SEQ ID NO: 19; and
   c) the VH CDR3 comprises the sequence of SEQ ID NO: 67;
[40] the antibody or antigen binding fragment thereof of [1] or [2], wherein
   a) the VH CDR1 comprises the sequence of SEQ ID NO: 18;
   b) the VH CDR2 comprises the sequence of SEQ ID NO: 19; and
   c) the VH CDR3 comprises the sequence of SEQ ID NO: 140;
[41] the antibody or antigen binding fragment thereof of [1] or [2], wherein
   a) the VH CDR1 comprises the sequence of SEQ ID NO: 18;
   b) the VH CDR2 comprises the sequence of SEQ ID NO: 19; and
   c) the VH CDR3 comprises the sequence of SEQ ID NO: 141;
[42] the antibody or antigen binding fragment thereof of [1] or [2], wherein
   a) the VH CDR1 comprises the sequence of SEQ ID NO: 18;
   b) the VH CDR2 comprises the sequence of SEQ ID NO: 19; and
   c) the VH CDR3 comprises the sequence of SEQ ID NO: 142;
[43] the antibody or antigen binding fragment thereof of [1] or [2], wherein
   a) the VH CDR1 comprises the sequence of SEQ ID NO: 18;
   b) the VH CDR2 comprises the sequence of SEQ ID NO: 19; and
   c) the VH CDR3 comprises the sequence of SEQ ID NO: 143;
[44] the antibody or antigen binding fragment thereof of [1] or [2], wherein
   a) the VH CDR1 comprises the sequence of SEQ ID NO: 18;
   b) the VH CDR2 comprises the sequence of SEQ ID NO: 19; and
   c) the VH CDR3 comprises the sequence of SEQ ID NO: 144;
[45] the antibody or antigen binding fragment thereof of any one of [1], [2] or [32] to [44], wherein
   a) the VL CDR1 comprises the sequence of GEKSLGSRAVQ (SEQ ID NO: 15);
   b) the VL CDR2 comprises the sequence of NNQDRPS (SEQ ID NO: 16); and
   c) the VL CDR3 comprises the sequence of HIWDSRVPTKWV (SEQ ID NO: 17);
[46] the antibody or antigen binding fragment thereof of any one of [1], [2] or [32] to [44], wherein
   a) the VL CDR1 comprises the sequence of GGESIGSRAVQ (SEQ ID NO: 23);
   b) the VL CDR2 comprises the sequence of NNQDRPP (SEQ ID NO: 24); and
   c) the VL CDR3 comprises the sequence of HIWDSRRPTNWV (SEQ ID NO: 25);
[47] the antibody or antigen binding fragment thereof of any one of [1], [2] or [32] to [44], wherein
   a) the VL CDR1 comprises the sequence of SEQ ID NO: 63;
   b) the VL CDR2 comprises the sequence of SEQ ID NO: 16; and
   c) the VL CDR3 comprises the sequence of SEQ ID NO: 25;
[48] the antibody or antigen binding fragment thereof of any one of [1], [2] or [32] to [44], wherein
   a) the VL CDR1 comprises the sequence of SEQ ID NO: 23;
   b) the VL CDR2 comprises the sequence of SEQ ID NO: 24; and
   c) the VL CDR3 comprises the sequence of SEQ ID NO: 68;
[49] the antibody or antigen binding fragment thereof of any one of [1], [2] or [32] to [44], wherein
   a) the VL CDR1 comprises the sequence of SEQ ID NO: 23;
   b) the VL CDR2 comprises the sequence of SEQ ID NO: 24; and
   c) the VL CDR3 comprises the sequence of SEQ ID NO: 146;
[50] the antibody or antigen binding fragment thereof of any one of [1], [2] or [32] to [44], wherein
   a) the VL CDR1 comprises the sequence of SEQ ID NO: 23;
   b) the VL CDR2 comprises the sequence of SEQ ID NO: 24; and
   c) the VL CDR3 comprises the sequence of SEQ ID NO: 147;
[51] the antibody or antigen binding fragment thereof of any one of [1], [2] or [32] to [44], wherein
   a) the VL CDR1 comprises the sequence of SEQ ID NO: 23;
   b) the VL CDR2 comprises the sequence of SEQ ID NO: 145; and
   c) the VL CDR3 comprises the sequence of SEQ ID NO: 68;
[52] the antibody or antigen binding fragment thereof of [1] or [2], wherein
   a) the VH CDR1 comprises the sequence of DSYWS (SEQ ID NO: 12);
   b) the VH CDR2 comprises the sequence of YVHKSGDTNYSPSLKS (SEQ ID NO: 13); and
   c) the VH CDR3 comprises the sequence of TLHGRRIYGIVAFNEWFTYFYMDV (SEQ ID NO: 14);
   d) the VL CDR1 comprises the sequence of GGESIGSRAVQ (SEQ ID NO: 23);
   e) the VL CDR2 comprises the sequence of NNQDRPP (SEQ ID NO: 24); and
   f) the VL CDR3 comprises the sequence of HIWDSRRPTNWV (SEQ ID NO: 25);
[53] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO: 19); and
   c) the VH CDR3 comprises the sequence of TLHGRRIYGVVAFNEWFTYFYWEV (SEQ ID NO: 20);
   d) the VL CDR1 comprises the sequence of GEKSLGSRAVQ (SEQ ID NO: 15);
   e) the VL CDR2 comprises the sequence of NNQDRPS (SEQ ID NO: 16); and
   f) the VL CDR3 comprises the sequence of HIWDSRVPTKWV (SEQ ID NO: 17);
[54] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO: 19); and
   c) the VH CDR3 comprises the sequence of TLHGRRIYGVVAFNEWFTYFYWEV (SEQ ID NO: 20);
   d) the VL CDR1 comprises the sequence of GGESIGSRAVQ (SEQ ID NO: 23);
   e) the VL CDR2 comprises the sequence of NNQDRPP (SEQ ID NO: 24); and
   f) the VL CDR3 comprises the sequence of HIWDSRRPTNWV (SEQ ID NO: 25);
[55] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO: 19); and
   c) the VH CDR3 comprises the sequence of TLHGRRIYGVVAFNELFTYFYWEV (SEQ ID NO: 21);
   d) the VL CDR1 comprises the sequence of GEKSLGSRAVQ (SEQ ID NO: 15);
   e) the VL CDR2 comprises the sequence of NNQDRPS (SEQ ID NO: 16); and
   f) the VL CDR3 comprises the sequence of HIWDSRVPTKWV (SEQ ID NO: 17);
[56] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO: 19); and
   c) the VH CDR3 comprises the sequence of TLHGRRIYGVVAFNELFTYFYWEV (SEQ ID NO: 21);
   d) the VL CDR1 comprises the sequence of GGESIGSRAVQ (SEQ ID NO: 23);
   e) the VL CDR2 comprises the sequence of NNQDRPP (SEQ ID NO: 24); and
   f) the VL CDR3 comprises the sequence of HIWDSRRPTNWV (SEQ ID NO: 25);
[57] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO: 19); and
   c) the VH CDR3 comprises the sequence of TLHGRRIYGVVAFNEFFTYFYWEV (SEQ ID NO: 22);
   d) the VL CDR1 comprises the sequence of GEKSLGSRAVQ (SEQ ID NO: 15);
   e) the VL CDR2 comprises the sequence of NNQDRPS (SEQ ID NO: 16); and
   f) the VL CDR3 comprises the sequence of HIWDSRVPTKWV (SEQ ID NO: 17);
[58] the antibody or antigen binding fragment thereof of [1] or [2], wherein
   a) the VH CDR1 comprises the sequence of SSYWS (SEQ ID NO: 18);
   b) the VH CDR2 comprises the sequence of YTHHSGDTNYAPSLKS (SEQ ID NO: 19); and
   c) the VH CDR3 comprises the sequence of TLHGRRIYGVVAFNEFFTYFYWEV (SEQ ID NO: 22);
   d) the VL CDR1 comprises the sequence of GGESIGSRAVQ (SEQ ID NO: 23);
   e) the VL CDR2 comprises the sequence of NNQDRPP (SEQ ID NO: 24); and
   f) the VL CDR3 comprises the sequence of HIWDSRRPTNWV (SEQ ID NO: 25);
[59] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 12, 13, 14, 63, 16, and 25, respectively;
[60] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 20, 63, 16, and 25, respectively;
[61] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 21, 63, 16, and 25, respectively;
[62] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 22, 63, 16, and 25, respectively;
[63] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 59, 63, 16, and 25, respectively;
[64] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 60, 63, 16, and 25, respectively;
[65] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 12, 61, 62, 63, 16, and 25, respectively;
[66] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 59, 15, 16, and 17, respectively;
[67] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 60, 15, 16, and 17, respectively;
[68] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 12, 61, 62, 15, 16, and 17, respectively;
[69] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 59, 23, 24, and 25, respectively;
[70] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 60, 23, 24, and 25, respectively;
[71] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 12, 61, 62, 23, 24, and 25, respectively;
[72] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 67, 15, 16, and 17, respectively;
[73] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 67, 23, 24, and 25, respectively;
[74] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 67, 63, 16, and 25, respectively;
[75] the antibody or antigen binding fragment thereof of [1] or [2], wherein the VH CDR1, VH CDR2, VH CDR3, VL CDR1, VL CDR2, and VL CDR3 comprises the sequence of SEQ ID NO: 18, 19, 67, 23, 24, and 67, respectively;
[76] the antibody or antigen binding fragment thereof of any one of [1] to [75], wherein the VH further comprises a VH FR1, VH FR2, VH FR3, and VH FR4, wherein
   (i) the VH FR1 comprises the sequence of
      QX1QLQESGPGLVKPX2ETLSLTCSVSGASIS wherein X1 is M or V, and X2 is S or G (SEQ ID NO: 77);
   (ii) the VH FR2 comprises the sequence of WX1RX2X3PGKGX4EWIG wherein X1 is I or L, X2 is R or E, X3 is S or T, and X4 is I, L or V (SEQ ID NO: 80);
   (iii) the VH FR3 comprises the sequence of
      RVX1X2X3LX4X5X6KNQVSLX7LX8X9X10TAADX11X12X13YYCAR wherein X1 is N, H, or T, X2 is L or I, X3 is S or G, X4 is D or H, X5 is T or P, X6 is S or E, X7 is S or K, X8 is V, T or K, X9 is A or S, X10 is A or V, X11 is S or T, X12 is G or A, and X13 is K or V (SEQ ID NO: 88); and/or
   (iv) the VH FR4 comprises the sequence of YX1X2X3WGX4GX5X6VTVSS wherein X1 is M or W, X2 is D, E or P, X3 is V or T, X4 is N or K, X5 is I or T, and X6 is Q or K (SEQ ID NO: 94);
[77] the antibody or antigen binding fragment thereof of [76], wherein the VH FR1 comprises the sequence of SEQ ID NO: 74;
[78] the antibody or antigen binding fragment thereof of [76], wherein the VH FR1 comprises the sequence of SEQ ID NO: 75;
[79] the antibody or antigen binding fragment thereof of [76], wherein the VH FR1 comprises the sequence of SEQ ID NO: 76;
[80] the antibody or antigen binding fragment thereof of any one of [76] to [79], wherein the VH FR2 comprises the sequence of SEQ ID NO: 78;
[81] the antibody or antigen binding fragment thereof of any one of [76] to [79], wherein the VH FR2 comprises the sequence of SEQ ID NO: 79;
[82] the antibody or antigen binding fragment thereof of any one of [76] to [79], wherein the VH FR2 comprises the sequence of SEQ ID NO: 148;
[83] the antibody or antigen binding fragment thereof of any one of [76] to [79], wherein the VH FR2 comprises the sequence of SEQ ID NO: 149;
[84] the antibody or antigen binding fragment thereof of any one of [76] to [83], wherein the VH FR3 comprises the sequence of SEQ ID NO: 81;
[85] the antibody or antigen binding fragment thereof of any one of [76] to [83], wherein the VH FR3 comprises the sequence of SEQ ID NO: 82;
[86] the antibody or antigen binding fragment thereof of any one of [76] to [83], wherein the VH FR3 comprises the sequence of SEQ ID NO: 83;
[87] the antibody or antigen binding fragment thereof of any one of [76] to [83], wherein the VH FR3 comprises the sequence of SEQ ID NO: 84;
[88] the antibody or antigen binding fragment thereof of any one of [76] to [83], wherein the VH FR3 comprises the sequence of SEQ ID NO: 85;
[89] the antibody or antigen binding fragment thereof of any one of [76] to [83], wherein the VH FR3 comprises the sequence of SEQ ID NO: 86;
[90] the antibody or antigen binding fragment thereof of any one of [76] to [83], wherein the VH FR3 comprises the sequence of SEQ ID NO: 87;
[91] the antibody or antigen binding fragment thereof of any one of [76] to [83], wherein the VH FR3 comprises the sequence of SEQ ID NO: 150;
[92] the antibody or antigen binding fragment thereof of any one of [76] to [83], wherein the VH FR3 comprises the sequence of SEQ ID NO: 151;
[93] the antibody or antigen binding fragment thereof of any one of [76] to [83], wherein the VH FR3 comprises the sequence of SEQ ID NO: 152;
[94] the antibody or antigen binding fragment thereof of any one of [76] to [93], wherein the VH FR4 comprises the sequence of SEQ ID NO: 89;
[95] the antibody or antigen binding fragment thereof of any one of [76] to [93], wherein the VH FR4 comprises the sequence of SEQ ID NO: 90;
[96] the antibody or antigen binding fragment thereof of any one of [76] to [93], wherein the VH FR4 comprises the sequence of SEQ ID NO: 91;
[97] the antibody or antigen binding fragment thereof of any one of [76] to [93], wherein the VH FR4 comprises the sequence of SEQ ID NO: 92;
[98] the antibody or antigen binding fragment thereof of any one of [76] to [93], wherein the VH FR4 comprises the sequence of SEQ ID NO: 93;
[99] the antibody or antigen binding fragment thereof of any one of [76] to [93], wherein the VH FR4 comprises the sequence of SEQ ID NO: 93;
[100] the antibody or antigen binding fragment thereof of any one of [76] to [93], wherein the VH FR4 comprises the sequence of SEQ ID NO: 153;
[101] the antibody or antigen binding fragment thereof of any one of [76] to [93], wherein the VH FR4 comprises the sequence of SEQ ID NO: 154;
[102] the antibody or antigen binding fragment thereof of [76], wherein the VH FR1, VH FR2, VH FR3, and VH FR4 comprises the sequence of SEQ ID NO: 74, 78, 81, 89, respectively;
[103] the antibody or antigen binding fragment thereof of [76], wherein the VH FR1, VH FR2, VH FR3, and VH FR4 comprises the sequence of SEQ ID NO: 75, 79, 83, 91, respectively;
[104] the antibody or antigen binding fragment thereof of [76], wherein the VH FR1, VH FR2, VH FR3, and VH FR4 comprises the sequence of SEQ ID NO: 75, 79, 83, 92, respectively;
[105] the antibody or antigen binding fragment thereof of [76], wherein the VH FR1, VH FR2, VH FR3, and VH FR4 comprises the sequence of SEQ ID NO: 76, 79, 83, 92, respectively;
[106] the antibody or antigen binding fragment thereof of [76], wherein the VH FR1, VH FR2, VH FR3, and VH FR4 comprises the sequence of SEQ ID NO: 75, 79, 84, 92, respectively;
[107] the antibody or antigen binding fragment thereof of [76], wherein the VH FR1, VH FR2, VH FR3, and VH FR4 comprises the sequence of SEQ ID NO: 74, 78, 81, 89, respectively;
[108] the antibody or antigen binding fragment thereof of [76], wherein the VH FR1, VH FR2, VH FR3, and VH FR4 comprises the sequence of SEQ ID NO: 75, 79, 82, 90, respectively;
[109] the antibody or antigen binding fragment thereof of [76], wherein the VH FR1, VH FR2, VH FR3, and VH FR4 comprises the sequence of SEQ ID NO: 75, 79, 85, 91, respectively;
[110] the antibody or antigen binding fragment thereof of [76], wherein the VH FR1, VH FR2, VH FR3, and VH FR4 comprises the sequence of SEQ ID NO: 75, 79, 85, 92, respectively;
[111] the antibody or antigen binding fragment thereof of [76], wherein the VH FR1, VH FR2, VH FR3, and VH FR4 comprises the sequence of SEQ ID NO: 75, 79, 83, 91, respectively;
[112] the antibody or antigen binding fragment thereof of [76], wherein the VH FR1, VH FR2, VH FR3, and VH FR4 comprises the sequence of SEQ ID NO: 75, 79, 83, 93, respectively;
[113] the antibody or antigen binding fragment thereof of [76], wherein the VH FR1, VH FR2, VH FR3, and VH FR4 comprises the sequence of SEQ ID NO: 75, 148, 150, 93, respectively;
[114] the antibody or antigen binding fragment thereof of [76], wherein the VH FR1, VH FR2, VH FR3, and VH FR4 comprises the sequence of SEQ ID NO: 75, 148, 151, 93, respectively;
[115] the antibody or antigen binding fragment thereof of [76], wherein the VH FR1, VH FR2, VH FR3, and VH FR4 comprises the sequence of SEQ ID NO: 75, 148, 83, 154, respectively;
[116] the antibody or antigen binding fragment thereof of [76], wherein the VH FR1, VH FR2, VH FR3, and VH FR4 comprises the sequence of SEQ ID NO: 75, 148, 83, 93, respectively;
[117] the antibody or antigen binding fragment thereof of [76], wherein the VH FR1, VH FR2, VH FR3, and VH FR4 comprises the sequence of SEQ ID NO: 75, 79, 150, 93, respectively;
[118] the antibody or antigen binding fragment thereof of [76], wherein the VH FR1, VH FR2, VH FR3, and VH FR4 comprises the sequence of SEQ ID NO: 75, 79, 152, 153, respectively;
[119] the antibody or antigen binding fragment thereof of [76], wherein the VH FR1, VH FR2, VH FR3, and VH FR4 comprises the sequence of SEQ ID NO: 75, 79, 151, 153, respectively;
[120] the antibody or antigen binding fragment thereof of [76], wherein the VH FR1, VH FR2, VH FR3, and VH FR4 comprises the sequence of SEQ ID NO: 75, 79, 152, 93, respectively;
[121] the antibody or antigen binding fragment thereof of [76], wherein the VH FR1, VH FR2, VH FR3, and VH FR4 comprises the sequence of SEQ ID NO: 75, 79, 83, 153, respectively;
[122] the antibody or antigen binding fragment thereof of [76], wherein the VH FR1, VH FR2, VH FR3, and VH FR4 comprises the sequence of SEQ ID NO: 75, 79, 151, 154, respectively;
[123] the antibody or antigen binding fragment thereof of [76], wherein the VH FR1, VH FR2, VH FR3, and VH FR4 comprises the sequence of SEQ ID NO: 75, 79, 86, 93, respectively;
[124] the antibody or antigen binding fragment thereof of [76], wherein the VH FR1, VH FR2, VH FR3, and VH FR4 comprises the sequence of SEQ ID NO: 75, 79, 87, 93, respectively;
[125] the antibody or antigen binding fragment thereof of [76], wherein the VH FR1, VH FR2, VH FR3, and VH FR4 comprises the sequence of SEQ ID NO: 75, 149, 152, 153, respectively;
[126] the antibody or antigen binding fragment thereof of any one of [1] to [125], wherein the VL further comprises a VL FR1, VL FR2, VL FR3, and VL FR4, wherein
   (i) the VL FR1 comprises the sequence of X1X2X3SVAPGETX4RIX5C wherein X1 is S or P, X2 is D or S, X3 is I, L or V, X4 is A or V, and X5 is S or T (SEQ ID NO: 99);
   (ii) the VL FR2 comprises the sequence of WYQX1RX2GQAPX3LIIY wherein X1 is Q or H, X2 is A or P, and X3 is S, K, R or P (SEQ ID NO: 103);
   (iii) the VL FR3 comprises the sequence of
      GIPERFSGSPDX1X2FGTTATLTIX3X4VEAGDEAX5YYC wherein X1 is S, L or I, X2 is P, A or D, X3 is T or S, X4 is S, R or N, and X5 is D or T (SEQ ID NO: 108); and/or
   (iv) the VL FR4 comprises the sequence of X1X2X3GTTLTVL wherein X1 is F or L, X2 is E or G, and X3 is G or P (SEQ ID NO: 111);
[127] the antibody or antigen binding fragment thereof of [126], wherein the VL FR1 comprises the sequence of SEQ ID NO: 95;
[128] the antibody or antigen binding fragment thereof of [126], wherein the VL FR1 comprises the sequence of SEQ ID NO: 96;
[129] the antibody or antigen binding fragment thereof of [126], wherein the VL FR1 comprises the sequence of SEQ ID NO: 97;
[130] the antibody or antigen binding fragment thereof of [126], wherein the VL FR1 comprises the sequence of SEQ ID NO: 98;
[131] the antibody or antigen binding fragment thereof of [126], wherein the VL FR1 comprises the sequence of SEQ ID NO: 155;
[132] the antibody or antigen binding fragment thereof of [126], wherein the VL FR1 comprises the sequence of SEQ ID NO: 156;
[133] the antibody or antigen binding fragment thereof of any one of [125] to [132], wherein the VL FR2 comprises the sequence of SEQ ID NO: 100;
[134] the antibody or antigen binding fragment thereof of any one of [125] to [132], wherein the VL FR2 comprises the sequence of SEQ ID NO: 101;
[135] the antibody or antigen binding fragment thereof of any one of [125] to [132], wherein the VL FR2 comprises the sequence of SEQ ID NO: 102;
[136] the antibody or antigen binding fragment thereof of any one of [125] to [132], wherein the VL FR2 comprises the sequence of SEQ ID NO: 157;
[137] the antibody or antigen binding fragment thereof of any one of [125] to [132], wherein the VL FR2 comprises the sequence of SEQ ID NO: 158;
[138] the antibody or antigen binding fragment thereof of any one of [125] to [132], wherein the VL FR2 comprises the sequence of SEQ ID NO: 159;
[139] the antibody or antigen binding fragment thereof of any one of [125] to [132], wherein the VL FR2 comprises the sequence of SEQ ID NO: 160;
[140] the antibody or antigen binding fragment thereof of any one of [125] to [139], wherein the VL FR3 comprises the sequence of SEQ ID NO: 104;
[141] the antibody or antigen binding fragment thereof of any one of [125] to [139], wherein the VL FR3 comprises the sequence of SEQ ID NO: 105;
[142] the antibody or antigen binding fragment thereof of any one of [125] to [139], wherein the VL FR3 comprises the sequence of SEQ ID NO: 106;
[143] the antibody or antigen binding fragment thereof of any one of [125] to [139], wherein the VL FR3 comprises the sequence of SEQ ID NO: 107;
[144] the antibody or antigen binding fragment thereof of any one of [125] to [139], wherein the VL FR3 comprises the sequence of SEQ ID NO: 161;
[145] the antibody or antigen binding fragment thereof of any one of [125] to [139], wherein the VL FR3 comprises the sequence of SEQ ID NO: 162;
[146] the antibody or antigen binding fragment thereof of any one of [125] to [139], wherein the VL FR3 comprises the sequence of SEQ ID NO: 163;
[147] the antibody or antigen binding fragment thereof of any one of [125] to [139], wherein the VL FR3 comprises the sequence of SEQ ID NO: 164;
[148] the antibody or antigen binding fragment thereof of any one of [125] to [147], wherein the VL FR4 comprises the sequence of SEQ ID NO: 109;
[149] the antibody or antigen binding fragment thereof of any one of [125] to [147], wherein the VL FR4 comprises the sequence of SEQ ID NO: 110;
[150] the antibody or antigen binding fragment thereof of any one of [125] to [147], wherein the VL FR4 comprises the sequence of SEQ ID NO: 165;
[151] the antibody or antigen binding fragment thereof of any one of [125] to [147], wherein the VL FR4 comprises the sequence of SEQ ID NO: 166;
[152] the antibody or antigen binding fragment thereof of any one of [125] to [147], wherein the VL FR4 comprises the sequence of SEQ ID NO: 167;
[153] the antibody or antigen binding fragment thereof of [126], wherein the VL FR1, VL FR2, VL FR3, and VL FR4 comprises the sequence of SEQ ID NO: 95, 100, 104, 109, respectively;
[154] the antibody or antigen binding fragment thereof of [126], wherein the VL FR1, VL FR2, VL FR3, and VL FR4 comprises the sequence of SEQ ID NO: 96, 101, 105, 109, respectively;
[155] the antibody or antigen binding fragment thereof of [126], wherein the VL FR1, VL FR2, VL FR3, and VL FR4 comprises the sequence of SEQ ID NO: 97, 101, 105, 109, respectively;
[156] the antibody or antigen binding fragment thereof of [126], wherein the VL FR1, VL FR2, VL FR3, and VL FR4 comprises the sequence of SEQ ID NO: 95, 100, 106, 109, respectively;
[157] the antibody or antigen binding fragment thereof of [126], wherein the VL FR1, VL FR2, VL FR3, and VL FR4 comprises the sequence of SEQ ID NO: 98, 102, 107, 110, respectively;
[158] the antibody or antigen binding fragment thereof of [126], wherein the VL FR1, VL FR2, VL FR3, and VL FR4 comprises the sequence of SEQ ID NO: 96, 157, 105, 110, respectively;
[159] the antibody or antigen binding fragment thereof of [126], wherein the VL FR1, VL FR2, VL FR3, and VL FR4 comprises the sequence of SEQ ID NO: 96, 157, 164, 110, respectively;
[160] the antibody or antigen binding fragment thereof of [126], wherein the VL FR1, VL FR2, VL FR3, and VL FR4 comprises the sequence of SEQ ID NO: 96, 157, 105, 166, respectively;
[161] the antibody or antigen binding fragment thereof of [126], wherein the VL FR1, VL FR2, VL FR3, and VL FR4 comprises the sequence of SEQ ID NO: 96, 160, 105, 146, respectively;
[162] the antibody or antigen binding fragment thereof of [126], wherein the VL FR1, VL FR2, VL FR3, and VL FR4 comprises the sequence of SEQ ID NO: 98, 102, 163, 110, respectively;
[163] the antibody or antigen binding fragment thereof of [126], wherein the VL FR1, VL FR2, VL FR3, and VL FR4 comprises the sequence of SEQ ID NO: 98, 102, 107, 166, respectively;
[164] the antibody or antigen binding fragment thereof of [126], wherein the VL FR1, VL FR2, VL FR3, and VL FR4 comprises the sequence of SEQ ID NO: 98, 159, 107, 110, respectively;
[165] the antibody or antigen binding fragment thereof of [126], wherein the VL FR1, VL FR2, VL FR3, and VL FR4 comprises the sequence of SEQ ID NO: 155, 102, 107, 165, respectively;
[166] the antibody or antigen binding fragment thereof of [126], wherein the VL FR1, VL FR2, VL FR3, and VL FR4 comprises the sequence of SEQ ID NO: 155, 158, 161, 110, respectively;
[167] the antibody or antigen binding fragment thereof of [126], wherein the VL FR1, VL FR2, VL FR3, and VL FR4 comprises the sequence of SEQ ID NO: 155, 102, 107, 167, respectively;
[168] the antibody or antigen binding fragment thereof of [126], wherein the VL FR1, VL FR2, VL FR3, and VL FR4 comprises the sequence of SEQ ID NO: 156, 157, 105, 165, respectively;
[169] the antibody or antigen binding fragment thereof of [126], wherein the VL FR1, VL FR2, VL FR3, and VL FR4 comprises the sequence of SEQ ID NO: 155, 101, 162, 110, respectively;
[170] the antibody or antigen binding fragment thereof of [126], wherein the VL FR1, VL FR2, VL FR3, and VL FR4 comprises the sequence of SEQ ID NO: 155, 157, 105, 167, respectively;
[171] the antibody or antigen binding fragment thereof of any one of [1] to [75], wherein the VH framework regions comprise one or more of a V at H2, G at H15, L at H37, E at H39, T at H40, L or I at H45, H or T at H68, I at H69, G at H70, H at H72, P at H73, E at H74, K at H81, K or T at H82A, S at H82B, V at H82C, Tat H87, A at H88, Vat H89, and N or K at H105, I at H107, K at H108, wherein the VH framework residues are numbered according to Kabat;
[172] the antibody or antigen binding fragment thereof of [171], wherein the VH framework regions comprise two or more, three or more, four or more, or five or more of a V at H2, G at H15, L at H37, E at H39, T at H40, L or I at H45, H or T at H68, I at H69, G at H70, H at H72, P at H73, E at H74, K at H81, K or T at H82A, Sat H82B, V at H82C, Tat H87, A at H88, V at H89, and N or K at H105, I at H107, K at H108, wherein the VH framework residues are numbered according to Kabat;
[173] the antibody or antigen binding fragment thereof of [171], wherein the VH framework regions comprise a V at H2, G at H15, L at H37, E at H39, T at H40, L or I at H45, H or T at H68, I at H69, G at H70, H at H72, P at H73, E at H74, K at H81, K or T at H82A, S at H82B, V at H82C, Tat H87, A at H88, V at H89, and N or K at H105, I at H107, K at H108, wherein the VH framework residues are numbered according to Kabat;
[174] the antibody or antigen binding fragment thereof of [171], wherein the VH framework regions comprise a G at H15, L at H37, E at H39, T at H40, H at H68, L at H69, G at H70, H at H72, P at H73, K or T at H82A, S at H82B, V at H82C, T at H87, A at H88, V at H89, and N or K at H105, wherein the VH framework residues are numbered according to Kabat;
[175] the antibody or antigen binding fragment thereof of [171], wherein the VH framework regions comprise a G at H15, L at H37, E at H39, Tat H40, Tat H68, I at H69, G or S at H70, H or D at H72, P at H73, K or T at H82A, S at H82B, V at H82C, T at H87, A at H88, V at H89, and N or K at H105, wherein the VH framework residues are numbered according to Kabat;
[176] the antibody or antigen binding fragment thereof of any one of [1] to [75] or [171] to [175], wherein the VH has at least about 70%, at least about 75%, at least about 80%, at least about 90%, or at least about 95% sequence identity to SEQ ID NO: 1;
[177] the antibody or antigen binding fragment thereof of any one of [1] to [75] or [171] to [175], wherein the VH has at least about 70%, at least about 75%, at least about 80%, at least about 90%, or at least about 95% sequence identity to SEQ ID NO: 3-9, 45, 47, 49-54, 65, 69, 70, or 112-126;
[178] the antibody or antigen binding fragment thereof of any one of [1] to [75] or [171] to [177], wherein the VL framework regions comprise one or more of a S at L2, Y at L3, V at L4, L at L5, T at L6, Q at L7, P at L8, P at L9, S at L10, V at L11, V at L19, T at L22, Q at L38, P at L40, K or Rat L45, I at L68, D at L69, S at L79, N or Rat L80, Tat L88, L at L101, E at 102L, and P at L103, wherein the VL framework residues are numbered according to Kabat;
[179] the antibody or antigen binding fragment thereof of [178], wherein the VL framework regions comprise two or more, three or more, four or more, or five or more of a S at L2, Y at L3, V at L4, L at L5, T at L6, Q at L7, P at L8, P at L9, S at L10, V at L11, V at L19, T at L22, Q at L38, P at L40, K or R at L45, I at L68, D at L69, S at L79, N or R at L80, Tat L88, L at L101, E at 102L, and P at L103, wherein the VL framework residues are numbered according to Kabat;
[180] the antibody or antigen binding fragment thereof of [178], wherein the VL framework regions comprise a S at L2, Y at L3, V at L4, L at L5, T at L6, Q at L7, P at L8, P at L9, S at L10, V at L11, V at L19, T at L22, Q at L38, P at L40, K or R at L45, I at L68, D at L69, S at L79, N or Rat L80, Tat L88, L at L101, Eat 102L, and P at L103, wherein the VL framework residues are numbered according to Kabat;
[181] the antibody or antigen binding fragment thereof of [178], wherein the VL framework regions comprise a V at L19, K or Rat L45, I at L68, D or A at L69, N at L80, Tat L88, L at L101, and P at L103, wherein the VL framework residues are numbered according to Kabat;
[182] the antibody or antigen binding fragment thereof of [178], wherein the VL framework regions comprise a V at L19, K or Rat L45, I at L68, D or A at L69, N at L80, Tat L88, L at L101, and P at L103, wherein the VL framework residues are numbered according to Kabat;
[183] the antibody or antigen binding fragment thereof of any one of [1] to [75] or [176] to [182], wherein the VL has at least about 70%, at least about 75%, at least about 80%, at least about 90%, or at least about 95% sequence identity to SEQ ID NO: 2;
[184] the antibody or antigen binding fragment thereof of any one of [1] to [75] or [176] to [182], wherein the VL has at least about 70%, at least about 75%, at least about 80%, at least about 90%, or at least about 95% sequence identity to SEQ ID NO: 10, 11, 46, 48, 55, 66, or 127-139;
[185] the antibody or antigen binding fragment thereof of any one of [1] to [184], wherein the VL comprises SEQ ID NO: 2;
[186] the antibody or antigen binding fragment thereof of any one of [1] to [184], wherein the VL comprises SEQ ID NO: 46 or 48;
[187] the antibody or antigen binding fragment thereof of any one of [1] to [184], wherein the VL comprises SEQ ID NO: 66;
[188] the antibody or antigen binding fragment thereof of any one of [1] to [184], wherein the VL comprises SEQ ID NO: 55;
[189] the antibody or antigen binding fragment thereof of any one of [1] to [184], wherein the VL comprises SEQ ID NO: 10 or 11;
[190] the antibody or antigen binding fragment thereof of any one of [1] to [184], wherein the VL comprises SEQ ID NO: 127-139;
[191] the antibody or antigen binding fragment thereof of any one of [1] to [190], wherein the VH comprises SEQ ID NO: 3-9, 45, 47, 49-54, 65, 69, 70, or 112-126;
[192] the antibody or antigen binding fragment thereof of any one of [1] to [190], wherein the VH comprises SEQ ID NO: 3-9;
[193] the antibody or antigen binding fragment thereof of any one of [1] to [190], wherein the VH comprises SEQ ID NO: 47, or 49-54;
[194] the antibody or antigen binding fragment thereof of any one of [1] to [190], wherein the VH comprises SEQ ID NO: 65;
[195] the antibody or antigen binding fragment thereof of any one of [1] to [190], wherein the VH comprises SEQ ID NO: 69;
[196] the antibody or antigen binding fragment thereof of any one of [1] to [190], wherein the VH comprises SEQ ID NO: 70;
[197] the antibody or antigen binding fragment thereof of any one of [1] to [190], wherein the VH comprises SEQ ID NO: 1;
[198] An antibody or antigen binding fragment thereof capable of binding to HIV gp120, wherein the antibody or antigen binding fragment thereof comprises a VH CDR1, VH CDR2, VH CDR3, VL CDR1, VL CDR2, VL CDR3, and VH and VL framework regions, wherein
   a) the VH CDR1 comprises the sequence of X1SYWS, wherein X1 is D or S (SEQ ID NO: 26);
   b) the VH CDR2 comprises the sequence of YX1HX2SGDTNYX3PSLKS wherein X1 is V or T, X2 is K or H, and X3 is S or A (SEQ ID NO: 27);
   c) the VH CDR3 comprises the sequence of
      TLHGRRX1YGX2VAFX3EX4FTYFYX5X6V wherein X1 is I or C, X2 is I or V, X3 is N or G, X4 is W, C, F or L, X5 is M or W, and X6 is D or E (SEQ ID NO: 28) or TLHGRRIYGX1VAFX2EX3FTYFYX4X5V wherein X1 is I or V, X2 is N or G, X3 is W, F or L, X4 is M or W, and X5 is D or E (SEQ ID NO: 64);
   d) the VL CDR1 comprises the sequence of GX1X2SX3GSRAVQ wherein X1 is E or G, X2 is K or E, and X3 is L or I (SEQ ID NO: 29);
   e) the VL CDR2 comprises the sequence of NNQDRPX1 wherein X1 is S or P (SEQ ID NO: 30); and
   f) the VL CDR3 comprises the sequence of HIWDSRX1PTX2WV wherein X1 is V or R, and X2 is K or N (SEQ ID NO: 31);
   and wherein the VH and VL framework regions comprise one or more of a V at H2, G at H15, L at H37, E at H39, T at H40, H at H68, G at H70, H at H72, P at H73, K at H81, K or Tat H82A, S at H82B, V at H82C, Tat H87, A at H88, V at H89, K at H105, S at L2, Y at L3, V at L4, L at L5, T at L6, Q at L7, P at L8, P at L9, S at L10, V at L11, V at L19, K at L45, I at L68, D at L69, N at L80, and T at L88, wherein the VH and VL framework residues are numbered according to Kabat;
[199] the antibody or antigen binding fragment thereof of [198], wherein the VH and VL framework regions comprise two or more, three or more, four or more, or five or more of V at H2, G at H15, L at H37, E at H39, T at H40, H at H68, G at H70, H at H72, P at H73, K at H81, K or T at H82A, S at H82B, V at H82C, T at H87, A at H88, V at H89, K at H105, S at L2, Y at L3, V at L4, L at L5, T at L6, Q at L7, P at L8, P at L9, S at L10, V at L11, V at L19, K at L45, I at L68, D at L69, N at L80, and T at L88;
[200] the antibody or antigen binding fragment thereof of [198], wherein the VH and VL framework regions comprise a G at H15, L at H37, E at H39, T at H40, H at H68, G at H70, H at H72, P at H73, T at H82A, S at H82B, V at H82C, T at H87, A at H88, V at H89, V at L19, K at L45, I at L68, D at L69, N at L80, and Tat L88;
[201] the antibody or antigen binding fragment thereof of any one of [198] to [200], wherein the VH and VL framework regions comprise one or more of a K at H81 and K at H105;
[202] the antibody or antigen binding fragment thereof of any one of [198] to [200], wherein the VH comprises the amino acid sequence of SEQ ID NO: 3-9, 45, 47, or 49-54;
[203] the antibody or antigen binding fragment thereof of any one of [198] to [202], wherein the VL comprises the amino acid sequence of SEQ ID NO: 10, 11, 46, 48, or 55;
[204] An antibody or antigen binding fragment thereof capable of binding to HIV gp120, wherein the antibody comprises a VH having at least about 70%, at least about 75%, at least about 80%, or at least about 90% sequence identity to SEQ ID NO: 1 and a VL having at least about 70%, at least about 75%, at least about 80%, or at least about 90% sequence identity SEQ ID NO: 2, wherein
   (i) the VH comprises one or more of a V at H2, G at H15, S at H31, I at H37, Eat H39, T at H40, T at H51, H at H53, A at H60, H at H68, G at H70, H at H72, P at H73, K at H81, K or T at H82A, S at H82B, V at H82C, T at H87, A at H88, Vat H89, Vat H100D, G at H100H, F or L at H100J, W at H100P, Eat H101, and K at H105, wherein the VH residues are numbered according to Kabat; and
   (ii) the VL comprises one or more of a S at L2, Y at L3, Vat L4, L at L5, Tat L6, Q at L7, P at L8, P at L9, S at L10, V at L11, V at L19, G at L25, E at L26, I at L28, K at L45, P at L56, I at L68, D or A at L69, N at L80, Tat L88, Rat L95C, and N at L95F, wherein the VL residues are numbered according to Kabat;
[205] the antibody or antigen binding fragment thereof of [204], wherein
   (i) the VH comprises two or more, three or more, four or more, or five or more of a V at H2, G at H15, S at H31, I at H37, E at H39, T at H40, T at H51, H at H53, A at H60, H at H68, G at H70, H at H72, P at H73, K at H81, K or T at H82A, Sat H82B, V at H82C, Tat H87, A at H88, V at H89, V at H100D, G at H100H, F or L at H100J, W at H100P, E at H101, and K at H105, wherein the VH residues are numbered according to Kabat; and
   (ii) the VL comprises two or more, three or more, four or more, or five or more of a S at L2, Y at L3, V at L4, L at L5, T at L6, Q at L7, P at L8, P at L9, S at L10, V at L11, V at L19, G at L25, E at L26, I at L28, K at L45, P at L56, I at L68, D or A at L69, N at L80, T at L88, Rat L95C, and N at L95F, wherein the VL residues are numbered according to Kabat;
[206] the antibody or antigen binding fragment thereof of [204], wherein
   (i) the VH comprises a G at H15, S at H31, L at H37, Eat H39, Tat H40, Tat H51, H at H53, A at H60, H at H68, G at H70, H at H72, P at H73, T at H82A, S at H82B, V at H82C, Tat H87, A at H88, V at H89, V at H100D, F or L at H100J, W at H100P, E at H101, and K at H105, wherein the VH residues are numbered according to Kabat; and
   (ii) the VL comprises a V at L19, G at L25, Eat L26, I at L28, K at L45, P at L56, I at L68, D at L69, N at L80, T at L88, Rat L95C, and N at L95F, wherein the VL residues are numbered according to Kabat;
[207] the antibody or antigen binding fragment thereof of any one of [204] to [206], wherein
   (a) the VH CDR1 comprises the sequence of X1SYWS, wherein X1 is D or S (SEQ ID NO: 26);
   (b) the VH CDR2 comprises the sequence of YX1HX2SGDTNYX3PSLKS wherein X1 is V or T, X2 is K or H, and X3 is S or A (SEQ ID NO: 27);
   (c) the VH CDR3 comprises the sequence of
      TLHGRRX1YGX2VAFX3EX4FTYFYX5X6V wherein X1 is I or C, X2 is I or V, X3 is N or G, X4 is W, C, F or L, X5 is M or W, and X6 is D or E (SEQ ID NO: 28) or TLHGRRIYGX1VAFX2EX3FTYFYX4X5V wherein X1 is I or V, X2 is N or G, X3 is W, F or L, X4 is M or W, and X5 is D or E (SEQ ID NO: 64);
   (d) the VL CDR1 comprises the sequence of GX1X2SX3GSRAVQ wherein X1 is E or G, X2 is K or E, and X3 is L or I (SEQ ID NO: 29);
   (e) the VL CDR2 comprises the sequence of NNQDRPX1 wherein X1 is S or P (SEQ ID NO: 30); and
   (f) the VL CDR3 comprises the sequence of HIWDSRX1PTX2WV wherein X1 is V or R, and X2 is K or N (SEQ ID NO: 31);
[208] the antibody or antigen binding fragment thereof of any one of [204] to [207], wherein the VH comprises the amino acid sequence of SEQ ID NO: 3-9, 45, 47, or 49-54;
[209] the antibody or antigen binding fragment thereof of any one of [204] to [208], wherein the VL comprises the amino acid sequence of SEQ ID NO: 10, 11, 46, 48, or 55;
[210] An antibody or antigen binding fragment thereof capable of binding to HIV gp120, wherein the antibody comprises a VH having at least about 70%, at least about 75%, at least about 80%, or at least about 90% sequence identity to SEQ ID NO: 1 and a VL having at least about 70%, at least about 75%, at least about 80%, or at least about 90% sequence identity SEQ ID NO: 2, wherein the VH comprises one or more of a V at H2, G at H15, S at H31, L at H37, E at H39, T at H40, L or I at H45, T at H51, H at H53, A at H60, H or Tat H68, I at H69, G at H70, H at H72, P at H73, Eat H74, K at H81, K or T at H82A, S at H82B, V at H82C, Tat H87, A at H88, V at H89, C at H100A, V at H100B, E at H100F, G at H100G, G at H100H, L, F, Y or C at H100J, Y at H100K, W at H100P, E or P at H101, Tat H102, and N or K at H105, I at H107, K at H108, wherein the VH residues are numbered according to Kabat;
[211] the antibody or antigen binding fragment thereof of [210], wherein the VH comprises two or more, three or more, four or more, or five or more of a V at H2, G at H15, S at H31, L at H37, E at H39, T at H40, L or I at H45, T at H51, H at H53, A at H60, H or T at H68, I at H69, G at H70, H at H72, P at H73, Eat H74, K at H81, K or Tat H82A, S at H82B, V at H82C, Tat H87, A at H88, V at H89, C at H100A, V at H100B, E at H100F, G at H100G, G at H100H, L, F, Y or C at H100J, Y at H100K, W at H100P, E or P at H101, T at H102, and N or K at H105, I at H107, K at H108;
[212] the antibody or antigen binding fragment thereof of [210], wherein the VH comprises a V at H2, G at H15, S at H31, L at H37, E at H39, T at H40, L or I at H45, T at H51, H at H53, A at H60, H or T at H68, I at H69, G at H70, H at H72, P at H73, E at H74, K at H81, K or Tat H82A, S at H82B, V at H82C, Tat H87, A at H88, V at H89, C at H100A, Vat H100B, E at H100F, G at H100G, G at H100H, L, F, Y or C at H100J, Y at H100K, W at H100P, E or P at H101, T at H102, and N or K at H105, I at H107, K at H108;
[213] the antibody or antigen binding fragment thereof of [210], wherein the VH comprises a G at H15, S at H31, L at H37, E at H39, T at H40, T at H51, H at H53, A at H60, H or T at H68, L or I at H69, G or S at H70, D or H at H72, P at H73, K or Tat H82A, S at H82B, V at H82C, Tat H87, A at H88, V at H89, L, F, W, Y or C at H100J, W or Mat H100P, E, D or P at H101, V or T at H102, and N or K at H105;
[214] the antibody or antigen binding fragment thereof of [210], wherein the VH comprises a G at H15, S at H31, L at H37, E at H39, T at H40, T at H51, H at H53, A at H60, H or T at H68, L or I at H69, G or S at H70, D or H at H72, P at H73, V at H82A, A at H82B, A at H82C, S at H87, G at H88, K at H89, L, F, W, Y or C at H100J, W or Mat H100P, E, D or P at H101, V or T at H102, and N or K at H105;
[215] the antibody or antigen binding fragment thereof of [210], wherein the VH comprises a G at H15, S at H31, L at H37, E at H39, T at H40, T at H51, H at H53, A at H60, H at H68, L at H69, G at H70, H at H72, P at H73, K or T at H82A, S at H82B, V at H82C, T at H87, A at H88, V at H89, L, F, W, Y or C at H100J, W or Mat H100P, E, D or P at H101, V or T at H102, and N or K at H105;
[216] the antibody or antigen binding fragment thereof of [210], wherein the VH comprises a G at H15, S at H31, L at H37, E at H39, T at H40, T at H51, H at H53, A at H60, T at H68, I at H69, G or S at H70, D at H72, P at H73, V at H82A, A at H82B, A at H82C, S at H87, G at H88, K at H89, L, F, W, Y or C at H100J, W or Mat H100P, E, D or P at H101, V or T at H102, and N or K at H105;
[217] the antibody or antigen binding fragment thereof of any one of [210] to [216], wherein the VH comprises MDV, WDV, MEV, WDV, or WPT at H100P, H101, and H102, respectively;
[218] the antibody or antigen binding fragment thereof of any one of [210] to [217], wherein the VL comprises one or more of a S at L2, Y at L3, Vat L4, L at L5, Tat L6, Q at L7, P at L8, P at L9, S at L10, V at L11, V at L19, T at L22, G at L25, E at L26, I at L28, Q at L38, P at L40, K or Rat L45, G at L55, P at L56, I at L68, D at L69, S at L79, N or Rat L80, Tat L88, Vat L93, Rat L95C, N or Q at L95F, Eat L97, L at L101, Eat 102L, and P at L103, wherein the VL residues are numbered according to Kabat;
[219] the antibody or antigen binding fragment thereof of [218], wherein the VL comprises a V at L19, T at L22, G at L25, E at L26, I at L28, P at L40, K or R at L45, P at L56, I at L68, D at L69, S at L79, N at L80, T at L88, Rat L95C, N at L95F, E at L97, L at L101, and P at L103;
[220] the antibody or antigen binding fragment thereof of [218], wherein the VL comprises two or more, three or more, four or more, or five or more of a V at L19, T or S at L22, G at L25, E at L26, I at L28, A or P at L40, K or Rat L45, P at L56, I at L68, D at L69, S or T at L79, N at L80, T at L88, R at L95C, N at L95F, E or V at L97, L or F at L101, and P or G at L103;
[221] the antibody or antigen binding fragment thereof of [218], wherein the VL comprises V at L19, T or S at L22, G at L25, E at L26, I at L28, A or P at L40, K or R at L45, P at L56, I at L68, D at L69, S or Tat L79, N at L80, T at L88, Rat L95C, N at L95F, E or V at L97, L or F at L101, and P or G at L103;
[222] the antibody or antigen binding fragment thereof of any one of [210] to [221], wherein the VH comprises a VH CDR3 comprising the sequence of TLHGRRX1YGX2VAFX3EX4X5TYFYX6X7X8 wherein X1 is I or C, X2 is I or V, X3 is N or G, X4 is W, C, F, L or Y, X5 is F or Y, X6 is M or W, X7 is D, P or E, and X8 is V or T (SEQ ID NO: 71) or TLHGRRIYGX1VAFX2EX3X4TYFYX5X6X7 wherein X1 is I or V, X2 is N or G, X3 is W, F, L or Y, X4 is F or Y, X5 is M or W, X6 is D, P or E, and X7 is V or T (SEQ ID NO: 72);
[223] the antibody or antigen binding fragment thereof of any one of [210] to [221], wherein the VH comprises
   (a) a VH CDR1 comprising the sequence of X1SYWS, wherein X1 is D or S (SEQ ID NO: 26);
   (b) a VH CDR2 comprising the sequence of YX1HX2SGDTNYX3PSLKS wherein X1 is V or T, X2 is K or H, and X3 is S or A (SEQ ID NO: 27); and
   (c) a VH CDR3 comprising the sequence of
      TLHGRRX1YGX2VAFX3EX4X5TYFYX6X7X8 wherein X1 is I or C, X2 is I or V, X3 is N or G, X4 is W, C, F, L or Y, X5 is F or Y, X6 is M or W, X7 is D, P or E, and X8 is V or T (SEQ ID NO: 71) or
      TLHGRRIYGX1VAFX2EX3X4TYFYX5X6X7 wherein X1 is I or V, X2 is N or G, X3 is W, F, L or Y, X4 is F or Y, X5 is M or W, X6 is D, P or E, and X7 is V or T (SEQ ID NO: 72);
[224] the antibody or antigen binding fragment thereof of any one of [210] to [223], wherein the VL comprises
   (a) a VL CDR1 comprising the sequence of GX1X2SX3GSRAVQ wherein X1 is E or G, X2 is K or E, and X3 is L or I (SEQ ID NO: 29);
   (b) a VL CDR2 comprising the sequence of NNQDRX1X2 wherein X1 is P or G, and X2 is S or P (SEQ ID NO: 168); and
   (c) a VL CDR3 comprising the sequence of HX1WDSRX2PTX3WX4 wherein X1 is I or V, X2 is V or R, X3 is K, Q or N, and X4 is V or E (SEQ ID NO: 73);
[225] the antibody or antigen binding fragment thereof of any one of [210] to [224], wherein the VL is SEQ ID NO: 2, 10, 11, 46, 48, 55, 66, or 127-139;
[226] the antibody or antigen binding fragment thereof of any one of [210] to [225], wherein the VH comprises SEQ ID NO: 3-9, 45, 47, 49-54, 65, 69, 70, or 112-126;
[227] the antibody or antigen binding fragment thereof of any one of [1] to [226], wherein at least one of the VH CDR1, VH CDR2, VH CDR3, VL CDR1, VL CDR2, and VL CDR3 comprises a different amino acid sequence than the corresponding CDR of the PGT-121 antibody;
[228] the antibody or antigen binding fragment thereof of any one of [1] to P26, which is not PGT-121 or an antigen binding fragment thereof;
[229] An antibody or antigen binding fragment thereof capable of binding to HIV gp120, wherein the antibody comprises
   (i) a VH comprising the amino acid sequence of SEQ ID NO: 3-9, 45, 47, 49-54, 65, 69, 70, or 112-126; and
   (ii) a VL comprising the amino acid sequence of SEQ ID NO: 10, 11, 46, 48, 55, 66, or 127-139;
[230] An antibody or antigen binding fragment thereof capable of binding to HIV gp120, wherein the antibody comprises
   (i) a VH comprising the amino acid sequence of SEQ ID NO: 3 and a VL comprising the amino acid sequence of SEQ ID NO: 10;
   (ii) a VH comprising the amino acid sequence of SEQ ID NO: 3 and a VL comprising the amino acid sequence of SEQ ID NO: 11;
   (iii) a VH comprising the amino acid sequence of SEQ ID NO: 4 and a VL comprising the amino acid sequence of SEQ ID NO: 10;
   (iv) a VH comprising the amino acid sequence of SEQ ID NO: 4 and a VL comprising the amino acid sequence of SEQ ID NO: 11;
   (v) a VH comprising the amino acid sequence of SEQ ID NO: 5 and a VL comprising the amino acid sequence of SEQ ID NO: 10;
   (vi) a VH comprising the amino acid sequence of SEQ ID NO: 5 and a VL comprising the amino acid sequence of SEQ ID NO: 11;
   (vii) a VH comprising the amino acid sequence of SEQ ID NO: 6 and a VL comprising the amino acid sequence of SEQ ID NO: 10;
   (viii) a VH comprising the amino acid sequence of SEQ ID NO: 6 and a VL comprising the amino acid sequence of SEQ ID NO: 11;
   (ix) a VH comprising the amino acid sequence of SEQ ID NO: 7 and a VL comprising the amino acid sequence of SEQ ID NO: 10;
   (x) a VH comprising the amino acid sequence of SEQ ID NO: 7 and a VL comprising the amino acid sequence of SEQ ID NO: 11;
   (xi) a VH comprising the amino acid sequence of SEQ ID NO: 8 and a VL comprising the amino acid sequence of SEQ ID NO: 10;
   (xii) a VH comprising the amino acid sequence of SEQ ID NO: 8 and a VL comprising the amino acid sequence of SEQ ID NO: 11;
   (xiii) a VH comprising the amino acid sequence of SEQ ID NO: 9 and a VL comprising the amino acid sequence of SEQ ID NO: 10;
   (xiv) a VH comprising the amino acid sequence of SEQ ID NO: 9 and a VL comprising the amino acid sequence of SEQ ID NO: 11;
   (xv) a VH comprising the amino acid sequence of SEQ ID NO: 65 and a VL comprising the amino acid sequence of SEQ ID NO: 55;
   (xvi) a VH comprising the amino acid sequence of SEQ ID NO: 69 and a VL comprising the amino acid sequence of SEQ ID NO: 55;
   (xvii) a VH comprising the amino acid sequence of SEQ ID NO: 70 and a VL comprising the amino acid sequence of SEQ ID NO: 55;
   (xviii) a VH comprising the amino acid sequence of SEQ ID NO: 65 and a VL comprising the amino acid sequence of SEQ ID NO: 66;
   (xix) a VH comprising the amino acid sequence of SEQ ID NO: 69 and a VL comprising the amino acid sequence of SEQ ID NO: 66;
   (xx) a VH comprising the amino acid sequence of SEQ ID NO: 70 and a VL comprising the amino acid sequence of SEQ ID NO: 66; or
   (xxi) a VH comprising the amino acid sequence of SEQ ID NO: 112-126 and a VL comprising the amino acid sequence of SEQ ID NO: 127-139;
[231] the antibody or antigen binding fragment thereof of any one of [1] to [230] capable of binding HIV gp120 at pH 4;0;
[232] the antibody or antigen binding fragment thereof of any one of [1] to [230] capable of binding HIV gp120 at pH 5;0;
[233] the antibody or antigen binding fragment thereof of any one of [1] to [230] capable of binding HIV gp120 at pH 4;5;
[234] the antibody or antigen binding fragment thereof of any one of [1] to [230] capable of binding HIV gp120 at pH 3;5;
[235] the antibody or antigen binding fragment thereof of any one of [1] to [230] capable of binding HIV gp120 at pH 3;0;
[236] the antibody or antigen binding fragment thereof of any one of [1] to [230] capable of binding HIV gp120 at pH 5;0 with a higher affinity than PGT-121;
[237] the antibody or antigen binding fragment thereof of any one of [1] to [230] capable of binding HIV gp120 at pH 4;5 with a higher affinity than PGT-121;
[238] the antibody or antigen binding fragment thereof of any one of [1] to [230] capable of binding HIV gp120 at pH 4;0 with a higher affinity than PGT-121;
[239] the antibody or antigen binding fragment thereof of any one of [1] to [230] capable of binding HIV gp120 at pH 3;5 with a higher affinity than PGT-121;
[240] the antibody or antigen binding fragment thereof of any one of [1] to [230] capable of binding HIV gp120 at pH 3;0 with a higher affinity than PGT-121;
[241] the antibody or antigen binding fragment thereof of any one of [1] to [230], which binds to HIV gp120 with a higher affinity than PGT-121;
[242] the antibody or antigen binding fragment thereof of any one of [1] to [230], which is capable of competing with PGT-121 for binding to HIV gp120;
[243] the antibody or antigen binding fragment thereof of any one of [1] to [230], which binds to the same epitope of HIV gp120 as PGT-121;
[244] the antibody or antigen binding fragment thereof of any one of [1] to [243], wherein the HIV gp120 is 92BR020 gp120;
[245] the antibody or antigen binding fragment thereof of any one of [1] to [244] that is a broadly neutralizing antibody;
[246] the antibody or antigen binding fragment thereof of any one of [1] to [244] capable of neutralizing at least two cross-clade isolates of HIV;
[247] the antibody or antigen binding fragment thereof of any one of [1] to [244] capable of neutralizing at least about 50%, 60%, 70%, 80%, 90%, or 100% of cross-clade HIV isolates in the 106-member indicator virus panel;
[248] the antibody or antigen binding fragment thereof of any one of [1] to [244] capable of neutralizing at least about 50%, 60%, 70%, 80%, 90%, or 100% of cross-clade PGT121 resistant HIV isolates in the 30-member indicator virus panel;
[249] the antibody or antigen binding fragment thereof of any one of [246] to [248], wherein the antibody or antigen binding fragment thereof is capable of neutralizing the cross-clade HIV isolates with a median IC50 equal to or less than about 0;1 microg/ml, 0;05 microg/ml, 0;025 microg/ml, 0;01 microg/ml, or 0;005 microg/ml;
[250] the antibody or antigen binding fragment thereof of any one of [1] to [249] that is not polyreactive;
[251] the antibody or antigen binding fragment thereof of [250], wherein polyreactivity is assessed by the HEp-2 polyreactivity assay;
[252] the antibody or antigen binding fragment thereof of any one of [1] to [251], wherein the antibody is a recombinant antibody, a chimeric antibody, a bispecific antibody, or a trispecific antibody;
[253] the antibody or antigen binding fragment thereof of any one of [1] to [251], wherein the antibody fragment comprises a single-chain Fv (scFv), F(ab) fragment, F(ab′)2 fragment, or an isolated VH domain;
[254] the antibody or antigen binding fragment thereof of any one of [1] to [253], further comprising a heavy and/or light chain constant region;
[255] the antibody or antigen binding fragment thereof of any one of [1] to [253], further comprising a human heavy and/or light chain constant region;
[256] the antibody or antigen binding fragment thereof of [255], wherein the heavy chain constant region is human immunoglobulin IgG1, IgG2, IgG3, IgG4, IgA1, or IgA2 constant region;
[257] the antibody or antigen binding fragment thereof of any one of [254] to [256], wherein the heavy chain constant region comprises a native amino acid sequence;
[258] the antibody or antigen binding fragment thereof of any one of [254] to [256], wherein the heavy chain constant region comprises a variant amino acid sequence;
[259] A pharmaceutical composition comprising the antibody or antigen binding fragment thereof of any one of [1] to [258] and a pharmaceutically acceptable excipient;
[260] An isolated polynucleotide encoding the antibody or antigen binding fragment thereof of any one of [1] to [258];
[261] the isolated polynucleotide of [260], which is a DNA;
[262] the isolated polynucleotide of [260], which is an mRNA;
[263] the isolated polynucleotide of [262], wherein the mRNA comprises a modified nucleotide;
[264] An isolated vector comprising the polynucleotide of [260];
[265] the isolated vector of [264], wherein the vector is a viral vector;
[266] A recombinant virus comprising the polynucleotide of [260];
[267] the recombinant virus of [266], which is a recombinant adeno-associated virus (AAV);
[268] A host cell comprising the polynucleotide of [260] or the vector of [264];
[269] the host cell of [268], which is E; coli, Pseudomonas, Bacillus, Streptomyces, yeast, CHO, YB/20, NS0, PER-C6, HEK-293T, NIH-3T3, HeLa, BHK, Hep G2, SP2/0, R1;1, B-W, L-M, COS 1, COS 7, BSC1, BSC40, BMT10 cell, plant cell, insect cell, or human cell in tissue culture;
[270] A method of producing the antibody or antigen binding fragment thereof of any one of [1] to [258] comprising culturing the host cell of [268] or [269] so that the antibody or antigen binding fragment thereof is expressed and the antibody or antigen binding fragment thereof is produced;
[271] A method of neutralizing an HIV virus comprising contacting the virus with a sufficient amount of the antibody or antigen binding fragment thereof of any one of [1] to [258];
[272] A method of reducing the likelihood of HIV infection in a subject exposed to HIV comprising administering to the subject a therapeutically sufficient amount of the antibody or antigen binding fragment thereof of any one of [1] to [258];
[273] A method of treating HIV/AIDS comprising administering to a subject in need thereof a therapeutically sufficient amount of the antibody or antigen binding fragment thereof of any one of [1] to [258];
[274] A method of reducing viral load comprising administering to a subject in need thereof a therapeutically sufficient amount of the antibody or antigen binding fragment thereof of any one of [1] to [258];
[275] the method of [272] to [274], wherein the administering to the subject is by at least one mode selected from oral, parenteral, subcutaneous, intramuscular, intravenous, vaginal, rectal, buccal, sublingual, and transdermal;
[276] the method of any one of [272] to [275], further comprising administering at least one additional therapeutic agent;
[277] the method of [276], wherein the additional therapeutic agent is an antiretroviral agent, a reservoir activator, or a second antibody;
[278] the method of [276], wherein the additional therapeutic agent comprises a broadly neutralizing antibody;
[279] the method of [276], wherein the additional therapeutic agent comprises two broadly neutralizing antibodies;
[280] the method of [276], wherein the additional therapeutic agent comprises three broadly neutralizing antibodies.
[281] A method of producing an engineered variant of PGT121 comprising
   (i) substituting one or more amino acid residues of the PGT121 VH to a V at H2, G at H15, S at H31, I at H37, E at H39, T at H40, T at H51, H at H53, A at H60, H at H68, G at H70, H at H72, P at H73, K at H81, K or T at H82A, S at H82B, V at H82C, Tat H87, A at H88, V at H89, V at H100D, G at H100H, F or L at H100J, W at H100P, E at H101, and K at H105, wherein the VH residues are numbered according to Kabat; and/or
   (ii) substituting one or more amino acid residues of the PGT121 VL to a S at L2, Y at L3, V at L4, L at L5, T at L6, Q at L7, P at L8, P at L9, S at L10, V at L11, V at L19, G at L25, E at L26, I at L28, K at L45, P at L56, I at L68, D or A at L69, N at L80, T at L88, R at L95C, and N at L95F, wherein the VL residues are numbered according to Kabat.
[282] A method of producing an engineered variant of PGT121 comprising
   (i) substituting one or more amino acid residues of the PGT121 VH to a V at H2, G at H15, S at H31, L at H37, E at H39, T at H40, L or I at H45, T at H51, H at H53, A at H60, H or T at H68, I at H69, G at H70, H at H72, P at H73, E at H74, K at H81, K or T at H82A, S at H82B, V at H82C, Tat H87, A at H88, Vat H89, C at H100A, V at H100B, E at H100F, G at H100G, G at H100H, L, F, Y or C at H100J, Y at H100K, W at H100P, E or P at H101, T at H102, and N or K at H105, I at H107, K at H108, wherein the VH residues are numbered according to Kabat; and/or
   (ii) substituting one or more amino acid residues of the PGT121 VL to a S at L2, Y at L3, V at L4, L at L5, T at L6, Q at L7, P at L8, P at L9, S at L10, V at L11, V at L19, Tat L22, G at L25, Eat L26, I at L28, Q at L38, P at L40, K or Rat L45, G at L55, P at L56, I at L68, D at L69, S at L79, N or R at L80, Tat L88, V at L93, R at L95C, N or Q at L95F, E at L97, L at L101, E at 102L, and P at L103, wherein the VL residues are numbered according to Kabat.

### BRIEF DESCRIPTION OF THE DRAWINGS

FIG. 1. pH dependence of the binding affinities of the PGT-121, PGT128, PGT135, 2G12, b12, and VRC01 bnAbs to gp120 (isolate: 92BR020).

FIG. 2. General design strategy combining directed evolution (yeast surface display, FACS and deep sequencing) with informatics analysis and computational modeling to generate enhanced engineered variant broadly neutralizing anti-HIV antibodies.

FIGS. 3A-3C. (A) Enhanced engineered variant VH and VL sequences. Sequences shown are IGLV3-21*1 SEQ ID NO: 56, PGT121 VL SEQ ID NO: 2, ePGT121.L4 VL SEQ ID NO: 46. (B) Pseudovirus neutralization assay. (C) pH dependence of binding affinities to 94UG103 and 92BR020 gp120.

FIGS. 4A and 4B. (A) Enhanced engineered variant VH and VL sequences. Sequences shown are IGHV4-59*03 SEQ ID NO: 57, PGT121 VH SEQ ID NO: 1, ePGT121.13.H1 VH SEQ ID NO: 47, IGLV3-21*1 SEQ ID NO: 56, PGT121 VL SEQ ID NO: 2, ePGT121.13.L1 VL SEQ ID NO: 48. (B) Pseudovirus neutralization assay using the 7-virus panel.

FIG. 5 Enhanced engineered variant VH and VL sequences. Sequences shown are IGHV4-59*03 SEQ ID NO: 57, PGT121 VH SEQ ID NO: 1, ePGT121.14.H1 VH SEQ ID NO: 49, ePGT121.14.H2 VH SEQ ID NO: 50, ePGT121.14.H3 VH SEQ ID NO: 51, ePGT121.14.H4 VH SEQ ID NO: 52, ePGT121.14.H5 VH SEQ ID NO: 53, ePGT121.14.H6 VH SEQ ID NO: 54, IGLV3-21*1 SEQ ID NO: 56, PGT121 VL SEQ ID NO: 2, ePGT121.14.L1 VL SEQ ID NO: 55.

FIGS. 6A and 6B. (A) Pseudovirus neutralization assay (7-virus panel) and (B) HEp-2 cell based polyreactivity assay results for ePGT121.14.H1.L1, ePGT121.14H2.L1, ePGT121.14.H3.L1, ePGT121.14H4.L1, ePGT121.14H5.L1 and ePGT121.14H6.L1 antibodies. Reversion of 6 amino acid residues to germline sequence in the framework 3 region of PGT-121.14.H1 VH to produce ePGT-121.14.H6 VH eliminates polyreactivity without impacting neutralization potency.

FIG. 7 Enhanced engineered variant VH and VL sequences. Sequences shown: IGHV4-59*03 SEQ ID NO: 57, PGT121 VH SEQ ID NO: 1, ePGT121.15.H1 VH SEQ ID NO: 3, ePGT121.15.H2 VH SEQ ID NO: 4, ePGT121.15.H3 VH SEQ ID NO: 5, ePGT121.15.H4 VH SEQ ID NO: 6, ePGT121.15.H5 VH SEQ ID NO: 7, ePGT121.15.H6 VH SEQ ID NO: 8, ePGT121.15.H7 VH SEQ ID NO: 9, IGLV3-21*1 SEQ ID NO: 56, PGT121 VL SEQ ID NO: 2, ePGT121.15.L1 VL SEQ ID NO: 10, ePGT121.15.L2 VL SEQ ID NO: 11.

FIG. 8. The enhanced engineered PGT-121 variant antibodies are not polyreactive. Results of a single antigen polyreactivity assay obtained with ePGT-121.15 variant antibodies comprising Light Chain 1 (LC1) are shown.

FIG. 9. Results of a single antigen polyreactivity assay obtained with ePGT-121.15 variant antibodies comprising Light Chain 2 (LC2) are shown.

FIG. 10. Neutralization breadth and potency of ePGT121.15.H1.L1 IC50 and IC80 data measured using the panel of 106 HIV isolates (see Table 5) is shown. Data obtained for the PGT-121 parent antibody is included as a reference.

FIG. 11. Neutralization breadth and potency of ePGT121.15.H1.L1. The % of isolates in a 106 member panel that were at least 80% neutralized (IC80) was plotted against antibody concentration (microg/mL). Concentration dependence (Breadth of coverage (% of isolates. Breadth improved from about 10% (PGT-121) to about 50% (PGT-121.15.1) at an IC80 of 0.01 microg/ml.

FIG. 12. Neutralization breadth and potency of ePGT121.15.H1.L1. Distribution of bnAbs on a potency (median IC50 μg/ml) vs. coverage (%) is shown.

FIG. 13. pH dependence of the binding affinities of ePGT121.15.H1.L1 and PGT121 to gp120 (isolate: 92BR020).

FIG. 14. SEC profile of isolated ePGT121.15.H1.L1, ePGT121.15.H1.L1, ePGT121.15.H2.L1, ePGT121.15.H3.L1, ePGT121.15.H4.L1, ePGT121.15.H5.L1, ePGT121.15.H6.L1, and ePGT121.15.H7.L1 variant antibodies comprising Light Chain 1 (LC1).

FIG. 15. SEC profile of isolated ePGT121.15.H1.L2, ePGT121.15.H1.L2, ePGT121.15.H2.L2, ePGT121.15.H3.L2, ePGT121.15.H4.L2, ePGT121.15.H5.L2, ePGT121.15.H6.L2, and ePGT121.15.H7.L2 variant antibodies comprising Light Chain 2 (LC2).

FIG. 16 Enhanced engineered variant VH domains. Sequences shown: ePGT121.15.H6 VH SEQ ID NO: 8, ePGT121.18H1 VH SEQ ID NO: 65, ePGT121.18.H2 VH SEQ ID NO: 112, ePGT121.18.H3 VH SEQ ID NO: 113, ePGT121.18.H4 VH SEQ ID NO: 114, ePGT121.18.H5 VH SEQ ID NO: 115, ePGT121.18.H6 VH SEQ ID NO: 116, ePGT121.18.H7 VH SEQ ID NO: 117, ePGT121.18.H8 VH SEQ ID NO: 118, ePGT121.18.H9 VH SEQ ID NO: 119, ePGT121.18.H10VHSEQ ID NO: 120, ePGT121.18.H11 VH SEQ ID NO: 121, ePGT121.18.H12 VH SEQ ID NO: 122, ePGT121.18.H13 VH SEQ ID NO: 123, ePGT121.18.H14 VH SEQ ID NO: 124, ePGT121.18.H15 VH SEQ ID NO: 125, ePGT121.18.H16 VH SEQ ID NO: 126, ePGT121.18.H17 VH SEQ ID NO: 69, ePGT121.18.H18 VH SEQ ID NO: 70.

FIG. 17 Enhanced engineered variant VL domains. Sequences shown: ePGT121.15_L1 VL SEQ ID NO: 10, ePGT121.17_L1 VL SEQ ID NO: 66, ePGT121.17_L2 VL SEQ ID NO: 127, ePGT121.17_L3 VL SEQ ID NO: 128, ePGT121.17_L4 VL SEQ ID NO: 129, ePGT121.17_L5 VL SEQ ID NO: 130, ePGT121.17_L6 VL SEQ ID NO: 131, ePGT121.17_L7 VL SEQ ID NO: 132, ePGT121.17_L8 VL SEQ ID NO: 133, ePGT121.17_L9 VL SEQ ID NO: 134, ePGT121.17_L10 VL SEQ ID NO: 135, ePGT121.17_L11 VL SEQ ID NO: 136, ePGT121.17_L12 VL SEQ ID NO: 137, ePGT121.17_L13 VL SEQ ID NO: 138, ePGT121.17_L14 VL, SEQ ID NO: 139.

FIG. 18. Neutralization breadth and potency of PGT-121, ePGT121v1 (comprising ePGT121.L15.H6 VH and ePGT121.L15.L1 VL), ePGT121v2 (comprising ePGT121.L18.H1 VH and ePGT121.L17.L1 VL), and ePGT121v3 (comprising ePGT121.L18.H17 VH and ePGT121.L17.L1 VL) antibodies. % of viral isolates in a 208-member cross-clade panel neutralized with an IC80 below the cut off value of 0.10 microg/ml and 0.01 microg/ml is shown.

FIG. 19. ePGT121 antibodies bind better to gp120 at low pH than PGT121. ePGT121v1 comprises ePGT121.L15.H6 VH and ePGT121.L15.L1 VL, ePGT121v2 comprises ePGT121.L18.H1 VH and ePGT121.L17.L1 VL.

### DETAILED DESCRIPTION

Provided herein are anti-HIV Env antibodies that were derived from the PGT121 parent antibody using directed-evolution and yeast display. In one embodiment, an antibody disclosed herein possesses one or more improved properties, for example, higher binding affinity to target antigen, higher binding affinity to target antigen at low pH, increased median neutralization IC₅₀ potency, and increased breadth of neutralization compared to PGT121. In some embodiments, the antibody does not demonstrate polyreactivity by HEp-2 assay and/or does not bind to solubilized membrane preparations. Due to their significantly improved properties, in one embodiment, antibodies disclosed herein confer sterilizing immunity at lower doses and consequently both reduce the cost per dose and increase the number of doses that can be produced by a manufacturer. Thus the improved antibodies disclosed herein can achieve substantially decreased cost of goods and significantly increased numbers of people who can be protected against HIV.

One aspect of the present disclosure relates to improved anti-HIV Env antibodies, and to nucleotide sequences encoding, compositions comprising, and kits comprising thereof. In another aspect, it relates to methods of treatment and prevention of HIV using an antibody disclosed herein. In another aspect, it relates to methods of diagnosing and monitoring of HIV infection using an antibody disclosed herein.

### I. Definitions

To facilitate an understanding of the present invention, a number of terms and phrases are defined below.

The terms “human immunodeficiency virus” or “HIV,” as used herein, refer generally to a retrovirus that is the causative agent for acquired immunodeficiency syndrome (AIDS), variants thereof (e.g., simian acquired immunodeficiency syndrome, SAIDS), and diseases, conditions, or opportunistic infections associated with AIDS or its variants, and includes HIV-Type 1 (HIV-1) and HIV-Type 2 (HIV-2) of any clade or strain therein, related retroviruses (e.g., simian immunodeficiency virus (SIV)), and variants thereof (e.g., engineered retroviruses, e.g., chimeric HIV viruses, e.g., simian-human immunodeficiency viruses (SHIVs)). In one embodiment, an HIV virus is an HIV-Type-1 virus. Previous names for HIV include human T-lymphotropic virus-Ill (HTLV-III), lymphadenopathy-associated virus (LAV), and AIDS-associated retrovirus (ARV).

As used herein, the term “clade” refers to related human immunodeficiency viruses (HIVs) classified according to their degree of genetic similarity. There are currently four known groups of HIV-1 isolates: M, N, O, and P. Group M (major strains) viruses are responsible for the majority of the global HIV epidemic. The other three groups, i.e., N, O and P are quite uncommon and only occur in Cameroon, Gabon and Equatorial Guinea. In one embodiment, an HIV virus is a Group M HIV virus. Within group M there are known to be at least nine genetically distinct subtypes or clades of HIV-1: subtypes or clades A, B, C, D, F, G, H, J and K. Additionally, different subtypes can combine genetic material to form a hybrid virus, known as a ‘circulating recombinant form’ (CRFs). Subtype/clade B is the dominant HIV subtype in the Americas, Western Europe and Australasia. Subtype/clade C is very common in the high AIDS prevalence countries of Southern Africa, as well as in the horn of Africa and India. Just under half of all people living with HIV have subtype C. In certain exemplary embodiments, methods described herein can be used to treat a subject (e.g., a human) infected with HIV (e.g., HIV-1) or to block or prevent HIV (e.g., HIV-1) infection in subject (e.g., a human) at risk of HIV transmission. The HIV may be of two, three, four, five, six, seven, eight, nine, ten, or more clades and/or two or more groups of HIV.

Acquired immune deficiency syndrome (“AIDS”) is a disease caused by the human immunodeficiency virus, or HIV.

As used herein, the term “envelope glycoprotein” or “Env” refers to the glycoprotein that is expressed on the surface of the envelope of HIV virions and the surface of the plasma membrane of HIV infected cells. “Envelope glycoprotein” or “Env” encompass, but are not limited to, native Env, an isoform of Env, or a variant of Env (e.g., SOSIP) derived from an HIV isolate, for example, BG505. Env is the sole virally encoded gene product on the surface of the virus and, as such, is the only target of neutralizing antibodies. Env is a trimer of heterodimers composed of two non-covalently associated subunits: the receptor-binding gp120 and the fusion machinery-containing gp41. Each subunit is derived from a gp160 precursor glycoprotein following cleavage by cellular furins. HIV-1 gp120 binds the CD4 molecule on the surface of human target T cells to initiate the viral entry process, and following co-receptor engagement, fusion is mediated by gp41. gp140 env is the uncleaved ectodomain of gp160. In one embodiment, Env is a 92BR020 Env polypeptide. In one embodiment, GenBank accession number AAT67490 and AAB05180 provide 92BR020 env gp160 polypeptide sequences. In one embodiment, 92BR020 Env comprises the amino acid sequence of SEQ ID NO: 58

(MRVKEMRKLWQHWWKGGILLLGMLMICSAKDNLWVTVYYGVPVWKEATT
TLFCASDAKAYKAEVHNVWATHACVPTDPNPQEIVLENVTENFNMWKNNM
VEQMHEDIISLWDQSLKPCVKLTPLCVTLNCIDLNNSTNNNNSSGVKTGI
DKGEIKNCSFNTTTSVKDKEKKEYALFYNLDVVQIGNDNTSYRLTSCNTS
VITQACPKVTFEPIPIHYCTPAGYAILKCNGKKFNGTGPCTNVSTVQCTH
GIKPVVSTQLLLNGSLAEEDIVIRSENLTNNAKTIIVQLKDPVDINCTRP
NNNTRKSIHIGPGRAFYATGDIIGDIRQAHCNLSRAQWNDTLSKIVTKLR
EQFENKTIKFQPPSGGDPEIVFHSFNCGGEFFYCNTTQLFNSTWTNNTEG
TSNTTGNDTITLPCRIKQIVNMWQEVGKAMYAPPIKGKIKCSSNITGLLL
TRDGGNNEMNTTEIFRPGGGDMRDNWRSELYKYKVVRIEPLGIAPTRAKR
RVVQREKRAVGTLGAMFLGFLGAAGSTMGAASVALTVQARQLLSGIVQQQ
NNLLRAIEAQQHMLQLTVWGIKQLQARVLAVERYLGDQQLLGIWGCSGKL
ICTTTVPWNTSWSNKSLDDIWTNMTWMEWKREIDNYTSLIYTLIEESQRQ
QEKNEQELLELDKWDSLWNWFTISKWLWYIKIFIMIVAGLVGLRIVFAVL
SIVNKVRQGYSPVSFQTRLPAQRGPDRPEEIEEEGGDRERERSGRLVTGL
LEIIWDDLRSLCLFSYRRLRDLLLIVARIVELLGRRGWEALKYWWNLLQY
WSQELKSSAVSLLNATAVAVAEGTDRIIEIAQRFFRGFLHIPRRIRQGLE
RALL).

The term “well-ordered Env trimer” or “well-ordered trimer” as used herein refers to an envelope glycoprotein trimer comprising three cleaved gp140 polypeptides that closely mimics the quaternary structure of the Env ectodomain on the surface of the envelope of HIV or SIV virions and the surface of the plasma membrane of HIV or SIV infected cells. In one embodiment, the gp120 and gp41 ectodomain is linked by a covalent linkage, for example, a disulfide bond. hi one embodiment, the gp140 polypeptide comprises one or more mutations to promote trimer formation. In one embodiment, the gp140polypeptide comprises one or more mutations to promote disulfide formation. In one embodiment the well-ordered trimer is an SOSIP gp140 trimer. Well-ordered SOSIP trimers have been disclosed in US Patent. Appl. Pub. No. 2014/0212458, Sanders, R. W. et al., PLoS Pathog. 9, e1003618 (2013) and Guenaga J., et al., Immunity 46(5):792-803.e3 (2017), each of which is incorporated by reference herein in its entirety. In one embodiment, a well ordered trimer is formed from a clade, A Env. in one embodiment, a well ordered trimer is formed from a clade B Env. In one embodiment, a well ordered trimer is formed from a clade C Lay. In one embodiment, a well ordered trimer is formed from a circulating recombinant form Env. In one embodiment, a well ordered trimer is 92BR020 SOSIP. In one embodiment, a well ordered trimer is 8(3505 SOSIP as disclosed in International Application No. PCT/US2018/041729, flied Jul. 12, 2018. which is incorporated herein by reference in its entirety for all purposes. In one embodiment, a well-ordered Env trimer is a native flexibly linked (NFL) trimer as described in Sharna, et al., Cell Reports, 11(4):539-50 (2015). In one embodiment, a well-ordered Env trimer is a DS-SOSIP as described m Chuang, et al., J. Virology, 91(10). pii: e02268-16 (2017). In one embodiment, a well ordered trimer is formed from a SIV Env. In one embodiment, a well ordered trimer is an SIV Env SOSIP. In one embodiment, a well ordered trimer is formed from an Env comprising a mutation (e.g., substitution or deletion) in the CD4 binding site. In one embodiment, a well ordered trimer is formed from an Env comprising a mutation (e.g., substitution or deletion) in the CD4 binding site wherein the mutation reduces or disrupts the binding between Env and CD4. In one embodiment, a well ordered trimer is a CRF or C108 SOSIP. See, e.g., Andrabi et al, Immunity 43(5): 959-973 (2015). In some embodiments, the gp120 and gp41 ectodomain is linked by a peptide linker, for example, a Gly-Ser linker, as described in Georgiev I S, et al., J. Virology 89(10): 5318-5329 (2015). In some embodiments, the well-ordered Env trimer is stable.

The term “antibody” means an immunoglobulin molecule (or a group of immunoglobulin molecules) that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the terms “antibody” and “antibodies” are terms of art and can be used interchangeably herein and refer to a molecule with an antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), affybodies, Fab fragments, F(ab′)2 fragments, disulfide-linked Fvs (sdFv), anti-idiotypic (anti-Id) antibodies (including, e.g., anti-anti-Id antibodies), bispecific antibodies, and multi-specific antibodies. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclasses (isotypes) thereof (e.g. IgG₁, IgG₂, IgG₃, IgG₄, IgA1 and IgA2), of immunoglobulin molecule, based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated or fused to other molecules such as toxins, radioisotopes, other polypeptides etc.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., HIV Env). The antigen-binding region can be derived from any animal species, such as mouse and humans.

As used herein, the terms “variable region” or “variable domain” are used interchangeably and are common in the art. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen (e.g., HIV Env). In certain embodiments, the variable region comprises 3 CDRs (CDR1, CDR2, and CDR3) and 4 framework regions (FR1, FR2, FR3, and FR4) in the order of FR1-CDR1-FR2-CDR2-FR3-CDR3-FR4 from the N terminus to the C terminus. In certain embodiments, the variable region is a human variable region. In certain embodiments, the variable region comprises human CDRs and human framework regions (FRs). In certain embodiments, the variable region comprises CDRs and framework regions (FRs) wherein one or more of the CDRs were modified by a substitution, deletion, or insertion relative to the CDRs of a parental antibody. In certain embodiments, the variable region comprises CDRs and framework regions (FRs) wherein one or more of the FRs were modified by a substitution, deletion, or insertion relative to the FRs of a parental antibody. In certain embodiments, the variable region comprises CDRs and framework regions (FRs) wherein one or more of the CDRs and one or more of the FRs were modified by a substitution, deletion, or insertion relative to the CDRs and FRs of a parental antibody. In certain embodiments, the parental antibody is PGT-121. In certain embodiments, the variable region comprises human CDRs and primate (e.g., non-human primate) framework regions (FRs).

There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.), “Kabat”); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs. It is understood that the identification of CDRs in a variable region also identifies the FRs as the sequences flanking the CDRs.

The Kabat numbering system is generally used when referring to a residue in the variable domain (approximately residues 1-107 of the light chain and residues 1-113 of the heavy chain) (e.g., Kabat et al., Sequences of Immunological Interest. (5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. (5th Ed., 1991, National Institutes of Health, Bethesda, Md.), “Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Rabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Rabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The terms “VL” and “VL domain” are used interchangeably to refer to the light chain variable region of an antibody.

The terms “VH” and “VH domain” are used interchangeably to refer to the heavy chain variable region of an antibody.

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “polyclonal antibody” describes a composition of different (diverse) antibody molecules which are capable of binding to or reacting with several different specific antigenic determinants on the same or on different antigens. Usually, the variability of a polyclonal antibody is located in the so-called variable regions of the polyclonal antibody, in particular in the CDR regions. In the present disclosure a mixture of two or more polyclonal antibodies (a polycomposition) is produced in one mixture from a polyclonal polycomposition cell line, which is produced from two or more parental polyclonal cell lines each expressing antibody molecules which are capable of binding to a distinct target, but it may also be a mixture of two or more polyclonal antibodies produced separately. A mixture of monoclonal antibodies providing the same antigen/epitope coverage as a polyclonal antibody described herein will be considered as an equivalent of a polyclonal antibody. When stating that a member of a polyclonal antibody binds to an antigen, it is herein meant to be binding with a binding constant below 100 nM, preferably below 10 nM, even more preferred below 1 nM.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. murine) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species (e.g., murine) that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some embodiments, a “humanized antibody” is a resurfaced antibody.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g., mouse) with the desired specificity, affinity, and capability while the constant regions are homologous to the sequences in antibodies or antigen-binding fragments thereof derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following. In certain embodiments, an anti-HIV Env antibody disclosed herein binds to HIV gp120 with a Kd of at least about 0.1 μM or less, at least about 0.01 μM or less, at least about 1 nM or less, or at least about 0.1 nM or less. In certain embodiments, an anti-HIV Env antibody disclosed herein binds to HIV gp120 with a Kd of at least about 0.01 μM or less. In certain embodiments, the HIV gp120 is 92BR020 gp120.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better”, the antibody's affinity for the antigen is <0.6 nM, i.e. 0.59 nM, 0.58 nM, 0.57 nM etc. or any value less than 0.6 nM.

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, Id.), or other assays known in the art. In a specific embodiment, molecules that immunospecifically bind to an antigen bind to the antigen with a Kd that is at least 2 logs, 2.5 logs, 3 logs, or 4 logs lower than the Kd when the molecules bind non-specifically to another antigen. In one example, the antibody may specifically bind to the 92BR020 Env polypeptide. In one example, the antibody may specifically bind to the 92BR020 gp120 polypeptide. In one example, the antibody may specifically bind to a 92BR020 SOSIP trimer. The antibody may bind to 92BR020 gp120 with a Kd at least 2 logs, 2.5 logs, 3 logs, or 4 logs lower than Kd of binding to other viral or non-viral polypeptides. An antibody that specifically binds to Env encompass, but are not limited to, antibodies that specifically bind to native Env, an isoform of Env, or a variant of Env (e.g., SOSIP) derived from an HIV isolate, for example, 92BR020. In one embodiment, the antibody specifically binds to 92BR020 Env. In one embodiment, the antibody specifically binds to 92BR020 SOSIP.

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

The term “broadly neutralizing antibody” or “bnAb,” as used herein, with respect to HIV (e.g., HIV-1), refers to an antibody that recognizes HIV Env of more than one isolate or strain of HIV and inhibits or prevents receptor binding of target cells as evaluated in an in vitro neutralization assay. In one embodiment, a broadly neutralizing antibody inhibits infection of a susceptible target cell by HIV. In one embodiment, a broadly neutralizing antibody specifically binds an HIV Env and inhibits infection of a susceptible target cell (e.g., TZM-bl) by an HIV pseudovirus comprising an Env polypeptide. HIV pseudovirus neutralization assays have been disclosed in the art, for example, in Walker, L. M. et al., Nature 477, 466-470 (2011), Li M., et al., J. Virol. 79:10108-10125 (2005), each of which is incorporated herein by reference in its entirety for all purposes. In one embodiment, a broadly neutralizing antibody neutralizes 2, 3, 4, 5, 6, 7, 8, 9, or more HIV strains or pseudoviruses. In one embodiment, a broadly neutralizing antibody neutralizes 2, 3, 4, 5, 6, 7, 8, 9, or more HIV strains or pseudoviruses that belong to the same or different clades. In one embodiment, a broadly neutralizing antibody is capable of neutralizing HIV strains or pseudoviruses from at least two different clades. In one embodiment, a broadly neutralizing antibody is capable of neutralizing at least one clade B strain or pseudovirus and one clade C strain or pseudovirus. In one embodiment, a broadly neutralizing antibody is capable of neutralizing more than one clade B strain or pseudovirus and more than one clade C strain or pseudovirus. In one embodiment, a broadly neutralizing antibody is capable of neutralizing an HIV strain or pseudovirus from at least one, at least two, at least three, at least four, at least five, at least six, at least seven, at least eight, at least nine, at least ten, at least eleven, at least twelve, at least thirteen, at least fourteen, or all fifteen clades represented in the 106 member panel in Table 5. In one embodiment, a broadly neutralizing antibody is capable of neutralizing an HIV strain or pseudovirus from at least one, at least two, at least three, at least four, at least five, at least six, at least seven, at least eight, at least nine, at least ten, at least eleven, at least twelve, at least thirteen, at least fourteen, or all fifteen clades selected from the group consisting of clades A, A (T/F), AC, ACD, B, B (T/F), BC, C, C (T/F), CD, CRF01_AE, CRF01_AE (T/F), CRF02_AG, D, and G. In one embodiment, a broadly neutralizing antibody is capable of neutralizing an HIV strain or pseudovirus from at least one, at least two, at least three, at least four, at least five, at least six, at least seven, at least eight, at least nine, at least ten, or all eleven clades represented in the 113 member panel in Table 6. In one embodiment, a broadly neutralizing antibody is capable of neutralizing an HIV strain or pseudovirus from at least one, at least two, at least three, at least four, at least five, at least six, at least seven, at least eight, at least nine, at least ten, or all eleven clades selected from the group consisting of clades A, AC, ACD, AE, AG, B, BC, C, CD, D, G.

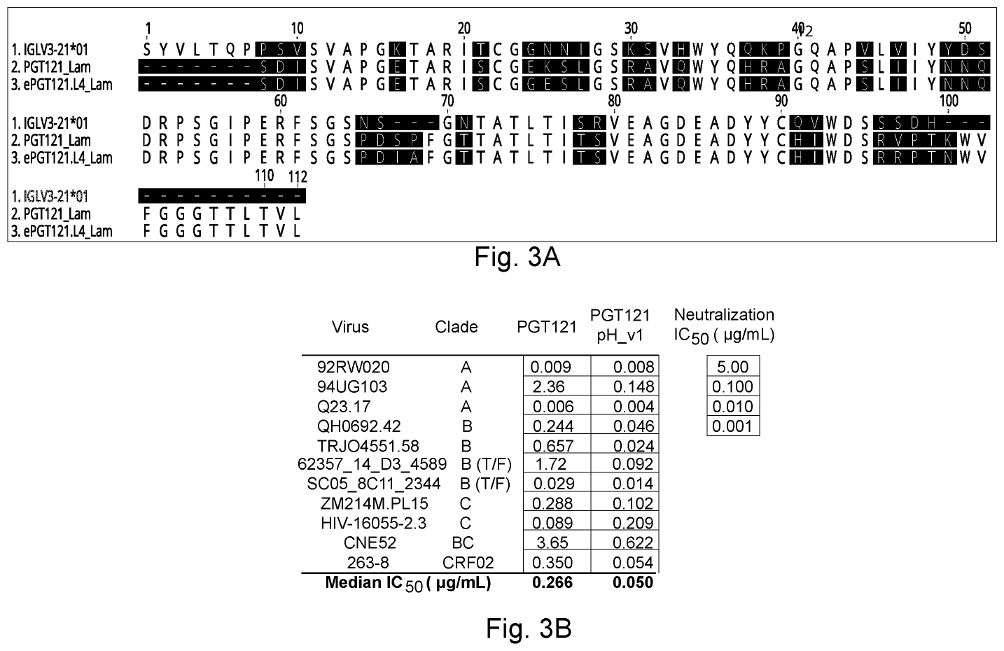

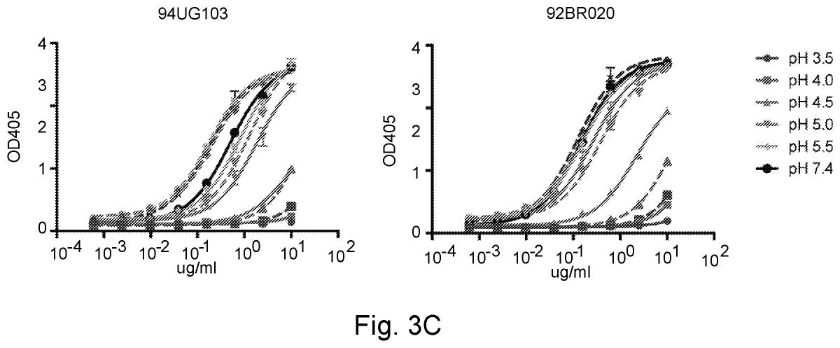

In one embodiment, the breadth of neutralization is tested on an indicator virus panel comprising cross-clade HIV isolates. In one embodiment, the virus panel comprises the 7 cross-clade isolates of 62357_14_D3_4589, ZM233M.PB6, ZM109F.PB4, CAP210.2.00.E8, CNE17, 928-28, and T251-18, as shown in FIG. 3. In one embodiment, the virus panel comprises the 12 cross-clade isolates of 92RW020, 94UG103, Q23.17, 0260.v5.c36, QH0692.42, TRJO4551.58, 62357_14_D3_4589, SC05_8C11_2344, ZM214M.PL15, HIV-16055-2.3, CNE52, and 263-8. In one embodiment, the virus panel comprises the 106 cross-clade isolates of 6535.3, QH0692.42, SC422661.8, PVO.4, TRO.11, AC10.0.29, RHPA4259.7, REJO4541.67, TRJO4551.58, WITO4160.33, CAAN5342.A2, WEAU_d15_410_5017, 1006_11_C3_1601, 1054_07_TC4_1499, 1056_10_TA11_1826, 1012_11_TC21_3257, 6240_08_TA5_4622, 6244_13_B5_4576, 62357_14_D3_4589, SC05_8C11_2344, Du156.12, Du172.17, Du422.1, ZM197M.PB7, ZM214M.PL15, ZM233M.PB6, ZM249M.PL1, ZM53M.PB12, ZM109F.PB4, ZM135M.PL10a, CAP45.2.00.G3, CAP210.2.00.E8, HIV-001428-2.42, HIV-0013095-2.11, HIV-16055-2.3, HIV-16845-2.22, Ce0393_C3, Ce1176_A3, Ce2010_F5, Ce1172_H1, Ce2060_G9, Ce703010054_2A2, BF1266.431a, 246F_C1G, 249M_B10, ZM247v1(Rev-), 7030102001E5(Rev-), 1394C9G1(Rev-), Ce704809221_1B3, CNE19, CNE20, CNE21, CNE17, CNE30, CNE52, CNE53, CNE58, MS208.A1, Q23.17, Q461.e2, Q769.d22, 0330.v4.c3, 0260.v5.c36, 191955_A11, 191084_B7-19, T257-31, 928-28, 263-8, T250-4, T251-18, T278-50, T255-34, 235-47, 620345.c01, C1080.c03, R2184.c04, R1166.c01, R3265.c06, C3347.c11, C4118.c09, CNE8, CNE5, BJOX009000.02.4, BJOX015000.11.5, BJOX010000.06.2, BJOX025000.01.1, BJOX028000.10.3, X1193_c1, X1254_c3, X2088_c9, X2131_C1_B5, P1981_C5_3, X1632_S2_B10, A07412M1.vrc12, 231965.c01, 3817.v2.c59, 6480.v4.c25, 6952.v1.c20, 6811.v7.c18, 89-F1225, 3301.v1.c24, 6041.v3.c23, 6540.v4.c1, 6545.v4.c1, 0815.v3.c3, and 3103.v3.c10, as shown in Table 5. In one embodiment, the virus panel comprises the 113 cross-clade isolates 62357, QH0692, REJO, TRJO, WITO, CNE17, CNE52, CNE58, 1394C9G, 249M B10, CAP210, CAP45, Du172.17, HIV-0013095, HIV-16055, HIV-16845, ZM135, ZM214, ZM249, 3817.v2.c59, BJOX 9000, BJOX 28000, 928-28, T211-9, T251-18, T255-34, T263-8, T278-50, 6535.3, SC422661.8, PVO.4, TRO.11, AC10.0.29, RHPA4259.7, WITO4160.33, CAAN5342.A2, WEAU_d15_410_5017, 1006_11_C3_1601, 1054_07_TC4_1499, 1056_10TA11_1826, 1012_11_TC21_3257, 6240_08_TA5_4622, 6244_13_B5_4576, SC05_8C11_2344, Du156.12, ZM197M.PB7, ZM233M.PB6, ZM233, 4C, ZM109F.PB4, HIV-001428-2.42, Ce0393_c3, Ce1176_A3, Ce2010_F5, Ce1172_H1, Ce2060_G9, Ce703010054_2A2, BF1266.431a, 246F C1G, ZM247v1(Rev-), 7030102001E5(Rev-), Ce704809221_1B3, CNE19, CNE20, CNE21, CNE53, MS208.A1, Q23.17, Q769.d22, 0330.v4.c3, 191955_A11, 191084 B7-19, T263-8, T255_34 at4C, T235-47, 620345.c01, C1080.c03, R2184.c04, A0742M1.vrc12, R1166.c01, R3265.c06, C3347.c11, C4118.c09, X1254_C3, X2088_C9, P1981_C5_3, X1632_S2_B10, 231965.c01, 6811.v7.c18, 89-F1_2_25, 3301.v1.c24, 6041.v3.c23, 6540.v4.c1, 6545.v4.c1, 0815.v3.c3, 3103.v3.c10, Du422.1, ZM53M.PB12, CNE30, Q461.c2, 0260.v5.c36, T257-31, T250-4, CNE8, CNE5, BJOX 15000, BJOX 10000, BJOX 25000, X1193_C1, P0402_C2_11, 3016.v5.c45, 6480.v4.c25, 6952.v1.c20, and 6041.v3.c23, as shown in Table 6. In one embodiment, a broadly neutralizing antibody is capable of neutralizing at least 4, 5, 6, or 7 of the cross-clade HIV isolates in the 7-member indicator virus panel. In one embodiment, a broadly neutralizing antibody is capable of neutralizing at least 6 of the cross-clade HIV isolates in the 7-member indicator virus panel. In one embodiment, a broadly neutralizing antibody is capable of neutralizing at least 6, 7, 8, 9, 10, 11, or 12 of the cross-clade HIV isolates in the 12-member indicator virus panel. In one embodiment, a broadly neutralizing antibody is capable of neutralizing at least 9 of the cross-clade HIV isolates in the 12-member indicator virus panel. In one embodiment, a broadly neutralizing antibody is capable of neutralizing at least 10 of the cross-clade HIV isolates in the 12-member indicator virus panel. In one embodiment, a broadly neutralizing antibody is capable of neutralizing at least 11 of the cross-clade HIV isolates in the 12-member indicator virus panel. In one embodiment, a broadly neutralizing antibody is capable of neutralizing 12 of the cross-clade HIV isolates in the 12-member indicator virus panel. In one embodiment, a broadly neutralizing antibody is capable of neutralizing at least about 50%, 60%, 70%, 80%, 90%, 95%, or 100% of cross-clade HIV isolates in the 106-member indicator virus panel. In one embodiment, a broadly neutralizing antibody is capable of neutralizing at least about 60% of cross-clade HIV isolates in the 106-member indicator virus panel. In one embodiment, a broadly neutralizing antibody is capable of neutralizing at least about 70% of cross-clade HIV isolates in the 106-member indicator virus panel. In one embodiment, a broadly neutralizing antibody is capable of neutralizing at least about 75% of cross-clade HIV isolates in the 106-member indicator virus panel. In one embodiment, a broadly neutralizing antibody is capable of neutralizing at least about 80% of cross-clade HIV isolates in the 106-member indicator virus panel. In one embodiment, a broadly neutralizing antibody is capable of neutralizing at least about 90% of cross-clade HIV isolates in the 106-member indicator virus panel. In one embodiment, a broadly neutralizing antibody is capable of neutralizing at least about 95% of cross-clade HIV isolates in the 106-member indicator virus panel. In one embodiment, a broadly neutralizing antibody is capable of neutralizing at least about 50%, 60%, 70%, 80%, 90%, 95%, or 100% of cross-clade HIV isolates in the 113-member indicator virus panel. In one embodiment, a broadly neutralizing antibody is capable of neutralizing at least about 60% of cross-clade HIV isolates in the 113-member indicator virus panel. In one embodiment, a broadly neutralizing antibody is capable of neutralizing at least about 70% of cross-clade HIV isolates in the 113-member indicator virus panel. In one embodiment, a broadly neutralizing antibody is capable of neutralizing at least about 75% of cross-clade HIV isolates in the 113-member indicator virus panel. In one embodiment, a broadly neutralizing antibody is capable of neutralizing at least about 80% of cross-clade HIV isolates in the 113-member indicator virus panel. In one embodiment, a broadly neutralizing antibody is capable of neutralizing at least about 90% of cross-clade HIV isolates in the 113-member indicator virus panel. In one embodiment, a broadly neutralizing antibody is capable of neutralizing at least about 95% of cross-clade HIV isolates in the 113-member indicator virus panel.

In one embodiment, the potency of neutralization by a broadly neutralizing antibody is expressed as the median IC₅₀ neutralization activity against a virus panel, for example the 7-virus panel, 12-virus pane, 106-virus panel, or 113-virus panel disclosed herein. In one embodiment, a broadly neutralizing antibody is capable of neutralizing at least about 4, 5, 6, or 7 of the cross-clade HIV isolates in the 7-member indicator virus panel with a median IC₅₀ equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, a broadly neutralizing antibody is capable of neutralizing at least 6 of the cross-clade HIV isolates in the 7-member indicator virus panel with a median IC₅₀ equal to or less than 0.05 microg/ml. In one embodiment, a broadly neutralizing antibody is capable of neutralizing at least 6, 7, 8, 9, 10, 11, or 12 of the cross-clade HIV isolates in the 12-member indicator virus panel with a median IC₅₀ equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, a broadly neutralizing antibody is capable of neutralizing at least 9 of the cross-clade HIV isolates in the 12-member indicator virus panel with a median IC₅₀ equal to or less than 0.05 microg/ml. In one embodiment, a broadly neutralizing antibody is capable of neutralizing at least 10 of the cross-clade HIV isolates in the 12-member indicator virus panel with a median IC₅₀ equal to or less than 0.05 microg/ml. In one embodiment, a broadly neutralizing antibody is capable of neutralizing at least 11 of the cross-clade HIV isolates in the 12-member indicator virus panel with a median IC₅₀ equal to or less than 0.05 microg/ml. In one embodiment, a broadly neutralizing antibody is capable of neutralizing at least about 50%, 60%, 70%, 80%, 90%, 95%, or 100% of cross-clade HIV isolates in the 106-member indicator virus panel with a median IC₅₀ equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, a broadly neutralizing antibody is capable of neutralizing at least about 70% of cross-clade HIV isolates in the 106-member indicator virus panel with a median IC₅₀ equal to or less than 0.05 microg/ml. In one embodiment, a broadly neutralizing antibody is capable of neutralizing at least about 75% of cross-clade HIV isolates in the 106-member indicator virus panel with a median IC₅₀ equal to or less than 0.05 microg/ml. In one embodiment, a broadly neutralizing antibody is capable of neutralizing at least about 80% of cross-clade HIV isolates in the 106-member indicator virus panel with a median IC₅₀ equal to or less than 0.05 microg/ml. In one embodiment, a broadly neutralizing antibody is capable of neutralizing at least about 50%, 60%, 70%, 80%, 90%, 95%, or 100% of cross-clade HIV isolates in the 113-member indicator virus panel with a median IC₅₀ equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, a broadly neutralizing antibody is capable of neutralizing at least about 70% of cross-clade HIV isolates in the 113-member indicator virus panel with a median IC₅₀ equal to or less than 0.05 microg/ml. In one embodiment, a broadly neutralizing antibody is capable of neutralizing at least about 75% of cross-clade HIV isolates in the 113-member indicator virus panel with a median IC₅₀ equal to or less than 0.05 microg/ml. In one embodiment, a broadly neutralizing antibody is capable of neutralizing at least about 80% of cross-clade HIV isolates in the 113-member indicator virus panel with a median IC₅₀ equal to or less than 0.05 microg/ml.

The term “IC₅₀” refers to the half maximal inhibitory concentration of an inhibitor, e.g., a broadly neutralizing antibody. For example, IC₅₀ is the concentration of an inhibitor, e.g., a broadly neutralizing antibody, where the response, e.g., infection by pseudovirus, is reduced by half.

The phrase “substantially similar,” or “substantially the same”, as used herein, denotes a sufficiently high degree of similarity between two numeric values (generally one associated with an antibody described herein and the other associated with a reference/comparator antibody) such that one of skill in the art would consider the difference between the two values to be of little or no biological and/or statistical significance within the context of the biological characteristic measured by said values (e.g., Kd values). The difference between said two values can be less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides described herein are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, Proc. Natl. Acad. Sci., 87:2264-2268 (1990), as modified in Karlin et al., Proc. Natl. Acad. Sci., 90:5873-5877 (1993), and incorporated into the NBLAST and XBLAST programs (Altschul et al., Nucleic Acids Res., 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Methods in Enzymology, 266:460-480 (1996)), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48): 444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100× (Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman (Advances in Applied Mathematics 2: 482 489 (1981)) to find the best segment of homology between two sequences. When using Bestfit or any other sequence alignment program to determine whether a particular sequence is, for instance, 95% identical to a reference sequence described herein, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies described herein do not abrogate the binding of the polypeptide or antibody containing the amino acid sequence, to the antigen(s). Methods of identifying nucleotide and amino acid conservative substitutions which do not eliminate antigen binding are well-known in the art (see, e.g., Brummell et al., Biochem. 32: 1180-1187 (1993); Kobayashi et al., Protein Eng. 12(10):879-884 (1999); and Burks et al., Proc. Natl. Acad. Sci. USA 94:412-417 (1997)).

Vectors that can be used include, but are not limited to, plasmids, bacterial vectors, and viral vectors. Viral vectors include cytomegalovirus (CMV) vectors. An advantage of these CMV vectors for use in therapeutic vaccine delivery is that they create a new CD8+ T cell epitope paradigm and induce more potent and enduring responses. It has been shown in animal models that vaccines based on these viral vectors can clear viral infections (Hansen, S. G. 2013. Science 340:1237874), and so these approaches have promise for a therapeutic vaccine, a setting in which tailored vaccines can be useful.

Other viral vectors can include poxvirus (vaccinia), including vaccinia Ankara and canarypox; adenoviruses, including adenovirus type 5 (Ad5); rubella; sendai virus; rhabdovirus; alphaviruses; and adeno-associated viruses. Alternatively, the vaccine antigens could be delivered as DNA, RNA or protein components of a vaccine.

As used herein, the terms “treatment” or “therapy” (as well as different forms thereof, including curative or palliative) refer to treatment of an infected person. As used herein, the term “treating” includes alleviating or reducing at least one adverse or negative effect or symptom of a condition, disease or disorder. This condition, disease or disorder can be HIV infection.

Terms such as “treating” or “treatment” or “to treat” or “alleviating” or “to alleviate” refer to therapeutic measures that cure, slow down, lessen symptoms of, and/or halt progression of a diagnosed pathologic condition or disorder, such as HIV or AIDS. Thus, those in need of treatment include those already diagnosed with or suspected of having the disorder. In certain embodiments, a subject is successfully “treated” for the disorder according to the methods described herein if the patient shows one or more of the following: a reduction in the number of or complete absence of viral load; a reduction in the viral burden; inhibition of or an absence of the virus into peripheral organs; relief of one or more symptoms associated with the disorder; reduced morbidity and mortality; improvement in quality of life; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

As used herein, the terms “prevention” or “prophylaxis” refer to preventing a subject from becoming infected with, or reducing the risk of a subject from becoming infected with, or halting transmission of, or the reducing the risk of transmission of a virus. Prophylactic or preventative measures refer to measures that prevent and/or slow the development of a targeted pathological condition or disorder. Thus, those in need of prophylactic or preventative measures include those prone to have the disorder and those in whom the disorder is to be prevented. In one embodiment, prevention encompasses passive immunization of a subject in need thereof comprising administering an effective amount of an antibody disclosed herein.

As employed above and throughout the disclosure the term “effective amount” refers to an amount effective, at dosages, and for periods of time necessary, to achieve the desired result with respect to the treatment of the relevant disorder, condition, or side effect. An “effective amount” can be determined empirically and in a routine manner, in relation to the stated purpose. It will be appreciated that the effective amount of components of the present invention will vary from patient to patient not only with the particular vaccine, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

The term “therapeutically effective amount” refers to an amount of an antibody, immunoconjugate, or other drug effective to “treat” a disease or disorder in a subject or mammal. To the extent an antibody can prevent growth and/or kill existing cells, it can be cytostatic and/or cytotoxic. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “subject,” “individual,” and “patient” are used interchangeably herein, and refer to an animal, for example a human, to whom treatment, including prophylactic treatment, with the antibody or pharmaceutical composition according to the present disclosure, is provided. In one embodiment, the subject, individual, or patient has been infected with HIV. In one embodiment, the subject, individual, or patient suffers from AIDS. In one embodiment, the subject, individual, or patient has been exposed to HIV. In one embodiment, the subject, individual, or patient is at risk of being exposed to HIV.

Administration “in combination with” one or more further therapeutic agents includes simultaneous (concurrent) or consecutive administration in any order.

The terms “pharmaceutically composition,” “pharmaceutical formulation,” “pharmaceutically acceptable formulation,” or “pharmaceutically acceptable composition” all of which are used interchangeably, refer to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. “Pharmaceutically acceptable” or “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The term “antiretroviral therapy” or “ART,” as used herein, refers to any of the therapies used to manage progression of a retrovirus (e.g., HIV) infection in a subject (e.g., a human), including, for example, nucleoside reverse transcriptase inhibitors (NRTIs), non-nucleoside reverse transcriptase inhibitors (NNRTIs), protease inhibitors (PIs), fusion inhibitors, entry inhibitors, maturation inhibitors, cellular inhibitors, integrase strand transfer inhibitors, and multi-class combinations. Such drugs include, but are not limited to, lamivudine and zidovudine, emtricitabine (FTC), zidovudine (ZDV), azidothymidine (AZT), lamivudine (3TC), zalcitabine, dideoxycytidine (ddC), tenofovir disoproxil fumarate (TDF), didanosine (ddl), stavudine (d4T), abacavir sulfate (ABC), etravirine, delavirdine (DLV), efavirenz (EFV), nevirapine (NVP), amprenavir (APV), tipranavir (TPV), indinavir (IDV), saquinavir, saquinavir mesylate (SQV), lopinavir (LPV), ritonavir (RTV), fosamprenavir calcium (FOS-APV), ritonavir, RTV, darunavir, atazanavir sulfate (ATV), nelfinavir mesylate (NFV), enfuvirtide, T-20, maraviroc and raltegravir. ART drugs can also include antibodies that target HIV proteins or cellular proteins associated with disease progression. Also included are immune-based therapies, such as IL-2, IL-12, and alpha-epibromide. Each of these drugs can be administered alone or in combination with any other ART drug or any HIV-specific neutralizing antibody, such as a broadly neutralizing antibody, which is incorporated by reference herein in its entirety for all purposes.

The term “reservoir activator,” as used herein, refers to an agent capable of activating a viral reservoir (e.g., an HIV reservoir). In one embodiment, a reservoir activator comprises a histone deacytelase (HDAC) inhibitor (e.g., romidepsin, vorinostat, and panobinostat), immunologic activator (e.g., cytokines and TLR agonists), or a dedicated small molecule drug.

The term “immunomodulator,” as used herein, refers to an agent, such as an antibody or peptide, which is capable of increasing, inducing, or extending an immune response (e.g., a cell-mediated immune response and/or a humoral immune response) when administered to a subject (e.g., a human, e.g., a human infected with HIV or at risk of an HIV infection or transmission). Immunomodulators include, but are not limited to immune checkpoint inhibitors, for example, a PD-1, PD-L1, LAG-3, or TIGIT antagonist. In one embodiment, an immunomodulator used in the methods described herein comprises an anti-PD-1 antibody, anti-PD-L1 antibody, anti-LAG3 antibody, or an anti-TIGIT antibody. An immunomodulator can be administered in conjunction with (e.g., prior to, concurrently with, or subsequent to, or within the context of a treatment regimen that includes the administration of a broadly neutralizing antibody described herein.

As used in this specification and the appended claims, the singular forms “a,” “an,” and “the” include plural referents unless the content clearly dictates otherwise. Thus, for example, reference to “a cell” includes a combination of two or more cells, and the like.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” as used herein when referring to a measurable value such as an amount, a temporal duration, and the like, is meant to encompass variations of up to ±20% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope described herein are approximations, the numerical values set forth in the specific examples are reported as precisely as possible. Any numerical value, however, inherently contain certain errors necessarily resulting from the standard deviation found in their respective testing measurements.

It is understood that wherever embodiments are described herein with the language “comprising,” otherwise analogous embodiments described in terms of “consisting of” and/or “consisting essentially of” are also provided.

### II. Engineered Anti-HIV Antibodies

In one aspect, provided herein is an engineered variant of the PGT-121 antibody that specifically binds to HIV Env (e.g., 92BR020 gp120). In one embodiment, the antibody specifically binds to a well-ordered HIV Env trimer. PGT-121 has been disclosed in U.S. Pat. No. 9,464,131, which is hereby incorporated by reference herein in its entirety.

In one aspect, provided herein is an engineered variant of the PGT-121 antibody that is capable of neutralizing the 92BR020 HIV isolate.

In one aspect, provided herein is an engineered variant of the PGT-121 antibody that is capable of neutralizing at least two cross-clade isolates of HIV. In one embodiment, the antibody is capable of neutralizing at least one clade B isolate and at least one clade C isolate.

In one embodiment, an antibody disclosed herein is a broadly neutralizing antibody.

In one embodiment, an antibody disclosed herein neutralizes 2, 3, 4, 5, 6, 7, 8, 9, or more HIV strains or pseudoviruses that belong to the same or different clades. In one embodiment, an antibody disclosed herein is capable of neutralizing HIV strains or pseudoviruses from at least two different clades. In one embodiment, an antibody disclosed herein is capable of neutralizing at least one clade B strain or pseudovirus and one clade C strain or pseudovirus. In one embodiment, an antibody disclosed herein is capable of neutralizing more than one clade B strain or pseudovirus and more than one clade C strain or pseudovirus. In one embodiment, an antibody disclosed herein is capable of neutralizing an HIV strain or pseudovirus from at least one, at least two, at least three, at least four, at least five, at least six, at least seven, at least eight, at least nine, at least ten, at least eleven, at least twelve, at least thirteen, at least fourteen, or all fifteen clades represented in the 106 member panel in Table 5. In one embodiment, an antibody disclosed herein is capable of neutralizing an HIV strain or pseudovirus from at least one, at least two, at least three, at least four, at least five, at least six, at least seven, at least eight, at least nine, at least ten, at least eleven, at least twelve, at least thirteen, at least fourteen, or all fifteen clades selected from the group consisting of clades A, A (T/F), AC, ACD, B, B (T/F), BC, C, C (T/F), CD, CRF01_AE, CRF01_AE (T/F), CRF02_AG, D, and G. In one embodiment, an antibody disclosed herein is capable of neutralizing an HIV strain or pseudovirus from at least one, at least two, at least three, at least four, at least five, at least six, at least seven, at least eight, at least nine, at least ten, or all eleven clades represented in the 113 member panel in Table 6. In one embodiment, an antibody disclosed herein is capable of neutralizing an HIV strain or pseudovirus from at least one, at least two, at least three, at least four, at least five, at least six, at least seven, at least eight, at least nine, at least ten, or all eleven clades selected from the group consisting of clades A, AC, ACD, AE, AG, B, BC, C, CD, D, G.

In one embodiments, an antibody disclosed herein has one or more improved properties. In one embodiment, an antibody disclosed herein has a higher binding affinity to HIV Env (e.g., 92BR020 gp120) than PGT-121. In one embodiment, an antibody disclosed herein has a higher binding affinity to HIV Env (e.g., 92BR020 gp120) at low pH (e.g., pH 4.0) than PGT-121. In one embodiment, an antibody disclosed herein has a higher binding affinity to HIV Env (e.g., 92BR020 gp120) at pH 5.0 than PGT-121. In one embodiment, an antibody disclosed herein has a higher binding affinity to HIV Env (e.g., 92BR020 gp120) at pH 4.5 than PGT-121. In one embodiment, an antibody disclosed herein has a higher binding affinity to HIV Env (e.g., 92BR020 gp120) at pH 4.0 than PGT-121. In one embodiment, an antibody disclosed herein has a higher binding affinity to HIV Env (e.g., 92BR020 gp120) at pH 3.5 than PGT-121. In one embodiment, an antibody disclosed herein has a higher binding affinity to HIV Env (e.g., 92BR020 gp120) at pH 3.0 than PGT-121. In one embodiment, an antibody disclosed herein has improved median neutralization IC₅₀ potency (i.e., lower median IC₅₀) than PGT-121. In one embodiment, an antibody disclosed herein has increased breadth of neutralization compared to PGT-121.

In one embodiment, an antibody disclosed herein is a broadly neutralizing anti-HIV Env antibody. In one embodiment, an antibody disclosed herein specifically binds to HIV Env. In one embodiment an antibody disclosed herein disclosed herein is capable of binding to HIV Env at pH 5.0. In one embodiment an antibody disclosed herein disclosed herein is capable of binding to HIV Env at pH 4.5. In one embodiment an antibody disclosed herein disclosed herein is capable of binding to HIV Env at pH 4.0. In one embodiment an antibody disclosed herein disclosed herein is capable of binding to HIV Env at pH 3.5. In one embodiment an antibody disclosed herein disclosed herein is capable of binding to HIV Env at pH 3.0. In one embodiment, an anti-HIV antibody disclosed herein is capable of binding to HIV Env in simulated vaginal fluid at pH 4.0. In one embodiment, an antibody disclosed herein specifically binds to a well-ordered HIV Env trimer. In one embodiment, an antibody disclosed herein is a monoclonal antibody. In one embodiment, an antibody disclosed herein is an F(ab) or F(ab′)2. In one embodiment, an antibody disclosed herein is a recombinant antibody, a chimeric antibody, an antibody fragment, a bispecific antibody, or a trispecific antibody.

In one embodiment, an antibody described herein comprises a VH, a VL, or a VH and VL comprising an amino acid sequence as shown in Table 1, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH, a VL, or a VH and VL comprising an amino acid sequence with at least about 80%, at least about 85%, at least about 90%, at least about 95%, at least about 97%, at least about 98%, at least about 99% or 100% identity to an amino acid sequence shown in Table 1, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120). In one embodiment, the antibody comprises a VH CDR1, VH CDR2, VH CDR3, VL CDR1, VL CDR2, and VL CDR3, wherein each of the VH CDR1, VH CDR2, VH CDR3, VL CDR1, VL CDR2, and VL CDR3 comprises an amino acid sequence as shown in Table 2.

In one embodiment, an antibody described herein comprises a VH CDR1, VH CDR2, VH CDR3, VL CDR1, VL CDR2, and VL CDR3, wherein one, two, three, four, five or six of the VH CDR1, VH CDR2, VH CDR3, VL CDR1, VL CDR2, and VL CDR3 comprise an amino acid sequence shown in Table 2, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein one, two, three, four, five or six of the VH CDR1, VH CDR2, VH CDR3, VL CDR1, VL CDR2, and VL CDR3 comprise an amino acid sequence with at least about 80%, at least about 85%, at least about 90%, at least about 95%, at least about 97%, at least about 98%, at least about 99% or 100% identity to an amino acid sequence shown in Table 2, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein one, two, three, four, five or six of the VH CDR1, VH CDR2, VH CDR3, VL CDR1, VL CDR2, and VL CDR3 comprise an amino acid sequence shown in Table 2 comprising 0, 1, 2, 3, 4, or 5 substitutions, deletions, or insertions, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR3 comprising an amino acid sequence as shown in Table 2, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR3 comprising an amino acid sequence with at least about 80%, at least about 85%, at least about 90%, at least about 95%, at least about 97%, at least about 98%, at least about 99% or 100% identity to an amino acid sequence shown in Table 2, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR3 comprising an amino acid sequence as shown in Table 2 comprising 0, 1, 2, 3, 4, or 5 substitutions, deletions, or insertions, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein each of the VH CDR1, VH CDR2, VH CDR3, VL CDR1, VL CDR2, and VL CDR3 comprises an amino acid sequence as shown in Table 2, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein each of the VH CDR1, VH CDR2, VH CDR3, VL CDR1, VL CDR2, and VL CDR3 comprises an amino acid sequence with at least about 80%, at least about 85%, at least about 90%, at least about 95%, at least about 97%, at least about 98%, at least about 99% or 100% identity to an amino acid sequence shown in Table 2, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein each of the VH CDR1, VH CDR2, VH CDR3, VL CDR1, VL CDR2, and VL CDR3 comprises an amino acid sequence as shown in Table 2 comprising 0, 1, 2, 3, 4, or 5 substitutions, deletions, or insertions, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein further comprises a VH FR1, VH FR2, VH FR3, VH FR4, VL FR1, VL FR2, VL FR3, and VL FR4, wherein one, two, three, four, five, six, seven or eight of the VH FR1, VH FR2, VH FR3, VH FR4, VL FR1, VL FR2, VL FR3, and VL FR4 comprise an amino acid sequence shown in Table 3.

In one embodiment, an antibody described herein further comprises a VH FR1, VH FR2, VH FR3, VH FR4, VL FR1, VL FR2, VL FR3, and VL FR4, wherein one, two, three, four, five, six, seven or eight of the VH FR1, VH FR2, VH FR3, VH FR4, VL FR1, VL FR2, VL FR3, and VL FR4 comprise an amino acid sequence with at least about 80%, at least about 85%, at least about 90%, at least about 95%, at least about 97%, at least about 98%, at least about 99% or 100% identity to an amino acid sequence shown in Table 3.

In one embodiment, an antibody described herein further comprises a VH FR1, VH FR2, VH FR3, VH FR4, VL FR1, VL FR2, VL FR3, and VL FR4, wherein one, two, three, four, five, six, seven or eight of the VH FR1, VH FR2, VH FR3, VH FR4, VL FR1, VL FR2, VL FR3, and VL FR4 comprise an amino acid sequence shown in Table 3 comprising 0, 1, 2, 3, 4, or 5 substitutions, deletions, or insertions.

In one embodiment, an antibody described herein further comprises a VH FR1, VH FR2, VH FR3, VH FR4, VL FR1, VL FR2, VL FR3, and VL FR4, wherein each of the VH FR1, VH FR2, VH FR3, VH FR4, VL FR1, VL FR2, VL FR3, and VL FR4 comprises an amino acid sequence as shown in Table 3.

In one embodiment, an antibody described herein further comprises a VH FR1, VH FR2, VH FR3, VH FR4, VL FR1, VL FR2, VL FR3, and VL FR4, wherein each of the VH FR1, VH FR2, VH FR3, VH FR4, VL FR1, VL FR2, VL FR3, and VL FR4 comprises an amino acid sequence with at least about 80%, at least about 85%, at least about 90%, at least about 95%, at least about 97%, at least about 98%, at least about 99% or 100% identity to an amino acid sequence shown in Table 3.

In one embodiment, an antibody described herein further comprises a VH FR1, VH FR2, VH FR3, VH FR4, VL FR1, VL FR2, VL FR3, and VL FR4, wherein each of the VH FR1, VH FR2, VH FR3, VH FR4, VL FR1, VL FR2, VL FR3, and VL FR4 comprises an amino acid sequence as shown in Table 3 comprising 0, 1, 2, 3, 4, or 5 substitutions, deletions, or insertions.

In one embodiment, an antibody described herein comprises a heavy chain, light chain, or a heavy and light chain comprising an amino acid sequence as shown in Table 2, wherein the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a heavy chain, light chain, or a heavy and light chain comprising an amino acid sequence with at least about 80%, at least about 85%, at least about 90%, at least about 95%, at least about 97%, at least about 98%, at least about 99% or 100% identity to an amino acid sequence shown in Table 1, wherein the antibody is not PGT-121. In one embodiment, the antibody is capable of neutralizing 92BR020. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120). In one embodiment, the antibody comprises a VH CDR1, VH CDR2, VH CDR3, VL CDR1, VL CDR2, and VL CDR3, wherein each of the VH CDR1, VH CDR2, VH CDR3, VL CDR1, VL CDR2, and VL CDR3 comprises an amino acid sequence as shown in Table 2.

Also provided herein are polypeptides that comprise an amino acid sequence having at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence, or is identical to the sequences listed in Tables 1, 2, 3, and 4, wherein the polypeptide is not a PGT-121 polypeptide.

TABLE 1
Variable heavy chain (VH) and light chain (VL) domains.

| | |
|---|---|
| PGT-121 VH | QMQLQESGPGLVKPSETLSLTCSVSGASISDSYWSWIRRSPGKGLEWIG |
| | YVHKSGDTNYSPSLKSRVNLSLDTSKNQVSLSLVAATAADSGKYYCA |
| | RTLHGRRWGIVAFNEWFTYFYMDVWGNGTQVTVSS (SEQ ID NO: 1) |
| ePGT121.15.H1 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVAFNEWFTYFYWEVWGNGTQVTVSS (SEQ ID NO: 3) |
| ePGT121.15.H2 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVAFNEWFTYFYWEVWGKGTQVTVSS (SEQ ID NO: 4) |
| ePGT121.15.H3 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVAFNELFTYFYWEVWGNGTQVTVSS (SEQ ID NO: 5) |
| ePGT121.15.H4 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVAFNELFTYFYWEVWGKGTQVTVSS (SEQ ID NO: 6) |
| ePGT121.15.H5 VH | QVQLQESGPGLVKPGETLSLTCSVSGASISSSYWSWLRETPGKGLEWIG |
| | YTHHSGDTNYAPSLKSRVHLGLHPSKNQVSLSLTSVTAADTAVYYCA |
| | RTLHGRRWGVVAFNELFTYFYWEVWGKGTQVTVSS (SEQ ID NO: 7) |
| ePGT121.15.H6 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVAFNEFFTYFYWEVWGKGTQVTVSS (SEQ ID NO: 8) |
| ePGT121.15.H7 VH | QMQLQESGPGLVKPGETLSLTCSVSGASISSSYWSWLRETPGKGLEWI |
| | GYTHHSGDTNYAPSLKSRVHLGLHPSKNQVSLKLKSVTAADTAVYYC |
| | ARTLHGRRWGVVAFNELFTYFYWEVWGKGTQVTVSS (SEQ ID NO: 9) |
| ePGT121.12.H1 VH | QMQLQESGPGLVKPSETLSLTCSVSGASISDSYWSWIRRSPGKGLEWIG |
| | YVHKSGDTNYSPSLKSRVNLSLDTSKNQVSLSLVAATAADSGKYYCA |
| | RTLHGRRWGIVAFNEWFTYFYMDVWGNGTQVTVSS (SEQ ID NO: 45) |
| ePGT121.13.H1 VH | QMQLQESGPGLVKPGETLSLTCSVSGASISDSYWSWLRETPGKGLEWI |
| | GYVHKSGDTNYAPSLKSRVNLGLDTSKNQVSLSLVAATAADSGKYYC |
| | ARTLHGRRWGVVAFNEWFTYFYWDVWGNGTQVTVSS (SEQ ID |
| | NO: 47) |
| ePGT121.14.H1 VH | QMQLQESGPGLVKPGETLSLTCSVSGASISSSYWSWLRETPGKGLEWI |
| | GYTHEISGDTNYAPSLKSRVHLGLHPSKNQVSLSLVAATAADSGKYYC |
| | ARTLHGRRIYGVVAFNEWFTYFYWEVWGNGTQVTVSS (SEQ ID |
| | NO :49) |
| ePGT121.14.H2 VH | QMQLQESGPGLVKPGETLSLTCSVSGASISSSYWSWLRETPGKGLEWI |
| | GYTHEISGDTNYAPSLKSRVHLGLHPSKNQVSLSLVAATAADSGKYYC |
| | ARTLHGRRIYGVVAFNEWFTYFYWEVWGKGTQVTVSS (SEQ ID |
| | NO: 50) |
| ePGT121.14.H3 VH | QMQLQESGPGLVKPGETLSLTCSVSGASISSSYWSWLRETPGKGLEWI |
| | GYTHEISGDTNYAPSLKSRVHLGLHPSKNQVSLSLVAATAADSGKYYC |
| | ARTLHGRRIYGVVAFGEWFTYFYWEVWGNGTQVTVSS (SEQ ID |
| | NO: 51) |
| ePGT121.14.H4 VH | QMQLQESGPGLVKPGETLSLTCSVSGASISSSYWSWLRETPGKGLEWI |
| | GYTHEISGDTNYAPSLKSRVHLGLHPSKNQVSLSLVAATAADSGKYYC |
| | ARTLHGRRCYGVVAFNECFTYFYWEVWGNGTQVTVSS (SEQ ID |
| | NO: 52) |
| ePGT121.14.H5 VH | QMQLQESGPGLVKPGETLSLTCSVSGASISSSYWSWLRETPGKGLEWI |
| | GYTHEISGDTNYAPSLKSRVHLGLHPSKNQVSLSLVAATAADSGKYYC |
| | ARTLHGRRIYGVVAFNELFTYFYWEVWGNGTQVTVSS (SEQ ID |
| | NO: 53) |
| ePGT121.14.H6 VH | QMQLQESGPGLVKPGETLSLTCSVSGASISSSYWSWLRETPGKGLEWI |
| | GYTHEISGDTNYAPSLKSRVHLGLHPSKNQVSLSLTSVTAADTAVYYC |
| | ARTLHGRRIYGVVAFNEWFTYFYWEVWGNGTQVTVSS (SEQ ID |
| | NO: 54) |
| ePGT121.18.H1 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRIYGVVAFNEYYTYFYWPTWGKGTQVTVSS (SEQ ID NO: |
| | 65) |
| ePGT121.18.H2 VH | QMQLQESGPGLVKPGETLSLTCSVSGASISSSYWSWLRETPGKGIEWIG |
| | YTHEISGDTNYAPSLKSRVHLGLHPEKNQVSLKLTSVTAADTAVYYCA |
| | RTLHGRRIYGVVAGNEYYTYFYWPTWGKGTQVTVSS (SEQ ID |
| | NO: 112) |
| ePGT121.18.H3 VH | QMQLQESGPGLVKPGETLSLTCSVSGASISSSYWSWLRETPGKGLEWI |
| | GYTHEISGDTNYAPSLKSRVHLGLHPEKNQVSLKLTSVTAADTAVYYC |
| | ARTLHGRRIYGVVAGGEYYTYFYWPTWGKGTQVTVSS (SEQ ID |
| | NO: 113) |
| ePGT121.18.H4 VH | QMQLQESGPGLVKPGETLSLTCSVSGASISSSYWSWLRETPGKGIEWIG |
| | YTHEISGDTNYAPSLKSRVHLGLHPSKNQVSLKLTSVTAADTAVYYCA |
| | RTLHGRRIYGVVAGNEFYTYFYWPTWGKGTQVTVSS (SEQ ID |
| | NO: 114) |
| ePGT121.18.H5 VH | QMQLQESGPGLVKPGETLSLTCSVSGASISSSYWSWLRETPGKGLEWI |
| | GYTHEISGDTNYAPSLKSRVHLGLHPEKNQVSLSLTSVTAADTAVYYC |
| | ARTLHGRRIYGVVAGNEYYTYFYWPTWGKGIQVTVSS (SEQ ID |
| | NO: 115) |
| ePGT121.18.H6 VH | QMQLQESGPGLVKPGETLSLTCSVSGASISSSYWSWLRETPGKGIEWIG |
| | YTHEISGDTNYAPSLKSRVHLGLHPSKNQVSLSLTSVTAADTAVYYCA |
| | RTLHGRRIYGVVAGNEFYTYFYWPTWGKGTKVTVSS (SEQ ID |
| | NO: 116) |
| ePGT121.18.H7 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVAGGEFYTYFYWPTWGKGTQVTVSS (SEQ ID |
| | NO: 117) |
| ePGT121.18.H8 VH | QMQLQESGPGLVKPGETLSLTCSVSGASISSSYWSWLRETPGKGLEWI |
| | GYTHHSGDTNYAPSLKSRVHLGLHPSKNQVSLKLTSVTAADTAVYYC |
| | ARTLHGRRWGVVAGNEYYTYFYWPTWGKGIQVTVSS (SEQ ID |
| | NO: 118) |
| ePGT121.18.H9 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVEGGEYYTYFYWPTWGKGTQVTVSS (SEQ ID |
| | NO: 119) |
| ePGT121.18.H10 VH | QMQLQESGPGLVKPGETLSLTCSVSGASISSSYWSWLRETPGKGLEWI |
| | GYTHHSGDTNYAPSLKSRVHLGLHPEKNQVSLSLTSVTAADTAVYYC |
| | ARTLHGRRWGVVAGNEFYTYFYWPTWGKGTQVTVSS (SEQ ID |
| | NO: 120) |
| ePGT121.18.H11 VH | QMQLQESGPGLVKPGETLSLTCSVSGASISSSYWSWLRETPGKGIEWIG |
| | YTHHSGDTNYAPSLKSRVHLGLHPSKNQVSLSLTSVTAADTAVYYCA |
| | RTLHGRRWGVVEGGEYYTYFYWPTWGKGTKVTVSS (SEQ ID |
| | NO: 121) |
| ePGT121.18.H12 VH | QMQLQESGPGLVKPGETLSLTCSVSGASISSSYWSWLRETPGKGVEWI |
| | GYTHHSGDTNYAPSLKSRVHLGLHPEKNQVSLSLTSVTAADTAVYYC |
| | ARTLHGRRWGVVAGNEFYTYFYWPTWGKGIQVTVSS (SEQ ID |
| | NO: 122) |
| ePGT121.18.H13 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVAGNEFYTYFYWPTWGKGIQVTVSS (SEQ ID |
| | NO: 123) |
| ePGT121.18.H14 VH | QMQLQESGPGLVKPGETLSLTCSVSGASISSSYWSWLRETPGKGIEWIG |
| | YTHHSGDTNYAPSLKSRVHLGLHPSKNQVSLSLTSVTAADTAVYYCA |
| | RTLHGRRWGVVAGGEYYTYFYWPTWGKGTQVTVSS (SEQ ID |
| | NO: 124) |
| ePGT121.18.H15 VH | QMQLQESGPGLVKPGETLSLTCSVSGASISSSYWSWLRETPGKGLEWI |
| | GYTHHSGDTNYAPSLKSRVHLGLHPSKNQVSLSLTSVTAADTAVYYC |
| | ARTLHGRRWGVVAGGEYYTYFYWPTWGKGIQVTVSS (SEQ ID |
| | NO: 125) |
| ePGT121.18.H16 VH | QMQLQESGPGLVKPGETLSLTCSVSGASISSSYWSWLRETPGKGLEWI |
| | GYTHHSGDTNYAPSLKSRVHLGLHPSKNQVSLKLTSVTAADTAVYYC |
| | ARTLHGRRWGVVAGNEFYTYFYWPTWGKGTKVTVSS (SEQ ID |
| | NO: 126) |
| ePGT121.18*H17 VH | QMQLQESGPGLVKPGETLSLTCSVSGASISSSYWSWLRETPGKGLEWI |
| | GYTHHSGDTNYAPSLKSRVTIGLDPSKNQVSLSLTSVTAADTAVYYCA |
| | RTLHGRRWGVVAFNEYYTYFYWPTWGKGTQVTVSS (SEQ ID NO: 69) |
| ePGT121.18.H18 VH | QMQLQESGPGLVKPGETLSLTCSVSGASISSSYWSWLRETPGKGLEWI |
| | GYTHHSGDTNYAPSLKSRVTISLDPSKNQVSLSLTSVTAADTAVYYCA |
| | RTLHGRRWGVVAFNEYYTYFYWPTWGKGTQVTVSS (SEQ ID NO: 70) |
| PGT-121 VL | SDISVAPGETARISCGEKSLGSRAVQWYQHRAGQAPSLIIYNNQDRPSG |
| | IPERFSGSPDSPFGTTATLTITSVEAGDEADYYCHIWDSRVPTKWVFGG |
| | GTTLTVL (SEQ ID NO: 2) |
| ePGT121.15_L1 VL | SDISVAPGETVRISCGGESIGSRAVQWYQHRAGQAPKLIIYNNQDRPPG |
| | IPERFSGSPDIDFGTTATLTITNVEAGDEATYYCHIWDSRRPTNWVFGG |
| | GTTLTVL (SEQ ID NO: 10) |
| ePGT121.15_L2 VL | SYVLTQPPSVSVAPGETVRISCGGESIGSRAVQWYQHRAGQAPKLITYN |
| | NQDRPPGIPERFSGSPDIDFGTTATLTITNVEAGDEATYYCHIWDSRRPT |
| | NWVFGGGTTLTVL (SEQ ID NO: 11) |
| ePGT121.12_L4 VL | SDISVAPGETARISCGGESLGSRAVQWYQHRAGQAPSLIIYNNQDRPSG |
| | IPERFSGSPDIAFGTTATLTITSVEAGDEADYYCHIWDSRRPTNWVFGG |
| | GTTLTVL (SEQ ID NO: 46) |
| ePGT121.13_L1 VL | SDISVAPGETVRISCGGESIGSRAVQWYQHRAGQAPKLIIYNNQDRPPG |
| | IPERFSGSPDIDFGTTATLTITNVEAGDEATYYCHIWDSRRPTNWVFGG |
| | GTTLTVL (SEQ ID NO: 48) |
| ePGT121.14_L1 VL | SDISVAPGETVRISCGGESIGSRAVQWYQHRAGQAPKLIIYNNQDRPPG |
| | IPERFSGSPDIDFGTTATLTITNVEAGDEATYYCHIWDSRRPTNWVFGG |
| | GTTLTVL (SEQ ID NO: 55) |
| ePGT121.17_L1 VL | SDISVAPGETVRITCGGESIGSRAVQWYQHRPGQAPRLIIYNNQDRPPGI |
| | PERFSGSPDIDFGTTATLTISNVEAGDEATYYCHIWDSRRPTNWELGPG |
| | TTLTVL (SEQ ID NO: 66) |
| ePGT121.17_L2 VL | SDISVAPGETVRISCGGESIGSRAVQWYQHRAGQAPRLIIYNNQDRPPGI |
| | PERFSGSPDIDFGTTATLTITNVEAGDEATYYCHIWDSRRPTNWELGPG |
| | TTLTVL (SEQ ID NO: 127) |
| ePGT121.17_L3 VL | SDLSVAPGETVRITCGGESIGSRAVQWYQHRPGQAPRLIIYNNQDRPPG |
| | IPERFSGSPDIDFGTTATLTISNVEAGDEATYYCHIWDSRRPTNWVFEPG |
| | TTLTVL (SEQ ID NO: 128) |
| ePGT121.17_L4 VL | SDLSVAPGETVRISCGGESIGSRAVQWYQHRAGQAPRLIIYNNQDRPPG |
| | IPERFSGSPDIDFGTTATLTITNVEAGDEATYYCHIWDSRRPTNWVFEP |
| | GTTLTVL (SEQ ID NO: 129) |
| ePGT121.17_L5 VL | SDLSVAPGETVRITCGGESIGSRAVQWYQHRPGQAPKLIIYNNQDRPPG |
| | IPERFSGSPDIDFGTTATLTISRVEAGDEATYYCHVWDSRRPTNWELGP |
| | GTTLTVL (SEQ ID NO: 130) |
| ePGT121.17_L6 VL | SDLSVAPGETVRISCGGESIGSRAVQWYQHRAGQAPKLIIYNNQDRPPG |
| | IPERFSGSPDIDFGTTATLTITRVEAGDEATYYCHVWDSRRPTNWELGP |
| | GTTLTVL (SEQ ID NO: 131) |
| ePGT121.17_L7 VL | SDI SVAPGETVRITCGGESIGSRAVQWYQHRPGQAPRLIIYNNQDRPPGI |
| | PERFSGSPDLDFGTTATLTISNVEAGDEATYYCHVWDSRRPTNWELGP |
| | GTTLTVL (SEQ ID NO: 132) |
| ePGT121.17_L8 VL | SDISVAPGETVRISCGGESIGSRAVQWYQHRAGQAPRLIIYNNQDRPPGI |
| | PERFSGSPDLDFGTTATLTITNVEAGDEATYYCHVWDSRRPTNWELGP |
| | GTTLTVL (SEQ ID NO: 133) |
| ePGT121.17_L9 VL | SDISVAPGETVRITCGGESIGSRAVQWYQHRPGQAPRLIIYNNQDRPPGI |
| | PERFSGSPDIDFGTTATLTISNVEAGDEATYYCHVWDSRRPTQWVLEG |
| | GTTLTVL (SEQ ID NO: 134) |
| ePGT121.17_L10 VL | SDISVAPGETVRISCGGESIGSRAVQWYQHRAGQAPRLIIYNNQDRPPGI |
| | PERFSGSPDIDFGTTATLTITNVEAGDEATYYCHVWDSRRPTQWVLEG |
| | GTTLTVL (SEQ ID NO: 135) |
| ePGT121.17_L11 VL | SDLSVAPGETVRITCGGESIGSRAVQWYQHRPGQAPRLIIYNNQDRGPG |
| | IPERFSGSPDIDFGTTATLTISNVEAGDEATYYCHIWDSRRPTNWELGG |
| | GTTLTVL (SEQ ID NO: 136) |
| ePGT121.17_L12 VL | SDLSVAPGETVRISCGGESIGSRAVQWYQHRAGQAPRLIIYNNQDRGP |
| | GIPERFSGSPDIDFGTTATLTITNVEAGDEATYYCHIWDSRRPTNWELG |
| | GGTTLTVL (SEQ ID NO: 137) |
| ePGT121.17_L13 VL | SDISVAPGETVRITCGGESIGSRAVQWYQQRPGQAPRLIIYNNQDRPPGI |
| | PERFSGSPDIDFGTTATLTISNVEAGDEATYYCHVWDSRRPTNWELGP |
| | GTTLTVL (SEQ ID NO: 138) |
| ePGT121.17_L14 VL | SDISVAPGETVRISCGGESIGSRAVQWYQQRAGQAPRLIIYNNQDRPPGI |
| | PERFSGSPDIDFGTTATLTITNVEAGDEATYYCHVWDSRRPTNWELGP |
| | GTTLTVL (SEQ ID NO: 139) |

TABLE 2
VH and VL CDR sequences. The CDRs have been determined according to Kabat.

| | CDR1 | CDR2 | CDR3 |
|---|---|---|---|
| PGT-121 VH | DSYWS | YVHKSGDTNYSPSLKS | TLHGRRIYGIVAFNEWFTYF |
| | (SEQ ID NO: 12) | (SEQ ID NO: 13) | YMDV (SEQ ID NO: 14) |
| ePGT121.15.H1 VH | SSYWS | YTHHSGDTNYAPSLKS | TLHGRRIYGVVAFNEWFTYF |
| | (SEQ ID NO: 18) | (SEQ ID NO: 19) | YWEV (SEQ ID NO: 20) |
| ePGT121.15.H2 VH | SSYWS | YTHHSGDTNYAPSLKS | TLHGRRIYGVVAFNEWFTYF |
| | (SEQ ID NO: 18) | (SEQ ID NO: 19) | YWEV (SEQ ID NO: 21) |
| ePGT121.15.H3 VH | SSYWS | YTHHSGDTNYAPSLKS | TLHGRRIYGVVAFNELFTYF |
| | (SEQ ID NO: 18) | (SEQ ID NO: 19) | YWEV (SEQ ID NO: 21) |
| ePGT121.15.H4 VH | SSYWS | YTHHSGDTNYAPSLKS | TLHGRRIYGVVAFNELFTYF |
| | (SEQ ID NO: 18) | (SEQ ID NO: 19) | YWEV (SEQ ID NO: 21) |
| ePGT121.15.H5 VH | SSYWS | YTHHSGDTNYAPSLKS | TLHGRRIYGVVAFNELFTYF |
| | (SEQ ID NO: 18) | (SEQ ID NO: 19) | YWEV (SEQ ID NO: 21) |
| ePGT121.15.H6 VH | SSYWS | YTHHSGDTNYAPSLKS | TLHGRRIYGVVAFNEFFTYF |
| | (SEQ ID NO: 18) | (SEQ ID NO: 19) | YWEV (SEQ ID NO: 22) |
| ePGT121.15.H7 VH | SSYWS | YTHHSGDTNYAPSLKS | TLHGRRIYGVVAFNELFTYF |
| | (SEQ ID NO: 18) | (SEQ ID NO: 19) | YWEV (SEQ ID NO: 21) |
| ePGT121.12.H1 VH | DSYWS | YVHKSGDTNYSPSLKS | TLHGRRIYGIVAFNEWFTYF |
| | (SEQ ID NO: 12) | (SEQ ID NO: 13) | YMDV (SEQ ID NO: 14) |
| ePGT121.13.H1 VH | DSYWS | YVHKSGDTNYAPSLKS | TLHGRRIYGVVAFNEWFTYF |
| | (SEQ ID NO: 12) | (SEQ ID NO: 61) | YWDV (SEQ ID NO: 62) |
| ePGT121.14.H1 VH | SSYWS | YTHHSGDTNYAPSLKS | TLHGRRIYGVVAFNEWFTYF |
| | (SEQ ID NO: 18) | (SEQ ID NO: 19) | YWEV (SEQ ID NO: 20) |
| ePGT121.14.H2 VH | SSYWS | YTHHSGDTNYAPSLKS | TLHGRRIYGVVAFNEWFTYF |
| | (SEQ ID NO: 18) | (SEQ ID NO: 19) | YWEV (SEQ ID NO: 20) |
| ePGT121.14.H3 VH | SSYWS | YTHHSGDTNYAPSLKS | TLHGRRIYGVVAFGEWFTYF |
| | (SEQ ID NO: 18) | (SEQ ID NO: 19) | YWEV (SEQ ID NO: 59) |
| ePGT121.14.H4 VH | SSYWS | YTHHSGDTNYAPSLKS | TLHGRRCYGVVAFNECFTYF |
| | (SEQ ID NO: 18) | (SEQ ID NO: 19) | YWEV (SEQ ID NO: 60) |
| ePGT121.14.H5 VH | SSYWS | YTHHSGDTNYAPSLKS | TLHGRRIYGVVAFNELFTYF |
| | (SEQ ID NO: 18) | (SEQ ID NO: 19) | YWEV (SEQ ID NO: 21) |
| ePGT121.14.H6 VH | SSYWS | YTHHSGDTNYAPSLKS | TLHGRRIYGVVAFNEWFTYF |
| | (SEQ ID NO: 18) | (SEQ ID NO: 19) | YWEV (SEQ ID NO: 20) |
| ePGT121.18.H1 VH | SSYWS | YTHHSGDTNYAPSLKS | TLHGRRIYGVVAFNEYYTYF |
| | (SEQ ID NO: 18) | (SEQ ID NO: 19) | YWPT (SEQ ID NO: 67) |
| ePGT121.18.H1 VH | SSYWS | YTHHSGDTNYAPSLKS | TLHGRRIYGVVAGNEYYTYF |
| | (SEQ ID NO: 18) | (SEQ ID NO: 19) | YWPT (SEQ ID NO: 140) |
| ePGT121.18.H3 VH | SSYWS | YTHHSGDTNYAPSLKS | TLHGRRIYGVVAGGEYYTYF |
| | (SEQ ID NO: 18) | (SEQ ID NO: 19) | YWPT (SEQ ID NO: 141) |
| ePGT121.18.H4 VH | SSYWS | YTHHSGDTNYAPSLKS | TLHGRRIYGVVAGNEFYTYF |
| | (SEQ ID NO: 18) | (SEQ ID NO: 19) | YWPT (SEQ ID NO: 142) |
| ePGT121.18.H5 VH | SSYWS | YTHHSGDTNYAPSLKS | TLHGRRIYGVVAGNEYYTYF |
| | (SEQ ID NO: 18) | (SEQ ID NO: 19) | YWPT (SEQ ID NO: 140) |
| ePGT121.18.H6 VH | SSYWS | YTHHSGDTNYAPSLKS | TLHGRRIYGVVAGNEFYTYF |
| | (SEQ ID NO: 18) | (SEQ ID NO: 19) | YWPT (SEQ ID NO: 142) |
| ePGT121.18.H7 VH | SSYWS | YTHHSGDTNYAPSLKS | TLHGRRIYGVVAGGEFYTYF |
| | (SEQ ID NO: 18) | (SEQ ID NO: 19) | YWPT (SEQ ID NO: 143) |
| ePGT121.18.H8 VH | SSYWS | YTHHSGDTNYAPSLKS | TLHGRRIYGVVAGNEYYTYF |
| | (SEQ ID NO: 18) | (SEQ ID NO: 19) | YWPT (SEQ ID NO: 140) |
| ePGT121.18.H9 VH | SSYWS | YTHHSGDTNYAPSLKS | TLHGRRIYGVVEGGEYYTYF |
| | (SEQ ID NO: 18) | (SEQ ID NO: 19) | YWPT (SEQ ID NO: 144) |
| ePGT121.18.H10 VH | SSYWS | YTHHSGDTNYAPSLKS | TLHGRRIYGVVAGNEFYTYF |
| | (SEQ ID NO: 18) | (SEQ ID NO: 19) | YWPT (SEQ ID NO: 142) |
| ePGT121.18.H11 VH | SSYWS | YTHHSGDTNYAPSLKS | TLHGRRIYGVVEGGEYYTYF |
| | (SEQ ID NO: 18) | (SEQ ID NO: 19) | YWPT (SEQ ID NO: 144) |
| ePGT121.18.H12 VH | SSYWS | YTHHSGDTNYAPSLKS | TLHGRRIYGVVAGNEFYTYF |
| | (SEQ ID NO: 18) | (SEQ ID NO: 19) | YWPT (SEQ ID NO: 142) |
| ePGT121.18.H13 VH | SSYWS | YTHHSGDTNYAPSLKS | TLHGRRIYGVVAGNEFYTYF |
| | (SEQ ID NO: 18) | (SEQ ID NO: 19) | YWPT (SEQ ID NO: 142) |
| ePGT121.18.H14 VH | SSYWS | YTHHSGDTNYAPSLKS | TLHGRRIYGVVAGNEYYTYF |
| | (SEQ ID NO: 18) | (SEQ ID NO: 19) | YWPT (SEQ ID NO: 140) |
| ePGT121.18.H15 VH | SSYWS | YTHHSGDTNYAPSLKS | TLHGRRIYGVVAGNEYYTYF |
| | (SEQ ID NO: 18) | (SEQ ID NO: 19) | YWPT (SEQ ID NO: 140) |
| ePGT121.18.H16 VH | SSYWS | YTHHSGDTNYAPSLKS | TLHGRRIYGVVAGNEFYTYF |
| | (SEQ ID NO: 18) | (SEQ ID NO: 19) | YWPT (SEQ ID NO: 142) |
| ePGT121.18.H17 VH | SSYWS | YTHHSGDTNYAPSLKS | TLHGRRIYGVVAFNEYYTYF |
| | (SEQ ID NO: 18) | (SEQ ID NO: 19) | YWPT (SEQ ID NO: 67) |
| ePGT121.18.H18 VH | SSYWS | YTHHSGDTNYAPSLKS | TLHGRRIYGVVAFNEYYTYF |
| | (SEQ ID NO: 18) | (SEQ ID NO: 19) | YWPT (SEQ ID NO: 67) |
| PGT121_VL | GEKSLGSRAVQ | NNQDRPS | HIWDSRVPTKWV |
| | (SEQ ID NO: 15) | (SEQ ID NO: 16) | (SEQ ID NO: 17) |
| ePGT121.15_L1 VL | GGESIGSRAVQ | NNQDRPP | HIWDSRRPTNWV |
| | (SEQ ID NO: 23) | (SEQ ID NO: 24) | (SEQ ID NO: 25) |
| ePGT121.15_L2 VL | GGESIGSRAVQ | NNQDRPP | HIWDSRRPTNWV |
| | (SEQ ID NO: 23) | (SEQ ID NO: 24) | (SEQ ID NO: 25) |
| ePGT121.12_L4 VL | GGESLGSRAVQ | NNQDRPS | HIWDSRRPTNWV |
| | (SEQ ID NO: 63) | (SEQ ID NO: 16) | (SEQ ID NO: 25) |
| ePGT121.13_L1 VL | GGESIGSRAVQ | NNQDRPP | HIWDSRRPTNWV |
| | (SEQ ID NO: 23) | (SEQ ID NO: 24) | (SEQ ID NO: 25) |
| ePGT121.14_L1 VL | GGESIGSRAVQ | NNQDRPP | HIWDSRRPTNWV |
| | (SEQ ID NO: 23) | (SEQ ID NO: 24) | (SEQ ID NO: 25) |
| ePGT121.17_L1 VL | GGESIGSRAVQ | NNQDRPP | HIWDSRRPTNWE |
| | (SEQ ID NO: 23) | (SEQ ID NO: 24) | (SEQ ID NO: 68) |
| ePGT121.17_L2 VL | GGESIGSRAVQ | NNQDRPP | HIWDSRRPTNWE |
| | (SEQ ID NO: 23) | (SEQ ID NO: 24) | (SEQ ID NO: 68) |
| ePGT121.17_L3 VL | GGESIGSRAVQ | NNQDRPP | HIWDSRRPTNWV |
| | (SEQ ID NO: 23) | (SEQ ID NO: 24) | (SEQ ID NO: 25) |
| ePGT121.17_L4 VL | GGESIGSRAVQ | NNQDRPP | HIWDSRRPTNWV |
| | (SEQ ID NO: 23) | (SEQ ID NO: 24) | (SEQ ID NO: 25) |
| ePGT121.17_L5 VL | GGESIGSRAVQ | NNQDRPP | HVWDSRRPTNWE |
| | (SEQ ID NO: 23) | (SEQ ID NO: 24) | (SEQ ID NO: 146) |
| ePGT121.17_L6 VL | GGESIGSRAVQ | NNQDRPP | HVWDSRRPTNWE |
| | (SEQ ID NO: 23) | (SEQ ID NO: 24) | (SEQ ID NO: 146) |
| ePGT121.17_L7 VL | GGESIGSRAVQ | NNQDRPP | HVWDSRRPTNWE |
| | (SEQ ID NO: 23) | (SEQ ID NO: 24) | (SEQ ID NO: 146) |
| ePGT121.17_L8 VL | GGESIGSRAVQ | NNQDRPP | HVWDSRRPTNWE |
| | (SEQ ID NO: 23) | (SEQ ID NO: 24) | (SEQ ID NO: 146) |
| ePGT121.17_L9 VL | GGESIGSRAVQ | NNQDRPP | HVWDSRRPTQWV |
| | (SEQ ID NO: 23) | (SEQ ID NO: 24) | (SEQ ID NO: 147) |
| ePGT121.17_L10 VL | GGESIGSRAVQ | NNQDRPP | HVWDSRRPTQWV |
| | (SEQ ID NO: 23) | (SEQ ID NO: 24) | (SEQ ID NO: 147) |
| ePGT121.17_L11 VL | GGESIGSRAVQ | NNQDRGP (SEQ ID | HIWDSRRPTNWE |
| | (SEQ ID NO: 23) | NO: 145) | (SEQ ID NO: 68) |
| ePGT121.17_L12 VL | GGESIGSRAVQ | NNQDRGP (SEQ ID | HIWDSRRPTNWE |
| | (SEQ ID NO: 23) | NO: 145) | (SEQ ID NO: 68) |
| ePGT121.17_L13 VL | GGESIGSRAVQ | NNQDRPP | HVWDSRRPTNWE |
| | (SEQ ID NO: 23) | (SEQ ID NO: 24) | (SEQ ID NO: 146) |
| ePGT121.17_L14 VL | GGESIGSRAVQ | NNQDRPP | HVWDSRRPTNWE |
| | (SEQ ID NO: 23) | (SEQ ID NO: 24) | (SEQ ID NO: 146) |

TABLE 3
VH and VL Framework (FR) sequences.
The FRs have been determined according to Kabat.

| | FR1 | FR2 | FR3 | FR4 |
|---|---|---|---|---|
| PGT-121 VH | QMQLQESGPGL | WIRRSPGKGLE | RVNLSLDTSKN | YMDVWGNGTQVTV |
| | VKPSETLSLTC | WIG | QVSLSLVAATA | SS (SEQ ID |
| | SVSGASIS | (SEQ ID NO: | ADSGKYYCAR | NO: 89) |
| | (SEQ ID NO: | 78) | (SEQ ID NO: | |
| | 74) | | 81) | |
| ePGT121.15.H1 VH | QMQLQESGPGL | WLRETPGKGLE | RVHLGLHPSKN | YWEVWGNGTQVTV |
| | VKPGETLSLTC | WIG | QVSLSLTSVTA | SS (SEQ ID |
| | SVSGASIS | (SEQ ID NO: | ADTAVYYCAR | NO: 91) |
| | (SEQ ID NO: | 79) | (SEQ ID NO: | |
| | 75) | | 83) | |
| ePGT121.15.H2 VH | QMQLQESGPGL | WLRETPGKGLE | RVHLGLHPSKN | YWEVWGKGTQVTV |
| | VKPGETLSLTC | WIG | QVSLSLTSVTA | SS (SEQ ID |
| | SVSGASIS | (SEQ ID NO: | ADTAVYYCAR | NO: 92) |
| | (SEQ ID NO: | 79) | (SEQ ID NO: | |
| | 75) | | 83) | |
| ePGT121.15.H3 VH | QMQLQESGPGL | WLRETPGKGLE | RVHLGLHPSKN | YWEVWGNGTQVTV |
| | VKPGETLSLTC | WIG | QVSLSLTSVTA | SS (SEQ ID |
| | SVSGASIS | (SEQ ID NO: | ADTAVYYCAR | NO: 91) |
| | (SEQ ID NO: | 79) | (SEQ ID NO: | |
| | 75) | | 83) | |
| ePGT121.15.H4 VH | QMQLQESGPGL | WLRETPGKGLE | RVHLGLHPSKN | YWEVWGKGTQVTV |
| | VKPGETLSLTC | WIG | QVSLSLTSVTA | SS (SEQ ID |
| | SVSGASIS | (SEQ ID NO: | ADTAVYYCAR | NO: 92) |
| | (SEQ ID NO: | 79) | (SEQ ID NO: | |
| | 75) | | 83) | |
| ePGT121.15.H5 VH | QVQLQESGPGL | WLRETPGKGLE | RVHLGLHPSKN | YWEVWGKGTQVTV |
| | VKPGETLSLTC | WIG | QVSLSLTSVTA | SS (SEQ ID |
| | SVSGASIS | (SEQ ID NO: | ADTAVYYCAR | NO: 92) |
| | (SEQ ID NO: | 79) | (SEQ ID NO: | |
| | 76 | | 83) | |
| ePGT121.15.H6 VH | QMQLQESGPGL | WLRETPGKGLE | RVHLGLHPSKN | YWEVWGKGTQVTV |
| | VKPGETLSLTC | WIG | QVSLSLTSVTA | SS (SEQ ID |
| | SVSGASIS | (SEQ ID NO: | ADTAVYYCAR | NO: 92) |
| | (SEQ ID NO: | 79) | (SEQ ID NO: | |
| | 75) | | 83) | |
| ePGT121.15.H7 VH | QMQLQESGPGL | WLRETPGKGLE | RVHLGLHPSKN | YWEVWGKGTQVTV |
| | VKPGETLSLTC | WIG | QVSLKLKSVTA | SS (SEQ ID |
| | SVSGASIS | (SEQ ID NO: | ADTAVYYCAR | NO: 92) |
| | (SEQ ID NO: | 79) | (SEQ ID NO: | |
| | 75) | | 84) | |
| ePGT121.12.H1 VH | QMQLQESGPGL | WIRRSPGKGLE | RVNLSLDTSKN | YMDVWGNGTQVTV |
| | VKPSETLSLTC | WIG | QVSLSLVAATA | SS (SEQ ID |
| | SVSGASIS | (SEQ ID NO: | ADSGKYYCAR | NO: 89) |
| | (SEQ ID NO: | 78) | (SEQ ID NO: | |
| | 74) | | 81) | |
| ePGT121.13.H1 VH | QMQLQESGPGL | WLRETPGKGLE | RVNLGLDTSKN | YWDVWGNGTQVTV |
| | VKPGETLSLTC | WIG | QVSLSLVAATA | SS (SEQ ID |
| | SVSGASIS | (SEQ ID NO: | ADSGKYYCAR | NO: 90) |
| | (SEQ ID NO: | 79) | (SEQ ID NO: | |
| | 75) | | 82) | |
| ePGT121.14.H1 VH | QMQLQESGPGL | WLRETPGKGLE | RVHLGLHPSKN | YWEVWGNGTQVTV |
| | VKPGETLSLTC | WIG | QVSLSLVAATA | SS (SEQ ID |
| | SVSGASIS | (SEQ ID NO: | ADSGKYYCAR | NO: 91) |
| | (SEQ ID NO: | 79) | (SEQ ID NO: | |
| | 75) | | 85) | |
| ePGT121.14.H2 VH | QMQLQESGPGL | WLRETPGKGLE | RVHLGLHPSKN | YWEVWGKGTQVTV |
| | VKPGETLSLTC | WIG | QVSLSLVAATA | SS (SEQ ID |
| | SVSGASIS | (SEQ ID NO: | ADSGKYYCAR | NO: 92) |
| | (SEQ ID NO: | 79) | (SEQ ID NO: | |
| | 75) | | 85) | |
| ePGT121.14.H3 VH | QMQLQESGPGL | WLRETPGKGLE | RVHLGLHPSKN | YWEVWGNGTQVTV |
| | VKPGETLSLTC | WIG | QVSLSLVAATA | SS (SEQ ID |
| | SVSGASIS | (SEQ ID NO: | ADSGKYYCAR | NO: 91) |
| | (SEQ ID NO: | 79) | (SEQ ID NO: | |
| | 75) | | 85) | |
| ePGT121.14.H4 VH | QMQLQESGPGL | WLRETPGKGLE | RVHLGLHPSKN | YWEVWGNGTQVTV |
| | VKPGETLSLTC | WIG | QVSLSLVAATA | SS (SEQ ID |
| | SVSGASIS | (SEQ ID NO: | ADSGKYYCAR | NO: 91) |
| | (SEQ ID NO: | 79) | (SEQ ID NO: | |
| | 75) | | 85) | |
| ePGT121.14.H5 VH | QMQLQESGPGL | WLRETPGKGLE | RVHLGLHPSKN | YWEVWGNGTQVTV |
| | VKPGETLSLTC | WIG | QVSLSLVAATA | SS (SEQ ID |
| | SVSGASIS | (SEQ ID NO: | ADSGKYYCAR | NO: 91) |
| | (SEQ ID NO: | 79) | (SEQ ID NO: | |
| | 75) | | 85) | |
| ePGT121.14.H6 VH | QMQLQESGPGL | WLRETPGKGLE | RVHLGLHPSKN | YWEVWGNGTQVTV |
| | VKPGETLSLTC | WIG | QVSLSLTSVTA | SS (SEQ ID |
| | SVSGASIS | (SEQ ID NO: | ADTAVYYCAR | NO: 91) |
| | (SEQ ID NO: | 79) | (SEQ ID NO: | |
| | 75) | | 83) | |
| ePGT121.18.H1 VH | QMQLQESGPGL | WLRETPGKGLE | RVHLGLHPSKN | YWPTWGKGTQVTV |
| | VKPGETLSLTC | WIG | QVSLSLTSVTA | SS (SEQ ID |
| | SVSGASIS | (SEQ ID NO: | ADTAVYYCAR | NO: 93) |
| | (SEQ ID NO: | 79) | (SEQ ID NO: | |
| | 75) | | 83) | |
| ePGT121.18.H2 VH | QMQLQESGPGL | WLRETPGKGIE | RVHLGLHPEKN | YWPTWGKGTQVTV |
| | VKPGETLSLTC | WIG | QVSLKLTSVTA | SS (SEQ ID |
| | SVSGASIS | (SEQ ID NO: | ADTAVYYCAR | NO: 93) |
| | (SEQ ID NO: | 148) | (SEQ ID NO: | |
| | 75) | | 150) | |
| ePGT121.18.H3 VH | QMQLQESGPGL | WLRETPGKGLE | RVHLGLHPEKN | YWPTWGKGTQVTV |
| | VKPGETLSLTC | WIG | QVSLKLTSVTA | SS (SEQ ID |
| | SVSGASIS | (SEQ ID NO: | ADTAVYYCAR | NO: 93) |
| | (SEQ ID NO: | 79) | (SEQ ID NO: | |
| | 75) | | 150) | |
| ePGT121.18.H4 VH | QMQLQESGPGL | WLRETPGKGIE | RVHLGLHPSKN | YWPTWGKGTQVTV |
| | VKPGETLSLTC | WIG | QVSLKLTSVTA | SS (SEQ ID |
| | SVSGASIS | (SEQ ID NO: | ADTAVYYCAR | NO: 93) |
| | (SEQ ID NO: | 148) | (SEQ ID NO: | |
| | 75) | | 151) | |
| ePGT121.18.H5 VH | QMQLQESGPGL | WLRETPGKGLE | RVHLGLHPEKN | YWPTWGKGIQVTV |
| | VKPGETLSLTC | WIG | QVSLSLTSVTA | SS |
| | SVSGASIS | (SEQ ID NO: | ADTAVYYCAR | (SEQ ID NO: |
| | (SEQ ID NO: | 79) | (SEQ ID NO: | 153) |
| | 75) | | 152) | |
| ePGT121.18.H6 VH | QMQLQESGPGL | WLRETPGKGIE | RVHLGLHPSKN | YWPTWGKGTKVTV |
| | VKPGETLSLTC | WIG | QVSLSLTSVTA | SS |
| | SVSGASIS | (SEQ ID NO: | ADTAVYYCAR | (SEQ ID NO: |
| | (SEQ ID NO: | 148) | (SEQ ID NO: | 154) |
| | 75) | | 83) | |
| ePGT121.18.H7 VH | QMQLQESGPGL | WLRETPGKGLE | RVHLGLHPSKN | YWPTWGKGTQVTV |
| | VKPGETLSLTC | WIG | QVSLSLTSVTA | SS (SEQ ID |
| | SVSGASIS | (SEQ ID NO: | ADTAVYYCAR | NO: 93) |
| | (SEQ ID NO: | 79) | (SEQ ID NO: | |
| | 75) | | 83) | |
| ePGT121.18.H8 VH | QMQLQESGPGL | WLRETPGKGLE | RVHLGLHPSKN | YWPTWGKGIQVTV |
| | VKPGETLSLTC | WIG | QVSLKLTSVTA | SS |
| | SVSGASIS | (SEQ ID NO: | ADTAVYYCAR | (SEQ ID NO: |
| | (SEQ ID NO: | 79) | (SEQ ID NO: | 153) |
| | 75) | | 151) | |
| ePGT121.18.H9 VH | QMQLQESGPGL | WLRETPGKGLE | RVHLGLHPSKN | YWPTWGKGTQVTV |
| | VKPGETLSLTC | WIG | QVSLSLTSVTA | SS (SEQ ID |
| | SVSGASIS | (SEQ ID NO: | ADTAVYYCAR | NO: 93) |
| | (SEQ ID NO: | 79) | (SEQ ID NO: | |
| | 75) | | 83) | |
| ePGT121.18.H10 VH | QMQLQESGPGL | WLRETPGKGLE | RVHLGLHPEKN | YWPTWGKGTQVTV |
| | VKPGETLSLTC | WIG | QVSLSLTSVTA | SS (SEQ ID |
| | SVSGASIS | (SEQ ID NO: | ADTAVYYCAR | NO: 93) |
| | (SEQ ID NO: | 79) | (SEQ ID NO: | |
| | 75) | | 152) | |
| ePGT121.18.H11 VH | QMQLQESGPGL | WLRETPGKGIE | RVHLGLHPSKN | YWPTWGKGTKVTV |
| | VKPGETLSLTC | WIG | QVSLSLTSVTA | SS |
| | SVSGASIS | (SEQ ID NO: | ADTAVYYCAR | (SEQ ID NO: |
| | (SEQ ID NO: | 148) | (SEQ ID NO: | 154) |
| | 75) | | 83) | |
| ePGT121.18.H12 VH | QMQLQESGPGL | WLRETPGKGVE | RVHLGLHPEKN | YWPTWGKGIQVTV |
| | VKPGETLSLTC | WIG | QVSLSLTSVTA | SS |
| | SVSGASIS | (SEQ ID NO: | ADTAVYYCAR | (SEQ ID NO: |
| | (SEQ ID NO: | 149) | (SEQ ID NO: | 153) |
| | 75) | | 152) | |
| ePGT121.18.H13 VH | QMQLQESGPGL | WLRETPGKGLE | RVHLGLHPSKN | YWPTWGKGIQVTV |
| | VKPGETLSLTC | WIG | QVSLSLTSVTA | SS |
| | SVSGASIS | (SEQ ID NO: | ADTAVYYCAR | (SEQ ID NO: |
| | (SEQ ID NO: | 79) | (SEQ ID NO: | 153) |
| | 75) | | 83) | |
| ePGT121.18.H14 VH | QMQLQESGPGL | WLRETPGKGIE | RVHLGLHPSKN | YWPTWGKGTQVTV |
| | VKPGETLSLTC | WIG | QVSLSLTSVTA | SS (SEQ ID |
| | SVSGASIS | (SEQ ID NO: | ADTAVYYCAR | NO: 93) |
| | (SEQ ID NO: | 148) | (SEQ ID NO: | |
| | 75) | | 83) | |
| ePGT121.18.H15 VH | QMQLQESGPGL | WLRETPGKGLE | RVHLGLHPSKN | YWPTWGKGIQVTV |
| | VKPGETLSLTC | WIG | QVSLSLTSVTA | SS |
| | SVSGASIS | (SEQ ID NO: | ADTAVYYCAR | (SEQ ID NO: |
| | (SEQ ID NO: | 79) | (SEQ ID NO: | 153) |
| | 75) | | 83) | |
| ePGT121.18.H16 VH | QMQLQESGPGL | WLRETPGKGLE | RVHLGLHPSKN | YWPTWGKGTKVTV |
| | VKPGETLSLTC | WIG | QVSLKLTSVTA | SS |
| | SVSGASIS | (SEQ ID NO: | ADTAVYYCAR | (SEQ ID NO: |
| | (SEQ ID NO: | 79) | (SEQ ID NO: | 154) |
| | 75) | | 151) | |
| ePGT121.18.H17 VH | QMQLQESGPGL | WLRETPGKGLE | RVTIGLDPSKN | YWPTWGKGTQVTV |
| | VKPGETLSLTC | WIG | QVSLSLTSVTA | SS (SEQ ID |
| | SVSGASIS | (SEQ ID NO: | ADTAVYYCAR | NO: 93) |
| | (SEQ ID NO: | 79) | (SEQ ID NO: | |
| | 75) | | 86) | |
| ePGT121.18.H18 VH | QMQLQESGPGL | WLRETPGKGLE | RVTISLDPSKN | YWPTWGKGTQVTV |
| | VKPGETLSLTC | WIG | QVSLSLTSVTA | SS (SEQ ID |
| | SVSGASIS | (SEQ ID NO: | ADTAVYYCAR | NO: 93) |
| | (SEQ ID NO: | 79) | (SEQ ID NO: | |
| | 75) | | 87) | |
| PGT-121 VL | SDISVAPGETA | WYQHRAGQAPS | GIPERFSGSPD | FGGGTTLTVL |
| | RISC (SEQ | LIIY (SEQ | SPFGTTATLTI | (SEQ ID NO: |
| | ID NO: 95) | ID NO: 100) | TSVEAGDEADY | 109) |
| | | | YC (SEQ ID | |
| | | | NO: 104) | |
| ePGT12115_L1 VL | SDISVAPGETV | WYQHRAGQAPK | GIPERFSGSPD | FGGGTTLTVL |
| | RISC (SEQ | LIIY (SEQ | IDFGTTATLTI | (SEQ ID NO: |
| | ID NO: 96) | ID NO: 101) | TNVEAGDEATY | 109) |
| | | | YC (SEQ ID | |
| | | | NO: 105) | |
| ePGT121.15_L2 VL | SYVLTQPPSVS | WYQHRAGQAPK | GIPERFSGSPD | FGGGTTLTVL |
| | VAPGETVRISC | LIIY (SEQ | IDFGTTATLTI | (SEQ ID NO: |
| | (SEQ ID | ID NO: 101) | TNVEAGDEATY | 109) |
| | NO: 97) | | YC (SEQ ID | |
| | | | NO: 105) | |
| ePGT121.12_L4 VL | SDISVAPGETA | WYQHRAGQAPS | GIPERFSGSPD | FGGGTTLTVL |
| | RISC (SEQ | LIIY (SEQ | IAFGTTATLTI | (SEQ ID NO: |
| | ID NO: 95) | ID NO: 100) | TSVEAGDEADY | 109) |
| | | | YC (SEQ ID | |
| | | | NO: 106) | |
| ePGT121.13_L1 VL | SDISVAPGETV | WYQHRAGQAPK | GIPERFSGSPD | FGGGTTLTVL |
| | RISC (SEQ | LIIY (SEQ | IDFGTTATLTI | (SEQ ID NO: |
| | ID NO: 96) | ID NO: 101) | TNVEAGDEATY | 109) |
| | | | YC (SEQ ID | |
| | | | NO: 105) | |
| ePGT121.14_L1 VL | SDISVAPGETV | WYQHRAGQAPK | GIPERFSGSPD | FGGGTTLTVL |
| | RISC (SEQ | LIIY (SEQ | IDFGTTATLTI | (SEQ ID NO: |
| | ID NO: 96) | ID NO: 101) | TNVEAGDEATY | 109) |
| | | | YC (SEQ ID | |
| | | | NO: 105) | |
| ePGT121.17_L1 VL | SDISVAPGETV | WYQHRPGQAPR | GIPERFSGSPD | LGPGTTLTVL |
| | RITC (SEQ | LIIY (SEQ | IDFGTTATLTI | (SEQ ID NO: |
| | ID NO: 98) | ID NO: 102) | SNVEAGDEATY | 110) |
| | | | YC (SEQ ID | |
| | | | NO: 107) | |
| ePGT121.17_L2 VL | SDISVAPGETV | WYQHRPGQAPR | GIPERFSGSPD | LGPGTTLTVL |
| | RISC (SEQ | LIIY (SEQ | IDFGTTATLTI | (SEQ ID NO: |
| | ID NO: 96) | ID NO: 157) | TNVEAGDEATY | 110) |
| | | | YC (SEQ ID | |
| | | | NO: 105) | |
| ePGT121.17_L3 VL | SDLSVAPGETV | WYQHRPGQAPR | GIPERFSGSPD | FEPGTTLTVL |
| | RITC (SEQ | LIIY (SEQ | IDFGTTATLTI | (SEQ ID NO: |
| | ID NO: 155) | ID NO: 102) | SNVEAGDEATY | 165) |
| | | | YC (SEQ ID | |
| | | | NO: 107) | |
| ePGT121.17_L4 VL | SDLSVAPGETV | WYQHRAGQAPR | GIPERFSGSPD | FEPGTTLTVL |
| | RISC (SEQ | LIIY (SEQ | IDFGTTATLTI | (SEQ ID NO: |
| | ID NO: 156) | ID NO: 157) | TNVEAGDEATY | 165) |
| | | | YC (SEQ ID | |
| | | | NO: 105) | |
| ePGT121.17_L5 VL | SDLSVAPGETV | WYQHRPGQAPK | GIPERFSGSPD | LGPGTTLTVL |
| | RITC (SEQ | LIIY (SEQ | IDFGTTATLTI | (SEQ ID NO: |
| | ID NO: 155) | ID NO: 158) | SRVEAGDEATY | 110) |
| | | | YC (SEQ ID | |
| | | | NO: 161) | |
| ePGT121.17_L6 VL | SDLSVAPGETV | WYQHRAGQAPK | GIPERFSGSPD | LGPGTTLTVL |
| | RISC (SEQ | LIIY (SEQ | IDFGTTATLTI | (SEQ ID NO: |
| | ID NO: 156) | ID NO: 101) | TRVEAGDEATY | 110) |
| | | | YC (SEQ ID | |
| | | | NO: 162) | |
| ePGT121.17_L7 VL | SDISVAPGETV | WYQHRPGQAPR | GIPERFSGSPD | LGPGTTLTVL |
| | RITC (SEQ | LIIY (SEQ | LDFGTTATLTI | (SEQ ID NO: |
| | ID NO: 98) | ID NO: 102) | SNVEAGDEATY | 110) |
| | | | YC (SEQ ID | |
| | | | NO: 163) | |
| ePGT121.17_L8 VL | SDISVAPGETV | WYQHRPGQAPR | GIPERFSGSPD | LGPGTTLTVL |
| | RISC (SEQ | LIIY (SEQ | LDFGTTATLTI | (SEQ ID NO: |
| | ID NO: 96) | ID NO: 157) | TNVEAGDEATY | 110) |
| | | | YC (SEQ ID | |
| | | | NO: 164) | |
| ePGT121.17_L9 VL | SDISVAPGETV | WYQHRPGQAPR | GIPERFSGSPD | LEGGTTLTVL |
| | RITC (SEQ | LIIY (SEQ | IDFGTTATLTI | (SEQ ID NO: |
| | ID NO: 98) | ID NO: 102) | SNVEAGDEATY | 166) |
| | | | YC (SEQ ID | |
| | | | NO: 107) | |
| ePGT121.17_L10 VL | SDISVAPGETV | WYQHRAGQAPR | GIPERFSGSPD | LEGGTTLTVL |
| | RISC (SEQ | LIIY (SEQ | IDFGTTATLTI | (SEQ ID NO: |
| | ID NO: 96) | ID NO: 157) | TNVEAGDEATY | 166) |
| | | | YC (SEQ ID | |
| | | | NO: 105) | |
| ePGT121.17_L11 VL | SDLSVAPGETV | WYQHRPGQAPR | GIPERFSGSPD | LGGGTTLTVL |
| | RITC (SEQ | LIIY (SEQ | IDFGTTATLTI | (SEQ ID NO: |
| | ID NO: 155) | ID NO: 102) | SNVEAGDEATY | 167) |
| | | | YC (SEQ ID | |
| | | | NO: 107) | |
| ePGT121.17_L12 VL | SDLSVAPGETV | WYQHRAGQAPR | GIPERFSGSPD | LGGGTTLTVL |
| | RISC (SEQ | LIIY (SEQ | IDFGTTATLTI | (SEQ ID NO: |
| | ID NO: 156) | ID NO: 157) | TNVEAGDEATY | 167) |
| | | | YC (SEQ ID | |
| | | | NO: 105) | |
| ePGT121.17_L13 VL | SDISVAPGETV | WYQQRPGQAPR | GIPERFSGSPD | LGPGTTLTVL |
| | RITC (SEQ | LIIY (SEQ | IDFGTTATLTI | (SEQ ID NO: |
| | ID NO: 98) | ID NO: 159) | SNVEAGDEATY | 110) |
| | | | YC (SEQ ID | |
| | | | NO: 107) | |
| ePGT121.17_L14 VL | SDISVAPGETV | WYQQRAGQAPR | GIPERFSGSPD | LGPGTTLTVL |
| | RISC (SEQ | LIIY (SEQ | IDFGTTATLTI | (SEQ ID NO: |
| | ID NO: 96) | ID NO: 160) | TNVEAGDEATY | 110) |
| | | | YC (SEQ ID | |
| | | | NO: 105) | |

TABLE 4
Heavy chain (HC) and Light chain (LC) sequences.

| | |
|---|---|
| PGT-121 HC | QMQLQESGPGLVKPSETLSLTCSVSGASISDSYWSWIRRSPGKGLEWIG |
| | YVHKSGDTNYSPSLKSRVNLSLDTSKNQVSLSLVAATAADSGKYYCA |
| | RTLHGRRIYGIVAFNEWFTYFYMDVWGNGTQVTVSSASTKGPSVFPL |
| | APSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 32) |
| ePGT121.15.HC1 HC | QMQLQESGPGLVKPGETLSLTCSVSGASISSSYWSWLRETPGKGLEWI |
| | GYTHEISGDTNYAPSLKSRVHLGLHPSKNQVSLSLTSVTAADTAVYYC |
| | ARTLHGRRIYGVVAFNEWFTYFYWEVWGNGTQVTVSSASTKGPSVFP |
| | LAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVLHEALHSHYTQKSLSLSPGK (SEQ ID NO: 34) |
| ePGT121.15.HC2 HC | QMQLQESGPGLVKPGETLSLTCSVSGASISSSYWSWLRETPGKGLEWI |
| | GYTHEISGDTNYAPSLKSRVHLGLHPSKNQVSLSLTSVTAADTAVYYC |
| | ARTLHGRRIYGVVAFNEWFTYFYWEVWGKGTQVTVSSASTKGPSVFP |
| | LAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVLHEALHSHYTQKSLSLSPGK (SEQ ID NO: 35) |
| ePGT121.15.HC3 HC | QMQLQESGPGLVKPGETLSLTCSVSGASISSSYWSWLRETPGKGLEWI |
| | GYTHEISGDTNYAPSLKSRVHLGLHPSKNQVSLSLTSVTAADTAVYYC |
| | ARTLHGRRIYGVVAFNELFTYFYWEVWGNGTQVTVSSASTKGPSVFP |
| | LAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVLHEALHSHYTQKSLSLSPGK (SEQ ID NO: 36) |
| ePGT121.15.HC4 HC | QMQLQESGPGLVKPGETLSLTCSVSGASISSSYWSWLRETPGKGLEWI |
| | GYTHEISGDTNYAPSLKSRVHLGLHPSKNQVSLSLTSVTAADTAVYYC |
| | ARTLHGRRIYGVVAFNELFTYFYWEVWGKGTQVTVSSASTKGPSVFP |
| | LAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVLHEALHSHYTQKSLSLSPGK (SEQ ID NO: 37) |
| ePGT121.15.HC5 HC | QMQLQESGPGLVKPGETLSLTCSVSGASISSSYWSWLRETPGKGLEWI |
| | GYTHEISGDTNYAPSLKSRVHLGLHPSKNQVSLSLTSVTAADTAVYYC |
| | ARTLHGRRIYGVVAFNELFTYFYWEVWGKGTQVTVSSASTKGPSVFP |
| | LAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVLHEALHSHYTQKSLSLSPGK (SEQ ID NO: 38) |
| ePGT121.15.HC6 HC | QMQLQESGPGLVKPGETLSLTCSVSGASISSSYWSWLRETPGKGLEWI |
| | GYTHEISGDTNYAPSLKSRVHLGLHPSKNQVSLSLTSVTAADTAVYYC |
| | ARTLHGRRIYGVVAFNEFFTYFYWEVWGKGTQVTVSSASTKGPSVFPL |
| | APSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVLHEALHSHYTQKSLSLSPGK (SEQ ID NO: 39) |
| ePGT121.15.HC7 HC | QMQLQESGPGLVKPGETLSLTCSVSGASISSSYWSWLRETPGKGLEWI |
| | GYTHEISGDTNYAPSLKSRVHLGLHPSKNQVSLKLKSVTAADTAVYYC |
| | ARTLHGRRIYGVVAFNELFTYFYWEVWGKGTQVTVSSASTKGPSVFP |
| | LAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKS |
| | RWQQGNVFSCSVLHEALHSHYTQKSLSLSPGK (SEQ ID NO: 40) |
| PGT-121 LC | SDISVAPGETARISCGEKSLGSRAVQWYQHRAGQAPSLIIYNNQDRPSG |
| | IPERFSGSPDSPFGTTATLTITSVEAGDEADYYCHIWDSRVPTKWVFGG |
| | GTTLTVLGQPKAAPSVTLFPPSSEELQANKATLVCLISDFYPGAVTVA |
| | WKADSSPVKAGVETTTPSKQSNNKYAASSYLSLTPEQWKSHRSYSCQ |
| | VTHEGSTVEKTVAPTECS (SEQ ID NO: 33) |
| ePGT121.15.LC1 LC | SDISVAPGETVRISCGGESIGSRAVQWYQHRAGQAPKLIIYNNQDRPPG |
| | IPERFSGSPDIDFGTTATLTITNVEAGDEATYYCHIWDSRRPTNWVFGG |
| | GTTLTVLGQPKAAPSVTLFPPSSEELQANKATLVCLISDFYPGAVTVA |
| | WKADSSPVKAGVETTTPSKQSNNKYAASSYLSLTPEQWKSHRSYSCQ |
| | VTHEGSTVEKTVAPTECS (SEQ ID NO: 41) |
| ePGT121.15.LC2 LC | SYVLTQPPSVSVAPGETVRISCGGESIGSRAVQWYQHRAGQAPKLIIYN |
| | NQDRPPGIPERFSGSPDIDFGTTATLTITNVEAGDEATYYCHIWDSRRPT |
| | NWVFGGGTTLTVLGQPKAAPSVTLFPPSSEELQANKATLVCLISDFYPG |
| | AVTVAWKADSSPVKAGVETTTPSKQSNNKYAASSYLSLTPEQWKSHR |
| | SYSCQVTHEGSTVEKTVAPTECS (SEQ ID NO: 42) |

In one aspect, provided herein are engineered variants of the PGT-121 antibody comprising a VH CDR1, VH CDR2, VH CDR3, VL CDR1, VL CDR2, and VL CDR3, wherein one or more of the VH CDR1, VH CDR2, VH CDR3, VL CDR1, VL CDR2, and VL CDR2 is a variant of the corresponding PGT-121 CDR. In one embodiment, the antibody further comprises a VH FR1, VH FR2, VH FR3, VH FR4, VL FR1, VL FR2, VL FR3, and VL FR4, wherein one or more of the VH FR1, VH FR2, VH FR3, VH FR4, VL FR1, VL FR2, VL FR3, and VL FR4 is a variant of the corresponding PGT-121 FR.

In one embodiment, an antibody described herein comprises a VH CDR1, VH CDR2, VH CDR3, VL CDR1, VL CDR2, and VL CDR3, wherein the VH CDR1 comprises the sequence of X1SYWS, wherein X1 is D or S (SEQ ID NO: 26); the VH CDR2 comprises the sequence of YX1HX2SGDTNYX3PSLKS wherein X1 is V or T, X2 is K or H, and X3 is S or A (SEQ ID NO: 27); the VH CDR3 comprises the sequence of TLHGRRX₁YGX₂VAFX₃EX₄X₅TYFYX₆X₇X₈ wherein X₁ is I or C, X2 is I or V, X3 is N or G, X4 is W, C, F, L or Y, X5 is F or Y, X6 is M or W, X₇ is D, P or E, and X₈ is V or T (SEQ ID NO: 71); the VL CDR1 comprises the sequence of GX1X2SX3GSRAVQ wherein X1 is E or G, X2 is K or E, and X3 is L or I (SEQ ID NO: 29); the VL CDR2 comprises the sequence of NNQDRX1X2 wherein X₁ is P or G, and X2 is S or P (SEQ ID NO: 168); and the VL CDR3 comprises the sequence of HX1WDSRX2PTX3WX4 wherein X₁ is I or V, X₂ is V or R, X₃ is K, Q or N, and X₄ is V or E (SEQ ID NO: 73); wherein at least one of the VH CDR1, VH CDR2, VH CDR3, VL CDR1, VL CDR2, and VL CDR3 comprises a different amino acid sequence than the corresponding CDR of the PGT-121 antibody. In one embodiment, an antibody described herein comprises a VH CDR1, VH CDR2, VH CDR3, VL CDR1, VL CDR2, and VL CDR3, wherein the VH CDR1 comprises the sequence of X1SYWS, wherein X1 is D or S (SEQ ID NO: 26); the VH CDR2 comprises the sequence of YX1HX2SGDTNYX3PSLKS wherein X1 is V or T, X2 is K or H, and X3 is S or A (SEQ ID NO: 27); the VH CDR3 comprises the sequence of TLHGRRIYGX₁VAFX₂EX₃X₄TYFYX₅X₆X₇ wherein X₁ is I or V, X₂ is N or G, X₃ is W, F, L or Y, X₄ is F or Y, X₅ is M or W, X₆ is D, P or E, and X₇ is V or T (SEQ ID NO: 72); the VL CDR1 comprises the sequence of GX1X2SX3GSRAVQ wherein X1 is E or G, X2 is K or E, and X3 is L or I (SEQ ID NO: 29); the VL CDR2 comprises the sequence of NNQDRX₁X₂ wherein X₁ is P or G, and X₂ is S or P (SEQ ID NO: 168); and the VL CDR3 comprises the sequence of HX₁WDSRX₂PTX₃WX₄ wherein X₁ is I or V, X₂ is V or R, X₃ is K, Q or N, and X₄ is V or E (SEQ ID NO: 73); wherein at least one of the VH CDR1, VH CDR2, VH CDR3, VL CDR1, VL CDR2, and VL CDR3 comprises a different amino acid sequence than the corresponding CDR of the PGT-121 antibody. In one embodiment, an antibody described herein comprises a VH CDR1, VH CDR2, VH CDR3, VL CDR1, VL CDR2, and VL CDR3, wherein the VH CDR1 comprises the sequence of X1SYWS, wherein X1 is D or S (SEQ ID NO: 26); the VH CDR2 comprises the sequence of YX1HX2SGDTNYX3PSLKS wherein X1 is V or T, X2 is K or H, and X3 is S or A (SEQ ID NO: 27); the VH CDR3 comprises the sequence of TLHGRRX1YGX2VAFX3EX4FTYFYX5X6V wherein X1 is I or C, X2 is I or V, X3 is N or G, X4 is W, C, F or L, X5 is M or W, and X6 is D or E (SEQ ID NO: 28); the VL CDR1 comprises the sequence of GX1X2SX3GSRAVQ wherein X1 is E or G, X2 is K or E, and X3 is L or I (SEQ ID NO: 29); the VL CDR2 comprises the sequence of NNQDRPX1 wherein X1 is S or P (SEQ ID NO: 30); and the VL CDR3 comprises the sequence of HIWDSRX1PTX2WV wherein X1 is V or R, and X2 is K or N (SEQ ID NO: 31); wherein at least one of the VH CDR1, VH CDR2, VH CDR3, VL CDR1, VL CDR2, and VL CDR3 comprises a different amino acid sequence than the corresponding CDR of the PGT-121 antibody. In one embodiment, an antibody described herein comprises a VH CDR1, VH CDR2, VH CDR3, VL CDR1, VL CDR2, and VL CDR3, wherein the VH CDR1 comprises the sequence of X1SYWS, wherein X1 is D or S (SEQ ID NO: 26); the VH CDR2 comprises the sequence of YX1HX2SGDTNYX3PSLKS wherein X1 is V or T, X2 is K or H, and X3 is S or A (SEQ ID NO: 27); the VH CDR3 comprises the sequence of TLHGRRIYGX1VAFX2EX3FTYFYX4X5V wherein X1 is I or V, X2 is N or G, X3 is W, F or L, X4 is M or W, and X5 is D or E (SEQ ID NO: 64); the VL CDR1 comprises the sequence of GX1X2SX3GSRAVQ wherein X1 is E or G, X2 is K or E, and X3 is L or I (SEQ ID NO: 29); the VL CDR2 comprises the sequence of NNQDRPX1 wherein X1 is S or P (SEQ ID NO: 30); and the VL CDR3 comprises the sequence of HIWDSRX1PTX2WV wherein X1 is V or R, and X2 is K or N (SEQ ID NO: 31); wherein at least one of the VH CDR1, VH CDR2, VH CDR3, VL CDR1, VL CDR2, and VL CDR3 comprises a different amino acid sequence than the corresponding CDR of the PGT-121 antibody. In one embodiment, the VH CDR1 comprises the sequence of DSYWS (SEQ ID NO: 12). In one embodiment, the VH CDR1 comprises the sequence of SSYWS (SEQ ID NO: 18). In one embodiment, the VH CDR2 comprises the sequence of YVHKSGDTNYSPSLKS (SEQ ID NO: 13). In one embodiment, the VH CDR2 comprises the sequence of YTHHSGDTNYAPSLKS (SEQ ID NO: 19). In one embodiment, the VH CDR2 comprises the sequence of YVHKSGDTNYAPSLKS (SEQ ID NO: 61). In one embodiment, the VH CDR3 comprises the sequence of TLHGRRIYGIVAFNEWFTYFYMDV (SEQ ID NO: 14). In one embodiment, the VH CDR3 comprises the sequence of TLHGRRIYGVVAFNEWFTYFYWEV (SEQ ID NO: 20). In one embodiment, the VH CDR3 comprises the sequence of TLHGRRIYGVVAFNELFTYFYWEV (SEQ ID NO: 21). In one embodiment, the VH CDR3 comprises the sequence of TLHGRRIYGVVAFNEFFTYFYWEV (SEQ ID NO: 22). In one embodiment, the VH CDR3 comprises the sequence of TLHGRRIYGVVAFGEWFTYFYWEV (SEQ ID NO: 59). In one embodiment, the VH CDR3 comprises the sequence of TLHGRRCYGVVAFNECFTYFYWEV (SEQ ID NO: 60). In one embodiment, the VH CDR3 comprises the sequence of TLHGRRIYGVVAFNEWFTYFYWDV (SEQ ID NO: 62). In one embodiment, the VH CDR3 comprises the sequence of TLHGRRIYGVVAFNEYYTYFYWPT (SEQ ID NO: 67). In one embodiment, the VH CDR3 comprises the sequence of TLHGRRIYGVVAGNEYYTYFYWPT (SEQ ID NO: 140). In one embodiment, the VH CDR3 comprises the sequence of TLHGRRIYGVVAGGEYYTYFYWPT (SEQ ID NO: 141). In one embodiment, the VH CDR3 comprises the sequence of TLHGRRIYGVVAGNEFYTYFYWPT (SEQ ID NO: 142). In one embodiment, the VH CDR3 comprises the sequence of TLHGRRIYGVVAGGEFYTYFYWPT (SEQ ID NO: 143). In one embodiment, the VH CDR3 comprises the sequence of TLHGRRIYGVVEGGEYYTYFYWPT (SEQ ID NO: 144). In one embodiment, the VL CDR1 comprises the sequence of GEKSLGSRAVQ (SEQ ID NO: 15). In one embodiment, the VL CDR1 comprises the sequence of GGESIGSRAVQ (SEQ ID NO: 23). In one embodiment, the VL CDR1 comprises the sequence of GGESLGSRAVQ (SEQ ID NO: 63). In one embodiment, the VL CDR2 comprises the sequence of NNQDRPS (SEQ ID NO: 16). In one embodiment, the VL CDR2 comprises the sequence of NNQDRPP (SEQ ID NO: 24). In one embodiment, the VL CDR2 comprises the sequence of NNQDRGP (SEQ ID NO: 145). In one embodiment, the VL CDR3 comprises the sequence of HIWDSRVPTKWV (SEQ ID NO: 17). In one embodiment, the VL CDR3 comprises the sequence of HIWDSRRPTNWV (SEQ ID NO: 25). In one embodiment, the VL CDR3 comprises the sequence of HIWDSRRPTNWE (SEQ ID NO: 68). In one embodiment, the VL CDR3 comprises the sequence of HVWDSRRPTNWE (SEQ ID NO: 146). In one embodiment, the VL CDR3 comprises the sequence of HVWDSRRPTQWV (SEQ ID NO: 147). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H1 VH (SEQ ID NO: 3). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H3 VH (SEQ ID NO: 5). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H6 VH (SEQ ID NO: 8). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.13.H1 VH (SEQ ID NO: 47). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.14.H3 VH (SEQ ID NO: 51). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.14.H4 VH (SEQ ID NO: 52). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H1 VH (SEQ ID NO: 65). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H17 VH (SEQ ID NO: 69). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H18 VH (SEQ ID NO: 70). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H2-16 VH (SEQ ID NO: 112-126). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18, the VH CDR2 comprises the amino acid sequence of SEQ ID NO: 19 or 61, and the VH CDR3 comprises the amino acid sequence of SEQ ID NO: 20-22, 59, 60 or 62. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18, the VH CDR2 comprises the amino acid sequence of SEQ ID NO: 19 or 61, and the VH CDR3 comprises the amino acid sequence of SEQ ID NO: 20-22, 59, 60, 62, 67, or 140-144. In one embodiment, an antibody described herein comprises a VH CDR1, VH CDR2, VH CDR3, VL CDR1, VL CDR2, and VL CDR3, wherein the VL CDR1 comprises the amino acid sequence of SEQ ID NO: 23 or 63, the VL CDR2 comprises the amino acid sequence of SEQ ID NO: 16 or 24, and the VL CDR3 comprises the amino acid sequence of SEQ ID NO: 25. In one embodiment, an antibody described herein comprises a VH CDR1, VH CDR2, VH CDR3, VL CDR1, VL CDR2, and VL CDR3, wherein the VL CDR1 comprises the amino acid sequence of SEQ ID NO: 23 or 63, the VL CDR2 comprises the amino acid sequence of SEQ ID NO: 16, 24 or 145, and the VL CDR3 comprises the amino acid sequence of SEQ ID NO: 25, 68, 146 or 147. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18, the VH CDR2 comprises the amino acid sequence of SEQ ID NO: 19 or 61, the VH CDR3 comprises the amino acid sequence of SEQ ID NO: 20-22, 59, 60 or 62, the VL CDR1 comprises the amino acid sequence of SEQ ID NO: 23 or 63, the VL CDR2 comprises the amino acid sequence of SEQ ID NO: 16 or 24, and the VL CDR3 comprises the amino acid sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18, the VH CDR2 comprises the amino acid sequence of SEQ ID NO: 19 or 61, the VH CDR3 comprises the amino acid sequence of SEQ ID NO: 20-22, 59, 60, 62, 67 or 140-144, the VL CDR1 comprises the amino acid sequence of SEQ ID NO: 23, 63 or 145, the VL CDR2 comprises the amino acid sequence of SEQ ID NO: 16 or 24, and the VL CDR3 comprises the amino acid sequence of SEQ ID NO: 25, 68, 146, or 147. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18 comprising 0, 1, 2, 3, 4, or 5 substitutions, insertions, or deletions, the VH CDR2 comprises the amino acid sequence of SEQ ID NO: 19 or 61 comprising 0, 1, 2, 3, 4, or 5 substitutions, insertions, or deletions, and the VH CDR3 comprises the amino acid sequence of SEQ ID NO: 20-22, 59, 60 or 62 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18 comprising 0, 1, 2, 3, 4, or 5 substitutions, insertions, or deletions, the VH CDR2 comprises the amino acid sequence of SEQ ID NO: 19 or 61 comprising 0, 1, 2, 3, 4, or 5 substitutions, insertions, or deletions, and the VH CDR3 comprises the amino acid sequence of SEQ ID NO: 20-22, 59, 60, 62, 67 or 140-144 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the amino acid sequence of SEQ ID NO: 23 or 63 comprising 0, 1, 2, 3, 4, or 5 substitutions, insertions, or deletions, the VL CDR2 comprises the amino acid sequence of SEQ ID NO: 16 or 24 comprising 0, 1, 2, 3, 4, or 5 substitutions, insertions, or deletions, and the VL CDR3 comprises the amino acid sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the amino acid sequence of SEQ ID NO: 23 or 63 comprising 0, 1, 2, 3, 4, or 5 substitutions, insertions, or deletions, the VL CDR2 comprises the amino acid sequence of SEQ ID NO: 16, 24 or 145 comprising 0, 1, 2, 3, 4, or 5 substitutions, insertions, or deletions, and the VL CDR3 comprises the amino acid sequence of SEQ ID NO: 25, 68, 146, or 147 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18 comprising 0, 1, 2, 3, 4, or 5 substitutions, insertions, or deletions, the VH CDR2 comprises the amino acid sequence of SEQ ID NO: 19 or 61 comprising 0, 1, 2, 3, 4, or 5 substitutions, insertions, or deletions, the VH CDR3 comprises the amino acid sequence of SEQ ID NO: 20-22, 59, 60 or 62 comprising 0, 1, 2, 3, 4, or 5 substitutions, insertions, or deletions, the VL CDR1 comprises the amino acid sequence of SEQ ID NO: 23 or 63 comprising 0, 1, 2, 3, 4, or 5 substitutions, insertions, or deletions, the VL CDR2 comprises the amino acid sequence of SEQ ID NO: 16 or 24 comprising 0, 1, 2, 3, 4, or 5 substitutions, insertions, or deletions, and the VL CDR3 comprises the amino acid sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18 comprising 0, 1, 2, 3, 4, or 5 substitutions, insertions, or deletions, the VH CDR2 comprises the amino acid sequence of SEQ ID NO: 19 or 61 comprising 0, 1, 2, 3, 4, or 5 substitutions, insertions, or deletions, the VH CDR3 comprises the amino acid sequence of SEQ ID NO: 20-22, 59, 60, 62, 67 or 140-144 comprising 0, 1, 2, 3, 4, or 5 substitutions, insertions, or deletions, the VL CDR1 comprises the amino acid sequence of SEQ ID NO: 23 or 63 comprising 0, 1, 2, 3, 4, or 5 substitutions, insertions, or deletions, the VL CDR2 comprises the amino acid sequence of SEQ ID NO: 16, 24 or 145 comprising 0, 1, 2, 3, 4, or 5 substitutions, insertions, or deletions, and the VL CDR3 comprises the amino acid sequence of SEQ ID NO: 25, 68, 146, or 147 comprising 0, 1, 2, 3, 4, or 5 substitutions, insertions, or deletions. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18, the VH CDR2 comprises the amino acid sequence of SEQ ID NO: 13, 19 or 61, and the VH CDR3 comprises the amino acid sequence of SEQ ID NO: 14, 20-22, 59, 60 or 62. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18, the VH CDR2 comprises the amino acid sequence of SEQ ID NO: 13, 19 or 61, and the VH CDR3 comprises the amino acid sequence of SEQ ID NO: 14, 20-22, 59, 60, 62, 67 or 140-144. In one embodiment, an antibody described herein comprises a VH CDR1, VH CDR2, VH CDR3, VL CDR1, VL CDR2, and VL CDR3, wherein the VL CDR1 comprises the amino acid sequence of SEQ ID NO: 15, 23 or 63, the VL CDR2 comprises the amino acid sequence of SEQ ID NO: 16 or 24, and the VL CDR3 comprises the amino acid sequence of SEQ ID NO: 17 or 25. In one embodiment, an antibody described herein comprises a VH CDR1, VH CDR2, VH CDR3, VL CDR1, VL CDR2, and VL CDR3, wherein the VL CDR1 comprises the amino acid sequence of SEQ ID NO: 15, 23 or 63, the VL CDR2 comprises the amino acid sequence of SEQ ID NO: 16, 24 or 145, and the VL CDR3 comprises the amino acid sequence of SEQ ID NO: 17, 25, 68, 146, or 147. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18, the VH CDR2 comprises the amino acid sequence of SEQ ID NO: 13, 19 or 61, the VH CDR3 comprises the amino acid sequence of SEQ ID NO: 14, 20-22, 59, 60 or 62, the VL CDR1 comprises the amino acid sequence of SEQ ID NO: 15, 23 or 63, the VL CDR2 comprises the amino acid sequence of SEQ ID NO: 16 or 24, and the VL CDR3 comprises the amino acid sequence of SEQ ID NO: 17 or 25. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18, the VH CDR2 comprises the amino acid sequence of SEQ ID NO: 13, 19 or 61, the VH CDR3 comprises the amino acid sequence of SEQ ID NO: 14, 20-22, 59, 60, 62, 67 or 140-144, the VL CDR1 comprises the amino acid sequence of SEQ ID NO: 15, 23 or 63, the VL CDR2 comprises the amino acid sequence of SEQ ID NO: 16, 24 or 145, and the VL CDR3 comprises the amino acid sequence of SEQ ID NO: 17, 25, 68, 146, or 147. In one embodiment, at least one of the VH CDR1, VH CDR2, VH CDR3, VL CDR1, VL CDR2, and VL CDR3 comprises a different amino acid sequence than the corresponding CDR of the PGT-121 antibody. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18 comprising 0, 1, 2, 3, 4, or 5 substitutions, insertions, or deletions, the VH CDR2 comprises the amino acid sequence of SEQ ID NO: 13, 19 or 61 comprising 0, 1, 2, 3, 4, or 5 substitutions, insertions, or deletions, and the VH CDR3 comprises the amino acid sequence of SEQ ID NO: 14, 20-22, 59, 60 or 62 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18 comprising 0, 1, 2, 3, 4, or 5 substitutions, insertions, or deletions, the VH CDR2 comprises the amino acid sequence of SEQ ID NO: 13, 19 or 61 comprising 0, 1, 2, 3, 4, or 5 substitutions, insertions, or deletions, and the VH CDR3 comprises the amino acid sequence of SEQ ID NO: 14, 20-22, 59, 60, 62, 67 or 140-144 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the amino acid sequence of SEQ ID NO: 15, 23 or 63 comprising 0, 1, 2, 3, 4, or 5 substitutions, insertions, or deletions, the VL CDR2 comprises the amino acid sequence of SEQ ID NO: 16 or 24 comprising 0, 1, 2, 3, 4, or 5 substitutions, insertions, or deletions, and the VL CDR3 comprises the amino acid sequence of SEQ ID NO: 17 or 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the amino acid sequence of SEQ ID NO: 15, 23 or 63 comprising 0, 1, 2, 3, 4, or 5 substitutions, insertions, or deletions, the VL CDR2 comprises the amino acid sequence of SEQ ID NO: 16, 24 or 145 comprising 0, 1, 2, 3, 4, or 5 substitutions, insertions, or deletions, and the VL CDR3 comprises the amino acid sequence of SEQ ID NO: 17, 25, 68, 146, or 147 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18 comprising 0, 1, 2, 3, 4, or 5 substitutions, insertions, or deletions, the VH CDR2 comprises the amino acid sequence of SEQ ID NO: 13, 19 or 61 comprising 0, 1, 2, 3, 4, or 5 substitutions, insertions, or deletions, the VH CDR3 comprises the amino acid sequence of SEQ ID NO: 14, 20-22, 59, 60 or 62 comprising 0, 1, 2, 3, 4, or 5 substitutions, insertions, or deletions, the VL CDR1 comprises the amino acid sequence of SEQ ID NO: 15, 23 or 63 comprising 0, 1, 2, 3, 4, or 5 substitutions, insertions, or deletions, the VL CDR2 comprises the amino acid sequence of SEQ ID NO: 16 or 24 comprising 0, 1, 2, 3, 4, or 5 substitutions, insertions, or deletions, and the VL CDR3 comprises the amino acid sequence of SEQ ID NO: 17 or 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the amino acid sequence of SEQ ID NO: 12 or 18 comprising 0, 1, 2, 3, 4, or 5 substitutions, insertions, or deletions, the VH CDR2 comprises the amino acid sequence of SEQ ID NO: 13, 19 or 61 comprising 0, 1, 2, 3, 4, or 5 substitutions, insertions, or deletions, the VH CDR3 comprises the amino acid sequence of SEQ ID NO: 14, 20-22, 59, 60, 62, 67 or 140-144 comprising 0, 1, 2, 3, 4, or 5 substitutions, insertions, or deletions, the VL CDR1 comprises the amino acid sequence of SEQ ID NO: 15, 23 or 63 comprising 0, 1, 2, 3, 4, or 5 substitutions, insertions, or deletions, the VL CDR2 comprises the amino acid sequence of SEQ ID NO: 16, 24 or 145 comprising 0, 1, 2, 3, 4, or 5 substitutions, insertions, or deletions, and the VL CDR3 comprises the amino acid sequence of SEQ ID NO: 17, 25, 68, 146, or 147 comprising 0, 1, 2, 3, 4, or 5 substitutions, insertions, or deletions. In one embodiment, at least one of the VH CDR1, VH CDR2, VH CDR3, VL CDR1, VL CDR2, and VL CDR3 comprises a different amino acid sequence than the corresponding CDR of the PGT-121 antibody. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO:19); and the VH CDR3 comprises the sequence of TLHGRRIYGVVAFNEWFTYFYWEV (SEQ ID NO: 20). In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO: 19); and the VH CDR3 comprises the sequence of TLHGRRIYGVVAFNELFTYFYWEV (SEQ ID NO: 21). In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO: 19); and the VH CDR3 comprises the sequence of TLHGRRIYGVVAFNEFFTYFYWEV (SEQ ID NO: 22). In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12; the VH CDR2 comprises the sequence of SEQ ID NO:61; and the VH CDR3 comprises the sequence of SEQ ID NO: 62. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO:19; and the VH CDR3 comprises the sequence of SEQ ID NO: 59. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO:19; and the VH CDR3 comprises the sequence of SEQ ID NO: 60. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO:19; and the VH CDR3 comprises the sequence of SEQ ID NO: 67. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; and the VH CDR3 comprises the sequence of SEQ ID NO: 140. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; and the VH CDR3 comprises the sequence of SEQ ID NO: 141. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; and the VH CDR3 comprises the sequence of SEQ ID NO: 142. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; and the VH CDR3 comprises the sequence of SEQ ID NO: 143. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; and the VH CDR3 comprises the sequence of SEQ ID NO: 144. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the sequence of DSYWS (SEQ ID NO: 12) comprising 0, 1, 2, 3, 4, or 5 substitutions, insertions, or deletions; the VH CDR2 comprises the sequence of YVHKSGDTNYSPSLKS (SEQ ID NO: 13) comprising 0, 1, 2, 3, 4, or 5 substitutions, insertions, or deletions; and the VH CDR3 comprises the sequence of TLHGRRIYGIVAFNEWFTYFYMDV (SEQ ID NO: 14) comprising 0, 1, 2, 3, 4, or 5 substitutions, insertions, or deletions, wherein at least one of the VH CDR1, VH CDR2, and VH CDR3 comprises a different amino acid sequence than the corresponding CDR of the PGT-121 antibody. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO:19) comprising 0, 1, 2, 3, 4, or 5 substitutions, insertions, or deletions; and the VH CDR3 comprises the sequence of TLHGRRIYGVVAFNEWFTYFYWEV (SEQ ID NO: 20) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO: 19) comprising 0, 1, 2, 3, 4, or 5 substitutions, insertions, or deletions; and the VH CDR3 comprises the sequence of TLHGRRIYGVVAFNELFTYFYWEV (SEQ ID NO: 21) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO: 19) comprising 0, 1, 2, 3, 4, or 5 substitutions, insertions, or deletions; and the VH CDR3 comprises the sequence of TLHGRRIYGVVAFNEFFTYFYWEV (SEQ ID NO: 22) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO: 19 comprising 0, 1, 2, 3, 4, or 5 substitutions, insertions, or deletions; and the VH CDR3 comprises the sequence of SEQ ID NO: 59 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO: 19 comprising 0, 1, 2, 3, 4, or 5 substitutions, insertions, or deletions; and the VH CDR3 comprises the sequence of SEQ ID NO: 60 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12 comprising 0, 1, 2, 3, 4, or 5 substitutions, insertions, or deletions; the VH CDR2 comprises the sequence of SEQ ID NO: 61 comprising 0, 1, 2, 3, 4, or 5 substitutions, insertions, or deletions; and the VH CDR3 comprises the sequence of SEQ ID NO: 62 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO:19 comprising 0, 1, 2, 3, 4, or 5 substitutions, insertions, or deletions; and the VH CDR3 comprises the sequence of SEQ ID NO: 67 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO: 19 comprising 0, 1, 2, 3, 4, or 5 substitutions, insertions, or deletions; and the VH CDR3 comprises the sequence of SEQ ID NO: 140 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO: 19 comprising 0, 1, 2, 3, 4, or 5 substitutions, insertions, or deletions; and the VH CDR3 comprises the sequence of SEQ ID NO: 141 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO: 19 comprising 0, 1, 2, 3, 4, or 5 substitutions, insertions, or deletions; and the VH CDR3 comprises the sequence of SEQ ID NO: 142 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO: 19 comprising 0, 1, 2, 3, 4, or 5 substitutions, insertions, or deletions; and the VH CDR3 comprises the sequence of SEQ ID NO: 143 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO: 19 comprising 0, 1, 2, 3, 4, or 5 substitutions, insertions, or deletions; and the VH CDR3 comprises the sequence of SEQ ID NO: 144 comprising 0, 1, 2, 3, 4, or 5 substitutions, insertions, or deletions. In one embodiment, at least one of the VH CDR1, VH CDR2, and VH CDR3 comprises a different amino acid sequence than the corresponding CDR of the PGT-121 antibody. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR3 comprises the sequence of TLHGRRIYGIVAFNEWFTYFYMDV (SEQ ID NO: 14) comprising 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR3 comprises the sequence of TLHGRRIYGVVAFNEWFTYFYWEV (SEQ ID NO: 20) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR3 comprises the sequence of TLHGRRIYGVVAFNELFTYFYWEV (SEQ ID NO: 21) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR3 comprises the sequence of TLHGRRIYGVVAFNEFFTYFYWEV (SEQ ID NO: 22) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR3 comprises the sequence of SEQ ID NO: 59 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR3 comprises the sequence of SEQ ID NO: 60 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR3 comprises the sequence of SEQ ID NO: 62 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR3 comprises the sequence of SEQ ID NO: 67 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR3 comprises the sequence of SEQ ID NO: 140-144 comprising 0, 1, 2, 3, 4, or 5 substitutions, insertions, or deletions. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the antibody comprises the VL CDR1, VL CDR2, and VL CDR3 of the ePGT-121.15.L1 VL (SEQ ID NO: 10). In one embodiment, an antibody described herein comprises a VH CDR1, VH CDR2, VH CDR3, VL CDR1, VL CDR2, and VL CDR3, wherein the antibody comprises the VL CDR1, VL CDR2, and VL CDR3 of the ePGT-121.15.L2 VL (SEQ ID NO: 11). In one embodiment, an antibody described herein comprises a VH CDR1, VH CDR2, VH CDR3, VL CDR1, VL CDR2, and VL CDR3, wherein the antibody comprises the VL CDR1, VL CDR2, and VL CDR3 of the ePGT-121.12.L4 VL (SEQ ID NO: 46). In one embodiment, an antibody described herein comprises a VH CDR1, VH CDR2, VH CDR3, VL CDR1, VL CDR2, and VL CDR3, wherein the antibody comprises the VL CDR1, VL CDR2, and VL CDR3 of the ePGT-121.17.L1 VL (SEQ ID NO: 66). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VL CDR1 comprises the sequence of GGESIGSRAVQ (SEQ ID NO: 23); the VL CDR2 comprises the sequence of NNQDRPP (SEQ ID NO: 24); and the VL CDR3 comprises the sequence of HIWDSRRPTNWV (SEQ ID NO: 25). In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 63; the VL CDR2 comprises the sequence of SEQ ID NO: 16; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 23; the VL CDR2 comprises the sequence of SEQ ID NO: 24; and the VL CDR3 comprises the sequence of SEQ ID NO: 68. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 23; the VL CDR2 comprises the sequence of SEQ ID NO: 24; and the VL CDR3 comprises the sequence of SEQ ID NO: 146. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 23; the VL CDR2 comprises the sequence of SEQ ID NO: 24; and the VL CDR3 comprises the sequence of SEQ ID NO: 147. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 23; the VL CDR2 comprises the sequence of SEQ ID NO: 145; and the VL CDR3 comprises the sequence of SEQ ID NO: 68. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VL CDR1 comprises the sequence of GEKSLGSRAVQ (SEQ ID NO: 15) comprising 0, 1, 2, 3, 4, or 5 substitutions, insertions, or deletions; the VL CDR2 comprises the sequence of NNQDRPS (SEQ ID NO: 16) comprising 0, 1, 2, 3, 4, or 5 substitutions, insertions, or deletions; and the VL CDR3 comprises the sequence of HIWDSRVPTKWV (SEQ ID NO: 17) comprising 0, 1, 2, 3, 4, or 5 substitutions, insertions, or deletions, wherein at least one of the VL CDR1, VL CDR2, and VL CDR3 comprises a different amino acid sequence than the corresponding CDR of the PGT-121 antibody. In one embodiment, an antibody described herein comprises a VH CDR1, VH CDR2, VH CDR3, VL CDR1, VL CDR2, and VL CDR3, wherein the VL CDR1 comprises the sequence of GGESIGSRAVQ (SEQ ID NO: 23) comprising 0, 1, 2, 3, 4, or 5 substitutions, insertions, or deletions; the VL CDR2 comprises the sequence of NNQDRPP (SEQ ID NO: 24) comprising 0, 1, 2, 3, 4, or 5 substitutions, insertions, or deletions; and the VL CDR3 comprises the sequence of HIWDSRRPTNWV (SEQ ID NO: 25) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 63 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 23 comprising 0, 1, 2, 3, 4, or 5 substitutions, insertions, or deletions; the VL CDR2 comprises the sequence of SEQ ID NO: 24 comprising 0, 1, 2, 3, 4, or 5 substitutions, insertions, or deletions; and the VL CDR3 comprises the sequence of SEQ ID NO: 68 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 23 comprising 0, 1, 2, 3, 4, or 5 substitutions, insertions, or deletions; the VL CDR2 comprises the sequence of SEQ ID NO: 24 comprising 0, 1, 2, 3, 4, or 5 substitutions, insertions, or deletions; and the VL CDR3 comprises the sequence of SEQ ID NO: 146 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 23 comprising 0, 1, 2, 3, 4, or 5 substitutions, insertions, or deletions; the VL CDR2 comprises the sequence of SEQ ID NO: 24 comprising 0, 1, 2, 3, 4, or 5 substitutions, insertions, or deletions; and the VL CDR3 comprises the sequence of SEQ ID NO: 147 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VL CDR1 comprises the sequence of SEQ ID NO: 23 comprising 0, 1, 2, 3, 4, or 5 substitutions, insertions, or deletions; the VL CDR2 comprises the sequence of SEQ ID NO: 145 comprising 0, 1, 2, 3, 4, or 5 substitutions, insertions, or deletions; and the VL CDR3 comprises the sequence of SEQ ID NO: 68 comprising 0, 1, 2, 3, 4, or 5 substitutions, insertions, or deletions. In one embodiment, at least one of the VL CDR1, VL CDR2, and VL CDR3 comprises a different amino acid sequence than the corresponding CDR of the PGT-121 antibody. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H1 VH (SEQ ID NO: 3) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H3 VH (SEQ ID NO: 5) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H6 VH (SEQ ID NO: 8) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.13.H1 VH (SEQ ID NO: 45) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.14.H3 VH (SEQ ID NO: 51) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.14.H4 VH (SEQ ID NO: 52) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H64 VH (SEQ ID NO: 8) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H1 VH (SEQ ID NO: 65) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H17 VH (SEQ ID NO: 69) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H18 VH (SEQ ID NO: 70) and the VL CDR1, VL CDR2, and VL CDR3 of the PGT-121 VL (SEQ ID NO: 2). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of SEQ ID NO: 65, 69, 70, or 112-126 and the VL CDR1, VL CDR2, and VL CDR3 of the SEQ ID NO: 66 or 127-139. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the PGT-121 VH (SEQ ID NO: 1), and the VL CDR1, VL CDR2, and VL CDR3 of the ePGT-121.15.L1 VL (SEQ ID NO: 10). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H1 VH (SEQ ID NO: 3) and the VL CDR1, VL CDR2, and VL CDR3 of the ePGT-121.15.L1 VL (SEQ ID NO: 10). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H3 VH (SEQ ID NO: 5) and the VL CDR1, VL CDR2, and VL CDR3 of the ePGT-121.15.L1 VL (SEQ ID NO: 10). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.13.H1 VH (SEQ ID NO: 45) and the VL CDR1, VL CDR2, and VL CDR3 of the ePGT-121.15.L1 VL (SEQ ID NO: 10). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.14.H3 VH (SEQ ID NO: 51) and the VL CDR1, VL CDR2, and VL CDR3 of the ePGT-121.15.L1 VL (SEQ ID NO: 10). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.14.H4 VH (SEQ ID NO: 52) and the VL CDR1, VL CDR2, and VL CDR3 of the ePGT-121.15.L1 VL (SEQ ID NO: 10). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H1 VH (SEQ ID NO: 65) and the VL CDR1, VL CDR2, and VL CDR3 of the ePGT-121.17.L1 VL (SEQ ID NO: 66). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H17 VH (SEQ ID NO: 69) and the VL CDR1, VL CDR2, and VL CDR3 of the ePGT-121.17.L1 VL (SEQ ID NO: 66). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.18.H18 VH (SEQ ID NO: 70) and the VL CDR1, VL CDR2, and VL CDR3 of the ePGT-121.17.L1 VL (SEQ ID NO: 66). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.17.L1 VL (SEQ ID NO: 66). In one embodiment, an antibody described herein comprises a VH CDR1, VH CDR2, VH CDR3, VL CDR1, VL CDR2, and VL CDR3, wherein the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.14.L1 VL (SEQ ID NO: 55). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO:19); the VH CDR3 comprises the sequence of TLHGRRIYGVVAFNEWFTYFYWEV (SEQ ID NO: 20), the VL CDR1 comprises the sequence of GEKSLGSRAVQ (SEQ ID NO: 15); the VL CDR2 comprises the sequence of NNQDRPS (SEQ ID NO: 16); and the VL CDR3 comprises the sequence of HIWDSRVPTKWV (SEQ ID NO: 17). In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO: 19); the VH CDR3 comprises the sequence of TLHGRRIYGVVAFNELFTYFYWEV (SEQ ID NO: 21); the VL CDR1 comprises the sequence of GEKSLGSRAVQ (SEQ ID NO: 15); the VL CDR2 comprises the sequence of NNQDRPS (SEQ ID NO: 16); and the VL CDR3 comprises the sequence of HIWDSRVPTKWV (SEQ ID NO: 17). In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO: 19); the VH CDR3 comprises the sequence of TLHGRRIYGVVAFNEFFTYFYWEV (SEQ ID NO: 22); the VL CDR1 comprises the sequence of GEKSLGSRAVQ (SEQ ID NO: 15); the VL CDR2 comprises the sequence of NNQDRPS (SEQ ID NO: 16); and the VL CDR3 comprises the sequence of HIWDSRVPTKWV (SEQ ID NO: 17). In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 59; the VL CDR1 comprises the sequence of SEQ ID NO: 15; the VL CDR2 comprises the sequence of SEQ ID NO: 16; and the VL CDR3 comprises the sequence of SEQ ID NO: 17. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 60; the VL CDR1 comprises the sequence of SEQ ID NO: 15; the VL CDR2 comprises the sequence of SEQ ID NO: 16; and the VL CDR3 comprises the sequence of SEQ ID NO: 17. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12; the VH CDR2 comprises the sequence of SEQ ID NO: 61; the VH CDR3 comprises the sequence of SEQ ID NO: 62; the VL CDR1 comprises the sequence of SEQ ID NO: 15; the VL CDR2 comprises the sequence of SEQ ID NO: 16; and the VL CDR3 comprises the sequence of SEQ ID NO: 17. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 67; the VL CDR1 comprises the sequence of SEQ ID NO: 23; the VL CDR2 comprises the sequence of SEQ ID NO: 24; and the VL CDR3 comprises the sequence of SEQ ID NO: 68. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the sequence of DSYWS (SEQ ID NO: 12); the VH CDR2 comprises the sequence of YVHKSGDTNYSPSLKS (SEQ ID NO: 13); the VH CDR3 comprises the sequence of TLHGRRIYGIVAFNEWFTYFYMDV (SEQ ID NO: 14); the VL CDR1 comprises the sequence of GGESIGSRAVQ (SEQ ID NO: 23); the VL CDR2 comprises the sequence of NNQDRPP (SEQ ID NO: 24); and the VL CDR3 comprises the sequence of HIWDSRRPTNWV (SEQ ID NO: 25). In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO:19); the VH CDR3 comprises the sequence of TLHGRRIYGVVAFNEWFTYFYWEV (SEQ ID NO: 20); the VL CDR1 comprises the sequence of GGESIGSRAVQ (SEQ ID NO: 23); the VL CDR2 comprises the sequence of NNQDRPP (SEQ ID NO: 24); and the VL CDR3 comprises the sequence of HIWDSRRPTNWV (SEQ ID NO: 25). In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO: 19); the VH CDR3 comprises the sequence of TLHGRRIYGVVAFNELFTYFYWEV (SEQ ID NO: 21); the VL CDR1 comprises the sequence of GGESIGSRAVQ (SEQ ID NO: 23); the VL CDR2 comprises the sequence of NNQDRPP (SEQ ID NO: 24); and the VL CDR3 comprises the sequence of HIWDSRRPTNWV (SEQ ID NO: 25). In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18); the VH CDR2 comprises the sequence of YTHHSGDTNYAPSLKS (SEQ ID NO: 19); the VH CDR3 comprises the sequence of TLHGRRIYGVVAFNEFFTYFYWEV (SEQ ID NO: 22); the VL CDR1 comprises the sequence of GGESIGSRAVQ (SEQ ID NO: 23); the VL CDR2 comprises the sequence of NNQDRPP (SEQ ID NO: 24); and the VL CDR3 comprises the sequence of HIWDSRRPTNWV (SEQ ID NO: 25). In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 59; the VL CDR1 comprises the sequence of SEQ ID NO: 23; the VL CDR2 comprises the sequence of SEQ ID NO: 24; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 60; the VL CDR1 comprises the sequence of SEQ ID NO: 23; the VL CDR2 comprises the sequence of SEQ ID NO: 24; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12; the VH CDR2 comprises the sequence of SEQ ID NO: 61; the VH CDR3 comprises the sequence of SEQ ID NO: 62; the VL CDR1 comprises the sequence of SEQ ID NO: 23; the VL CDR2 comprises the sequence of SEQ ID NO: 24; and the VL CDR3 comprises the sequence of SEQ ID NO: 25. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12; the VH CDR2 comprises the sequence of SEQ ID NO: 13; the VH CDR3 comprises the sequence of SEQ ID NO: 14; the VL CDR1 comprises the sequence of SEQ ID NO: 63; the VL CDR2 comprises the sequence of SEQ ID NO: 16; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 20; the VL CDR1 comprises the sequence of SEQ ID NO: 63; the VL CDR2 comprises the sequence of SEQ ID NO: 16; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 21; the VL CDR1 comprises the sequence of SEQ ID NO: 63; the VL CDR2 comprises the sequence of SEQ ID NO: 16; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 22; the VL CDR1 comprises the sequence of SEQ ID NO: 63; the VL CDR2 comprises the sequence of SEQ ID NO: 16; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 59; the VL CDR1 comprises the sequence of SEQ ID NO: 63; the VL CDR2 comprises the sequence of SEQ ID NO: 16; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18; the VH CDR2 comprises the sequence of SEQ ID NO: 19; the VH CDR3 comprises the sequence of SEQ ID NO: 60; the VL CDR1 comprises the sequence of SEQ ID NO: 63; the VL CDR2 comprises the sequence of SEQ ID NO: 16; and the VL CDR3 comprises the sequence of SEQ ID NO: 25. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12; the VH CDR2 comprises the sequence of SEQ ID NO: 61; the VH CDR3 comprises the sequence of SEQ ID NO: 62; the VL CDR1 comprises the sequence of SEQ ID NO: 63; the VL CDR2 comprises the sequence of SEQ ID NO: 16; and the VL CDR3 comprises the sequence of SEQ ID NO: 25. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3 comprising the sequence of SEQ ID NO: 18, 19, 20, 23, 24, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 20, 15, 16, 17, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 20, 63, 16, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 20, 23, 24, 68, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 21, 23, 24, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 21, 15, 16, 17, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 21, 63, 16, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 21, 23, 24, 68, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 22, 23, 24, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 22, 15, 16, 17, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 22, 63, 16, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 22, 23, 24, 68, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 59, 23, 24, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 59, 15, 16, 17, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 59, 63, 16, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 59, 23, 24, 68, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 60, 23, 24, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 60, 15, 16, 17, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 60, 63, 16, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 60, 23, 24, 68, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 67, 23, 24, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 67, 15, 16, 17, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 67, 63, 16, 25, respectively. In one embodiment, an antibody described herein comprises a VH CDR1, VH CDR2, VH CDR3, VL CDR1, VL CDR2, and VL CDR3 comprising the sequence of SEQ ID NO: 18, 19, 67, 23, 24, 68, respectively. In one embodiment, an antibody described herein comprises a VH CDR1, VH CDR2, VH CDR3, VL CDR1, VL CDR2, and VL CDR3 comprising the sequence of SEQ ID NO: 12, 61, 62, 23, 24, 25, respectively. In one embodiment, an antibody described herein comprises a VH CDR1, VH CDR2, VH CDR3, VL CDR1, VL CDR2, and VL CDR3 comprising the sequence of SEQ ID NO: 12, 61, 62, 15, 16, 17, respectively. In one embodiment, an antibody described herein comprises a VH CDR1, VH CDR2, VH CDR3, VL CDR1, VL CDR2, and VL CDR3 comprising the sequence of SEQ ID NO: 12, 61, 62, 63, 16, 25, respectively. In one embodiment, an antibody described herein comprises a VH CDR1, VH CDR2, VH CDR3, VL CDR1, VL CDR2, and VL CDR3 comprising the sequence of SEQ ID NO: 12, 61, 62, 23, 24, 68, respectively. In one embodiment, an antibody described herein comprises a VH CDR1, VH CDR2, VH CDR3, VL CDR1, VL CDR2, and VL CDR3 comprising the sequence of SEQ ID NO: 12, 13, 14, 23, 24, 25, respectively. In one embodiment, an antibody described herein comprises a VH CDR1, VH CDR2, VH CDR3, VL CDR1, VL CDR2, and VL CDR3 comprising the sequence of SEQ ID NO: 12, 13, 14, 63, 16, 25, respectively. In one embodiment, an antibody described herein comprises a VH CDR1, VH CDR2, VH CDR3, VL CDR1, VL CDR2, and VL CDR3 comprising the sequence of SEQ ID NO: 12, 13, 14, 23, 24, 68, respectively. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO:19) comprising 0, 1, 2, 3, 4, or 5 substitutions, insertions, or deletions; the VH CDR3 comprises the sequence of TLHGRRIYGVVAFNEWFTYFYWEV (SEQ ID NO: 20) comprising 0, 1, 2, 3, 4, or 5 substitutions, insertions, or deletions, the VL CDR1 comprises the sequence of GEKSLGSRAVQ (SEQ ID NO: 15) comprising 0, 1, 2, 3, 4, or 5 substitutions, insertions, or deletions; the VL CDR2 comprises the sequence of NNQDRPS (SEQ ID NO: 16) comprising 0, 1, 2, 3, 4, or 5 substitutions, insertions, or deletions; and the VL CDR3 comprises the sequence of HIWDSRVPTKWV (SEQ ID NO: 17) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO: 19) comprising 0, 1, 2, 3, 4, or 5 substitutions, insertions, or deletions; the VH CDR3 comprises the sequence of TLHGRRIYGVVAFNELFTYFYWEV (SEQ ID NO: 21) comprising 0, 1, 2, 3, 4, or 5 substitutions, insertions, or deletions; the VL CDR1 comprises the sequence of GEKSLGSRAVQ (SEQ ID NO: 15) comprising 0, 1, 2, 3, 4, or 5 substitutions, insertions, or deletions; the VL CDR2 comprises the sequence of NNQDRPS (SEQ ID NO: 16) comprising 0, 1, 2, 3, 4, or 5 substitutions, insertions, or deletions; and the VL CDR3 comprises the sequence of HIWDSRVPTKWV (SEQ ID NO: 17) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO: 19) comprising 0, 1, 2, 3, 4, or 5 substitutions, insertions, or deletions; the VH CDR3 comprises the sequence of TLHGRRIYGVVAFNEFFTYFYWEV (SEQ ID NO: 22) comprising 0, 1, 2, 3, 4, or 5 substitutions, insertions, or deletions; the VL CDR1 comprises the sequence of GEKSLGSRAVQ (SEQ ID NO: 15) comprising 0, 1, 2, 3, 4, or 5 substitutions, insertions, or deletions; the VL CDR2 comprises the sequence of NNQDRPS (SEQ ID NO: 16) comprising 0, 1, 2, 3, 4, or 5 substitutions, insertions, or deletions; and the VL CDR3 comprises the sequence of HIWDSRVPTKWV (SEQ ID NO: 17) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO:19 comprising 0, 1, 2, 3, 4, or 5 substitutions, insertions, or deletions; the VH CDR3 comprises the sequence of SEQ ID NO: 59 comprising 0, 1, 2, 3, 4, or 5 substitutions, insertions, or deletions, the VL CDR1 comprises the sequence of SEQ ID NO: 15 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 17 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO:19 comprising 0, 1, 2, 3, 4, or 5 substitutions, insertions, or deletions; the VH CDR3 comprises the sequence of SEQ ID NO: 60 comprising 0, 1, 2, 3, 4, or 5 substitutions, insertions, or deletions, the VL CDR1 comprises the sequence of SEQ ID NO: 15 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 17 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12 comprising 0, 1, 2, 3, 4, or 5 substitutions, insertions, or deletions; the VH CDR2 comprises the sequence of SEQ ID NO:61 comprising 0, 1, 2, 3, 4, or 5 substitutions, insertions, or deletions; the VH CDR3 comprises the sequence of SEQ ID NO: 62 comprising 0, 1, 2, 3, 4, or 5 substitutions, insertions, or deletions, the VL CDR1 comprises the sequence of SEQ ID NO: 15 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 17 comprising 0, 1, 2, 3, 4, or 5 substitutions, insertions, or deletions. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120) comprising 0, 1, 2, 3, 4, or 5 substitutions, insertions, or deletions.

In one embodiment, an antibody described herein comprises a VH CDR1, VH CDR2, VH CDR3, VL CDR1, VL CDR2, and VL CDR3, wherein the VH CDR1 comprises the sequence of DSYWS (SEQ ID NO: 12) comprising 0, 1, 2, 3, 4, or 5 substitutions, insertions, or deletions; the VH CDR2 comprises the sequence of YVHKSGDTNYSPSLKS (SEQ ID NO: 13) comprising 0, 1, 2, 3, 4, or 5 substitutions, insertions, or deletions; the VH CDR3 comprises the sequence of TLHGRRIYGIVAFNEWFTYFYMDV (SEQ ID NO: 14) comprising 0, 1, 2, 3, 4, or 5 substitutions, insertions, or deletions; the VL CDR1 comprises the sequence of GGESIGSRAVQ (SEQ ID NO: 23) comprising 0, 1, 2, 3, 4, or 5 substitutions, insertions, or deletions; the VL CDR2 comprises the sequence of NNQDRPP (SEQ ID NO: 24) comprising 0, 1, 2, 3, 4, or 5 substitutions, insertions, or deletions; and the VL CDR3 comprises the sequence of HIWDSRRPTNWV (SEQ ID NO: 25) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO:19) comprising 0, 1, 2, 3, 4, or 5 substitutions, insertions, or deletions; the VH CDR3 comprises the sequence of TLHGRRIYGVVAFNEWFTYFYWEV (SEQ ID NO: 20) comprising 0, 1, 2, 3, 4, or 5 substitutions, insertions, or deletions; the VL CDR1 comprises the sequence of GGESIGSRAVQ (SEQ ID NO: 23) comprising 0, 1, 2, 3, 4, or 5 substitutions, insertions, or deletions; the VL CDR2 comprises the sequence of NNQDRPP (SEQ ID NO: 24) comprising 0, 1, 2, 3, 4, or 5 substitutions, insertions, or deletions; and the VL CDR3 comprises the sequence of HIWDSRRPTNWV (SEQ ID NO: 25) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO: 19) comprising 0, 1, 2, 3, 4, or 5 substitutions, insertions, or deletions; the VH CDR3 comprises the sequence of TLHGRRIYGVVAFNELFTYFYWEV (SEQ ID NO: 21) comprising 0, 1, 2, 3, 4, or 5 substitutions, insertions, or deletions; the VL CDR1 comprises the sequence of GGESIGSRAVQ (SEQ ID NO: 23) comprising 0, 1, 2, 3, 4, or 5 substitutions, insertions, or deletions; the VL CDR2 comprises the sequence of NNQDRPP (SEQ ID NO: 24) comprising 0, 1, 2, 3, 4, or 5 substitutions, insertions, or deletions; and the VL CDR3 comprises the sequence of HIWDSRRPTNWV (SEQ ID NO: 25) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO: 19) comprising 0, 1, 2, 3, 4, or 5 substitutions, insertions, or deletions; the VH CDR3 comprises the sequence of TLHGRRIYGVVAFNEFFTYFYWEV (SEQ ID NO: 22) comprising 0, 1, 2, 3, 4, or 5 substitutions, insertions, or deletions; the VL CDR1 comprises the sequence of GGESIGSRAVQ (SEQ ID NO: 23) comprising 0, 1, 2, 3, 4, or 5 substitutions, insertions, or deletions; the VL CDR2 comprises the sequence of NNQDRPP (SEQ ID NO: 24) comprising 0, 1, 2, 3, 4, or 5 substitutions, insertions, or deletions; and the VL CDR3 comprises the sequence of HIWDSRRPTNWV (SEQ ID NO: 25) comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO:19 comprising 0, 1, 2, 3, 4, or 5 substitutions, insertions, or deletions; the VH CDR3 comprises the sequence of SEQ ID NO: 59 comprising 0, 1, 2, 3, 4, or 5 substitutions, insertions, or deletions, the VL CDR1 comprises the sequence of SEQ ID NO: 23 comprising 0, 1, 2, 3, 4, or 5 substitutions, insertions, or deletions; the VL CDR2 comprises the sequence of SEQ ID NO: 24 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO:19 comprising 0, 1, 2, 3, 4, or 5 substitutions, insertions, or deletions; the VH CDR3 comprises the sequence of SEQ ID NO: 60 comprising 0, 1, 2, 3, 4, or 5 substitutions, insertions, or deletions, the VL CDR1 comprises the sequence of SEQ ID NO: 23 comprising 0, 1, 2, 3, 4, or 5 substitutions, insertions, or deletions; the VL CDR2 comprises the sequence of SEQ ID NO: 24 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12 comprising 0, 1, 2, 3, 4, or 5 substitutions, insertions, or deletions; the VH CDR2 comprises the sequence of SEQ ID NO:61 comprising 0, 1, 2, 3, 4, or 5 substitutions, insertions, or deletions; the VH CDR3 comprises the sequence of SEQ ID NO: 62 comprising 0, 1, 2, 3, 4, or 5 substitutions, insertions, or deletions, the VL CDR1 comprises the sequence of SEQ ID NO: 23 comprising 0, 1, 2, 3, 4, or 5 substitutions, insertions, or deletions; the VL CDR2 comprises the sequence of SEQ ID NO: 24 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3, wherein the VH CDR1 comprises the sequence of DSYWS (SEQ ID NO: 12) comprising 0, 1, 2, 3, 4, or 5 substitutions, insertions, or deletions; the VH CDR2 comprises the sequence of YVHKSGDTNYSPSLKS (SEQ ID NO: 13) comprising 0, 1, 2, 3, 4, or 5 substitutions, insertions, or deletions; the VH CDR3 comprises the sequence of TLHGRRIYGIVAFNEWFTYFYMDV (SEQ ID NO: 14) comprising 0, 1, 2, 3, 4, or 5 substitutions, insertions, or deletions; the VL CDR1 comprises the sequence of SEQ ID NO: 63 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO:19) comprising 0, 1, 2, 3, 4, or 5 substitutions, insertions, or deletions; the VH CDR3 comprises the sequence of TLHGRRIYGVVAFNEWFTYFYWEV (SEQ ID NO: 20) comprising 0, 1, 2, 3, 4, or 5 substitutions, insertions, or deletions; the VL CDR1 comprises the sequence of SEQ ID NO: 63 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO: 19) comprising 0, 1, 2, 3, 4, or 5 substitutions, insertions, or deletions; the VH CDR3 comprises the sequence of TLHGRRIYGVVAFNELFTYFYWEV (SEQ ID NO: 21) comprising 0, 1, 2, 3, 4, or 5 substitutions, insertions, or deletions; the VL CDR1 comprises the sequence of SEQ ID NO: 63 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SSYWS (SEQ ID NO: 18) comprising 0, 1, 2, 3, 4, or 5 substitutions, insertions, or deletions; the VH CDR2 comprises the sequence of YTHHSGDTNYAPSLKS (SEQ ID NO: 19) comprising 0, 1, 2, 3, 4, or 5 substitutions, insertions, or deletions; the VH CDR3 comprises the sequence of TLHGRRIYGVVAFNEFFTYFYWEV (SEQ ID NO: 22) comprising 0, 1, 2, 3, 4, or 5 substitutions, insertions, or deletions; the VL CDR1 comprises the sequence of SEQ ID NO: 63 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO:19 comprising 0, 1, 2, 3, 4, or 5 substitutions, insertions, or deletions; the VH CDR3 comprises the sequence of SEQ ID NO: 59 comprising 0, 1, 2, 3, 4, or 5 substitutions, insertions, or deletions, the VL CDR1 comprises the sequence of SEQ ID NO: 63 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 18 comprising 0, 1, 2, 3, 4, or 5 substitutions, insertions, or deletions; the VH CDR2 comprises the sequence of SEQ ID NO:19 comprising 0, 1, 2, 3, 4, or 5 substitutions, insertions, or deletions; the VH CDR3 comprises the sequence of SEQ ID NO: 60 comprising 0, 1, 2, 3, 4, or 5 substitutions, insertions, or deletions, the VL CDR1 comprises the sequence of SEQ ID NO: 63 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3, wherein the VH CDR1 comprises the sequence of SEQ ID NO: 12 comprising 0, 1, 2, 3, 4, or 5 substitutions, insertions, or deletions; the VH CDR2 comprises the sequence of SEQ ID NO:61 comprising 0, 1, 2, 3, 4, or 5 substitutions, insertions, or deletions; the VH CDR3 comprises the sequence of SEQ ID NO: 62 comprising 0, 1, 2, 3, 4, or 5 substitutions, insertions, or deletions, the VL CDR1 comprises the sequence of SEQ ID NO: 63 comprising 0, 1, 2, 3, 4, or 5 substitutions, insertions, or deletions; the VL CDR2 comprises the sequence of SEQ ID NO: 16 comprising 0, 1, 2, 3, 4, or 5 substitutions, insertions, or deletions; and the VL CDR3 comprises the sequence of SEQ ID NO: 25 comprising 0, 1, 2, 3, 4, or 5 substitutions, insertions, or deletions. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, and VL CDR3 comprising the sequence of SEQ ID NO: 18, 19, 20, 23, 24, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 20, 15, 16, 17, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 20, 63, 16, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 20, 23, 24, 68, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 21, 23, 24, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 21, 15, 16, 17, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 21, 63, 16, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 21, 23, 24, 68, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 22, 23, 24, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 22, 15, 16, 17, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 22, 63, 16, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 22, 23, 24, 68, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 59, 23, 24, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 59, 15, 16, 17, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 59, 63, 16, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 59, 23, 24, 68, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 60, 23, 24, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 60, 15, 16, 17, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 60, 63, 16, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 60, 23, 24, 68, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 67, 23, 24, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 67, 15, 16, 17, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 67, 63, 16, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 18, 19, 67, 23, 24, 68, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 12, 61, 62, 23, 24, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 12, 61, 62, 15, 16, 17, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 12, 61, 62, 63, 16, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 12, 61, 62, 23, 24, 68, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 12, 13, 14, 23, 24, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 12, 13, 14, 63, 16, 25, respectively, each comprising 0, 1, 2, 3, 4, or 5 substitutions, insertions, or deletions. In one embodiment, an antibody described herein comprises a VH CDR1, VH CDR2, VH CDR3, VL CDR1, VL CDR2, and VL CDR3 comprising the sequence of SEQ ID NO: 12, 13, 14, 23, 24, 68, respectively, each comprising 0, 1, 2, 3, 4, or 5 substitutions, insertions, or deletions. In one embodiment, at least one of the VH CDR1, VH CDR2, VH CDR3, VL CDR1, VL CDR2, and VL CDR3 comprises a different amino acid sequence than the corresponding CDR of the PGT-121 antibody. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein further comprises a VH FR1, VH FR2, VH FR3, and VH FR4, wherein the VH FR1 comprises the sequence of QX₁QLQESGPGLVKPX₂ETLSLTCSVSGASIS wherein X₁ is M or V, and X2 is S or G (SEQ ID NO: 77); the VH FR2 comprises the sequence of WX₁RX₂X₃PGKGX₄EWIG wherein X₁ is I or L, X₂ is R or E, X₃ is S or T, and X₄ is I, L or V (SEQ ID NO: 80); the VH FR3 comprises the sequence of RVX₁X₂X₃LX₄X₅X₆KNQVSLX₇LX₈X₉X₁₀TAADX₁₁X₁₂X₁₃YYCAR wherein X₁ is N, H, or T, X₂ is L or I, X₃ is S or G, X₄ is D or H, X₅ is T or P, X₆ is S or E, is S or K, X₈ is V, T or K, X₉ is A or S, X₁₀ is A or V, X₁₁ is S or T, X₁₂ is G or A, and X₁₃ is K or V (SEQ ID NO: 88); and the VH FR1 comprises the sequence of YX₁X₂X₃WGX₄GX₅X₆VTVSS wherein X₁ is M or W, X₂ is D, E or P, X₃ is V or T, X₄ is N or K, X₅ is I or T, and X₆ is Q or K (SEQ ID NO: 94). In one embodiment, the VH FR1 comprises the sequence of SEQ ID NO: 74. In one embodiment, VH FR1 comprises the sequence of SEQ ID NO: 75. In one embodiment, the VH FR1 comprises the sequence of SEQ ID NO: 76. In one embodiment, the VH FR2 comprises the sequence of SEQ ID NO: 78. In one embodiment, the VH FR2 comprises the sequence of SEQ ID NO: 79. In one embodiment, the VH FR2 comprises the sequence of SEQ ID NO: 148 or 149. In one embodiment, the VH FR3 comprises the sequence of SEQ ID NO: 81. In one embodiment, the VH FR3 comprises the sequence of SEQ ID NO: 82. In one embodiment, VH FR3 comprises the sequence of SEQ ID NO: 83. In one embodiment, the VH FR3 comprises the sequence of SEQ ID NO: 84. In one embodiment, the VH FR3 comprises the sequence of SEQ ID NO: 85. In one embodiment, the VH FR3 comprises the sequence of SEQ ID NO: 86. In one embodiment, the VH FR3 comprises the sequence of SEQ ID NO: 87. In one embodiment, the VH FR3 comprises the sequence of SEQ ID NO: 150-152. In one embodiment, the VH FR4 comprises the sequence of SEQ ID NO: 89. In one embodiment, the VH FR4 comprises the sequence of SEQ ID NO: 90. In one embodiment, the VH FR4 comprises the sequence of SEQ ID NO: 91. In one embodiment, the VH FR4 comprises the sequence of SEQ ID NO: 92. In one embodiment, the VH FR4 comprises the sequence of SEQ ID NO: 93. In one embodiment, the VH FR4 comprises the sequence of SEQ ID NO: 93. In one embodiment, the VH FR4 comprises the sequence of SEQ ID NO: 153 or 154.

In one embodiment, the VH FR1 comprises the sequence of SEQ ID NO: 74 comprising 0, 1, 2, 3, 4, or 5 substitutions, insertions, or deletions. In one embodiment, VH FR1 comprises the sequence of SEQ ID NO: 75 comprising 0, 1, 2, 3, 4, or 5 substitutions, insertions, or deletions. In one embodiment, the VH FR1 comprises the sequence of SEQ ID NO: 76 comprising 0, 1, 2, 3, 4, or 5 substitutions, insertions, or deletions. In one embodiment, the VH FR2 comprises the sequence of SEQ ID NO: 78 comprising 0, 1, 2, 3, 4, or 5 substitutions, insertions, or deletions. In one embodiment, the VH FR2 comprises the sequence of SEQ ID NO: 79 comprising 0, 1, 2, 3, 4, or 5 substitutions, insertions, or deletions. In one embodiment, the VH FR2 comprises the sequence of SEQ ID NO: 148 or 149 comprising 0, 1, 2, 3, 4, or 5 substitutions, insertions, or deletions. In one embodiment, the VH FR3 comprises the sequence of SEQ ID NO: 81 comprising 0, 1, 2, 3, 4, or 5 substitutions, insertions, or deletions. In one embodiment, the VH FR3 comprises the sequence of SEQ ID NO: 82 comprising 0, 1, 2, 3, 4, or 5 substitutions, insertions, or deletions. In one embodiment, VH FR3 comprises the sequence of SEQ ID NO: 83 comprising 0, 1, 2, 3, 4, or 5 substitutions, insertions, or deletions. In one embodiment, the VH FR3 comprises the sequence of SEQ ID NO: 84 comprising 0, 1, 2, 3, 4, or 5 substitutions, insertions, or deletions. In one embodiment, the VH FR3 comprises the sequence of SEQ ID NO: 85 comprising 0, 1, 2, 3, 4, or 5 substitutions, insertions, or deletions. In one embodiment, the VH FR3 comprises the sequence of SEQ ID NO: 86 comprising 0, 1, 2, 3, 4, or 5 substitutions, insertions, or deletions. In one embodiment, the VH FR3 comprises the sequence of SEQ ID NO: 87 comprising 0, 1, 2, 3, 4, or 5 substitutions, insertions, or deletions. In one embodiment, the VH FR3 comprises the sequence of SEQ ID NO: 150-152 comprising 0, 1, 2, 3, 4, or 5 substitutions, insertions, or deletions. In one embodiment, the VH FR4 comprises the sequence of SEQ ID NO: 89 comprising 0, 1, 2, 3, 4, or 5 substitutions, insertions, or deletions. In one embodiment, the VH FR4 comprises the sequence of SEQ ID NO: 90 comprising 0, 1, 2, 3, 4, or 5 substitutions, insertions, or deletions. In one embodiment, the VH FR4 comprises the sequence of SEQ ID NO: 91 comprising 0, 1, 2, 3, 4, or 5 substitutions, insertions, or deletions. In one embodiment, the VH FR4 comprises the sequence of SEQ ID NO: 92 comprising 0, 1, 2, 3, 4, or 5 substitutions, insertions, or deletions. In one embodiment, the VH FR4 comprises the sequence of SEQ ID NO: 93 comprising 0, 1, 2, 3, 4, or 5 substitutions, insertions, or deletions. In one embodiment, the VH FR4 comprises the sequence of SEQ ID NO: 93 comprising 0, 1, 2, 3, 4, or 5 substitutions, insertions, or deletions. In one embodiment, the VH FR4 comprises the sequence of SEQ ID NO: 153 or 154 comprising 0, 1, 2, 3, 4, or 5 substitutions, insertions, or deletions.

In one embodiment, the VH FR1, VH FR2, VH FR3, and VH FR4 comprises the sequence of SEQ ID NO: 74, 78, 81, and 89, respectively. In one embodiment, the VH FR1, VH FR2, VH FR3, and VH FR4 comprises the sequence of SEQ ID NO: 75, 79, 83, and 91, respectively. In one embodiment, the VH FR1, VH FR2, VH FR3, and VH FR4 comprises the sequence of SEQ ID NO: 75, 79, 83, and 92, respectively. In one embodiment, the VH FR1, VH FR2, VH FR3, and VH FR4 comprises the sequence of SEQ ID NO: 76, 79, 83, and 92, respectively. In one embodiment, the VH FR1, VH FR2, VH FR3, and VH FR4 comprises the sequence of SEQ ID NO: 75, 79, 84, and 92, respectively. In one embodiment, the VH FR1, VH FR2, VH FR3, and VH FR4 comprises the sequence of SEQ ID NO: 74, 78, 81, and 89, respectively. In one embodiment, the VH FR1, VH FR2, VH FR3, and VH FR4 comprises the sequence of SEQ ID NO: 75, 79, 82, and 90, respectively. In one embodiment, the VH FR1, VH FR2, VH FR3, and VH FR4 comprises the sequence of SEQ ID NO: 75, 79, 85, and 91, respectively. In one embodiment, the VH FR1, VH FR2, VH FR3, and VH FR4 comprises the sequence of SEQ ID NO: 75, 79, 85, and 92, respectively. In one embodiment, the VH FR1, VH FR2, VH FR3, and VH FR4 comprises the sequence of SEQ ID NO: 75, 79, 83, and 91, respectively. In one embodiment, the VH FR1, VH FR2, VH FR3, and VH FR4 comprises the sequence of SEQ ID NO: 75, 79, 83, and 93, respectively. In one embodiment, the VH FR1, VH FR2, VH FR3, and VH FR4 comprises the sequence of SEQ ID NO: 75, 79, 86, 93, respectively. In one embodiment, the VH FR1, VH FR2, VH FR3, and VH FR4 comprises the sequence of SEQ ID NO: 75, 79, 87, and 93, respectively.

In one embodiment, the VH FR1, VH FR2, VH FR3, and VH FR4 comprises the sequence of SEQ ID NO: 74, 78, 81, 89, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 83, 91, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 83, 92, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 76, 79, 83, 92, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 84, 92, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 74, 78, 81, 89, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 82, 90, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 85, 91, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 85, 92, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 83, 91, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 83, 93, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 86, 93, respectively, each comprising 0, 1, 2, 3, 4, or 5 substitutions, insertions, or deletions. In one embodiment, the VH FR1, VH FR2, VH FR3, and VH FR4 comprises the sequence of SEQ ID NO: 75, 79, 87, 93, respectively, each comprising 0, 1, 2, 3, 4, or 5 substitutions, insertions, or deletions.

In one embodiment, an antibody described herein further comprises a VL FR1, VL FR2, VL FR3, and VL FR4, wherein the VL FR1 comprises the sequence of X₁X₂X₃SVAPGETX₄RIX₅C wherein X₁ is S or P, X₂ is D or S, X₃ is I, L or V, X₄ is A or V, and X₅ is S or T (SEQ ID NO: 99); the VL FR2 comprises the sequence of WYQX₁RX₂GQAPX₃LIIY wherein X₁ is Q or H, X₂ is A or P, and X₃ is S, K, R or P (SEQ ID NO: 103); the VL FR3 comprises the sequence of GIPERFSGSPDX₁X₂FGTTATLTIX₃X₄VEAGDEAX₅YYC wherein X₁ is S, L or I, X₂ is P, A or D, X₃ is T or S, X4 is S, R or N, and X₅ is D or T (SEQ ID NO: 108); and the VL FR4 comprises the sequence of X₁X₂X₃GTTLTVL wherein X₁ is F or L, X₂ is E or G, and X3 is G or P (SEQ ID NO: 111). In one embodiment, the VL FR1 comprises the sequence of SEQ ID NO: 95. In one embodiment, the VL FR1 comprises the sequence of SEQ ID NO: 96. In one embodiment, the VL FR1 comprises the sequence of SEQ ID NO: 97. In one embodiment, the VL FR1 comprises the sequence of SEQ ID NO: 98. In one embodiment, the VL FR1 comprises the sequence of SEQ ID NO: 155 or 156. In one embodiment, the VL FR2 comprises the sequence of SEQ ID NO: 100. In one embodiment, the VL FR2 comprises the sequence of SEQ ID NO: 101. In one embodiment, the VL FR2 comprises the sequence of SEQ ID NO: 102. In one embodiment, the VL FR2 comprises the sequence of SEQ ID NO: 157-160. In one embodiment, the VL FR3 comprises the sequence of SEQ ID NO: 104. In one embodiment, the VL FR3 comprises the sequence of SEQ ID NO: 105. In one embodiment, the VL FR3 comprises the sequence of SEQ ID NO: 106. In one embodiment, the VL FR3 comprises the sequence of SEQ ID NO: 107. In one embodiment, the VL FR3 comprises the sequence of SEQ ID NO: 161-164. In one embodiment, the VL FR4 comprises the sequence of SEQ ID NO: 109. In one embodiment, the VL FR4 comprises the sequence of SEQ ID NO: 110. In one embodiment, the VL FR4 comprises the sequence of SEQ ID NO: 165-167.

In one embodiment, the VL FR1 comprises the sequence of SEQ ID NO: 95 comprising 0, 1, 2, 3, 4, or 5 substitutions, insertions, or deletions. In one embodiment, the VL FR1 comprises the sequence of SEQ ID NO: 96 comprising 0, 1, 2, 3, 4, or 5 substitutions, insertions, or deletions. In one embodiment, the VL FR1 comprises the sequence of SEQ ID NO: 97 comprising 0, 1, 2, 3, 4, or 5 substitutions, insertions, or deletions. In one embodiment, the VL FR1 comprises the sequence of SEQ ID NO: 98 comprising 0, 1, 2, 3, 4, or 5 substitutions, insertions, or deletions. In one embodiment, the VL FR1 comprises the sequence of SEQ ID NO: 155 or 156 comprising 0, 1, 2, 3, 4, or 5 substitutions, insertions, or deletions. In one embodiment, the VL FR2 comprises the sequence of SEQ ID NO: 100 comprising 0, 1, 2, 3, 4, or 5 substitutions, insertions, or deletions. In one embodiment, the VL FR2 comprises the sequence of SEQ ID NO: 101 comprising 0, 1, 2, 3, 4, or 5 substitutions, insertions, or deletions. In one embodiment, the VL FR2 comprises the sequence of SEQ ID NO: 102 comprising 0, 1, 2, 3, 4, or 5 substitutions, insertions, or deletions. In one embodiment, the VL FR2 comprises the sequence of SEQ ID NO: 157-160 comprising 0, 1, 2, 3, 4, or 5 substitutions, insertions, or deletions. In one embodiment, the VL FR3 comprises the sequence of SEQ ID NO: 104 comprising 0, 1, 2, 3, 4, or 5 substitutions, insertions, or deletions. In one embodiment, the VL FR3 comprises the sequence of SEQ ID NO: 105 comprising 0, 1, 2, 3, 4, or 5 substitutions, insertions, or deletions. In one embodiment, the VL FR3 comprises the sequence of SEQ ID NO: 106 comprising 0, 1, 2, 3, 4, or 5 substitutions, insertions, or deletions. In one embodiment, the VL FR3 comprises the sequence of SEQ ID NO: 107 comprising 0, 1, 2, 3, 4, or 5 substitutions, insertions, or deletions. In one embodiment, the VL FR3 comprises the sequence of SEQ ID NO: 161-164 comprising 0, 1, 2, 3, 4, or 5 substitutions, insertions, or deletions. In one embodiment, the VL FR4 comprises the sequence of SEQ ID NO: 109 comprising 0, 1, 2, 3, 4, or 5 substitutions, insertions, or deletions. In one embodiment, the VL FR4 comprises the sequence of SEQ ID NO: 110 comprising 0, 1, 2, 3, 4, or 5 substitutions, insertions, or deletions. In one embodiment, the VL FR4 comprises the sequence of SEQ ID NO: 165-167 comprising 0, 1, 2, 3, 4, or 5 substitutions, insertions, or deletions.

In one embodiment, the VL FR1, VL FR2, VL FR3, and VL FR4 comprises the sequence of SEQ ID NO: 95, 100, 104, and 109, respectively. In one embodiment, the VL FR1, VL FR2, VL FR3, and VL FR4 comprises the sequence of SEQ ID NO: 96, 101, 105, and 109, respectively. In one embodiment, the VL FR1, VL FR2, VL FR3, and VL FR4 comprises the sequence of SEQ ID NO: 97, 101, 105, and 109, respectively. In one embodiment, the VL FR1, VL FR2, VL FR3, and VL FR4 comprises the sequence of SEQ ID NO: 95, 100, 106, and 109, respectively. In one embodiment, the VL FR1, VL FR2, VL FR3, and VL FR4 comprises the sequence of SEQ ID NO: 98, 102, 107, and 110, respectively.

In one embodiment, the VL FR1, VL FR2, VL FR3, and VL FR4 comprises the sequence of SEQ ID NO: 95, 100, 104, 109, respectively, each comprising 0, 1, 2, 3, 4, or 5 substitutions, insertions, or deletions. In one embodiment, the VL FR1, VL FR2, VL FR3, and VL FR4 comprises the sequence of SEQ ID NO: 96, 101, 105, 109, respectively, each comprising 0, 1, 2, 3, 4, or 5 substitutions, insertions, or deletions. In one embodiment, the VL FR1, VL FR2, VL FR3, and VL FR4 comprises the sequence of SEQ ID NO: 97, 101, 105, 109, respectively, each comprising 0, 1, 2, 3, 4, or 5 substitutions, insertions, or deletions. In one embodiment, the VL FR1, VL FR2, VL FR3, and VL FR4 comprises the sequence of SEQ ID NO: 95, 100, 106, 109, respectively, each comprising 0, 1, 2, 3, 4, or 5 substitutions, insertions, or deletions. In one embodiment, the VL FR1, VL FR2, VL FR3, and VL FR4 comprises the sequence of SEQ ID NO: 98, 102, 107, 110, respectively, each comprising 0, 1, 2, 3, 4, or 5 substitutions, insertions, or deletions.

In one embodiment, an antibody described herein comprises VH framework regions comprising one or more of a V at H2, G at H15, L at H37, E at H39, T at H40, L or I at H45, H or T at H68, I at H69, G at H70, H at H72, P at H73, Eat H74, K at H81, K or Tat H82A, S at H82B, V at H82C, Tat H87, A at H88, V at H89, and N or K at H105, I at H107, K at H108, wherein the VH framework residues are numbered according to Kabat. In one embodiment, the VH framework regions comprise two or more, three or more, four or more, or five or more of a V at H2, G at H15, L at H37, Eat H39, Tat H40, L or I at H45, H or Tat H68, I at H69, G at H70, H at H72, P at H73, E at H74, K at H81, K or Tat H82A, S at H82B, V at H82C, T at H87, A at H88, V at H89, and N or K at H105, I at H107, K at H108, wherein the VH framework residues are numbered according to Kabat. In one embodiment, the VH framework regions comprise a V at H2, G at H15, L at H37, E at H39, Tat H40, L or I at H45, H or Tat H68, I at H69, G at H70, H at H72, P at H73, E at H74, K at H81, K or Tat H82A, Sat H82B, Vat H82C, Tat H87, A at H88, Vat H89, and N or K at H105, I at H107, K at H108, wherein the VH framework residues are numbered according to Kabat. In one embodiment, the VH framework regions comprise a G at H15, L at H37, E at H39, Tat H40, H at H68, L at H69, G at H70, H at H72, P at H73, K or T at H82A, S at H82B, V at H82C, T at H87, A at H88, V at H89, and N or K at H105, wherein the VH framework residues are numbered according to Kabat. In one embodiment, the VH framework regions comprise a G at H15, L at H37, E at H39, T at H40, T at H68, I at H69, G or S at H70, H or D at H72, P at H73, K or T at H82A, S at H82B, V at H82C, T at H87, A at H88, V at H89, and N or K at H105, wherein the VH framework residues are numbered according to Kabat. In one embodiment, the VH has at least about 70%, at least about 75%, at least about 80%, at least about 90%, or at least about 95% sequence identity to SEQ ID NO: 1. In one embodiment, the VH has at least about 70%, at least about 75%, at least about 80%, at least about 90%, or at least about 95% sequence identity to SEQ ID NO: 3-9, 45, 47, 49-54, 65, 69, 70 or 112-126.

In one embodiment, an antibody described herein comprises VL framework regions comprising one or more of a S at L2, Y at L3, V at L4, L at L5, T at L6, Q at L7, P at L8, P at L9, S at L10, V at L11, V at L19, T at L22, Q at L38, P at L40, K or R at L45, I at L68, D at L69, S at L79, N or R at L80, T at L88, L at L101, E at 102L, and P at L103, wherein the VL framework residues are numbered according to Kabat. In one embodiment, the VL framework regions comprise two or more, three or more, four or more, or five or more of a S at L2, Y at L3, V at L4, L at L5, T at L6, Q at L7, P at L8, P at L9, S at L10, V at L11, V at L19, T at L22, Q at L38, P at L40, K or Rat L45, I at L68, D at L69, S at L79, N or Rat L80, Tat L88, L at L101, Eat 102L, and P at L103, wherein the VL framework residues are numbered according to Kabat. In one embodiment, the VL framework regions comprise a S at L2, Y at L3, V at L4, L at L5, T at L6, Q at L7, P at L8, P at L9, S at L10, V at L11, V at L19, T at L22, Q at L38, P at L40, K or R at L45, I at L68, D at L69, S at L79, N or Rat L80, Tat L88, L at L101, Eat 102L, and P at L103, wherein the VL framework residues are numbered according to Kabat. In one embodiment, the VL framework regions comprise a V at L19, K or R at L45, I at L68, D or A at L69, N at L80, T at L88, L at L101, and P at L103, wherein the VL framework residues are numbered according to Kabat. In one embodiment, the VL framework regions comprise a V at L19, K or R at L45, I at L68, D or A at L69, N at L80, T at L88, L at L101, and P at L103, wherein the VL framework residues are numbered according to Kabat. In one embodiment, the VL has at least about 70%, at least about 75%, at least about 80%, at least about 90%, or at least about 95% sequence identity to SEQ ID NO: 2. In one embodiment, the VL has at least about 70%, at least about 75%, at least about 80%, at least about 90%, or at least about 95% sequence identity to SEQ ID NO: 10, 11, 46, 48, 55, 66 or 127-139.

In one aspect, provided herein are engineered variants of the PGT-121 antibody comprising a VH CDR1, VH CDR2, VH CDR3, VL CDR1, VL CDR2, VL CDR3, VH framework and VL framework wherein one or both of the VH framework and the VL framework comprises a variant of the corresponding PGT-121 framework.

In one embodiment, an antibody described herein comprises a VH CDR1, VH CDR2, VH CDR3, VL CDR1, VL CDR2, VL CDR3, VH framework and VL framework wherein the VH framework comprises one or more of a V at H2, G at H15, L at H37, E at H39, T at H40, H at H68, G at H70, H at H72, P at H73, K at H81, K or Tat H82A, S at H82B, V at H82C, T at H87, A at H88, V at H89, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a V at H2, G at H15, L at H37, E at H39, T at H40, H at H68, G at H70, H at H72, P at H73, K at H81, K or Tat H82A, S at H82B, V at H82C, T at H87, A at H88, V at H89, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a V at H2, G at H15, L at H37, E at H39, T at H40, H at H68, G at H70, H at H72, P at H73, K at H81, K or Tat H82A, S at H82B, V at H82C, Tat H87, A at H88, V at H89, K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, Tat H40, H at H68, G at H70, H at H72, P at H73, T at H82A, S at H82B, V at H82C, T at H87, A at H88, and V at H89, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, T at H40, H at H68, G at H70, H at H72, P at H73, T at H82A, S at H82B, V at H82C, T at H87, A at H88, and V at H89, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, E at H39, T at H40, H at H68, G at H70, H at H72, P at H73, Tat H82A, S at H82B, V at H82C, Tat H87, A at H88, and V at H89, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, T at H40, H at H68, G at H70, H at H72, P at H73, T at H82A, S at H82B, V at H82C, T at H87, A at H88, V at H89, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, T at H40, H at H68, G at H70, H at H72, P at H73, T at H82A, S at H82B, V at H82C, T at H87, A at H88, V at H89, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, E at H39, T at H40, H at H68, G at H70, H at H72, P at H73, T at H82A, S at H82B, V at H82C, T at H87, A at H88, V at H89, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, Tat H40, A at H60, H at H68, G at H70, H at H72, P at H73, Tat H82A, S at H82B, Vat H82C, Tat H87, A at H88, Vat H89, W at H100P, P at H101, and T at H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, Tat H40, A at H60, H at H68, G at H70, H at H72, P at H73, T at H82A, S at H82B, V at H82C, T at H87, A at H88, V at H89, W at H100P, P at H101, and T at H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, E at H39, T at H40, A at H60, H at H68, G at H70, H at H72, P at H73, T at H82A, S at H82B, V at H82C, T at H87, A at H88, V at H89, W at H100P, P at H101, and Tat H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, T at H40, A at H60, H at H68, G at H70, H at H72, P at H73, T at H82A, S at H82B, V at H82C, T at H87, A at H88, V at H89, W at H100P, P at H101, T at H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, T at H40, A at H60, H at H68, G at H70, H at H72, P at H73, T at H82A, S at H82B, V at H82C, T at H87, A at H88, V at H89, W at H100P, P at H101, T at H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, E at H39, T at H40, A at H60, H at H68, G at H70, H at H72, P at H73, T at H82A, S at H82B, V at H82C, T at H87, A at H88, V at H89, W at H100P, P at H101, T at H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, T at H40, A at H60, T at H68, I at 69, G at H70, D at H72, P at H73, T at H82A, S at H82B, V at H82C, T at H87, A at H88, V at H89, W at H100P, P at H101, and T at H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, T at H40, A at H60, T at H68, I at 69, G at H70, D at H72, P at H73, T at H82A, S at H82B, V at H82C, T at H87, A at H88, V at H89, W at H100P, P at H101, and T at H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, E at H39, T at H40, A at H60, T at H68, I at 69, G at H70, D at H72, P at H73, Tat H82A, S at H82B, V at H82C, T at H87, A at H88, V at H89, W at H100P, P at H101, and T at H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, T at H40, A at H60, T at H68, I at 69, G at H70, D at H72, P at H73, T at H82A, S at H82B, V at H82C, Tat H87, A at H88, Vat H89, W at H100P, P at H101, Tat H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, T at H40, A at H60, T at H68, I at 69, G at H70, D at H72, P at H73, T at H82A, S at H82B, V at H82C, T at H87, A at H88, V at H89, W at H100P, P at H101, T at H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, E at H39, T at H40, A at H60, T at H68, I at 69, G at H70, D at H72, P at H73, T at H82A, S at H82B, V at H82C, T at H87, A at H88, V at H89, W at H100P, P at H101, T at H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, Tat H40, A at H60, T at H68, I at 69, S at H70, D at H72, P at H73, Tat H82A, S at H82B, V at H82C, Tat H87, A at H88, V at H89, W at H100P, P at H101, and T at H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, T at H40, A at H60, T at H68, I at 69, S at H70, D at H72, P at H73, T at H82A, S at H82B, V at H82C, T at H87, A at H88, V at H89, W at H100P, P at H101, and T at H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, Eat H39, Tat H40, A at H60, Tat H68, I at 69, S at H70, D at H72, P at H73, Tat H82A, S at H82B, V at H82C, T at H87, A at H88, V at H89, W at H100P, P at H101, and T at H102, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, T at H40, A at H60, T at H68, I at 69, S at H70, D at H72, P at H73, Tat H82A, S at H82B, V at H82C, T at H87, A at H88, V at H89, W at H100P, P at H101, T at H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, T at H40, A at H60, T at H68, I at 69, S at H70, D at H72, P at H73, T at H82A, S at H82B, V at H82C, T at H87, A at H88, V at H89, W at H100P, P at H101, Tat H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, E at H39, T at H40, A at H60, T at H68, I at 69, S at H70, D at H72, P at H73, T at H82A, S at H82B, V at H82C, T at H87, A at H88, V at H89, W at H100P, P at H101, T at H102, and K at H105, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises one or more of a G at H15, L at H37, E at H39, T at H40, and G at H70, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises two or more, three or more, four or more, five or more, six or more, seven or more, eight or more, nine or more, or ten or more of a G at H15, L at H37, E at H39, Tat H40, and G at H70, wherein the VH framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH framework comprises a G at H15, L at H37, E at H39, Tat H40, G at H70, wherein the VH framework residues are numbered according to Kabat. In one embodiment, the VH framework further comprises V at H2. In one embodiment, the VH framework further comprises T at H82A, S at H82B, V at H82C, T at H87, A at H88, V at H89. In one embodiment, the VH framework further comprises K at 81 and K at 82A. In one embodiment, the VH framework further comprises K at H105. In one embodiment, the VH framework further comprises one or more of a H at 68, H at 72, and P at 73. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, VL CDR3, VH framework and VL framework wherein the VL framework comprises one or more of a S at L2, Y at L3, V at L4, L at L5, T at L6, Q at L7, P at L8, P at L9, S at L10, V at L11, V at L19, K at L45, I at L68, D at L69, N at L80, and T at L88, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises two or more, three or more, four or more, or five or more of a S at L2, Y at L3, V at L4, L at L5, T at L6, Q at L7, P at L8, P at L9, S at L10, V at L11, V at L19, K at L45, I at L68, D at L69, N at L80, and T at L88, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises a S at L2, Y at L3, V at L4, L at L5, T at L6, Q at L7, P at L8, P at L9, S at L10, V at L11, V at L19, K at L45, I at L68, D at L69, N at L80, and T at L88, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises one or more of a V at L19, K at L45, I at L68, D at L69, N at L80, and T at L88, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises two or more, three or more, four or more, or five or more of a V at L19, K at L45, I at L68, D at L69, N at L80, and Tat L88, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises a V at L19, K at L45, I at L68, D at L69, N at L80, and T at L88, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises one or more of a V at L19, T at L22, Rat L45, I at L68, D at L69, S at L79, N at L80, T at L88, and P at L103, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises two or more, three or more, four or more, or five or more of a V at L19, Tat L22, Rat L45, I at L68, D at L69, S at L79, N at L80, T at L88, and P at L103, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises a V at L19, T at L22, R at L45, I at L68, D at L69, S at L79, N at L80, T at L88, and P at L103, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises one or more of a I at L68 and A at L69, wherein the VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VL framework comprises I at L68 and A at L69, wherein the VL framework residues are numbered according to Kabat. In embodiment, the VL framework further comprises one or more of S at L2, Y at L3, V at L4, L at L5, Tat L6, Q at L7, P at L8, P at L9, S at L10, and V at L11. In one embodiment, the VH framework further comprises one or more of a K at H81 and K at H105. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, VL CDR3, VH framework and VL framework wherein the VH and VL frameworks comprise one or more of a G at H15, L at H37, E at H39, Tat H40, H at H68, G at H70, H at H72, P at H73, T at H82A, S at H82B, V at H82C, T at H87, A at H88, V at H89, V at L19, K at L45, I at L68, D at L69, N at L80, and T at L88, wherein the VH and VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH and VL frameworks comprise two or more, three or more, four or more, five or more, six or more, seven or more, eight or more, nine or more, or ten or more of a G at H15, L at H37, E at H39, Tat H40, H at H68, G at H70, H at H72, P at H73, Tat H82A, S at H82B, Vat H82C, Tat H87, A at H88, V at H89, Vat L19, K at L45, I at L68, D at L69, N at L80, and T at L88, wherein the VH and VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH and VL frameworks comprise a G at H15, L at H37, E at H39, T at H40, H at H68, G at H70, H at H72, P at H73, T at H82A, S at H82B, V at H82C, Tat H87, A at H88, V at H89, V at L19, K at L45, I at L68, D at L69, N at L80, and T at L88, wherein the VH and VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH and VL frameworks comprise a G at H15, L at H37, E at H39, T at H40, H at H68, G at H70, H at H72, P at H73, T at H82A, S at H82B, V at H82C, T at H87, A at H88, V at H89, K at H105, V at L19, K at L45, I at L68, D at L69, N at L80, and T at L88, wherein the VH and VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH and VL frameworks comprise one or more of a G at H15, L at H37, E at H39, T at H40, A at H60, T at H68, I at 69, G or S at H70, D at H72, P at H73, Tat H82A, S at H82B, V at H82C, Tat H87, A at H88, V at H89, W at H100P, P at H101, T at H102, V at L19, T at L22, R at L45, I at L68, D at L69, Sat L79, N at L80, T at L88, and P at L103, wherein the VH and VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH and VL frameworks comprise two or more, three or more, four or more, five or more, six or more, seven or more, eight or more, nine or more, or ten or more of a G at H15, L at H37, E at H39, T at H40, A at H60, T at H68, I at 69, G or S at H70, D at H72, P at H73, T at H82A, S at H82B, V at H82C, T at H87, A at H88, V at H89, W at H100P, P at H101, Tat H102, V at L19, Tat L22, Rat L45, I at L68, D at L69, S at L79, N at L80, T at L88, and P at L103, wherein the VH and VL framework residues are numbered according to Kabat. In one embodiment, an antibody described herein comprises a VH CDR1, VH CDR2, VH CDR3, VL CDR1, VL CDR2, VL CDR3, VH framework and VL framework wherein the VH and VL frameworks comprise a G at H15, L at H37, E at H39, T at H40, A at H60, T at H68, I at 69, G or S at H70, D at H72, P at H73, T at H82A, S at H82B, V at H82C, Tat H87, A at H88, Vat H89, W at H100P, P at H101, Tat H102, Vat L19, Tat L22, R at L45, I at L68, D at L69, S at L79, N at L80, Tat L88, and P at L103, wherein the VH and VL framework residues are numbered according to Kabat. In one embodiment, the VH framework further comprises one or more of a K at H105. In one embodiment, the VH framework further comprises one or more of a K at H81 and K at H82A. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.15.H1 VH (SEQ ID NO: 3). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.15.H2 VH (SEQ ID NO: 4). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.15.H3 VH (SEQ ID NO: 5). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.15.H4 VH (SEQ ID NO: 6). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.15.H5 VH (SEQ ID NO: 7). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.15.H6 VH (SEQ ID NO: 8). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.15.H7 VH (SEQ ID NO: 9). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.18.H1 VH (SEQ ID NO: 65). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.18.H17 VH (SEQ ID NO: 69). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.18.H18 VH (SEQ ID NO: 70). In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.18.H2-16 VH. In one embodiment, an antibody described herein comprises a VH CDR1, VH CDR2, VH CDR3, VL CDR1, VL CDR2, VL CDR3, VH framework and VL framework wherein the antibody comprises the VH framework of the ePGT121.13.H1, ePGT121.14.H1, ePGT121.14.H2, ePGT121.14.H3, ePGT121.14.H4, ePGT121.14.H5, ePGT121.14.H6, ePGT121.18.H1, ePGT121.184.H17, or ePGT121.18.H18 VH. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the VH CDR1, VH CDR2, VH CDR3, VL CDR1, VL CDR2, and VL CDR3 comprises the sequence of SEQ ID NO: 18, 19, 67, 23, 24, and 68, respectively. In one embodiment, the VH CDR1, VH CDR2, VH CDR3, VL CDR1, VL CDR2, and VL CDR3 comprises the sequence of SEQ ID NO: 18, 19, 22, 23, 24, and 68, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.14.L1 VL (SEQ ID NO: 55). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.17.L1 VL (SEQ ID NO: 66). In embodiment, the antibody comprises the VH CDR1, VH CDR2, and VH CDR3 of the ePGT121.18.H1 VH (SEQ ID NO: 65) and the VL CDR1, VL CDR2, and VL CDR3 of the ePGT-121.17.L1 VL (SEQ ID NO: 66). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, VL CDR3, VH framework and VL framework wherein the antibody comprises the VL framework of the ePGT121.15.L1 VL (SEQ ID NO: 10). In one embodiment, an antibody described herein comprises a VH CDR1, VH CDR2, VH CDR3, VL CDR1, VL CDR2, VL CDR3, VH framework and VL framework wherein the antibody comprises the VL framework of the ePGT121.15.L2 VL (SEQ ID NO: 11). In one embodiment, an antibody described herein comprises a VH CDR1, VH CDR2, VH CDR3, VL CDR1, VL CDR2, VL CDR3, VH framework and VL framework wherein the antibody comprises the VL framework of the ePGT121.14.L1 VL (SEQ ID NO: 55). In one embodiment, an antibody described herein comprises a VH CDR1, VH CDR2, VH CDR3, VL CDR1, VL CDR2, VL CDR3, VH framework and VL framework wherein the antibody comprises the VL framework of the ePGT121.17.L1 VL (SEQ ID NO: 66). In one embodiment, an antibody described herein comprises a VH CDR1, VH CDR2, VH CDR3, VL CDR1, VL CDR2, VL CDR3, VH framework and VL framework wherein the antibody comprises the VL framework of the ePGT121.17.L2-14 VL. In one embodiment, an antibody described herein comprises a VH CDR1, VH CDR2, VH CDR3, VL CDR1, VL CDR2, VL CDR3, VH framework and VL framework wherein the antibody comprises the VL framework of the ePGT121.12.L4, ePGT121.13.L1, or ePGT121.14.L1 VL. In one embodiment, the VH framework further comprises one or more of a K at H81 and K at H105. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the VH CDR1, VH CDR2, VH CDR3, VL CDR1, VL CDR2, and VL CDR3 comprises the sequence of SEQ ID NO: 18, 19, 67, 23, 24, and 68, respectively. In one embodiment, the VH CDR1, VH CDR2, VH CDR3, VL CDR1, VL CDR2, and VL CDR3 comprises the sequence of SEQ ID NO: 18, 19, 22, 23, 24, and 68, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.14.L1 VL (SEQ ID NO: 55). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.17.L1 VL (SEQ ID NO: 66). In embodiment, the antibody comprises the VH CDR1, VH CDR2, and VH CDR3 of the ePGT121.18.H1 VH (SEQ ID NO: 65) and the VL CDR1, VL CDR2, and VL CDR3 of the ePGT-121.17.L1 VL (SEQ ID NO: 66). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.15.H1 VH (SEQ ID NO: 3) and (ii) the VL framework of the ePGT121.15.L1 VL (SEQ ID NO: 10) or the ePGT121.15.L2 VL (SEQ ID NO: 11). In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.15.H2 VH (SEQ ID NO: 4) and (ii) the VL framework of the ePGT121.15.L1 VL (SEQ ID NO: 10) or the ePGT121.15.L2 VL (SEQ ID NO: 11). In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.15.H3 VH (SEQ ID NO: 5) and (ii) the VL framework of the ePGT121.15.L1 VL (SEQ ID NO: 10) or the ePGT121.15.L2 VL (SEQ ID NO: 11). In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.15.H4 VH (SEQ ID NO: 6) and (ii) the VL framework of the ePGT121.15.L1 VL (SEQ ID NO: 10) or the ePGT121.15.L2 VL (SEQ ID NO: 11). In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.15.H5 VH (SEQ ID NO: 7) and (ii) the VL framework of the ePGT121.15.L1 VL (SEQ ID NO: 10) or the ePGT121.15.L2 VL (SEQ ID NO: 11). In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.15.H6 VH (SEQ ID NO: 8) and (ii) the VL framework of the ePGT121.15.L1 VL (SEQ ID NO: 10) or the ePGT121.15.L2 VL (SEQ ID NO: 11). In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.15.H7 VH (SEQ ID NO: 9) and (ii) the VL framework of the ePGT121.15.L1 VL (SEQ ID NO: 10) or the ePGT121.15.L2 VL (SEQ ID NO: 11). In one embodiment, the antibody comprises the VL framework of the ePGT121.12.L4, ePGT121.13.L1, or ePGT121.14.L1 VL. In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.13.H1, ePGT121.14.H1, ePGT121.14.H2, ePGT121.14.H3, ePGT121.14.H4, ePGT121.14.H5, or ePGT121.14.H6 VH and (ii) the VL framework of the ePGT121.15.L1, ePGT121.15.L2, ePGT121.12.L4, ePGT121.13.L1, or ePGT121.14.L1 VL. In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.18.H1, ePGT121.18.H17, or ePGT121.18.H18 VH and (ii) the VL framework of the ePGT121.17.L1 VL. In one embodiment, an antibody described herein comprises a VH CDR1, VH CDR2, VH CDR3, VL CDR1, VL CDR2, VL CDR3, VH framework and VL framework wherein the antibody comprises (i) the VH framework of the ePGT121.18.H2-16 VH and (ii) the VL framework of the ePGT121.17.L2-14 VL. In one embodiment, the VH framework further comprises one or more of a K at H81 and K at H105. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the VH CDR1, VH CDR2, VH CDR3, VL CDR1, VL CDR2, and VL CDR3 comprises the sequence of SEQ ID NO: 18, 19, 67, 23, 24, and 68, respectively. In one embodiment, the VH CDR1, VH CDR2, VH CDR3, VL CDR1, VL CDR2, and VL CDR3 comprises the sequence of SEQ ID NO: 18, 19, 22, 23, 24, and 68, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.14.L1 VL (SEQ ID NO: 55). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.17.L1 VL (SEQ ID NO: 66). In embodiment, the antibody comprises the VH CDR1, VH CDR2, and VH CDR3 of the ePGT121.18.H1 VH (SEQ ID NO: 65) and the VL CDR1, VL CDR2, and VL CDR3 of the ePGT-121.17.L1 VL (SEQ ID NO: 66). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH CDR1, VH CDR2, VH CDR3, VL CDR1, VL CDR2, VL CDR3, VH framework and VL framework wherein the antibody comprises a VH framework with at least about 70%, at least about 80%, at least about 90%, at least about 95%, at least about 96%, at least about 97%, at least about 98%, or at least about 99% sequence identity to the VH framework of the ePGT121.15.H1, ePGT121.15.H2, ePGT121.15.H3, ePGT121.15H.4, ePGT121.15.H5, ePGT121.15.H6, ePGT121.15.H7, ePGT121.13.H1, ePGT121.14.H1, ePGT121.14.H2, ePGT121.14.H3, ePGT121.14.H4, ePGT121.14.H5, ePGT121.14.H6 VH. In one embodiment, an antibody described herein comprises a VH CDR1, VH CDR2, VH CDR3, VL CDR1, VL CDR2, VL CDR3, VH framework and VL framework wherein the antibody comprises a VH framework with at least about 70%, at least about 80%, at least about 90%, at least about 95%, at least about 96%, at least about 97%, at least about 98%, or at least about 99% sequence identity to the VH framework of the ePGT121.18.H1, ePGT121.18.H17, ePGT121.18.H18 VH. In one embodiment, an antibody described herein comprises a VH CDR1, VH CDR2, VH CDR3, VL CDR1, VL CDR2, VL CDR3, VH framework and VL framework wherein the antibody comprises a VH framework with at least about 90%, sequence identity to the VH framework of the ePGT121.15.H1, ePGT121.15.H2, ePGT121.15.H3, ePGT121.15H.4, ePGT121.15.H5, ePGT121.15.H6, ePGT121.15.H7, ePGT121.13.H1, ePGT121.14.H1, ePGT121.14.H2, ePGT121.14.H3, ePGT121.14.H4, ePGT121.14.H5, ePGT121.14.H6 VH. In one embodiment, an antibody described herein comprises a VH CDR1, VH CDR2, VH CDR3, VL CDR1, VL CDR2, VL CDR3, VH framework and VL framework wherein the antibody comprises a VH framework with at least about 90%, sequence identity to the VH framework of the ePGT121.18.H1, ePGT121.18.H17, ePGT121.18.H18 VH. In one embodiment, an antibody described herein comprises a VH CDR1, VH CDR2, VH CDR3, VL CDR1, VL CDR2, VL CDR3, VH framework and VL framework wherein the antibody comprises a VH framework with at least about 90% sequence identity to the VH framework of ePGT121.15.H6 VH (SEQ ID NO: 8). In one embodiment, an antibody described herein comprises a VH CDR1, VH CDR2, VH CDR3, VL CDR1, VL CDR2, VL CDR3, VH framework and VL framework wherein the antibody comprises a VH framework with at least about 90% sequence identity to the VH framework of ePGT121.18.H1 VH (SEQ ID NO: 65). In one embodiment, an antibody described herein comprises a VH CDR1, VH CDR2, VH CDR3, VL CDR1, VL CDR2, VL CDR3, VH framework and VL framework wherein the antibody comprises a VH framework with at least about 90% sequence identity to the VH framework of ePGT121.18.H17 VH (SEQ ID NO: 69). In one embodiment, an antibody described herein comprises a VH CDR1, VH CDR2, VH CDR3, VL CDR1, VL CDR2, VL CDR3, VH framework and VL framework wherein the antibody comprises a VH framework with at least about 90% sequence identity to the VH framework of ePGT121.18.H18 VH (SEQ ID NO: 70). In one embodiment, an antibody described herein comprises a VH CDR1, VH CDR2, VH CDR3, VL CDR1, VL CDR2, VL CDR3, VH framework and VL framework wherein the antibody comprises a VL framework with at least about 70%, at least about 80%, at least about 90%, at least about 95%, at least about 96%, at least about 97%, at least about 98%, or at least about 99% sequence identity to the VL framework of the ePGT121.15.L1, ePGT121.15.L2, ePGT121.12.L4, ePGT121.13.L1, or ePGT121.14.L1 VL. In one embodiment, an antibody described herein comprises a VH CDR1, VH CDR2, VH CDR3, VL CDR1, VL CDR2, VL CDR3, VH framework and VL framework wherein the antibody comprises a VL framework with at least about 70%, at least about 80%, at least about 90%, at least about 95%, at least about 96%, at least about 97%, at least about 98%, or at least about 99% sequence identity to the VL framework of the ePGT121.17_L1 VL. In one embodiment, an antibody described herein comprises a VH CDR1, VH CDR2, VH CDR3, VL CDR1, VL CDR2, VL CDR3, VH framework and VL framework wherein the antibody comprises a VL framework with at least about 90% sequence identity to the VL framework of the ePGT121.15.L1, ePGT121.15.L2, ePGT121.12.L4, ePGT121.13.L1, or ePGT121.14.L1 VL. In one embodiment, an antibody described herein comprises a VH CDR1, VH CDR2, VH CDR3, VL CDR1, VL CDR2, VL CDR3, VH framework and VL framework wherein the antibody comprises a VL framework with at least about 90% sequence identity to the VL framework of the ePGT121.17_L1 VL. In one embodiment, an antibody described herein comprises a VH CDR1, VH CDR2, VH CDR3, VL CDR1, VL CDR2, VL CDR3, VH framework and VL framework wherein the antibody comprises a VL framework with at least about 90% sequence identity to the VL framework of the ePGT121.15.L1 VL. In one embodiment, an antibody described herein comprises a VH CDR1, VH CDR2, VH CDR3, VL CDR1, VL CDR2, VL CDR3, VH framework and VL framework wherein the antibody comprises (i) a VH framework with at least about 90% sequence identity to the VH framework of ePGT121.15.H6 VH (SEQ ID NO: 8) and (ii) a VL framework with at least about 90% sequence identity to the VL framework of the ePGT121.15.L1 VL (SEQ ID NO: 10). In one embodiment, an antibody described herein comprises a VH CDR1, VH CDR2, VH CDR3, VL CDR1, VL CDR2, VL CDR3, VH framework and VL framework wherein the antibody comprises (i) a VH framework with at least about 90% sequence identity to the VH framework of ePGT121.18.H1 VH (SEQ ID NO: 65) and (ii) a VL framework with at least about 90% sequence identity to the VL framework of the ePGT121.17.L1 VL (SEQ ID NO: 66). In one embodiment, an antibody described herein comprises a VH CDR1, VH CDR2, VH CDR3, VL CDR1, VL CDR2, VL CDR3, VH framework and VL framework wherein the antibody comprises (i) a VH framework with at least about 90% sequence identity to the VH framework of ePGT121.18.H17 VH (SEQ ID NO: 69) and (ii) a VL framework with at least about 90% sequence identity to the VL framework of the ePGT121.17.L1 VL (SEQ ID NO: 66). In one embodiment, an antibody described herein comprises a VH CDR1, VH CDR2, VH CDR3, VL CDR1, VL CDR2, VL CDR3, VH framework and VL framework wherein the antibody comprises (i) a VH framework with at least about 90% sequence identity to the VH framework of ePGT121.18.H18 VH (SEQ ID NO: 70) and (ii) a VL framework with at least about 90% sequence identity to the VL framework of the ePGT121.17.L1 VL (SEQ ID NO: 66). In one embodiment, the VH framework further comprises one or more of a K at H81 and K at H105. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the VH CDR1, VH CDR2, VH CDR3, VL CDR1, VL CDR2, and VL CDR3 comprises the sequence of SEQ ID NO: 18, 19, 67, 23, 24, and 68, respectively. In one embodiment, the VH CDR1, VH CDR2, VH CDR3, VL CDR1, VL CDR2, and VL CDR3 comprises the sequence of SEQ ID NO: 18, 19, 22, 23, 24, and 68, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.14.L1 VL (SEQ ID NO: 55). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.17.L1 VL (SEQ ID NO: 66). In embodiment, the antibody comprises the VH CDR1, VH CDR2, and VH CDR3 of the ePGT121.18.H1 VH (SEQ ID NO: 65) and the VL CDR1, VL CDR2, and VL CDR3 of the ePGT-121.17.L1 VL (SEQ ID NO: 66). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH comprising an amino acid sequence with at least about 70%, at least about 80%, at least about 90%, at least about 95%, at least about 96%, at least about 97%, at least about 98%, or at least about 99% sequence identity to SEQ ID NO: 3-9, 45, 47, or 49-54. In one embodiment, an antibody described herein comprises a VH comprising an amino acid sequence with at least about 90% sequence identity to SEQ ID NO: 3-9, 45, 47, or 49-54. In one embodiment, an antibody described herein comprises a VH comprising an amino acid sequence with at least about 70%, at least about 80%, at least about 90%, at least about 95%, at least about 96%, at least about 97%, at least about 98%, or at least about 99% sequence identity to SEQ ID NO: 8. In one embodiment, an antibody described herein comprises a VH comprising an amino acid sequence with at least about 70%, at least about 80%, at least about 90%, at least about 95%, at least about 96%, at least about 97%, at least about 98%, or at least about 99% sequence identity to SEQ ID NO: 65. In one embodiment, an antibody described herein comprises a VH comprising an amino acid sequence with at least about 70%, at least about 80%, at least about 90%, at least about 95%, at least about 96%, at least about 97%, at least about 98%, or at least about 99% sequence identity to SEQ ID NO: 69. In one embodiment, an antibody described herein comprises a VH comprising an amino acid sequence with at least about 70%, at least about 80%, at least about 90%, at least about 95%, at least about 96%, at least about 97%, at least about 98%, or at least about 99% sequence identity to SEQ ID NO: 70. In one embodiment, an antibody described herein comprises a VH comprising an amino acid sequence with at least about 70%, at least about 80%, at least about 90%, at least about 95%, at least about 96%, at least about 97%, at least about 98%, or at least about 99% sequence identity to SEQ ID NO: 112-126. In one embodiment, an antibody described herein comprises a VH comprising an amino acid sequence with at least about 90% sequence identity to SEQ ID NO: 8. In one embodiment, an antibody described herein comprises a VH comprising an amino acid sequence with at least about 90% sequence identity to SEQ ID NO: 65. In one embodiment, an antibody described herein comprises a VH comprising an amino acid sequence with at least about 90% sequence identity to SEQ ID NO: 69. In one embodiment, an antibody described herein comprises a VH comprising an amino acid sequence with at least about 90% sequence identity to SEQ ID NO: 70. In one embodiment, an antibody described herein comprises a VH comprising an amino acid sequence with at least about 90% sequence identity to SEQ ID NO: 112-126. In one embodiment, the antibody comprises a VH framework comprising one or more, two or more, three or more, four or more, five or more, six or more, seven or more, eight or more, nine or more, or ten or more of a G at H15, L at H37, E at H39, T at H40, H at H68, G at H70, H at H72, P at H73, T at H82A, S at H82B, V at H82C, T at H87, A at H88, V at H89, wherein the VH framework residues are numbered according to Kabat. In one embodiment, the antibody comprises a VH framework comprising a G at H15, L at H37, E at H39, T at H40, H at H68, G at H70, H at H72, P at H73, T at H82A, S at H82B, V at H82C, Tat H87, A at H88, V at H89, wherein the VH framework residues are numbered according to Kabat. In one embodiment, the antibody comprises a VH framework comprising a G at H15, L at H37, E at H39, T at H40, H at H68, G at H70, H at H72, P at H73, T at H82A, S at H82B, V at H82C, T at H87, A at H88, V at H89, and K at H105, wherein the VH framework residues are numbered according to Kabat. In one embodiment, the antibody comprises a VH framework comprising one or more, two or more, three or more, four or more, five or more, six or more, seven or more, eight or more, nine or more, or ten or more of a G at H15, L at H37, E at H39, T at H40, A at H60, T at H68, I at 69, G or S at H70, D at H72, P at H73, T at H82A, S at H82B, V at H82C, Tat H87, A at H88, V at H89, W at H100P, P at H101, Tat H102, wherein the VH framework residues are numbered according to Kabat. In one embodiment, the antibody comprises a VH framework comprising a G at H15, L at H37, E at H39, T at H40, A at H60, T at H68, I at 69, G or S at H70, D at H72, P at H73, Tat H82A, S at H82B, V at H82C, Tat H87, A at H88, V at H89, W at H100P, P at H101, T at H102, wherein the VH framework residues are numbered according to Kabat. In one embodiment, the antibody comprises a VH framework comprising a G at H15, L at H37, E at H39, T at H40, A at H60, T at H68, I at 69, G or S at H70, D at H72, P at H73, Tat H82A, S at H82B, Vat H82C, Tat H87, A at H88, Vat H89, W at H100P, P at H101, T at H102, and K at H105, wherein the VH framework residues are numbered according to Kabat. In one embodiment, the VH framework further comprises K at H105. In one embodiment, the VH framework further comprises K at H81 and K at H82A. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the VH CDR1, VH CDR2, VH CDR3, VL CDR1, VL CDR2, and VL CDR3 comprises the sequence of SEQ ID NO: 18, 19, 67, 23, 24, and 68, respectively. In one embodiment, the VH CDR1, VH CDR2, VH CDR3, VL CDR1, VL CDR2, and VL CDR3 comprises the sequence of SEQ ID NO: 18, 19, 22, 23, 24, and 68, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.14.L1 VL (SEQ ID NO: 55). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.17.L1 VL (SEQ ID NO: 66). In embodiment, the antibody comprises the VH CDR1, VH CDR2, and VH CDR3 of the ePGT121.18.H1 VH (SEQ ID NO: 65) and the VL CDR1, VL CDR2, and VL CDR3 of the ePGT-121.17.L1 VL (SEQ ID NO: 66). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VL comprising an amino acid sequence with at least about 70%, at least about 80%, at least about 90%, at least about 95%, at least about 96%, at least about 97%, at least about 98%, or at least about 99% sequence identity to SEQ ID NO: 10. In one embodiment, an antibody described herein comprises a VL comprising an amino acid sequence with at least about 90% sequence identity to SEQ ID NO: 10. In one embodiment, an antibody described herein comprises a VL comprising an amino acid sequence with at least about 70%, at least about 80%, at least about 90%, at least about 95%, at least about 96%, at least about 97%, at least about 98%, or at least about 99% sequence identity to SEQ ID NO: 11. In one embodiment, an antibody described herein comprises a VL comprising an amino acid sequence with at least about 90% sequence identity to SEQ ID NO: 11. In one embodiment, an antibody described herein comprises a VL comprising an amino acid sequence with at least about 70%, at least about 80%, at least about 90%, at least about 95%, at least about 96%, at least about 97%, at least about 98%, or at least about 99% sequence identity to SEQ ID NO: 66. In one embodiment, an antibody described herein comprises a VL comprising an amino acid sequence with at least about 90% sequence identity to SEQ ID NO: 66. In one embodiment, an antibody described herein comprises a VL comprising an amino acid sequence with at least about 70%, at least about 80%, at least about 90%, at least about 95%, at least about 96%, at least about 97%, at least about 98%, or at least about 99% sequence identity to SEQ ID NO: 127-139. In one embodiment, an antibody described herein comprises a VL comprising an amino acid sequence with at least about 90% sequence identity to SEQ ID NO: 127-139. In one embodiment, an antibody described herein comprises a VL comprising an amino acid sequence with at least about 70%, at least about 80%, at least about 90%, at least about 95%, at least about 96%, at least about 97%, at least about 98%, or at least about 99% sequence identity to SEQ ID NO: 46, 48, or 55. In one embodiment, an antibody described herein comprises a VL comprising an amino acid sequence with at least about 90% sequence identity to SEQ ID NO: 46, 48, or 55. In one embodiment, the antibody comprises a VL framework comprising one or more, two or more, three or more, four or more, or five or more of a V at L19, K at L45, I at L68, D at L69, N at L80, and Tat L88, wherein the VL framework residues are numbered according to Kabat. In one embodiment, the antibody comprises a VL framework comprising a V at L19, K at L45, I at L68, D at L69, N at L80, and T at L88, wherein the VL framework residues are numbered according to Kabat. In one embodiment, the antibody comprises a VL framework comprising one or more, two or more, three or more, four or more, or five or more of a V at L19, T at L22, Rat L45, I at L68, D at L69, S at L79, N at L80, T at L88, and P at L103, wherein the VL framework residues are numbered according to Kabat. In one embodiment, the antibody comprises a VL framework comprising a V at L19, T at L22, R at L45, I at L68, D at L69, S at L79, N at L80, T at L88, and P at L103, wherein the VL framework residues are numbered according to Kabat. In one embodiment, the VH framework further comprises K at H105. In one embodiment, the VH framework further comprises K at H81 and K at H82A. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the VH CDR1, VH CDR2, VH CDR3, VL CDR1, VL CDR2, and VL CDR3 comprises the sequence of SEQ ID NO: 18, 19, 67, 23, 24, and 68, respectively. In one embodiment, the VH CDR1, VH CDR2, VH CDR3, VL CDR1, VL CDR2, and VL CDR3 comprises the sequence of SEQ ID NO: 18, 19, 22, 23, 24, and 68, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.14.L1 VL (SEQ ID NO: 55). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.17.L1 VL (SEQ ID NO: 66). In embodiment, the antibody comprises the VH CDR1, VH CDR2, and VH CDR3 of the ePGT121.18.H1 VH (SEQ ID NO: 65) and the VL CDR1, VL CDR2, and VL CDR3 of the ePGT-121.17.L1 VL (SEQ ID NO: 66). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises (i) a VH comprising an amino acid sequence with at least about 70%, at least about 80%, at least about 90%, at least about 95%, at least about 96%, at least about 97%, at least about 98%, or at least about 99% sequence identity to SEQ ID NO: 3-9, 45, 47, or 49-54 and (ii) a VL comprising an amino acid sequence with at least about 70%, at least about 80%, at least about 90%, at least about 95%, at least about 96%, at least about 97%, at least about 98%, or at least about 99% sequence identity to SEQ ID NO: 10, 11, 46, 48, or 55. In one embodiment, an antibody described herein comprises (i) a VH comprising an amino acid sequence with at least about 90% sequence identity to SEQ ID NO: 8 and (ii) a VL comprising an amino acid sequence with at least about 90% sequence identity to SEQ ID NO: 10. In one embodiment, an antibody described herein comprises (i) a VH comprising an amino acid sequence with at least about 70%, at least about 80%, at least about 90%, at least about 95%, at least about 96%, at least about 97%, at least about 98%, or at least about 99% sequence identity to SEQ ID NO: 8, 65, 69 or 70 and (ii) a VL comprising an amino acid sequence with at least about 70%, at least about 80%, at least about 90%, at least about 95%, at least about 96%, at least about 97%, at least about 98%, or at least about 99% sequence identity to SEQ ID NO: 55 or 66. In one embodiment, an antibody described herein comprises (i) a VH comprising an amino acid sequence with at least about 90% sequence identity to SEQ ID NO: 65 and (ii) a VL comprising an amino acid sequence with at least about 90% sequence identity to SEQ ID NO: 66. In one embodiment, an antibody described herein comprises (i) a VH comprising an amino acid sequence with at least about 90% sequence identity to SEQ ID NO: 69 and (ii) a VL comprising an amino acid sequence with at least about 90% sequence identity to SEQ ID NO: 66. In one embodiment, an antibody described herein comprises (i) a VH comprising an amino acid sequence with at least about 90% sequence identity to SEQ ID NO: 70 and (ii) a VL comprising an amino acid sequence with at least about 90% sequence identity to SEQ ID NO: 66. In one embodiment, the antibody comprises a VH framework and VL framework comprising one or more, two or more, three or more, four or more, five or more, six or more, seven or more, eight or more, nine or more, or ten or more of a G at H15, L at H37, E at H39, T at H40, H at H68, G at H70, H at H72, P at H73, T at H82A, S at H82B, V at H82C, T at H87, A at H88, V at H89, V at L19, K at L45, I at L68, D at L69, N at L80, and T at L88, wherein the VH and VL framework residues are numbered according to Kabat. In one embodiment, the antibody comprises a VH framework and VL framework comprising a G at H15, L at H37, E at H39, T at H40, H at H68, G at H70, H at H72, P at H73, T at H82A, S at H82B, V at H82C, Tat H87, A at H88, V at H89, V at L19, K at L45, I at L68, D at L69, N at L80, and T at L88, wherein the VH and VL framework residues are numbered according to Kabat. In one embodiment, the antibody comprises a VH framework and VL framework comprising a G at H15, L at H37, E at H39, T at H40, H at H68, G at H70, H at H72, P at H73, T at H82A, S at H82B, V at H82C, Tat H87, A at H88, V at H89, K at 104, V at L19, K at L45, I at L68, D at L69, N at L80, and T at L88, wherein the VH and VL framework residues are numbered according to Kabat. In one embodiment, the antibody comprises a VH framework and VL framework comprising one or more, two or more, three or more, four or more, five or more, six or more, seven or more, eight or more, nine or more, or ten or more of a G at H15, L at H37, E at H39, T at H40, A at H60, T at H68, I at 69, G or S at H70, D at H72, P at H73, T at H82A, S at H82B, V at H82C, Tat H87, A at H88, V at H89, W at H100P, P at H101, Tat H102, V at L19, T at L22, R at L45, I at L68, D at L69, S at L79, N at L80, T at L88, and P at L103, wherein the VH and VL framework residues are numbered according to Kabat. In one embodiment, the antibody comprises a VH framework and VL framework comprising a G at H15, L at H37, E at H39, T at H40, A at H60, Tat H68, I at 69, G or S at H70, D at H72, P at H73, T at H82A, S at H82B, V at H82C, Tat H87, A at H88, V at H89, W at H100P, P at H101, Tat H102, V at L19, Tat L22, Rat L45, I at L68, D at L69, S at L79, N at L80, T at L88, and P at L103, wherein the VH and VL framework residues are numbered according to Kabat. In one embodiment, the VH framework further comprises K at H105. In one embodiment, the VH framework further comprises K at H81 and K at H82A. In one embodiment, the VH CDR1, VH CDR2, VH CDR3, VL CDR1, VL CDR2, and VL CDR3 comprises the sequence of SEQ ID NO: 26, 27, 28, 29, 30, and 31, respectively. In one embodiment, the VH CDR1, VH CDR2, VH CDR3, VL CDR1, VL CDR2, and VL CDR3 comprises the sequence of SEQ ID NO: 26, 27, 64, 29, 30, and 31, respectively. In one embodiment, the VH CDR1, VH CDR2, VH CDR3, VL CDR1, VL CDR2, and VL CDR3 comprises the sequence of SEQ ID NO: 18, 19, 67, 23, 24, and 68, respectively. In one embodiment, the VH CDR1, VH CDR2, VH CDR3, VL CDR1, VL CDR2, and VL CDR3 comprises the sequence of SEQ ID NO: 18, 19, 22, 23, 24, and 68, respectively. In one embodiment, the antibody comprises the PGT-121 VH CDR1, VH CDR2, VH CDR3, VL CDR1, VL CDR2, and VL CDR3. In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.15.L1 VL (SEQ ID NO: 10). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.14.L1 VL (SEQ ID NO: 55). In embodiment, the antibody comprises the VH CDR1, VH CDR2, and VH CDR3 of the ePGT121.15.H6 VH (SEQ ID NO: 8) and the VL CDR1, VL CDR2, and VL CDR3 of the ePGT-121.17.L1 VL (SEQ ID NO: 66). In embodiment, the antibody comprises the VH CDR1, VH CDR2, and VH CDR3 of the ePGT121.18.H1 VH (SEQ ID NO: 65) and the VL CDR1, VL CDR2, and VL CDR3 of the ePGT-121.17.L1 VL (SEQ ID NO: 66). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody disclosed herein comprises a VH having at least about 70%, at least about 75%, at least about 80%, or at least about 90% sequence identity to SEQ ID NO: 1 and a VL having at least about 70%, at least about 75%, at least about 80%, or at least about 90% sequence identity SEQ ID NO: 2, wherein the VH comprises one or more of a V at H2, G at H15, S at H31, L at H37, E at H39, T at H40, L or I at H45, T at H51, H at H53, A at H60, H or T at H68, I at H69, G at H70, H at H72, P at H73, E at H74, K at H81, K or Tat H82A, S at H82B, V at H82C, Tat H87, A at H88, V at H89, C at H100A, Vat H100B, E at H100F, G at H100G, G at H100H, L, F, Y or C at H100J, Y at H100K, W at H100P, E or P at H101, Tat H102, and N or K at H105, I at H107, K at H108, wherein the VH residues are numbered according to Kabat. In one embodiment, the VH comprises two or more, three or more, four or more, or five or more of a V at H2, G at H15, Sat H31, L at H37, E at H39, T at H40, L or I at H45, T at H51, H at H53, A at H60, H or Tat H68, I at H69, G at H70, H at H72, P at H73, E at H74, K at H81, K or T at H82A, S at H82B, V at H82C, Tat H87, A at H88, V at H89, C at H100A, V at H100B, E at H100F, G at H100G, G at H100H, L, F, Y or C at H100J, Y at H100K, W at H100P, E or P at H101, T at H102, and N or K at H105, I at H107, K at H108. In one embodiment, the VH comprises a V at H2, G at H15, S at H31, L at H37, E at H39, T at H40, L or I at H45, T at H51, H at H53, A at H60, H or T at H68, I at H69, G at H70, H at H72, P at H73, E at H74, K at H81, K or Tat H82A, S at H82B, V at H82C, Tat H87, A at H88, V at H89, C at H100A, V at H100B, E at H100F, G at H100G, G at H100H, L, F, Y or C at H100J, Y at H100K, W at H100P, E or P at H101, Tat H102, and N or K at H105, I at H107, K at H108. In one embodiment, the VH comprises a G at H15, S at H31, L at H37, E at H39, T at H40, T at H51, H at H53, A at H60, H or T at H68, L or I at H69, G or S at H70, D or H at H72, P at H73, K or T at H82A, S at H82B, V at H82C, T at H87, A at H88, V at H89, L, F, W, Y or C at H100J, W or M at H100P, E, D or P at H101, V or T at H102, and N or K at H105. In one embodiment, the VH comprises a G at H15, S at H31, L at H37, E at H39, T at H40, T at H51, H at H53, A at H60, H or T at H68, L or I at H69, G or S at H70, D or H at H72, P at H73, V at H82A, A at H82B, A at H82C, S at H87, G at H88, K at H89, L, F, W, Y or C at H100J, W or Mat H100P, E, D or P at H101, V or T at H102, and N or K at H105. In one embodiment, the VH comprises a G at H15, S at H31, L at H37, E at H39, T at H40, T at H51, H at H53, A at H60, H at H68, L at H69, G at H70, H at H72, P at H73, K or T at H82A, S at H82B, V at H82C, Tat H87, A at H88, Vat H89, L, F, W, Y or C at H100J, W or Mat H100P, E, D or P at H101, V or T at H102, and N or K at H105. In one embodiment, the VH comprises a G at H15, S at H31, L at H37, E at H39, T at H40, T at H51, H at H53, A at H60, T at H68, I at H69, G or S at H70, D at H72, P at H73, V at H82A, A at H82B, A at H82C, S at H87, G at H88, K at H89, L, F, W, Y or C at H100J, W or M at H100P, E, D or P at H101, V or T at H102, and N or K at H105. In one embodiment, the VH comprises MDV, WDV, MEV, WDV, or WPT at H100P, H101, and H102, respectively. In one embodiment, the VL comprises one or more of a S at L2, Y at L3, V at L4, L at L5, T at L6, Q at L7, P at L8, P at L9, S at L10, V at L11, V at L19, T at L22, G at L25, E at L26, I at L28, Q at L38, P at L40, K or R at L45, G at L55, P at L56, I at L68, D at L69, Sat L79, N or R at L80, T at L88, V at L93, R at L95C, N or Q at L95F, E at L97, L at L101, E at 102L, and P at L103, wherein the VL residues are numbered according to Kabat. In one embodiment, the VL comprises a V at L19, Tat L22, G at L25, E at L26, I at L28, P at L40, K or Rat L45, P at L56, I at L68, D at L69, S at L79, N at L80, T at L88, R at L95C, N at L95F, E at L97, L at L101, and P at L103. In one embodiment, the VL comprises two or more, three or more, four or more, or five or more of a V at L19, T or S at L22, G at L25, Eat L26, I at L28, A or P at L40, K or Rat L45, P at L56, I at L68, D at L69, S or Tat L79, N at L80, Tat L88, Rat L95C, N at L95F, E or V at L97, L or F at L101, and P or G at L103. In one embodiment, the VL comprises V at L19, T or S at L22, G at L25, E at L26, I at L28, A or P at L40, K or Rat L45, P at L56, I at L68, D at L69, S or T at L79, N at L80, T at L88, R at L95C, N at L95F, E or V at L97, L or F at L101, and P or G at L103. In one embodiment, the VH comprises a VH CDR3 comprising the sequence of TLHGRRX₁YGX₂VAFX₃EX₄X₅TYFYX₆X₇X₈ wherein X₁ is I or C, X₂ is I or V, X₃ is N or G, X₄ is W, C, F, L or Y, X₅ is F or Y, X₆ is M or W, X₇ is D, P or E, and X₈ is V or T (SEQ ID NO: 71) or TLHGRRIYGX₁VAFX₂EX₃X₄TYFYX₅X₆X₇ wherein X₁ is I or V, X₂ is N or G, X₃ is W, F, L or Y, X₄ is F or Y, X₅ is M or W, X₆ is D, P or E, and X₇ is V or T (SEQ ID NO: 72). In one embodiment, the VH comprises a VH CDR1 comprising the sequence of X₁SYWS, wherein X₁ is D or S (SEQ ID NO: 26); a VH CDR2 comprising the sequence of YX₁HX₂SGDTNYX₃PSLKS wherein X₁ is V or T, X2 is K or H, and X₃ is S or A (SEQ ID NO: 27); and a VH CDR3 comprising the sequence of TLHGRRX₁YGX₂VAFX₃EX₄X₅TYFYX₆X₇X₈ wherein X₁ is I or C, X₂ is I or V, X₃ is N or G, X₄ is W, C, F, L or Y, X₅ is F or Y, X₆ is M or W, X₇ is D, P or E, and X₈ is V or T (SEQ ID NO: 71) or TLHGRRIYGX₁VAFX₂EX₃X₄TYFYX₅X₆X₇ wherein X₁ is I or V, X₂ is N or G, X₃ is W, F, L or Y, X₄ is F or Y, X₅ is M or W, X₆ is D, P or E, and X₇ is V or T (SEQ ID NO: 72). In one embodiment, the VL comprises a VL CDR1 comprising the sequence of GX₁X₂SX₃GSRAVQ wherein X₁ is E or G, X₂ is K or E, and X₃ is L or I (SEQ ID NO: 29); a VL CDR2 comprising the sequence of NNQDRX₁X₂ wherein X₁ is P or G, and X₂ is S or P (SEQ ID NO: 168); and a VL CDR3 comprising the sequence of HX₁WDSRX₂PTX₃WX₄ wherein X₁ is I or V, X₂ is V or R, X₃ is K, Q or N, and X₄ is V or E (SEQ ID NO: 73). In one embodiment, the VL is SEQ ID NO: 2, 10, 11, 46, 48, 55, 66 or 127-139. In one embodiment, the VH comprises SEQ ID NO: 3-9, 45, 47, 49-54, 65, 69, 70 or 112-126. In one embodiment, at least one of the VH CDR1, VH CDR2, VH CDR3, VL CDR1, VL CDR2, and VL CDR3 comprises a different amino acid sequence than the corresponding CDR of the PGT-121 antibody. In one embodiment, the antibody is not PGT-121. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a VH of SEQ ID NO: 3-9, 45, 47, or 49-54. In one embodiment, an antibody described herein comprises a VH of SEQ ID NO: 3-9, 45, 47, 49-54, or 65. In one embodiment, an antibody described herein comprises a VH of SEQ ID NO: 3-9, 45, 47, 49-54, 65, 69, 70, or 112-126. In one embodiment, an antibody described herein comprises a VH of SEQ ID NO: 8. In one embodiment, an antibody described herein comprises a VH of SEQ ID NO: 65. In one embodiment, an antibody described herein comprises a VH of SEQ ID NO: 69. In one embodiment, an antibody described herein comprises a VH of SEQ ID NO: 70. In one embodiment, an antibody described herein comprises a VL of SEQ ID NO: 10. In one embodiment, an antibody described herein comprises a VL of SEQ ID NO: 11. In one embodiment, an antibody described herein comprises a VL of SEQ ID NO: 66. In one embodiment, an antibody described herein comprises a VL of SEQ ID NO: 46, 48, or 55. In one embodiment, an antibody described herein comprises a VL of SEQ ID NO: 127-139. In one embodiment, an antibody described herein comprises (i) a VH of SEQ ID NO: 3-9, 45, 47, or 49-54 and (ii) a VL of SEQ ID NO: 10, 11, 46, 48, or 55. In one embodiment, an antibody described herein comprises (i) a VH of SEQ ID NO: 3-9, 45, 47, 49-54, or 65 and (ii) a VL of SEQ ID NO: 10, 11, 46, 48, 55, or 66. In one embodiment, an antibody described herein comprises (i) a VH of SEQ ID NO: 3-9, 45, 47, 49-54, 65, 69, 70 or 112-126 and (ii) a VL of SEQ ID NO: 10, 11, 46, 48, 55, 66 or 127-139. In one embodiment, an antibody described herein comprises (i) a VH of SEQ ID NO: 3 and (ii) a VL of SEQ ID NO: 10 or 11. In one embodiment, an antibody described herein comprises (i) a VH of SEQ ID NO: 4 and (ii) a VL of SEQ ID NO: 10 or 11. In one embodiment, an antibody described herein comprises (i) a VH of SEQ ID NO: 5 and (ii) a VL of SEQ ID NO: 10 or 11. In one embodiment, an antibody described herein comprises (i) a VH of SEQ ID NO: 6 and (ii) a VL of SEQ ID NO: 10 or 11. In one embodiment, an antibody described herein comprises (i) a VH of SEQ ID NO: 7 and (ii) a VL of SEQ ID NO: 10 or 11. In one embodiment, an antibody described herein comprises (i) a VH of SEQ ID NO: 8 and (ii) a VL of SEQ ID NO: 10 or 11. In one embodiment, an antibody described herein comprises (i) a VH of SEQ ID NO: 8 and (ii) a VL of SEQ ID NO: 66. In one embodiment, an antibody described herein comprises (i) a VH of SEQ ID NO: 9 and (ii) a VL of SEQ ID NO: 10 or 11. In one embodiment, an antibody described herein comprises (i) a VH of SEQ ID NO: 8 and (ii) a VL of SEQ ID NO: 10. In one embodiment, an antibody described herein comprises (i) a VH of SEQ ID NO: 65 and (ii) a VL of SEQ ID NO: 66. In one embodiment, an antibody described herein comprises (i) a VH of SEQ ID NO: 69 and (ii) a VL of SEQ ID NO: 66. In one embodiment, an antibody described herein comprises (i) a VH of SEQ ID NO: 70 and (ii) a VL of SEQ ID NO: 66. In one embodiment, an antibody described herein comprises (i) a VH of SEQ ID NO: 65 and (ii) a VL of SEQ ID NO: 55. In one embodiment, an antibody described herein comprises (i) a VH of SEQ ID NO: 69 and (ii) a VL of SEQ ID NO: 55. In one embodiment, an antibody described herein comprises (i) a VH of SEQ ID NO: 70 and (ii) a VL of SEQ ID NO: 55. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein comprises a heavy chain of SEQ ID NO: 34-40. In one embodiment, an antibody described herein comprises a heavy chain of SEQ ID NO: 39. In one embodiment, an antibody described herein comprises a light chain of SEQ ID NO: 41. In one embodiment, an antibody described herein comprises a light chain of SEQ ID NO: 42. In one embodiment, an antibody described herein comprises (i) a heavy chain of SEQ ID NO: 34-40 and (ii) a light chain of SEQ ID NO: 41 or 42. In one embodiment, an antibody described herein comprises (i) a heavy chain of SEQ ID NO: 34 and (ii) a light chain of SEQ ID NO: 41 or 42. In one embodiment, an antibody described herein comprises (i) a heavy chain of SEQ ID NO: 35 and (ii) a light chain of SEQ ID NO: 41 or 42. In one embodiment, an antibody described herein comprises (i) a heavy chain of SEQ ID NO: 36 and (ii) a light chain of SEQ ID NO: 41 or 42. In one embodiment, an antibody described herein comprises (i) a heavy chain of SEQ ID NO: 37 and (ii) a light chain of SEQ ID NO: 41 or 42. In one embodiment, an antibody described herein comprises (i) a heavy chain of SEQ ID NO: 38 and (ii) a light chain of SEQ ID NO: 41 or 42. In one embodiment, an antibody described herein comprises (i) a heavy chain of SEQ ID NO: 39 and (ii) a light chain of SEQ ID NO: 41 or 42. In one embodiment, an antibody described herein comprises (i) a heavy chain of SEQ ID NO: 40 and (ii) a light chain of SEQ ID NO: 41 or 42. In one embodiment, an antibody described herein comprises (i) a heavy chain of SEQ ID NO: 39 and (ii) a light chain of SEQ ID NO: 10. In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120). In one embodiment, the antibody is capable of neutralizing 92BR020. In one embodiment, the antibody is capable of neutralizing at least two cross-clade isolates of HIV. In one embodiment, the antibody specifically binds to HIV Env (e.g., 92BR020 gp120).

In one embodiment, an antibody described herein is not polyreactive. In one embodiment, polyreactivity is assessed by the HEp-2 polyreactivity assay.

In one embodiment, an antibody described herein is a recombinant antibody, a chimeric antibody, a bispecific antibody, or a trispecific antibody. In one embodiment, the antibody fragment comprises a single-chain Fv (scFv), F(ab) fragment, F(ab′)2 fragment, or an isolated VH domain.

In one embodiment, an antibody described herein comprises a heavy and/or light chain constant region. In one embodiment, an antibody described herein comprises a human heavy and/or light chain constant region. In one embodiment, the heavy chain constant region is human immunoglobulin IgG1, IgG2, IgG3, IgG4, IgA1, or IgA2 constant region. In one embodiment, the heavy chain constant region is human immunoglobulin IgG1 constant region. In one embodiment, the heavy chain constant region comprises a native amino acid sequence. In one embodiment, the heavy chain constant region comprises the amino acid sequence of SEQ ID NO: 43. In one embodiment, the heavy chain constant region comprises a variant amino acid sequence. In one embodiment, the heavy chain constant region comprises the amino acid sequence of SEQ ID NO: 44.

In one embodiment, the antibody disclosed herein is not PGT-121. In one embodiment, the antibody disclosed herein comprises VH CDR1, VH CDR2, VH CDR3, VL CDR1, VL CDR2, and VL CDR3, wherein one or more of the VH CDR1, VH CDR2, VH CDR3, VL CDR1, VL CDR2, and VL CDR3 does not comprise the amino acid sequence of the corresponding PGT-121 CDR. In one embodiment, the antibody disclosed herein comprises a VH and a VL, wherein one or both of the VH and VL does not comprise the amino acid sequence of the corresponding PGT-121 VH and VL.

In one embodiment, an antibody disclosed herein is capable of neutralizing at least 5, at least 6 or 7 of the cross-clade HIV isolates in the 7-member indicator virus panel. In one embodiment, an antibody disclosed herein is capable of neutralizing at least 6 of the cross-clade HIV isolates in the 7-member indicator virus panel. In one embodiment, an antibody disclosed herein is capable of neutralizing at least 8, at least 9, at least 10, at least 11 or 12 of the cross-clade HIV isolates in the 12-member indicator virus panel. In one embodiment, an antibody disclosed herein is capable of neutralizing at least 9 of the cross-clade HIV isolates in the 12-member indicator virus panel. In one embodiment, an antibody disclosed herein is capable of neutralizing at least about 50%, at least about 60%, at least about 70%, at least about 80%, at least about 90%, or 100% of cross-clade HIV isolates in the 106-member indicator virus panel. In one embodiment, an antibody disclosed herein is capable of neutralizing at least about 50%, of cross-clade HIV isolates in the 106-member indicator virus panel. In one embodiment, an antibody disclosed herein is capable of neutralizing at least about 60%, of cross-clade HIV isolates in the 106-member indicator virus panel. In one embodiment, an antibody disclosed herein is capable of neutralizing at least about 62%, of cross-clade HIV isolates in the 106-member indicator virus panel. In one embodiment, an antibody disclosed herein is capable of neutralizing at least about 65%, of cross-clade HIV isolates in the 106-member indicator virus panel. In one embodiment, an antibody disclosed herein is capable of neutralizing at least about 67%, of cross-clade HIV isolates in the 106-member indicator virus panel. In one embodiment, an antibody disclosed herein is capable of neutralizing at least about 68%, of cross-clade HIV isolates in the 106-member indicator virus panel. In one embodiment, an antibody disclosed herein is capable of neutralizing at least about 69%, of cross-clade HIV isolates in the 106-member indicator virus panel. In one embodiment, an antibody disclosed herein is capable of neutralizing at least about 70% of cross-clade HIV isolates in the 106-member indicator virus panel. In one embodiment, an antibody disclosed herein is capable of neutralizing at least about 71% of cross-clade HIV isolates in the 106-member indicator virus panel. In one embodiment, an antibody disclosed herein is capable of neutralizing at least about 72% of cross-clade HIV isolates in the 106-member indicator virus panel. In one embodiment, an antibody disclosed herein is capable of neutralizing at least about 73% of cross-clade HIV isolates in the 106-member indicator virus panel. In one embodiment, an antibody disclosed herein is capable of neutralizing at least about 74% of cross-clade HIV isolates in the 106-member indicator virus panel. In one embodiment, an antibody disclosed herein is capable of neutralizing at least about 75% of cross-clade HIV isolates in the 106-member indicator virus panel. In one embodiment, an antibody disclosed herein is capable of neutralizing at least about 77% of cross-clade HIV isolates in the 106-member indicator virus panel. In one embodiment, an antibody disclosed herein is capable of neutralizing at least about 80% of cross-clade HIV isolates in the 106-member indicator virus panel. In one embodiment, an antibody disclosed herein is capable of neutralizing at least about 85% of cross-clade HIV isolates in the 106-member indicator virus panel. In one embodiment, an antibody disclosed herein is capable of neutralizing at least about 50%, at least about 60%, at least about 70%, at least about 80%, at least about 90%, or 100% of cross-clade HIV isolates in the 113-member indicator virus panel. In one embodiment, an antibody disclosed herein is capable of neutralizing at least about 50% of cross-clade HIV isolates in the 113-member indicator virus panel. In one embodiment, an antibody disclosed herein is capable of neutralizing at least about 55% of cross-clade HIV isolates in the 113-member indicator virus panel. In one embodiment, an antibody disclosed herein is capable of neutralizing at least about 60% of cross-clade HIV isolates in the 113-member indicator virus panel. In one embodiment, an antibody disclosed herein is capable of neutralizing at least about 65% of cross-clade HIV isolates in the 113-member indicator virus panel. In one embodiment, an antibody disclosed herein is capable of neutralizing at least about 70% of cross-clade HIV isolates in the 113-member indicator virus panel. In one embodiment, an antibody disclosed herein is capable of neutralizing at least about 75% of cross-clade HIV isolates in the 113-member indicator virus panel. In one embodiment, an antibody disclosed herein is capable of neutralizing at least about 80% of cross-clade HIV isolates in the 113-member indicator virus panel. In one embodiment, an antibody disclosed herein is capable of neutralizing the cross-clade HIV isolates with a median IC50 equal to or less than about 0.1 microg/ml, about 0.05 microg/ml, about 0.025 microg/ml, about 0.01 microg/ml, or about 0.005 microg/ml. In one embodiment, an antibody disclosed herein is capable of neutralizing the cross-clade HIV isolates with a median IC50 equal to or less than about 0.05 microg/ml. In one embodiment, an antibody disclosed herein is capable of neutralizing the cross-clade HIV isolates of the 106 member virus panel of Table 5 with a median IC50 equal to or less than about 0.05 microg/ml. In one embodiment, an antibody disclosed herein is capable of neutralizing the cross-clade HIV isolates of the 106 member virus panel of Table 5 with a median IC50 equal to or less than about 0.03 microg/ml. In one embodiment, an antibody disclosed herein is capable of neutralizing the cross-clade HIV isolates of the 106 member virus panel of Table 5 with a median IC50 equal to or less than about 0.02 microg/ml. In one embodiment, an antibody disclosed herein is capable of neutralizing the cross-clade HIV isolates of the 106 member virus panel of Table 5 with a median IC50 equal to or less than about 0.01 microg/ml. In one embodiment, an antibody disclosed herein is capable of neutralizing the cross-clade HIV isolates of the 106 member virus panel of Table 5 with a median IC50 equal to or less than about 0.009 microg/ml. In one embodiment, an antibody disclosed herein is capable of neutralizing the cross-clade HIV isolates of the 106 member virus panel of Table 5 with a median IC50 equal to or less than about 0.008 microg/ml. In one embodiment, an antibody disclosed herein is capable of neutralizing the cross-clade HIV isolates of the 106 member virus panel of Table 5 with a median IC50 equal to or less than about 0.007 microg/ml. In one embodiment, an antibody disclosed herein is capable of neutralizing the cross-clade HIV isolates of the 106 member virus panel of Table 5 with a median IC50 equal to or less than about 0.006 microg/ml. In one embodiment, an antibody disclosed herein is capable of neutralizing the cross-clade HIV isolates of the 106 member virus panel of Table 5 with a median IC50 equal to or less than about 0.005 microg/ml. In one embodiment, an antibody disclosed herein is capable of neutralizing the cross-clade HIV isolates of the 106 member virus panel of Table 5 with a median IC50 equal to or less than about 0.004 microg/ml. In one embodiment, an antibody disclosed herein is capable of neutralizing the cross-clade HIV isolates of the 106 member virus panel of Table 5 with a median IC50 equal to or less than about 0.003 microg/ml. In one embodiment, an antibody disclosed herein is capable of neutralizing the cross-clade HIV isolates of the 106 member virus panel of Table 5 with a median IC50 equal to or less than about 0.002 microg/ml. In one embodiment, an antibody disclosed herein is capable of neutralizing the cross-clade HIV isolates of the 106 member virus panel of Table 5 with a median IC50 equal to or less than about 0.001 microg/ml. In one embodiment, an antibody disclosed herein is capable of neutralizing at least about 50%, 60%, 70%, 80%, 90%, 95%, or 100% of cross-clade HIV isolates in the 106-member indicator virus panel with a median IC₅₀ equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing at least about 70% of cross-clade HIV isolates in the 106-member indicator virus panel with a median IC₅₀ equal to or less than 0.05 microg/ml. In one embodiment, an antibody disclosed herein is capable of neutralizing at least about 70% of cross-clade HIV isolates in the 106-member indicator virus panel with a median IC₅₀ equal to or less than 0.01 microg/ml. In one embodiment, an antibody disclosed herein is capable of neutralizing at least about 70% of cross-clade HIV isolates in the 106-member indicator virus panel with a median IC₅₀ equal to or less than 0.005 microg/ml. In one embodiment, an antibody disclosed herein is capable of neutralizing at least about 70% of cross-clade HIV isolates in the 106-member indicator virus panel with a median IC₅₀ equal to or less than 0.001 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC50 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC50 equal to or less than 0.05 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC50 equal to or less than 0.01 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC50 equal to or less than 0.005 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC50 equal to or less than 0.001 microg/ml. In one embodiment, an antibody disclosed herein is capable of neutralizing the clade A HIV isolates in the 106-member indicator virus panel with a median IC50 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing the clade A HIV isolates in the 106-member indicator virus panel with a median IC50 equal to or less than 0.05 microg/ml. In one embodiment, an antibody disclosed herein is capable of neutralizing the clade A HIV isolates in the 106-member indicator virus panel with a median IC50 equal to or less than 0.01 microg/ml. In one embodiment, an antibody disclosed herein is capable of neutralizing the clade A HIV isolates in the 106-member indicator virus panel with a median IC50 equal to or less than 0.005 microg/ml. In one embodiment, an antibody disclosed herein is capable of neutralizing the clade A HIV isolates in the 106-member indicator virus panel with a median IC50 equal to or less than 0.001 microg/ml. In one embodiment, an antibody disclosed herein is capable of neutralizing the clade B HIV isolates in the 106-member indicator virus panel with a median IC50 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing the clade B HIV isolates in the 106-member indicator virus panel with a median IC50 equal to or less than 0.05 microg/ml. In one embodiment, an antibody disclosed herein is capable of neutralizing the clade B HIV isolates in the 106-member indicator virus panel with a median IC50 equal to or less than 0.01 microg/ml. In one embodiment, an antibody disclosed herein is capable of neutralizing the clade B HIV isolates in the 106-member indicator virus panel with a median IC50 equal to or less than 0.005 microg/ml. In one embodiment, an antibody disclosed herein is capable of neutralizing the clade B HIV isolates in the 106-member indicator virus panel with a median IC50 equal to or less than 0.001 microg/ml. In one embodiment, an antibody disclosed herein is capable of neutralizing the clade C HIV isolates in the 106-member indicator virus panel with a median IC50 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing the clade C HIV isolates in the 106-member indicator virus panel with a median IC50 equal to or less than 0.05 microg/ml. In one embodiment, an antibody disclosed herein is capable of neutralizing the clade C HIV isolates in the 106-member indicator virus panel with a median IC50 equal to or less than 0.01 microg/ml. In one embodiment, an antibody disclosed herein is capable of neutralizing the clade C HIV isolates in the 106-member indicator virus panel with a median IC50 equal to or less than 0.005 microg/ml. In one embodiment, an antibody disclosed herein is capable of neutralizing the clade C HIV isolates in the 106-member indicator virus panel with a median IC50 equal to or less than 0.001 microg/ml. In one embodiment, an antibody disclosed herein is capable of neutralizing the cross-clade HIV isolates with a median IC80 equal to or less than about 0.1 microg/ml, about 0.05 microg/ml, about 0.025 microg/ml, about 0.01 microg/ml, or about 0.005 microg/ml. In one embodiment, an antibody disclosed herein is capable of neutralizing the cross-clade HIV isolates with a median IC80 equal to or less than about 0.05 microg/ml. In one embodiment, an antibody disclosed herein is capable of neutralizing the cross-clade HIV isolates of the 106 member virus panel of Table 5 with a median IC80 equal to or less than about 0.05 microg/ml. In one embodiment, an antibody disclosed herein is capable of neutralizing the cross-clade HIV isolates of the 106 member virus panel of Table 5 with a median IC80 equal to or less than about 0.03 microg/ml. In one embodiment, an antibody disclosed herein is capable of neutralizing the cross-clade HIV isolates of the 106 member virus panel of Table 5 with a median IC80 equal to or less than about 0.02 microg/ml. In one embodiment, an antibody disclosed herein is capable of neutralizing the cross-clade HIV isolates of the 106 member virus panel of Table 5 with a median IC80 equal to or less than about 0.01 microg/ml. In one embodiment, an antibody disclosed herein is capable of neutralizing the cross-clade HIV isolates of the 106 member virus panel of Table 5 with a median IC80 equal to or less than about 0.009 microg/ml. In one embodiment, an antibody disclosed herein is capable of neutralizing the cross-clade HIV isolates of the 106 member virus panel of Table 5 with a median IC80 equal to or less than about 0.008 microg/ml. In one embodiment, an antibody disclosed herein is capable of neutralizing the cross-clade HIV isolates of the 106 member virus panel of Table 5 with a median IC80 equal to or less than about 0.007 microg/ml. In one embodiment, an antibody disclosed herein is capable of neutralizing the cross-clade HIV isolates of the 106 member virus panel of Table 5 with a median IC80 equal to or less than about 0.006 microg/ml. In one embodiment, an antibody disclosed herein is capable of neutralizing the cross-clade HIV isolates of the 106 member virus panel of Table 5 with a median IC80 equal to or less than about 0.005 microg/ml. In one embodiment, an antibody disclosed herein is capable of neutralizing the cross-clade HIV isolates of the 106 member virus panel of Table 5 with a median IC80 equal to or less than about 0.004 microg/ml. In one embodiment, an antibody disclosed herein is capable of neutralizing the cross-clade HIV isolates of the 106 member virus panel of Table 5 with a median IC80 equal to or less than about 0.003 microg/ml. In one embodiment, an antibody disclosed herein is capable of neutralizing the cross-clade HIV isolates of the 106 member virus panel of Table 5 with a median IC80 equal to or less than about 0.002 microg/ml. In one embodiment, an antibody disclosed herein is capable of neutralizing the cross-clade HIV isolates of the 106 member virus panel of Table 5 with a median IC80 equal to or less than about 0.001 microg/ml. In one embodiment, an antibody disclosed herein is capable of neutralizing at least about 50%, 60%, 70%, 80%, 90%, 95%, or 100% of cross-clade HIV isolates in the 106-member indicator virus panel with a median IC80 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing at least about 70% of cross-clade HIV isolates in the 106-member indicator virus panel with a median IC80 equal to or less than 0.05 microg/ml. In one embodiment, an antibody disclosed herein is capable of neutralizing at least about 70% of cross-clade HIV isolates in the 106-member indicator virus panel with a median IC80 equal to or less than 0.01 microg/ml. In one embodiment, an antibody disclosed herein is capable of neutralizing at least about 70% of cross-clade HIV isolates in the 106-member indicator virus panel with a median IC80 equal to or less than 0.005 microg/ml. In one embodiment, an antibody disclosed herein is capable of neutralizing at least about 70% of cross-clade HIV isolates in the 106-member indicator virus panel with a median IC80 equal to or less than 0.001 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC80 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC80 equal to or less than 0.05 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC80 equal to or less than 0.01 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC80 equal to or less than 0.005 microg/ml. In one embodiment, an antibody disclosed herein is capable of neutralizing the clade A, A (T/F), AC, ACD, B, B (T/F), BC, C, C (T/F), CD, CRF01_AE, CRF01_AE (T/F), CRF02_AG, D, or G HIV isolates in the 106-member indicator virus panel with a median IC80 equal to or less than 0.001 microg/ml. In one embodiment, an antibody disclosed herein is capable of neutralizing the clade A HIV isolates in the 106-member indicator virus panel with a median IC80 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing the clade A HIV isolates in the 106-member indicator virus panel with a median IC80 equal to or less than 0.05 microg/ml. In one embodiment, an antibody disclosed herein is capable of neutralizing the clade A HIV isolates in the 106-member indicator virus panel with a median IC80 equal to or less than 0.01 microg/ml. In one embodiment, an antibody disclosed herein is capable of neutralizing the clade A HIV isolates in the 106-member indicator virus panel with a median IC80 equal to or less than 0.005 microg/ml. In one embodiment, an antibody disclosed herein is capable of neutralizing the clade A HIV isolates in the 106-member indicator virus panel with a median IC80 equal to or less than 0.001 microg/ml. In one embodiment, an antibody disclosed herein is capable of neutralizing the clade B HIV isolates in the 106-member indicator virus panel with a median IC80 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing the clade B HIV isolates in the 106-member indicator virus panel with a median IC80 equal to or less than 0.05 microg/ml. In one embodiment, an antibody disclosed herein is capable of neutralizing the clade B HIV isolates in the 106-member indicator virus panel with a median IC80 equal to or less than 0.01 microg/ml. In one embodiment, an antibody disclosed herein is capable of neutralizing the clade B HIV isolates in the 106-member indicator virus panel with a median IC80 equal to or less than 0.005 microg/ml. In one embodiment, an antibody disclosed herein is capable of neutralizing the clade B HIV isolates in the 106-member indicator virus panel with a median IC80 equal to or less than 0.001 microg/ml. In one embodiment, an antibody disclosed herein is capable of neutralizing the clade C HIV isolates in the 106-member indicator virus panel with a median IC80 equal to or less than about 0.1 microg/ml, 0.07 microg/ml, 0.06 microg/ml, 0.05 microg/ml, 0.025 microg/ml, 0.01 microg/ml or 0.005 microg/ml. In one embodiment, an antibody disclosed herein is capable of neutralizing the clade C HIV isolates in the 106-member indicator virus panel with a median IC80 equal to or less than 0.05 microg/ml. In one embodiment, an antibody disclosed herein is capable of neutralizing the clade C HIV isolates in the 106-member indicator virus panel with a median IC80 equal to or less than 0.01 microg/ml. In one embodiment, an antibody disclosed herein is capable of neutralizing the clade C HIV isolates in the 106-member indicator virus panel with a median IC80 equal to or less than 0.005 microg/ml. In one embodiment, an antibody disclosed herein is capable of neutralizing the clade C HIV isolates in the 106-member indicator virus panel with a median IC80 equal to or less than 0.001 microg/ml.

In one embodiment, an antibody disclosed herein is capable of neutralizing a PGT-121 resistant HIV isolate. In one embodiment, an antibody disclosed herein is capable of neutralizing at least 2, at least 3, at least 4, or at least 5 PGT-121 resistant HIV isolates. In one embodiment, an antibody disclosed herein is capable of neutralizing at least 2 cross-clade PGT-121 resistant HIV isolates. In one embodiment, an antibody disclosed herein is capable of neutralizing at least 2, at least 3, at least 4, or at least 5 PGT-121 resistant HIV isolates. In one embodiment, the PGT-121 resistant HIV isolate is HIV-0013095-2.11 (clade C), CNE30 (clade BC), CNE58 (clade BC), 249M B10 (clade C), HIV-0013095 (clade C), HIV-16055 (clade C), ZM249 (clade C), BJOX 28000 (clade AE), T278-50 (clade AG), Ce2060_G9 (clade C), 231965.c01 (clade D), 6540.v4.c1 (clade AC), and 6041.v3.c23 (clade AC). In one embodiment, an antibody disclosed herein is capable of neutralizing the PGT-121 resistant HIV isolates with a median IC50 equal to or less than about 0.1 microg/ml, about 0.05 microg/ml, about 0.025 microg/ml, about 0.01 microg/ml, or about 0.005 microg/ml. In one embodiment, an antibody disclosed herein is capable of neutralizing the PGT-121 resistant HIV isolates with a median IC50 equal to or less than about 0.05 microg/ml. In one embodiment, an antibody disclosed herein is capable of neutralizing the PGT-121 resistant HIV isolates with a median IC80 equal to or less than about 0.1 microg/ml, about 0.05 microg/ml, about 0.025 microg/ml, about 0.01 microg/ml, or about 0.005 microg/ml. In one embodiment, an antibody disclosed herein is capable of neutralizing the PGT-121 resistant HIV isolates with a median IC80 equal to or less than about 0.05 microg/ml.

In one embodiment, an antibody disclosed herein is capable of neutralizing a larger fraction of the cross-clade HIV isolates in the 7-member indicator virus panel than PGT-121. In one embodiment, an antibody disclosed herein is capable of neutralizing a larger fraction of the cross-clade HIV isolates in the 12-member indicator virus panel than PGT-121. In one embodiment, an antibody disclosed herein is capable of neutralizing a larger fraction of the cross-clade HIV isolates in the 106-member indicator virus panel than PGT-121. In one embodiment, an antibody disclosed herein is capable of neutralizing a larger fraction of the cross-clade HIV isolates in the 113-member indicator virus panel than PGT-121.

In one embodiment, an antibody disclosed herein is capable of neutralizing cross-clade HIV isolates in the 7-member indicator virus panel with a lower median IC50 than PGT-121. In one embodiment, an antibody disclosed herein is capable of neutralizing cross-clade HIV isolates in the 12-member indicator virus panel with a lower median IC50 than PGT-121. In one embodiment, an antibody disclosed herein is capable of neutralizing cross-clade HIV isolates in the 106-member indicator virus panel with a lower median IC50 than PGT-121. In one embodiment, an antibody disclosed herein is capable of neutralizing cross-clade HIV isolates in the 113-member indicator virus panel shown with a lower median IC50 than PGT-121.

In another aspect, provided herein are antibodies that bind the same or an overlapping epitope of Env as an antibody described herein (e.g., ePGT121.15.H6.L1 antibody comprising the ePGT121.15.H6 VH and ePGT121.15.L1 VL). In certain embodiments, the epitope of an antibody can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189: 1-23; Chayen N E (1997) Structure 5: 1269-1274; McPherson A (1976) J Biol Chem 251: 6300-6303). Antibody:antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al.; U.S. Patent Application No. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies may be accomplished using any method known to one of skill in the art. See, e.g., Champe M et al., (1995) supra and Cunningham B C & Wells J A (1989) supra for a description of mutagenesis techniques, including alanine scanning mutagenesis techniques. In a specific embodiment, the epitope of an antibody is determined using alanine scanning mutagenesis studies. Usually, binding to the antigen is reduced or disrupted when a residue within the epitope is substituted to alanine. In one embodiment, the Kd of binding to the antigen is increased by about 5-fold, 10-fold, 20-fold, 10-fold or more when a residue within the epitope is substituted for alanine. In one embodiment, binding affinity is determined by ELISA. In addition, antibodies that recognize and bind to the same or overlapping epitopes of Env (e.g., an epitope of 92BR020 Env) can be identified using routine techniques such as an immunoassay, for example, by showing the ability of one antibody to block the binding of another antibody to a target antigen, i.e., a competitive binding assay.

In specific aspects, provided herein is an antibody, which immunospecifically binds to the same epitope as an antibody comprising the ePGT121.15.H1 VH and ePGT-121.15.L1 VL, the ePGT121.15.H2 VH and ePGT121.15.L1 VL, the ePGT121.15.H3 VH and ePGT121.15.L1 VL, the ePGT121.15.H4 VH and ePGT121.15.L1 VL, the ePGT121.15.H5 VH and ePGT121.15.L1 VL, the ePGT121.15.H6 VH and ePGT121.15.L1 VL, the ePGT121.15.H7 VH and ePGT121.15.L1 VL, ePGT121.15.H1 VH and ePGT121.15.L2 VL, the ePGT121.15.H2 VH and ePGT121.15.L2 VL, the ePGT121.15.H3 VH and ePGT121.15.L2 VL, the ePGT121.15.H4 VH and ePGT121.15.L2 VL, the ePGT121.15.H5 VH and ePGT121.15.L2 VL, the ePGT121.15.H6 VH and ePGT121.15.L2 VL, the ePGT121.15.H7 VH and ePGT-121.15.L2 VL for specific binding to Env (e.g., an epitope of 92BR020 Env). Assays known to one of skill in the art or described herein (e.g., X-ray crystallography, hydrogen/deuterium exchange coupled with mass spectrometry (e.g., liquid chromatography electrospray mass spectrometry), alanine scanning, ELISA assays, etc.) can be used to determine if two antibodies bind to the same epitope.

In one embodiment, an antibody described herein immunospecifically binds to the same epitope as that bound by an antibody comprising the ePGT-121.15.1 VH and ePGT121.15.L1 VL, the ePGT-121.15.2 VH and ePGT121.15.L1 VL, the ePGT-121.15.3 VH and ePGT121.15.L1 VL, the ePGT-121.15.4 VH and ePGT121.15.L1 VL, the ePGT-121.15.5 VH and ePGT121.15.L1 VL, the ePGT-121.15.6 VH and ePGT121.15.L1 VL, the ePGT-121.15.7 VH and ePGT121.15.L1 VL, ePGT-121.15.1 VH and ePGT121.15.L2 VL, the ePGT-121.15.2 VH and ePGT121.15.L2 VL, the ePGT-121.15.3 VH and ePGT121.15.L2 VL, the ePGT-121.15.4 VH and ePGT121.15.L2 VL, the ePGT-121.15.5 VH and ePGT121.15.L2 VL, the ePGT-121.15.6 VH and ePGT121.15.L2 VL, the ePGT-121.15.7 VH and ePGT121.15.L2 VL.

In one embodiment, an antibody described herein immunospecifically binds to an epitope that overlaps the epitope bound by an antibody comprising the ePGT-121.15.H1 VH and ePGT121.15.L1 VL, the ePGT-121.15.H2 VH and ePGT121.15.L1 VL, the ePGT-121.15.H3 VH and ePGT121.15.L1 VL, the ePGT-121.15.H4 VH and ePGT121.15.L1 VL, the ePGT-121.15.H5 VH and ePGT121.15.L1 VL, the ePGT-121.15.H6 VH and ePGT121.15.L1 VL, the ePGT-121.15.H7 VH and ePGT121.15.L1 VL, ePGT-121.15.H1 VH and ePGT121.15.L2 VL, the ePGT-121.15.H2 VH and ePGT121.15.L2 VL, the ePGT-121.15.H3 VH and ePGT121.15.L2 VL, the ePGT-121.15.H4 VH and ePGT121.15.L2 VL, the ePGT-121.15.H5 VH and ePGT121.15.L2 VL, the ePGT-121.15.H6 VH and ePGT121.15.L2 VL, the ePGT-121.15.H7 VH and ePGT121.15.L2 VL.

In some embodiments, an antibody described herein is capable of competing with PGT-121 for binding to HIV gp120. In some embodiments, the HIV gp120 is 92BR020 gp120.

In a specific embodiment, an antibody described herein immunospecifically binds to the same epitope as that bound by an antibody comprising ePGT121.15.H6 VH and ePGT121.15.L1 VL or an epitope that overlaps the epitope.

In one aspect, provided herein is a method for producing an engineered variant of the PGT-121 antibody comprising substituting one or more amino acid residues of the PGT-121 VH to a V at H2, G at H15, S at H31, I at H37, E at H39, T at H40, T at H51, H at H53, A at H60, H at H68, G at H70, H at H72, P at H73, K at H81, K or Tat H82A, S at H82B, V at H82C, Tat H87, A at H88, Vat H89, Vat H100D, G at H100H, F or L at H100J, W at H100P, E at H101, and K at H105, wherein the VH residues are numbered according to Kabat; and/or substituting one or more amino acid residues of the PGT-121 VL to a S at L2, Y at L3, V at L4, L at L5, Tat L6, Q at L7, P at L8, P at L9, S at L10, V at L11, V at L19, G at L25, E at L26, I at L28, K at L45, P at L56, I at L68, D or A at L69, N at L80, T at L88, R at L95C, and N at L95F, wherein the VL residues are numbered according to Kabat. In one aspect, provided herein is a method for producing an engineered variant of the PGT-121 antibody comprising substituting one or more amino acid residues of the PGT-121 VH to a Vat H2, G at H15, S at H31, L at H37, Eat H39, Tat H40, L or I at H45, T at H51, H at H53, A at H60, H or T at H68, I at H69, G at H70, H at H72, P at H73, E at H74, K at H81, K or T at H82A, S at H82B, V at H82C, T at H87, A at H88, V at H89, C at H100A, V at H100B, E at H100F, G at H100G, G at H100H, L, F, Y or C at H100J, Y at H100K, W at H100P, E or P at H101, Tat H102, and N or K at H105, I at H107, K at H108 wherein the VH residues are numbered according to Kabat; and/or substituting one or more amino acid residues of the PGT-121 VL to a S at L2, Y at L3, V at L4, L at L5, T at L6, Q at L7, P at L8, P at L9, S at L10, V at L11, V at L19, Tat L22, G at L25, Eat L26, I at L28, Q at L38, P at L40, K or Rat L45, G at L55, P at L56, I at L68, D at L69, S at L79, N or Rat L80, T at L88, V at L93, Rat L95C, N or Q at L95F, E at L97, L at L101, E at 102L, and P at L103, wherein the VL residues are numbered according to Kabat. In one embodiment, the engineered variant antibody is capable of neutralizing 92BR020. In one embodiment, the engineered variant antibody is capable of neutralizing at least two cross-clade isolates of HIV. In one embodiment, the engineered variant antibody specifically binds to HIV Env (e.g., 92BR020 gp120).

In certain embodiments, the epitope of an antibody described herein is used as an immunogen to produce antibodies.

The affinity or avidity of an antibody for an antigen can be determined experimentally using any suitable method well known in the art, e.g., flow cytometry, enzyme-linked immunoabsorbent assay (ELISA), or radioimmunoassay (RIA), or kinetics (e.g., BIACORE™ analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., KD or Kd, Kon, Koff) are made with standardized solutions of antibody and antigen, and a standardized buffer, as known in the art and such as the buffer described herein. In some embodiments, an antibody described herein binds to HIV gp120 with a higher affinity than PGT-121. In some embodiments, the HIV gp120 is 92BR020 gp120.

In some embodiments, an antibody described herein is a monoclonal antibody. Monoclonal antibodies can be made using recombinant DNA methods, for example, as described in U.S. Pat. No. 4,816,567. The polynucleotides encoding a monoclonal antibody can be amplified from a suitable source or chemically synthetized. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells.

The polynucleotide(s) encoding a monoclonal antibody can be modified in a number of different manners using recombinant DNA technology to generate alternative antibodies. In some embodiments, the constant domains of the light and heavy chains can be substituted for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

Methods for engineering antibodies can also be used and are well known in the art. An engineered antibody can have one or more amino acid residues substituted, deleted or inserted. These sequence modifications can be used to reduce immunogenicity or reduce, enhance or modify binding, affinity, on-rate, off-rate, avidity, specificity, half-life, or any other suitable characteristic, as known in the art. Antibodies can also be engineered to eliminate development liabilities by altering or eliminating sequence elements targeted for post-translational modification including glycosylation sites, oxidation sites, or deamination sites. In general, the CDR residues are directly and most substantially involved in influencing antibody binding. Accordingly, part or all of the CDR sequences are maintained while the variable framework and constant regions can be engineered by introducing substitutions, insertions, or deletions.

Antibodies disclosed herein can also optionally be engineered with retention of high affinity for the antigen and other favorable biological properties. To achieve this goal, engineered antibodies can be prepared by a process of analysis of the parental sequences and various conceptual engineered products using three-dimensional models of the parental and engineered sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In certain embodiments an antibody fragment is provided. Various techniques are known for the production of antibody fragments. Traditionally, these fragments are derived via proteolytic digestion of intact antibodies (for example Morimoto et al., 1993, Journal of Biochemical and Biophysical Methods 24:107-117; Brennan et al., 1985, Science, 229:81). In certain embodiments, antibody fragments are produced recombinantly. Fab, Fv, and scFv antibody fragments can all be expressed in and secreted from E. coli or other host cells, thus allowing the production of large amounts of these fragments. Such antibody fragments can also be isolated from antibody phage libraries. The antibody fragment can also be linear antibodies as described in U.S. Pat. No. 5,641,870, for example, and can be monospecific or bispecific. Other techniques for the production of antibody fragments will be apparent to the skilled practitioner.

In certain embodiments, the variable domains in both the heavy and light chains are altered by at least partial replacement of one or more CDRs and, if necessary, by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It may not be necessary to replace all of the CDRs with the complete CDRs from the donor variable region to transfer the antigen-binding capacity of one variable domain to another. Rather, it may only be necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that the modified antibodies described herein will comprise antibodies (e.g., full-length antibodies or antigen-binding fragments thereof) in which at least a fraction of one or more of the constant region domains has been deleted or otherwise altered so as to provide desired biochemical characteristics such as increased serum half-life when compared with an antibody of approximately the same antigen-binding activity comprising a native or unaltered constant region. In some embodiments, the constant region of the modified antibodies will comprise a human constant region. Modifications to the constant region compatible with this invention comprise additions, deletions or substitutions of one or more amino acids in one or more domains. That is, the modified antibodies described herein can comprise alterations or modifications to one or more of the three heavy chain constant domains (CH1, CH2 or CH3) and/or to the light chain constant domain (CL). In some embodiments, modified constant regions wherein one or more domains are partially or entirely deleted are contemplated. In some embodiments, the modified antibodies will comprise domain deleted constructs or variants wherein the entire CH2 domain has been removed (ΔCH2 constructs). In some embodiments, the omitted constant region domain will be replaced by a short amino acid spacer (e.g., 10 residues) that provides some of the molecular flexibility typically imparted by the absent constant region.

It will be noted that in certain embodiments, the modified antibodies can be engineered to fuse the CH3 domain directly to the hinge region of the respective modified antibodies. In other constructs it may be desirable to provide a peptide spacer between the hinge region and the modified CH2 and/or CH3 domains. For example, compatible constructs could be expressed wherein the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic, or even omitted altogether, so as to maintain the desired biochemical qualities of the modified antibodies.

Besides the deletion of whole constant region domains, it will be appreciated that the antibodies described herein can be provided by the partial deletion or substitution of a few or even a single amino acid. For example, it may be desirable to simply delete that part of one or more constant region domains that control the effector function (e.g., complement C1Q binding) to be modulated. Such partial deletions of the constant regions can improve selected characteristics of the antibody (serum half-life) while leaving other desirable functions associated with the subject constant region domain intact. Moreover, as alluded to above, the constant regions of the disclosed antibodies can be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. Certain embodiments can comprise the addition of one or more amino acids to the constant region to enhance desirable characteristics such as decreasing or increasing effector function or provide for more cytotoxin or carbohydrate attachment. In such embodiments it can be desirable to insert or replicate specific sequences derived from selected constant region domains. In further embodiments, an antibody disclosed herein comprises a variant IgG Fc region (e.g., variant IgG1 Fc region) comprising the M428L and N434S substitutions to improve the recycling of the antibody via the antibody salvage pathway.

The present invention further embraces variants and equivalents which are substantially homologous to the antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

The polypeptides provided herein can be recombinant polypeptides or synthetic polypeptides comprising an antibody, or fragment thereof. It will be recognized in the art that some amino acid sequences described herein can be varied without significant effect of the structure or function of the protein. Thus, the invention further includes variations of the polypeptides which show substantial activity or which include regions of an antibody, or fragment thereof, against an HIV Env protein. Such mutants include deletions, insertions, inversions, repeats, and type substitutions.

The polypeptides and analogs can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in REMINGTON'S PHARMACEUTICAL SCIENCES, 21th ed., Mack Publishing Co., Easton, Pa. (2005).

### III. Polynucleotides

In certain aspects, provided herein are polynucleotides comprising a nucleotide sequence or nucleotide sequences encoding an antibody described herein (e.g., a variable light chain and/or variable heavy chain region) or an antigen binding fragment thereof and vectors, e.g., vectors comprising such polynucleotides. In one embodiment, the vectors can be used for recombinant expression of an antibody described herein in host cells (e.g., E. coli and mammalian cells). In one embodiment, the vectors can be used for administration of an antibody described herein to a patient in need thereof. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one aspect, provided herein are isolated polynucleotides encoding the heavy chain variable region or heavy chain of an antibody described herein.

In one aspect, provided herein are isolated polynucleotides encoding the light chain variable region or light chain of an antibody described herein.

In one aspect, provided herein are isolated polynucleotides encoding the heavy chain variable region or heavy chain of an antibody described herein and the light chain variable region or light chain of an antibody described herein.

In one embodiment, the polynucleotide encodes the ePGT121.15.H1, ePGT121.15.H2, ePGT121.15.H3, ePGT121.15H.4, ePGT121.15.H5, ePGT121.15.H6, ePGT121.15.H7, ePGT121.13.H1, ePGT121.14.H1, ePGT121.14.H2, ePGT121.14.H3, ePGT121.14.H4, ePGT121.14.H5, or ePGT121.14.H6 heavy chain variable region. In one embodiment, the polynucleotide encodes the ePGT-121.15.H6 heavy chain variable region. In one embodiment, the polynucleotide encodes the ePGT-121.18.H1 heavy chain variable region. In one embodiment, the polynucleotide encodes the ePGT-121.18.H17 heavy chain variable region. In one embodiment, the polynucleotide encodes the ePGT-121.18.H18 heavy chain variable region.

In one embodiment, the polynucleotide encodes a heavy chain variable region comprising the amino acid sequence of SEQ ID NO: 3-9, 45, 47, or 49-54. In one embodiment, the polynucleotide encodes a heavy chain variable region comprising the amino acid sequence of SEQ ID NO: 3-9, 45, 47, 49-54, 65, 69, 70 or 112-126. In one embodiment, the polynucleotide encodes the heavy chain variable region comprising the amino acid sequence of SEQ ID NO: 8. In one embodiment, the polynucleotide encodes the heavy chain variable region comprising the amino acid sequence of SEQ ID NO: 65. In one embodiment, the polynucleotide encodes the heavy chain variable region comprising the amino acid sequence of SEQ ID NO: 69. In one embodiment, the polynucleotide encodes the heavy chain variable region comprising the amino acid sequence of SEQ ID NO: 70.

In one embodiment, the polynucleotide encodes a heavy chain comprising the amino acid sequence of SEQ ID NO: 34-40. In one embodiment, the polynucleotide encodes the heavy chain comprising the amino acid sequence of SEQ ID NO: 39.

In one embodiment, the polynucleotide encodes the ePGT121.15.L1, ePGT121.15.L2, ePGT121.12.L4, ePGT121.13.L1, or ePGT121.14.L1 light chain variable region. In one embodiment, the polynucleotide encodes the ePGT121.15.L1, ePGT121.15.L2, ePGT121.12.L4, ePGT121.13.L1, ePGT121.14.L1, or ePGT121.17.L1 light chain variable region. In one embodiment, the polynucleotide encodes the ePGT121.17.L1 light chain variable region.

In one embodiment, the polynucleotide encodes a light chain variable region comprising the amino acid sequence of SEQ ID NO: 10, 11, 46, 48 or 55. In one embodiment, the polynucleotide encodes a light chain variable region comprising the amino acid sequence of SEQ ID NO: 10, 11, 46, 48, 55, 66 or 127-139. In one embodiment, the polynucleotide encodes a light chain variable region comprising the amino acid sequence of SEQ ID NO: 10. In one embodiment, the polynucleotide encodes a light chain variable region comprising the amino acid sequence of SEQ ID NO: 66.

In one embodiment, the polynucleotide encodes a light chain comprising the amino acid sequence of SEQ ID NO: 41 or 42.

In one embodiment, an isolated polynucleotide described herein encodes an antibody described herein and comprises an mRNA. In one embodiment, the mRNA comprises at least one modified nucleotide. In one embodiment, a modified mRNA encoding an antibody disclosed herein is for administering to a subject to treat or prevent HIV infection.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific embodiment, a nucleic acid molecule(s) encoding an antibody or fusion polypeptide described herein is isolated or purified.

In particular aspects, provided herein are polynucleotides comprising nucleotide sequences encoding antibodies described herein, as well as antibodies that compete with such antibodies for binding to HIV, or which binds to the same epitope as that of such antibodies.

In certain aspects, provided herein are polynucleotides comprising a nucleotide sequence encoding the light chain or heavy chain of an antibody described herein. The polynucleotides can comprise nucleotide sequences encoding a light chain comprising the VL of antibodies described herein (see, e.g., Table 1). The polynucleotides can comprise nucleotide sequences encoding a heavy chain comprising the VH of antibodies described herein (see, e.g., Table 1). In specific embodiments, a polynucleotide described herein encodes a VH domain comprising the amino acid sequence set forth in SEQ ID NO: 3-9, 45, 47, 49-54, 65, 69, 70 or 112-126. In specific embodiments, a polynucleotide described herein encodes a VH domain comprising the amino acid sequence set forth in SEQ ID NO: 65. In specific embodiments, a polynucleotide described herein encodes a VH domain comprising the amino acid sequence set forth in SEQ ID NO: 8. In specific embodiments, a polynucleotide described herein encodes a VH domain comprising the amino acid sequence set forth in SEQ ID NO: 69. In specific embodiments, a polynucleotide described herein encodes a VH domain comprising the amino acid sequence set forth in SEQ ID NO: 70. In specific embodiments, a polynucleotide described herein encodes a VL domain comprising the amino acid sequence set forth in SEQ ID NO: 10, 11, 46, 48, 55, 66 or 127-139. In specific embodiments, a polynucleotide described herein encodes a VL domain comprising the amino acid sequence set forth in SEQ ID NO: 66. In one embodiment, the antibody is a chimeric antibody.

In particular embodiments, provided herein are polynucleotides comprising a nucleotide sequence encoding an antibody comprising three VL chain CDRs, e.g., containing VL CDR1, VL CDR2, and VL CDR3 of any one of antibodies described herein (e.g., see Table 2). In specific embodiments, provided herein are polynucleotides comprising three VH chain CDRs, e.g., containing VH CDR1, VH CDR2, and VH CDR3 of any one of antibodies described herein (e.g., see Table 2). In specific embodiments, provided herein are polynucleotides comprising a nucleotide sequence encoding an anti-Env antibody comprising three VL CDRs, e.g., containing VL CDR1, VL CDR2, and VL CDR3 of any one of antibodies described herein (e.g., see Table 2) and three VH chain CDRs, e.g., containing VH CDR1, VH CDR2, and VH CDR3 of any one of antibodies described herein (e.g., see Table 2).

In specific aspects, provided herein is a polynucleotide comprising a nucleotide sequence encoding an antibody comprising a light chain and a heavy chain, e.g., a separate light chain and heavy chain. With respect to the light chain, in a specific embodiment, a polynucleotide provided herein comprises a nucleotide sequence encoding a kappa light chain. In another specific embodiment, a polynucleotide provided herein comprises a nucleotide sequence encoding a lambda light chain. In yet another specific embodiment, a polynucleotide provided herein comprises a nucleotide sequence encoding an antibody described herein comprising a human kappa light chain or a human lambda light chain. In a particular embodiment, a polynucleotide provided herein comprises a nucleotide sequence encoding an antibody, which immunospecifically binds to Env, wherein the antibody comprises a light chain, and wherein the amino acid sequence of the VL domain can comprise the amino acid sequence set forth in Table 1, and wherein the constant region of the light chain comprises the amino acid sequence of a human kappa light chain constant region. In another particular embodiment, a polynucleotide provided herein comprises a nucleotide sequence encoding an antibody, which immunospecifically binds to Env, and comprises a light chain, wherein the amino acid sequence of the VL domain can comprise the amino acid sequence set forth in Table 1, and wherein the constant region of the light chain comprises the amino acid sequence of a human lambda light chain constant region. For example, human constant region sequences can be those described in U.S. Pat. No. 5,693,780.

In a particular embodiment, a polynucleotide provided herein comprises a nucleotide sequence encoding an antibody described herein, which immunospecifically binds to Env, wherein the antibody comprises a heavy chain, wherein the amino acid sequence of the VH domain can comprise the amino acid sequence set forth in Table 1, and wherein the constant region of the heavy chain comprises the amino acid sequence of a human alpha or gamma heavy chain constant region.

In yet another specific embodiment, a polynucleotide provided herein comprises a nucleotide sequence encoding an antibody described herein, which immunospecifically binds Env, wherein the antibody comprises a VL domain and a VH domain comprising any amino acid sequences described herein, and wherein the constant regions comprise the amino acid sequences of the constant regions of a human IgA₁, human IgA₂′ human IgG₁ (e.g., allotype 1, 17, or 3), human IgG₂, or human IgG₄.

In yet another specific embodiment, a polynucleotide provided herein comprises a nucleotide sequences encoding an anti-Env antibody or a fragment thereof that are optimized, e.g., by codon/RNA optimization, replacement with heterologous signal sequences, and elimination of mRNA instability elements. Methods to generate optimized nucleic acids encoding an anti-Env antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

In certain embodiments, an optimized polynucleotide sequence encoding an anti-Env antibody described herein or a fragment thereof (e.g., VL domain or VH domain) can hybridize to an antisense (e.g., complementary) polynucleotide of an unoptimized polynucleotide sequence encoding an anti-Env antibody described herein or a fragment thereof (e.g., VL domain or VH domain). In specific embodiments, an optimized nucleotide sequence encoding an anti-Env antibody described herein or a fragment hybridizes under high stringency conditions to antisense polynucleotide of an unoptimized polynucleotide sequence encoding an anti-Env antibody described herein or a fragment thereof. In a specific embodiment, an optimized nucleotide sequence encoding an anti-Env antibody described herein or a fragment thereof hybridizes under high stringency, intermediate or lower stringency hybridization conditions to an antisense polynucleotide of an unoptimized nucleotide sequence encoding an anti-Env antibody described herein or a fragment thereof. Information regarding hybridization conditions has been described, see, e.g., U.S. Patent Application Publication No. US 2005/0048549 (e.g., paragraphs 72-73), which is incorporated herein by reference.

The polynucleotides can be obtained, and the nucleotide sequence of the polynucleotides determined, by any method known in the art. Nucleotide sequences encoding antibodies described herein, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17: 242-246), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or fragment thereof described herein can be generated from nucleic acid from a suitable source (e.g., PBMCs) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, for example, to generate chimeric and humanized antibodies.

If a clone containing a nucleic acid encoding a particular antibody or fragment thereof is not available, but the sequence of the antibody molecule or fragment thereof is known, a nucleic acid encoding the immunoglobulin or fragment can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-Env antibodies described herein can be readily isolated and sequenced using conventional procedures (e.g., by using oligonucleotide probes that are capable of binding specifically to genes encoding the heavy and light chains of the anti-Env antibodies). PBMCs can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-Env antibodies in the recombinant host cells.

The DNA also can be modified, for example, by substituting the coding sequence for human heavy and light chain constant domains with a coding sequence for a non-immunoglobulin polypeptide, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3.

### IV. Vectors, Cells, and Methods of Producing a Broadly Neutralizing Agent

In certain aspects, provided herein are cells (e.g., host cells) expressing (e.g., recombinantly) antibodies described herein which specifically bind to Env and related polynucleotides and expression vectors. Provided herein are vectors (e.g., expression vectors) comprising polynucleotides comprising nucleotide sequences encoding anti-Env antibodies or a fragment thereof described herein. In one embodiment, the vectors can be used for recombinant expression of an antibody described herein in host cells (e.g., mammalian cells). In one embodiment, the vectors can be used for administration of an antibody described herein to a patient in need thereof. Also provided herein are host cells comprising such vectors for recombinantly expressing anti-Env antibodies described herein. In a particular aspect, provided herein are methods for producing an antibody described herein, comprising expressing such antibody in a host cell. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In certain aspects, provided herein is an isolated vector comprising a polynucleotide described herein. In one embodiment, the vector is a viral vector.

In certain aspects, provided herein is a recombinant virus comprising a polynucleotide described herein. In one embodiment, the recombinant virus encodes an antibody described herein. In one embodiment, the recombinant virus is an adeno-associated virus (AAV). In one embodiment, the recombinant virus is for administration to a subject to prevent or treat HIV infection. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In certain aspects, provided herein is a host cell comprising a polynucleotide described herein, or a vector described herein. In one embodiment, the vector encodes an antibody described herein. In one embodiment, a vector described herein comprises a first vector encoding a VH described herein and a second vector encoding a VL described herein. In one embodiment, a vector described herein comprises a first nucleotide sequence encoding a VH described herein and a second nucleotide sequence encoding a VL described herein. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one embodiment, the host cell is selected from the group consisting of E. coli, Pseudomonas, Bacillus, Streptomyces, yeast, CHO, YB/20, NS0, PER-C6, HEK-293T, NIH-3T3, Helga, BHK, Hep G2, SP2/0, R1.1, B-W, L-M, COS 1, COS 7, BSC1, BSC40, BMT10 cell, plant cell, insect cell, and human cell in tissue culture. In one embodiment, the host cell is CHO.

In certain aspects, provided herein is a method of producing an antibody that binds to HIV comprising culturing a host cell described herein so that the polynucleotide is expressed and the antibody is produced. In one embodiment, the method further comprises recovering the antibody.

The isolated polypeptides, i.e., anti-HIV Env antibodies described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., Proc. Nat'l. Acad. Sci. USA 81:5662-5066 (1984) and U.S. Pat. No. 4,588,585.

In some embodiments a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest will be inserted into an expression vector and operatively linked to an expression control sequence appropriate for expression of the protein in a desired host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies or fragments thereof. Recombinant expression vectors are replicable DNA constructs which have synthetic or cDNA-derived DNA fragments encoding a polypeptide chain of an antibody or fragment thereof operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, Helga, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein are CHO cells, for example CHO cells from the CHO GS System™ (Lonza). In a particular embodiment, cells for expressing antibodies described herein are human cells, e.g., human cell lines. In a specific embodiment, a mammalian expression vector is p0ptiVEC™ or pcDNA3.3. In a particular embodiment, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In certain embodiments, antibodies described herein are produced by CHO cells or NS0 cells. In a specific embodiment, the expression of nucleotide sequences encoding antibodies described herein which immunospecifically bind Env is regulated by a constitutive promoter, inducible promoter or tissue specific promoter.

For applications where it is desired that the antibodies described herein be expressed in vivo, for example in a subject in need of treatment with an antibody described herein, any vector that allows for the expression of the antibodies and is safe for use in vivo may be used. In one embodiment, the vector is a viral vector. Viral vectors can include poxvirus (vaccinia), including vaccinia Ankara and canarypox; adenoviruses, including adenovirus type 5 (Ad5); rubella; sendai virus; rhabdovirus; alphaviruses; and adeno-associated viruses. In one embodiment, the viral vector is an adeno-associated virus. Alternatively, a polynucleotide encoding the antibody could be delivered as DNA or RNA to the subject for in vivo expression of the antibody.

Suitable host cells for expression of a polypeptide of interest such as an antibody described herein include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems are also advantageously employed to express a recombinant protein such as an antibody described herein. Expression of recombinant proteins in mammalian cells can be performed because such proteins are generally correctly folded, appropriately modified and completely functional. Examples of suitable mammalian host cell lines include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein, e.g., an antibody, into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further an agent. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

In specific embodiments, an antibody described herein is isolated or purified. Generally, an isolated antibody is one that is substantially free of other antibodies with different antigenic specificities than the isolated antibody. For example, in a particular embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody. When the polypeptide (e.g., antibody described herein) is recombinantly produced, it is also generally substantially free of culture medium, i.e., culture medium represents less than about 20%, 10%, 2%, 1%, 0.5%, or 0.1% of the volume of the protein preparation. When the polypeptide (e.g., antibody described herein) is produced by chemical synthesis, it is generally substantially free of chemical precursors or other chemicals, i.e., it is separated from chemical precursors or other chemicals which are involved in the synthesis of the protein. Accordingly, such preparations of the polypeptide (e.g., antibody described herein) have less than about 30%, 20%, 10%, or 5% (by dry weight) of chemical precursors or compounds other than the polypeptide of interest. In one embodiment, antibodies described herein are isolated or purified.

### V. Pharmaceutical Compositions

Compositions comprising the antibodies or antigen-binding fragments described herein (e.g., ePGT121.15.H6.L1 antibody comprising the ePGT121.15.H6 VH and ePGT121.15.L1 VL) are also provided. Further provided herein are compositions comprising a polynucleotide or polynucleotides encoding the antibodies or antigen-binding fragments described herein. In one embodiment, the polynucleotide comprises mRNA. In one embodiment, the composition is a pharmaceutical composition. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one embodiment, the composition is a lyophilized composition. In one embodiment, the composition is formulated for topical administration, and in certain embodiments the composition is formulated for vaginal or rectal administration.

In certain aspects, provided herein is a pharmaceutical composition comprising an antibody described herein (e.g., ePGT121.15.H6.L1 antibody) and a pharmaceutically acceptable excipient. In one embodiment, the antibody is an intact antibody. In one embodiment, the antibody is an antigen binding antibody fragment. In one embodiment, the composition is formulated for topical administration, and in certain embodiments the composition is formulated for vaginal or rectal administration. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In another embodiment, the disclosure provides a pharmaceutical composition comprising an antibody described herein (e.g., ePGT121.15.H6.L1 antibody). Such compositions are intended for prevention and treatment of HIV infection. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In further embodiments of the present disclosure, a composition comprising the antibody described herein can additionally be combined with other compositions for the treatment of HIV infection or the prevention of HIV transmission.

In some embodiments, an antibody described herein may be administered within a pharmaceutically-acceptable diluent, carrier, or excipient, in unit dose form. Conventional pharmaceutical practice may be employed to provide suitable formulations or compositions to administer to individuals being treated for HIV infection. In one embodiment, the administration is prophylactic. Any appropriate route of administration may be employed, for example, administration may be parenteral, intravenous, intra-arterial, subcutaneous, intramuscular, intraperitoneal, intranasal, aerosol, suppository, oral administration, vaginal, or anal.

The pharmaceutical compositions described herein are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (21st ed.), ed. A. R. Gennaro, 2005, Lippincott Williams & Wilkins, Philadelphia, Pa., and Encyclopedia of Pharmaceutical Technology, eds. J. Swarbrick and J. C. Boylan, 2013, Marcel Dekker, New York, N.Y.).

The injection compositions are prepared in customary manner under sterile conditions; the same applies also to introducing the compositions into ampoules or vials and sealing the containers.

Pharmaceutical compositions according to the invention may be, for example, in unit dose form, such as in the form of ampoules, vials, suppositories, tablets, pills, or capsules. The formulations can be administered to human individuals in therapeutically or prophylactic effective amounts (e.g., amounts which prevent, eliminate, or reduce a pathological condition) to provide therapy for a disease or condition. The preferred dosage of therapeutic agent to be administered is likely to depend on such variables as the type and extent of the disorder, the overall health status of the particular patient, the formulation of the compound excipients, and its route of administration.

In certain embodiments, the compositions described herein can be formulated for topical administration, and in certain embodiments the composition is formulated for vaginal or rectal administration. The composition may be formulated as a gel, or formulated as a topical cream, ointment, lotion or foam formulation. Useful formulations are known in the art, for example, as disclosed in U.S. Patent Appl. Pub. No. 20130022619, which is incorporated by reference herein in its entirety for all purposes.

In certain embodiments, the composition may further comprise a pharmaceutically acceptable excipient, a lubricant, or an antiviral agent.

The topical formulations of the present invention can be used to prevent HIV infection in a human, or to inhibit transmission of the HIV virus from an infected human to another human. The topical formulations of the present invention can inhibit the growth or replication of HIV. The topical formulations are useful in the prophylactic treatment of humans who are at risk for HIV infection. The topical formulations also can be used to treat objects or materials, such as contraceptive devices (for example condoms or intrauterine devices), medical equipment, supplies, or fluids, including biological fluids, such as blood, blood products, and tissues, to prevent or inhibit viral infection of a human. Such topical formulations also are useful to prevent transmission, such as sexual transmission of viral infections, e.g., HIV, which is the primary way in which HIV is transmitted globally. The methods of prevention or inhibition or retardation of transmission of viral infection, e.g., HIV infection, in accordance with the present invention, comprise vaginal, rectal, penile or other topical treatment with an antiviral effective amount of a topical preparation of the present invention, alone or in combination with another antiviral compound as described herein.

In one embodiment the composition is in the form of a cream, lotion, gel, or foam that is applied to the affected skin or epithelial cavity, and preferably spread over the entire skin or epithelial surface which is at risk of contact with bodily fluids. Such formulations, which are suitable for vaginal or rectal administration, may be present as aqueous or oily suspensions, solutions or emulsions (liquid formulations) containing in addition to the active ingredient, such carriers as are known in the art to be appropriate. These formulations are useful to protect not only against sexual transmission of HIV, but also to prevent infection of a baby during passage through the birth canal. Thus the vaginal administration can take place prior to sexual intercourse, during sexual intercourse, and immediately prior to childbirth.

As a vaginal formulation, the active ingredient may be used in conjunction with a spermicide and may be employed with a condom, diaphragm, sponge or other contraceptive device. Examples of suitable spermicides include nonylphenoxypolyoxyethylene glycol (nonoxynol 9), benzethonium chloride, and chlorindanol. Suitably, the pH of the composition is 4.5 to 8.5. Vaginal compositions preferably have a pH of 4.5 to 6, most preferably about 5.

Vaginal formulations include suppositories (for example, gel-covered creams), tablets and films. The suppositories can be administered by insertion with an applicator using methods well known in the art.

Vaginal formulations further include vaginal ring devices formulated for sustained release. See, e.g., Morrow et al., Eur J Pharm Biopharm. 77(1):3-10 (2011), Zhao et al., Antimicrob Agents Chemother. 61(7) pii: e02465-16 (2017).

Buccal formulations include creams, ointments, gels, tablets or films that comprise ingredients that are safe when administered via the mouth cavity. Buccal formulations can also comprise a taste-masking or flavoring agent.

The present compositions may be associated with a contraceptive device or article, such as a vaginal ring device, an intrauterine device (IUD), vaginal diaphragm, vaginal sponge, pessary, condom, etc.

In one embodiment the compositions described herein are used in conjunction with condoms, to enhance the risk-reducing effectiveness of condoms and provide maximum protection for users. The composition can either be coated onto condoms during manufacture, and enclosed within conventional watertight plastic or foil packages that contain one condom per package, or it can be manually applied by a user to either the inside or the outside of a condom, immediately before use. As used herein, “condom” refers to a barrier device which is used to provide a watertight physical barrier between male and female genitalia during sexual intercourse, and which is removed after intercourse. This term includes conventional condoms that cover the penis; it also includes so-called “female condoms” which are inserted into the vaginal cavity prior to intercourse.

In another embodiment a composition described herein is in the form of an intra-vaginal pill, an intra-rectal pill, or a suppository. The suppository or pill should be inserted into the vaginal or rectal cavity in a manner that permits the suppository or pill, as it dissolves or erodes, to coat the vaginal or rectal walls with a prophylactic layer of an antibody described herein.

In certain embodiments, the composition may further comprise a pharmaceutically acceptable excipient, a lubricant, or an antiviral agent.

Compositions used in the methods of this invention may also comprise other active agents, such as another agent to prevent HIV infection, and agents that protect individuals from conception and other sexually transmitted diseases. Thus, in another embodiment the compositions used in this invention further comprise a second anti-HIV agent, a virucide effective against viral infections other than HIV, and/or a spermicide.

The compositions used in this invention may also contain a lubricant that facilitates application of the composition to the desired areas of skin and epithelial tissue, and reduces friction during sexual intercourse. In the case of a pill or suppository, the lubricant can be applied to the exterior of the dosage form to facilitate insertion.

In the cream or ointment embodiments of the present invention, the topical formulation comprises one or more lubricants. The gels and foams of the present invention optionally can include one or more lubricants.

Non-limiting examples of useful lubricants include cetyl esters wax, hydrogenated vegetable oil, magnesium stearate, methyl stearate, mineral oil, polyoxyethylene-polyoxypropylene copolymer, polyethylene glycol, polyvinyl alcohol, sodium lauryl sulfate, white wax, or mixtures of two or more of the above.

The gel formulations of the present invention comprise one or more gelling agents. Non-limiting examples of useful gelling agents include carboxylic acid polymers including acrylic acid polymers crosslinked with cross links such as allyl ethers of sucrose (e.g. carbomer brand thickeners), cetostearyl alcohol, hydroxymethyl cellulose, polyoxyethylene-polyoxypropylene copolymer, sodium carboxymethylcellulose, polyvinyl pyrrolidone, or mixtures of two or more thereof.

### VI. Uses and Methods

### Therapeutic Uses and Methods:

In one aspect, provided herein is a method of treating HIV or inhibiting transmission of HIV. In one embodiment, the method of inhibiting transmission of HIV comprises administering to a subject in need thereof an effective amount of an antibody described herein (e.g., ePGT121.15.H6.L1 antibody comprising the ePGT121.15.H6 VH and ePGT121.15.L1 VL), a pharmaceutical composition described herein, an isolated polynucleotide described herein, or a recombinant virus described herein. In one embodiment, the method of inhibiting transmission of HIV comprises administering to a subject in need thereof an effective amount of an antibody described herein (e.g., ePGT121.15.H6.L1 antibody). In one embodiment, the subject has been exposed to HIV. In one embodiment, the subject is at risk of being exposed to HIV. In one embodiment, the subject at risk of being exposed to HIV is a health care worker, a sexual partner of an HIV infected individual, or a sex worker. In one embodiment, the subject that has been exposed to HIV or is at risk of being exposed to HIV is a newborn. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one aspect, provided herein is a method of reducing the risk of a subject becoming infected with HIV comprising administering to the subject in need thereof an effective amount of an antibody described herein (e.g., ePGT121.15.H6.L1 antibody), a pharmaceutical composition described herein, an isolated polynucleotide described herein, or a recombinant virus described herein. In one embodiment, the subject is administered an antibody disclosed herein. In one embodiment, the subject has been exposed to HIV. In one embodiment, the subject is at risk of being exposed to HIV. In one embodiment, the subject at risk of being exposed to HIV is a health care worker, a sexual partner of an HIV infected individual, or a sex worker. In one embodiment, the subject that has been exposed to HIV or is at risk of being exposed to HIV is a newborn. In one aspect, provided herein is an antibody (e.g., ePGT121.15.H6.L1 antibody), a pharmaceutical composition, an isolated polynucleotide, or a recombinant virus for reducing the risk of a subject becoming infected with HIV. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one aspect, provided herein is a method for passively immunizing a subject comprising administering to the subject in need thereof an effective amount of an antibody described herein (e.g., ePGT121.15.H6.L1 antibody), a pharmaceutical composition described herein, an isolated polynucleotide described herein, or a recombinant virus described herein. In one embodiment, the subject is administered an antibody disclosed herein. In one embodiment, the subject has been exposed to HIV. In one embodiment, the subject is at risk of being exposed to HIV. In one embodiment, the subject at risk of being exposed to HIV is a health care worker, a sexual partner of an HIV infected individual, or a sex worker. In one embodiment, the subject that has been exposed to HIV or is at risk of being exposed to HIV is a newborn. In one aspect, provided herein is provided herein is an antibody (e.g., ePGT121.15.H6.L1 antibody), a pharmaceutical composition, an isolated polynucleotide, or a recombinant virus for passively immunizing a subject. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

Further provided herein is a method of neutralizing an HIV virus comprising contacting the virus with an effective amount of an antibody described herein (e.g., ePGT121.15.H6.L1 antibody). In one embodiment, the virus is comprised by a composition, for example, a fluid, including a biological fluid, such as blood or blood product. In certain embodiments, the method comprises adding an antibody described herein to a composition comprising HIV in a sufficient amount or concentration to neutralize the HIV. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

Further provided herein is a method of reducing viral load comprising administering to a subject in need thereof a therapeutically sufficient amount of an antibody described herein. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one embodiment of a method described herein, the antibody can be a chimeric antibody, engineered antibody, recombinant antibody, or a monoclonal antibody described herein. In one embodiment, the antibody is a full antibody, an F(ab) fragment, or an F(ab)2 fragment described herein. In a specific embodiment, the antibody is an engineered monoclonal antibody described herein. In a specific embodiment, the antibody is a recombinant monoclonal antibody described herein. In a specific embodiment, the antibody is a chimeric monoclonal antibody described herein. In a specific embodiment, the antibody is a F(ab) described herein. In a specific embodiment, the antibody is a F(ab′)2 fragment described herein.

In one embodiment, a method of preventing HIV infection provided herein comprises administering to a subject in need thereof a therapeutically sufficient amount of an antibody described herein (e.g., ePGT121.15.H6.L1 antibody), a pharmaceutical composition described herein, an isolated polynucleotide described herein, or a recombinant virus described herein. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one embodiment, a method of treating HIV/AIDS provided herein comprises administering to a subject in need thereof a therapeutically sufficient amount of an antibody described herein (e.g., ePGT121.15.H6.L1 antibody), a pharmaceutical composition described herein, an isolated polynucleotide described herein, or a recombinant virus described herein. In one embodiment, a method of treating HIV/AIDS comprises administering an antibody described herein. In one embodiment, a method of treating HIV/AIDS comprises administering a pharmaceutical composition described herein. In one embodiment, a method of treating HIV/AIDS comprises administering an isolated polynucleotide described herein. In one embodiment, a method of treating HIV/AIDS comprises administering a recombinant virus described herein. In one aspect, provided herein is an antibody, a pharmaceutical composition, an isolated polynucleotide, or a recombinant virus for treating HIV/AIDS. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one embodiment, the administering to the subject is by at least one mode selected from oral, parenteral, subcutaneous, intramuscular, intravenous, vaginal, rectal, buccal, sublingual, and transdermal

In one embodiment, a method of treatment described herein further comprises administering at least one additional therapeutic agent. In one embodiment, the additional therapeutic agent comprises an antiretroviral therapy (ART) agent, a reservoir activator, an immunomodulator, a second antibody, or a second and third antibody. In one embodiment, the additional therapeutic agent comprises a second antibody. In one embodiment, the additional therapeutic agent comprises a second and third antibody. In one embodiment, the additional therapeutic agent comprises a second and optionally third antibody which is an anti-HIV antibody. In one embodiment, the additional therapeutic agent comprises a second and optionally third antibody which is an anti-HIV Env antibody. In one embodiment, the additional therapeutic agent comprises a second and optionally third anti-HIV Env antibody which binds to an HIV Env epitope region different from the HIV Env epitope region bound by an antibody disclosed herein. In one embodiment, the additional therapeutic agent comprises a second and optionally third anti-HIV Env antibody which binds to the CD4 binding site (CD4bs) epitope region, V1N2-glycan site (V2g) epitope region, or gp41 MPER epitope region. In one embodiment, the additional therapeutic agent comprises a second anti-HIV Env antibody which binds to the CD4 binding site (CD4bs) epitope region. In one embodiment, the additional therapeutic agent comprises a second anti-HIV Env antibody which binds to the V1N2-glycan site (V2g) epitope region. In one embodiment, the additional therapeutic agent comprises a second anti-HIV Env antibody which binds to the gp41 MPER epitope region.

In certain embodiments, the subject is at risk for exposure to HIV. In some embodiments, the subject is infected with HIV. In some embodiments, the subject is diagnosed with AIDS. In certain embodiments, the subject at risk for exposure to HIV is a health care worker. In certain embodiments, the subject at risk for exposure to HIV is a sex worker. In certain embodiments, the subject at risk for exposure to HIV is a sexual partner of an HIV infected individual. In certain embodiments, the subject at risk for exposure to HIV is a newborn.

The invention also features methods of blocking HIV infection in a subject (e.g., a human) at risk of HIV transmission. For example, in one aspect, the subject may be a fetus of an HIV-infected pregnant female and the method includes administering to the HIV-infected pregnant female an antibody described herein (e.g., ePGT121.15.H6.L1 antibody), thereby blocking the HIV infection in the fetus. In other instances, the subject is a newborn having an HIV-infected mother, a subject at risk of HIV transmission following a needle stick injury, or a subject at risk of HIV transmission following a sexual exposure to an HIV-infected individual. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In instances when the subject is a newborn having an HIV-infected mother, the newborn can be administered an antibody described herein (e.g., ePGT121.15.H6.L1 antibody) peripartum and/or postpartum, for example, prior to, during, and/or following breastfeeding from the HIV-infected mother, in order to block an HIV infection in the newborn. In one embodiment, the antibody comprises the ePGT121.18.H1 VH and ePGT121.17.L1 VL.

In instances when the subject is at risk of HIV transmission following a sexual exposure to an HIV-infected individual, the subject can be administered an antibody described herein (e.g., ePGT121.15.H6.L1 antibody) following the sexual exposure in order to block an HIV infection in the subject. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In some embodiments, an antibody described herein can be used as a microbicides to prevent mucosal HIV acquisition. In some embodiments, an antibody described herein is used to prevent vaginal or rectal acquisition of HIV. In some embodiments, an antibody described herein can be used as a microbicides to reduce the likelihood of mucosal HIV acquisition. In some embodiments, an antibody described herein is used to reduce the likelihood of vaginal or rectal acquisition of HIV.

In any of the methods described above, further administration of ART and/or an immunomodulator and/or a second antibody is contemplated. For example, the AWL and/or immunomodulator and/or a second antibody can be administered in conjunction with, prior to, concurrently with, subsequent to, or within the context of a treatment regimen that includes administration of an antibody described herein.

An antibody described herein, or a pharmaceutical composition described herein can be delivered to a subject by a variety of routes, such as oral, parenteral, subcutaneous, intravenous, intradermal, transdermal, intranasal, vaginal, or anal. In one embodiment, the antibody or pharmaceutical composition is administered intravenously, vaginally, or anally.

The amount of an antibody described herein, or a pharmaceutical composition described herein which will be effective in the treatment and/or prevention of a condition will depend on the nature of the disease, and can be determined by standard clinical techniques.

The precise dose to be employed in a pharmaceutical composition will also depend on the route of administration, and the seriousness of the disease, and should be decided according to the judgment of the practitioner and each subject's circumstances. For example, effective doses may also vary depending upon means of administration, target site, physiological state of the patient (including age, body weight and health), whether the patient is human or an animal, other medications administered, or whether treatment is prophylactic or therapeutic. Usually, the patient is a human but non-human mammals including transgenic mammals can also be treated. Treatment dosages are optimally titrated to optimize safety and efficacy.

In certain embodiments, an in vitro assay is employed to help identify optimal dosage ranges. Effective doses may be extrapolated from dose response curves derived from in vitro or animal model test systems.

### Detection & Diagnostic Uses

An antibody described herein can be used to detect HIV and/or assay HIV levels in a biological sample using classical immunohistological methods known to those of skill in the art, including immunoassays, such as the enzyme linked immunosorbent assay (ELISA), immunoprecipitation, or Western blotting. An antibody described herein can also be used as an imaging agent, for example, a tissue-penetrating imaging agent. In one embodiment, an antibody described herein is conjugated with a detectable label. Suitable assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labels can be used to label an antibody or fusion polypeptide described herein. Alternatively, a second antibody that recognizes an antibody described herein can be labeled and used in combination with the antibody described herein to detect HIV levels.

As used herein, the term “biological sample” refers to any biological sample obtained from a subject, cell line, tissue, or other source potentially comprising HIV. Methods for obtaining tissue biopsies and body fluids from animals (e.g., humans) are well known in the art.

In another embodiment, an antibody described herein can be used to detect levels of HIV, which levels can then be linked to certain disease symptoms. An antibody described herein may carry a detectable or functional label. An antibody described herein can carry a fluorescence label. Exemplary fluorescence labels include, for example, reactive and conjugated probes, e.g., Aminocoumarin, Fluorescein and Texas red, Alexa Fluor dyes, Cy dyes and DyLight dyes. An antibody described herein can carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Te, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac and ¹⁸⁶Re. When radioactive labels are used, currently available counting procedures known in the art may be utilized to identify and quantitate the specific binding of an antibody described herein to HIV. In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an antibody described herein under conditions that allow for the formation of a complex between the antibody and HIV. Any complexes formed between the antibody and HIV are detected and compared in the sample and the control. An antibody described herein can also be used to purify HIV via immunoaffinity purification.

In some aspects, provided herein are methods for in vitro detecting HIV in a sample, comprising contacting said sample with an antibody described herein. In some aspects, provided herein is the use of an antibody described herein, for in vitro detecting HIV in a sample. In one aspect, provided herein is an antibody or pharmaceutical composition described herein for use in the detection of HIV in a subject. In one aspect, provided herein is an antibody or pharmaceutical composition described herein for use as a diagnostic. In one preferred embodiment, the antibody comprises a detectable label. In one embodiment, the subject is a human. In one embodiment, the method of detecting HIV in a sample comprises contacting the sample with an antibody described herein.

In some embodiments, the present disclosure provides methods of purifying HIV from a sample. In some embodiments, the method of purifying HIV from a sample comprises contacting the sample with an antibody described herein under conditions that allow the antibody to bind to HIV. In some embodiments, the antibody comprises a tag, for example, hexa-histidine tag or FLAG-tag to facilitate the purification of HIV.

### VII. Kits

Provided herein are kits comprising one or more antibodies described herein or one or more fusion polypeptides (e.g., ePGT121.15.H6.L1 antibody comprising the ePGT121.15.H6 VH and ePGT121.15.L1 VL) described herein. In some embodiments, a pharmaceutical pack or kit described herein comprises one or more containers filled with one or more of the ingredients of the pharmaceutical compositions described herein, such as one or more antibodies described herein. In some embodiments, a kit contains an antibody described herein or a pharmaceutical composition described herein, and a second prophylactic or therapeutic agent used in the treatment or prevention of HIV. In one embodiment, the second agent is an antiretroviral agent. In one embodiment, the second agent is a reservoir activator. In one embodiment, the second agent is an immunomodulator. In one embodiment, the second agent is one or more anti-HIV antibody. In one embodiment, the second agent is one or more anti-HIV Env antibody that binds to an HIV Env epitope region different from the HIV Env epitope region bound by an antibody disclosed herein. In one embodiment, the second agent is one or more anti-HIV Env antibody that binds to the CD4 binding site (CD4bs) epitope region, V1N2-glycan site (V2g) epitope region, or gp41 MPER epitope region. In some embodiments, a kit contains an antibody described herein or a pharmaceutical composition described herein, and a reagent used in the detection of HIV. In one embodiment, the detection reagent comprises DNA primers for the detection of HIV. Optionally associated with such container(s) can be a notice in the form prescribed by a governmental agency regulating the manufacture, use or sale of pharmaceuticals or biological products, which notice reflects approval by the agency of manufacture, use or sale for human administration. In one embodiment, the antibody comprises the ePGT121.15.H6 VH and ePGT121.15.L1 VL. In one embodiment, the antibody comprises the ePGT121.15.H6 VH and ePGT121.17.L1 VL. In one embodiment, the antibody comprises the ePGT121.18.H1 VH and ePGT121.17.L1 VL. In one embodiment, the antibody comprises the ePGT121.18.H17 VH and ePGT121.17.L1 VL. In one embodiment, the antibody comprises the ePGT121.18.H18 VH and ePGT121.17.L1 VL.

In one embodiment, a kit described herein comprises an antibody described herein or a pharmaceutical composition described herein and a) a detection reagent, b) an HIV antigen, c) a notice that reflects approval for use or sale for human administration, or d) any combination thereof.

In one embodiment, a kit described herein comprises a fusion polypeptide described herein or a pharmaceutical composition described herein and a) a detection reagent, b) an HIV antigen, c) a notice that reflects approval for use or sale for human administration, or d) any combination thereof.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

All documents, patent, and patent applications cited herein are hereby incorporated by reference, and may be employed in the practice described herein.

### EXAMPLES

### Example 1. pH Dependence of Gp120 Binding by Broadly Neutralizing Anti-HIV Antibodies

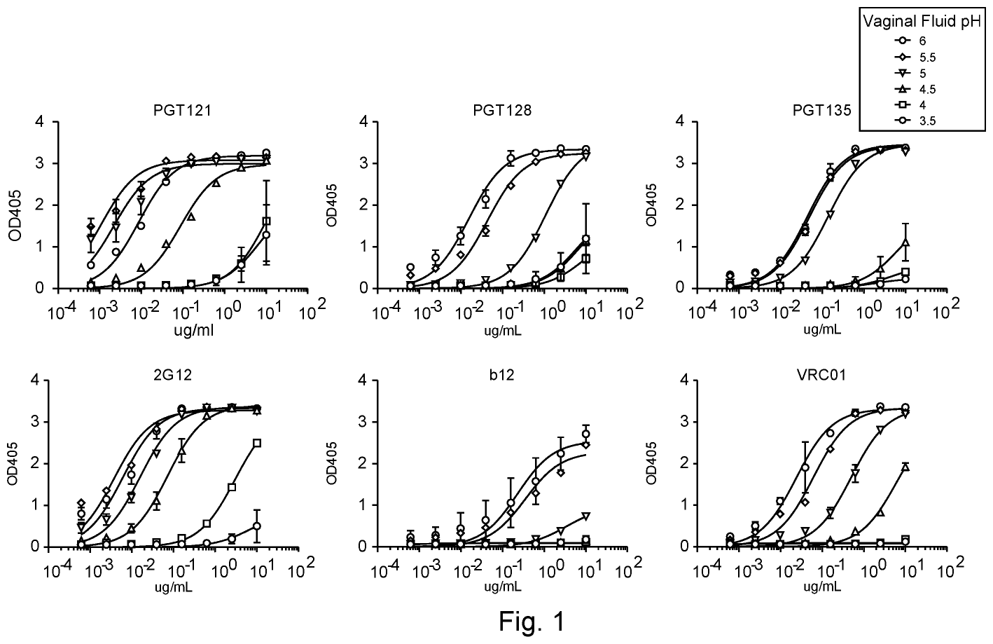

The overall objective of this project was to develop an enhanced engineered variant broadly neutralizing anti-HIV antibody (bnAb) that is suitable, among other uses, for targeted vaginal delivery. The unique microenvironment of the vagina might provide challenges for bnAbs to effectively neutralize HIV at the site of infection, specifically the lower pH in the vagina could hinder antibody binding to HIV Env. To test this, the binding affinities of the PGT-121, PGT128, PGT135, 2G12, b12, and VRC01 bnAbs to gp120 (isolate: 92BR020) was measured at various pH levels to determine which bnAb had the highest binding affinity at low pH (FIG. 1). PGT-121 was identified as the antibody that retains the highest binding affinity to HIV gp120 at low pH.

Affinity measurements at different pHs were performed substantially as described in Sok, D. et al., Nature 548(7665):108-111 (2017). Briefly, ELISA plates were first coated with an anti-05 gp120 antibody at 4° C. in 1×PBS overnight. Plates were then washed 5× with PBS+0.05% tween and blocked with 3% BSA in 1×PBS at room temperature for 1 hr. Mutant pseudovirus supernatants were lysed with 1% NP40 and then captured on ELISA plates at 37° C. for 2 hr. Plates were washed 5× with PBS+0.05% tween and then serial dilutions of mAbs were incubated in PBS at different pHs or simulated vaginal fluid (SVF) for 1 hr at RT and then washed 5× with PBS+0.05% tween before addition of goat anti-human IgG F(ab′)2 conjugated to alkaline phosphatase secondary. The plates were incubated at room temperature for 1 h and washed 5× with PBS+0.05% tween. Plates were then developed by adding 50 microL of alkaline phosphatase substrate (Sigma) dissolved in alkaline phosphatase staining buffer (pH 9.8), according to the manufacturer's instructions. The optical density at 405 nm was read on a microplate reader (Molecular Devices). SVF was made with citric acid instead of lactic acid to improve buffering at higher pH levels.

### Example 2. General Design Strategy to Generate Enhanced Engineered Variant Broadly Neutralizing Anti-HIV Antibodies

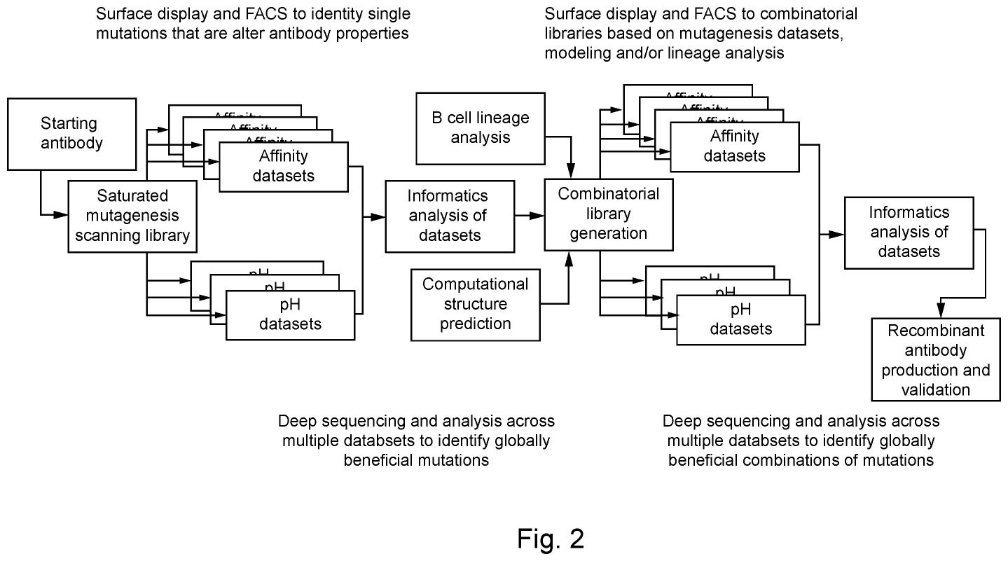

The primary objective in the design of enhanced bnAbs was to increase neutralization potency. The general design strategy combined directed evolution (yeast surface display, FACS and deep sequencing) with informatics analysis and computational modeling. J. Jardine et al., Rational HIV immunogen design to target specific germline B cell receptors. Science 340, 711-716 (2013); J. G. Jardine et al., HIV-1 broadly neutralizing antibody precursor B cells revealed by germline-targeting immunogen. Science 351, 1458-1463 (2016). The process was broken down into two overall steps—identification of single beneficial mutations followed by identification of combinations of beneficial mutations (FIG. 2). In the first step, yeast surface display, FACS and deep sequencing was used to analyze the effect all possible single mutations in the antibody for binding to multiple Env variants in physiological conditions (PBS) and low pH conditions (synthetic vaginal fluid simulant). Mutations from this scanning dataset were combined with structural prediction and other informatics analysis, which was then used to make a combinatorial library containing ˜10⁷ bnAb variants containing multiple mutations that were shown experimentally or predicted computationally to improve bnAb functionality. In the second step, this combinatorial library was displayed on yeast and again screened for improved binding to multiple Env variants in both physiological conditions and low pH conditions. Following several rounds of enrichment, the selected clones were deep sequenced and analyzed to identify enhanced engineered variant bnAbs. Then the bnAb variants were recombinantly produced as IgG for characterization.

To account for the diversity in HIV, the combinatorial library was selected for improved affinity against multiple gp120 isolates deriving from clades CRF02_AG, A, and C, which predominantly affect adolescent girls and young women in sub-Saharan Africa. Separate deep sequencing was performed on the combinatorial antibody library enriched on individual isolates to produce multiple datasets. The deep sequencing datasets were informatically analyzed to identify mutations that enriched across multiple datasets, rather than in a small subset of datasets. Enrichment across multiple datasets indicated that the mutations were broadly beneficial mutations, whereas enrichment in a small subset of datasets indicated that the mutations would only yield strain-specific improvements. Accordingly, the enhanced engineered antibodies identified were optimized against a panel of diverse Env variants.

Lastly, the sequence of enhanced engineered antibody candidates were analyzed for known development liabilities (post-translational modification, oxidation sites, deamination sites, etc.) that could be easily substituted for other mutations based on the mutational datasets.

Identification of single beneficial mutations: A strategy recently developed for protein/protein interface design known as barcode-enhanced saturated mutagenesis scanning was used to identify beneficial point mutations that improve PGT-121 antibody function. See, WO/2016/205704, incorporated by reference herein in its entirety. Much like alanine scanning, point mutations were made throughout the entire PGT-121 VH and VL region sequences. However, rather than just mutating to alanine, mini-libraries that sampled all 20 possible amino acids by using the degenerate codon “NNK” were created. The NNK position was flanked by a “barcode” by introducing a silent nucleotide change on either side to aid in downstream informatics analysis. These libraries were displayed on the surface of yeast and were split into many fractions, each of which was optimized against a single condition, i.e., binding to a gp120 polypeptide from a single HIV isolate. The recovered clones were then sequenced and the results were informatically binned by barcode position and enrichment values were calculated based on an unselected control dataset. A comprehensive “fingerprint” was created for each mutation, with experimental data on whether a given mutation was beneficial, detrimental or had no effect on the condition being screened, i.e., binding to the gp120 from an HIV isolate. A mutation was considered beneficial for binding to the gp120 from the HIV isolate if it was enriched, i.e., had a higher frequency among the clones that were selected on the gp120 from the HIV isolate compared to its frequency in the unselected control dataset. A mutation was considered detrimental for binding to the gp120 from the HIV isolate if it was negatively enriched or depleted, i.e., had a lower frequency among the clones that were selected on the gp120 from the HIV isolate compared to its frequency in the unselected control dataset. And a mutation was considered to have no effect for binding to the gp120 from the HIV isolate if it had the same frequency among the clones that were selected on the HIV isolate as in the unselected control dataset.

Identification of beneficial combinations of mutations: The beneficial mutation dataset generated as described above was combined with structural prediction and other informatics analysis to make a combinatorial library containing ˜10⁷ PGT-121 variants containing multiple mutations that were shown experimentally or predicted computationally to improve PGT-121 functionality. When designing the combinatorial library, the original amino acid residue was always included at a position sampled so that one or two fixed mutations could not completely disrupt all the clones in the population. Analysis from previous work and from preliminary work with PGT-121 suggested that not all individually beneficial mutations could function synergistically, and typically less than half of the mutations were selected in the final sequence. The libraries were screened for binding to diverse Env variants. The combinatorial library was split into many fractions, each of which was optimized against a single condition, i.e., binding to a single HIV isolate. The selected clones from each pool were deep sequenced to generate multiple affinity datasets. The deep sequence datasets were analyzed to identify enhanced engineered variant PGT-121 antibodies. The enhanced engineered variant PGT-121 antibodies were then reformatted and expressed as IgG for neutralization and biophysical characterization.

### Example 3. Generation of Enhanced Engineered Variant Broadly Neutralizing Anti-HIV Antibodies

Generation of enhanced engineered VH domain: The enhanced engineered ePGT121.12.H1.L4 antibody comprises the ePGT121.12.H1 VH (SEQ ID NO: 45), which is identical to the parent PGT121 VH (SEQ ID NO: 1), and the ePGT121.12.L4 VL (SEQ ID NO: 46). ePGT121.12.H1.L4 was generated by pairing the original PGT121 heavy chain (“H1”) with light chain variants. The light chain variants were generated using combinatorial libraries that were designed by shuffling the sequence variation present in the PGT-121, PGT-122, PGT-123, PGT-124, PGT-133, and PGT-134 antibodies, each of which has been disclosed in U.S. Pat. No. 9,464,131, which is hereby incorporated by reference herein in its entirety. The combinatorial library was displayed as single chain variable fragments (scFVs comprising the light chain variable region, a Gly-Ser linker, and heavy chain variable region) on the surface of yeast. Samples of the library were screened for binding to 92BR020 and 94UG103, both in phosphate buffered saline and simulated vaginal fluid. The engineered ePGT121.L4 VL sequence (FIG. 3A) has been selected for further use. Pseudovirus neutralization results generated with the ePGT121.12.H1.L4 antibody against a 12-member virus panel are shown in FIG. 3B. pH dependence of ePGT121.12.H1.L4 binding affinities to 94UG103 and 92BR020 gp120 are shown in FIG. 3C. ePGT121.12.H1.L4 has improved cross-clade virus neutralization properties, with regards to both breadth and potency, than the PGT-121 parent. ePGT121.12.H1.L4 also possesses higher binding affinity to 94UG103 and 92BR020 gp120 than PGT-121 at reduced pH.

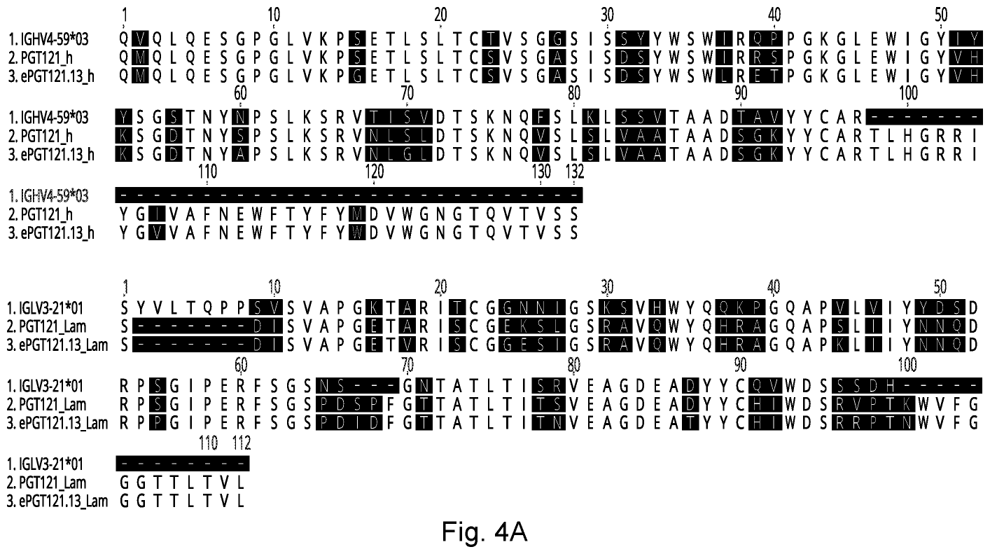

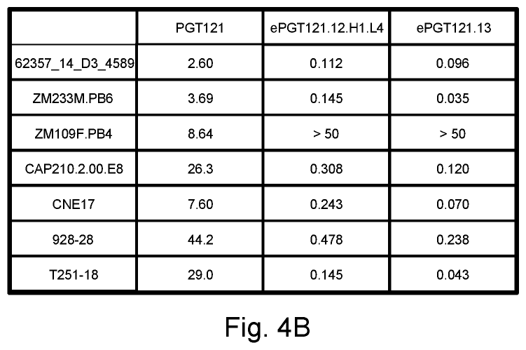

The enhanced engineered ePGT121.13.H1.L1 antibody comprises the ePGT121.13.H1 VH (SEQ ID NO: 47) and ePGT121.13.L1 VH (SEQ ID NO: 48). FIG. 4A. ePGT121.13.H1.L1 was generated by further engineering ePGT121.12.H1.L4 using double barcoded saturated mutagenesis scanning. NNK libraries, where every amino acid is sampled at each residue of the variable gene (NNK scanning), were generated for heavy and light chain variable genes. Heavy chain libraries were displayed on the surface of yeast in combination with the ePGT121.12.L4 VL and selected for high-affinity binding to gp120 protein derived from HIV isolates 92BR020, A97, B04 and JRFL gp120 in PBS and SVF. Combinations of these enriched mutations were then used to create a second NNK library, which was then selected for high-affinity to 92BR020, A97, B04 and JRFL gp120 in PBS and SVF. Light chain library generation and screening was performed similarly. NGS sequencing and data processing was done as described above. ePGT121.13.H1.L1 was selected for further engineering. Pseudovirus neutralization results generated with the ePGT121.13.H1.L1 antibody against a 7-member virus panel are shown in FIG. 4B. ePGT121.13.H1.L1 has improved neutralization properties, with regards to both breadth and potency, compared to PGT-121 or ePGT.121.12.H1.L4.

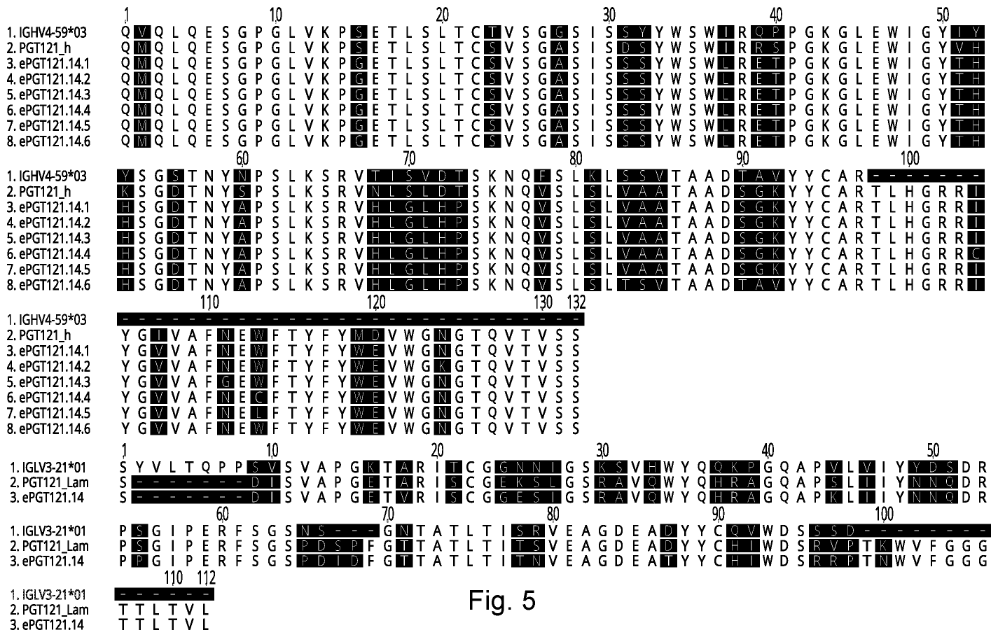

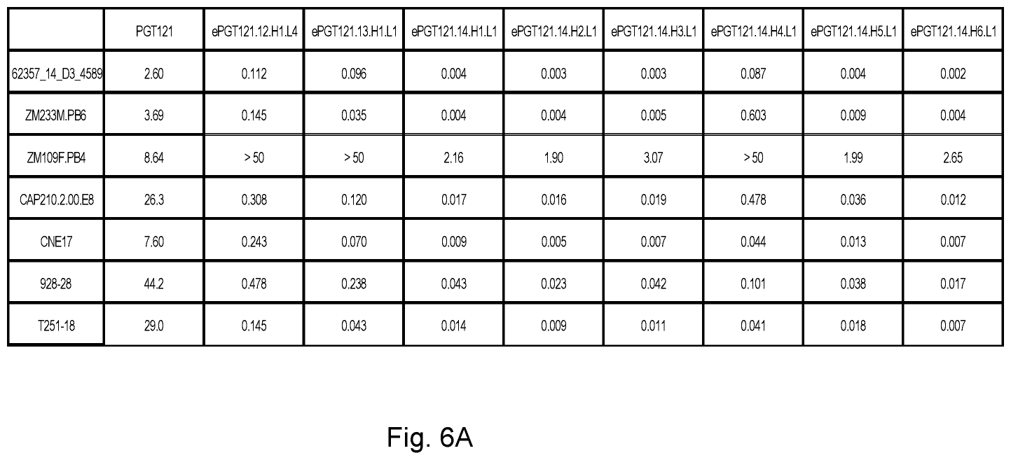

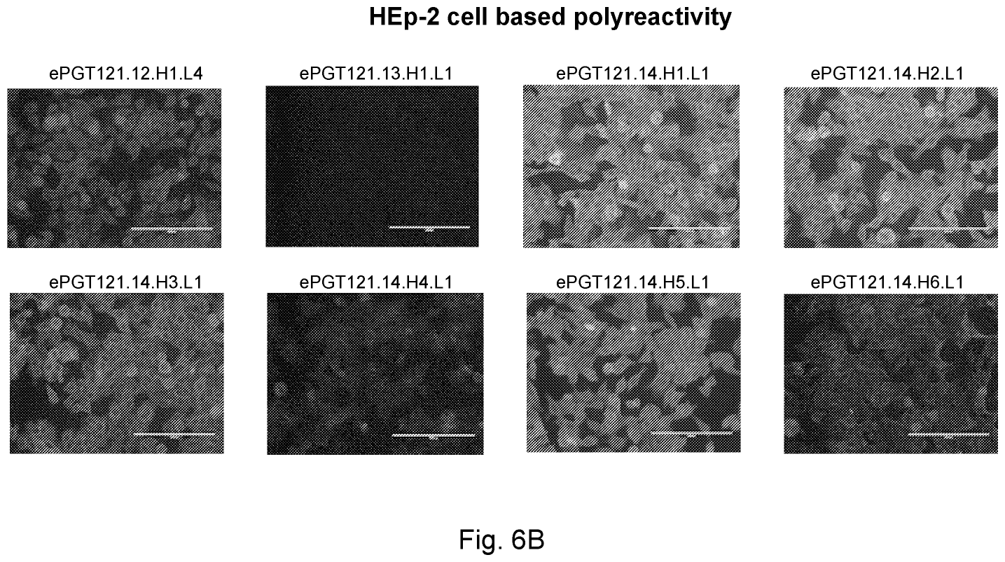

The ePGT121.14 antibody variants were generated using the ePGT121.13. H1 VH and ePGT121.13.L1 VL as a base constructs. FIG. 5. To select for single mutations that enhanced affinity, a Fab display library comprising NNK scan variant of ePGT121.13.H1 VH was displayed on yeast in combination with ePGT.13.L1. Antibody variants were selected for binding to 92BR010, A97, B04, JR-FL gp120 in PBS and simulated vaginal fluid. Combinatorial NNK libraries were then prepared of the heavy chain variable region constructs, displayed on the surface as Fab, and screened for binding to 16055 gp120, 92BR020 gp120, 92RW020 gp120, 92TH021 gp120, 93IN905 gp120, A97 gp120, CN54 gp120, DU179 gp120, IAVI C22 gp120, JR-FL gp120, JR-FL NFL trimer, BG505 SOSIP trimer. Selection generated the ePGT121.14.H1.L1 improved variant antibody comprising the ePGT121.14.H1 VH (SEQ ID NO: 49) and ePGT.121.14.L1 VL (SEQ ID NO: 55), which is identical to ePGT121.13.L1 VH (SEQ ID NO: 48). Pseudovirus neutralization results generated with the ePGT121.14.H1.L1 antibody against the 7-member virus panel are shown in FIG. 6A. ePGT121.13.H1.L1 has improved neutralization properties, with regards to both breadth and potency, compared to PGT-121, ePGT.121.12.H1.L4, and ePGT121.13.H1.L1. Polyreactivity of ePGT121.14.H1.L1 was tested using the HEp-2 cell based assay. FIG. 6B. Unlike PGT-121, ePGT.121.12.H1.L4, and ePGT121.13.H1.L1, the ePGT121.14.H1.L1 antibody was found to be polyreactive.

Several variants of the ePGT.121.14.H1 VH were generated to eliminate potential sequence liabilities that may pose problems for the development of the enhanced engineered PGT-121 antibodies as a pharmaceutical agent. ePGT.121.14.H2 VH (SEQ ID NO: 50) comprises the N to K substitution at residue 124 to eliminate a potential glycosylation site and thus improve homogeneity during manufacturing. ePGT.121.14.H3 VH (SEQ ID NO: 51) comprises the N to G substitution at residue 111 in VH CDR3 to eliminate the potential for asparagine cyclization that may lead to degradation. ePGT.121.14.H4 VH (SEQ ID NO: 52) comprises the I to C and W to C substitutions at residues 104 and 113, respectively, in the VH CDR3 to introduce a disulphide across VH CDR3 that would both remove the potential for tryptophan oxidation as well as potentially stabilize the CDR-H3 loop in a conformation favorable for binding. ePGT.121.14.H5 VH (SEQ ID NO: 53) comprises the W to L substitution at residue 113 in the VH CDR3 to eliminate the potential for tryptophan oxidation. ePGT.121.14.H6 VH (SEQ ID NO: 54) comprises the VAA to TSV and SGK to TAV substitutions at residues 83-85 and 90-92, respectively, to revert residues in framework 3 to germline sequences and eliminate polyreactivity. Antibodies comprising the ePGT.121.14.H1, ePGT.121.14.H2, ePGT.121.14.H3, ePGT.121.14.H4, ePGT.121.14.H5, and ePGT.121.14.H6 VH in combination with the ePGT121.14.L1 VL were generated and tested for pseudovirus neutralization against the 7-member virus panel and for polyreactivity using the HEp-2 cell based assay. FIGS. 6A and 6B. All variants tested displayed improved neutralization breadth and potentcy compared to PGT-121. The ePGT121.14.H2.L1, ePGT121.14.H3.L1, ePGT121.14.H5.L1, and ePGT121.14.H6.L1 variants maintained the superior neutralization properties of the ePGT121.14.H1.L1 antibody. Additionally, ePGT121.14.H4.L1 and ePGT121.14.H6.L1 did not possess the polyreactivity observed with ePGT121.14.H1.L1. Thus, the VAA to TSV and SGK to TAV substitutions at residues 83-85 and 90-92, respectively, of SEQ ID NO: 54 eliminated polyreactivity without significant effect on the breadth or potency of neutralization. Similarly, the I to C and W to C substitutions at residues 104 and 113, respectively, of SEQ ID NO: 52 eliminated polyreactivity with a modest effect on neutralization breadth and potency, being more potent than unmodified PGT121, but less potent than ePGT121.14.H6.L1.

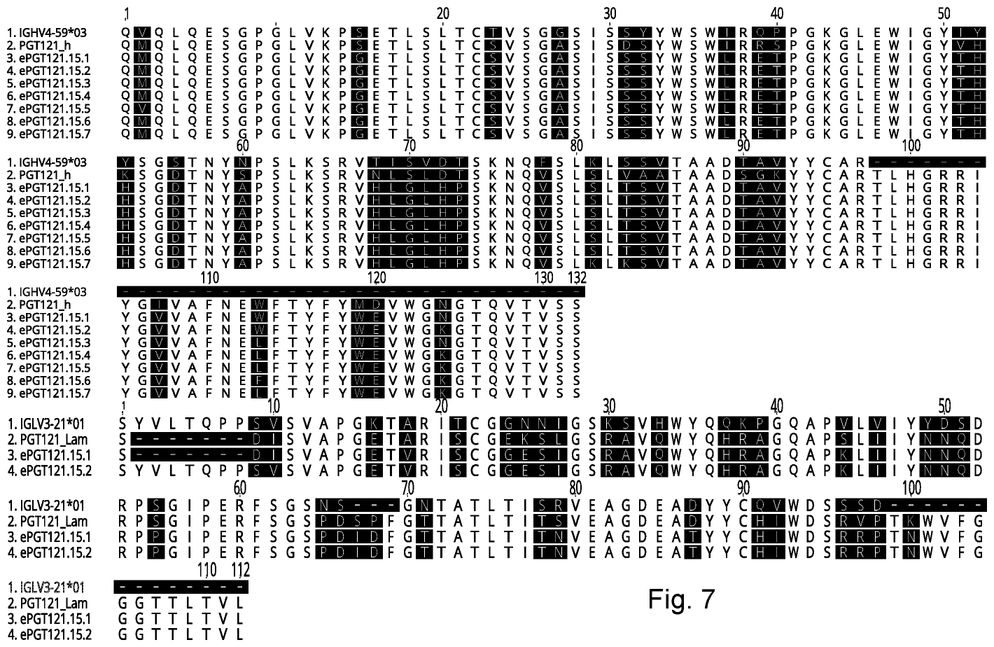

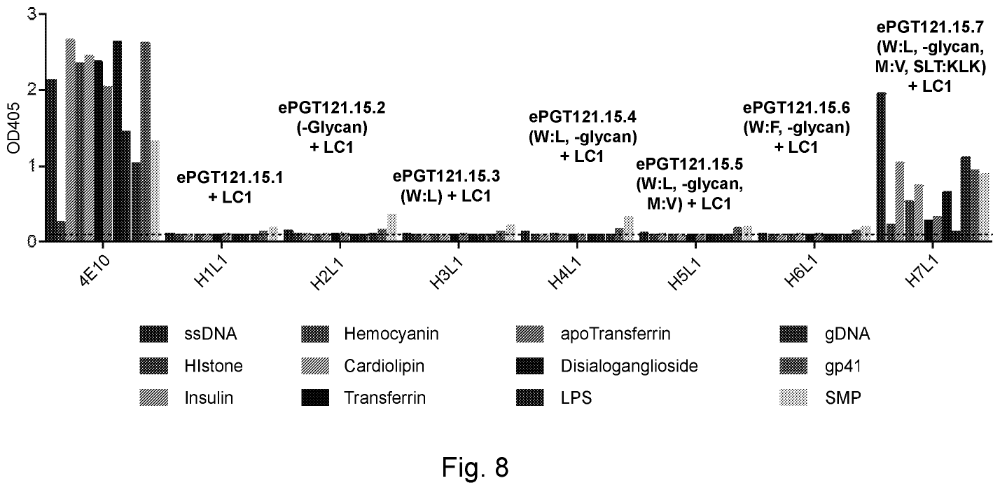

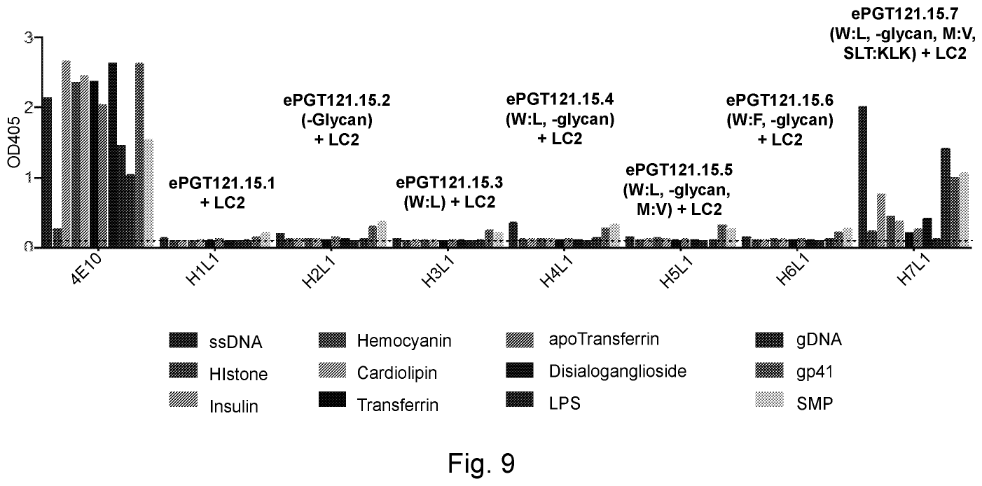

Additional variants were ordered based on ePGT121.14.H6 to remove additional liabilities. A second light chain variant was also tested to restore the N-term deletion. The heavy chain and light chain variable sequences tested are shown in FIG. 7. The ePGT121.15.H1 VH (SEQ ID NO: 3) has the same amino acid sequence as ePGT121.14.H6, and therefore it includes the TSV and TAV amino acid sequences at residues 83-85 and 90-92. ePGT121.15.H2 VH (SEQ ID NO: 4) additionally comprises the N to K substitution at residue 124 to eliminate a potential glycosylation site and thus improve homogeneity during manufacturing. ePGT121.15.H3 VH (SEQ ID NO: 5) additionally comprises the W to L substitution at residue 113 in the VH CDR3 to eliminate the potential for tryptophan oxidation. ePGT121.15.H4 VH (SEQ ID NO: 6) additionally comprises both (i) the W to L substitution at residue 113 in the VH CDR3, and (ii) the N to K substitution at residue 124. ePGT121.15.H5 VH (SEQ ID NO: 7) additionally comprises (i) the M to V substitution at residue 2 to improve homogeneity by eliminating the potential for oxidation, (ii) the W to L substitution at residue 113 in the VH CDR3, and (iii) the N to K substitution at residue 124. ePGT121.15.H6 VH (SEQ ID NO: 8) additionally comprises (i) the W to F substitution at residue 113 in the VH CDR3 to improve homogeneity by eliminating the potential for tryptophan oxidation, and (ii) the N to K substitution at residue 124. ePGT121.15.H7 VH (SEQ ID NO: 9) comprises (i) the KLKSV amino acid sequence at residues 81-85, the TAV amino acid sequence at residues 90-92, (iii) the W to F substitution at residue 113 in the VH CDR3, and (iv) the N to K substitution at residue 124. The ePGT121.15.L1 VL (SEQ ID NO: 10) has the same amino acid sequence as ePGT121.14.L1. ePGT121.15.L1 VL (SEQ ID NO: 11) comprises an addition of seven N-terminal amino acid residues encoded by the corresponding germline sequence to improve developability. Recombinant antibodies comprising the ePGT121.15.H1, ePGT121.15.H2, ePGT121.15.H3, ePGT121.15.H4, ePGT121.15.H5, or ePGT121.15.H6 VH in combination with either the ePGT121.15.L1 or ePGT121.15.L2 VL did not display polyreactivity in a reactivity assay that includes human autoantigens and a solubilized membrane preparation (SMP) from extracted from CHO cells. FIGS. 8 and 9.

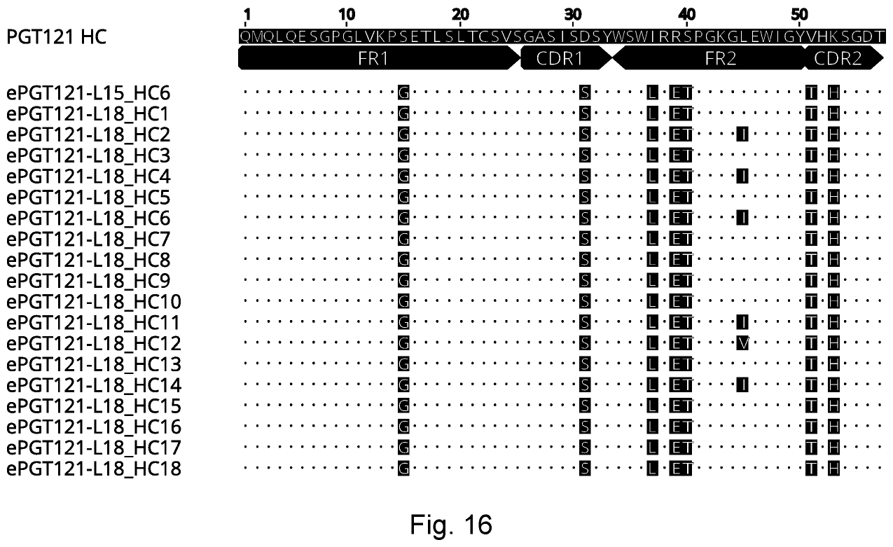

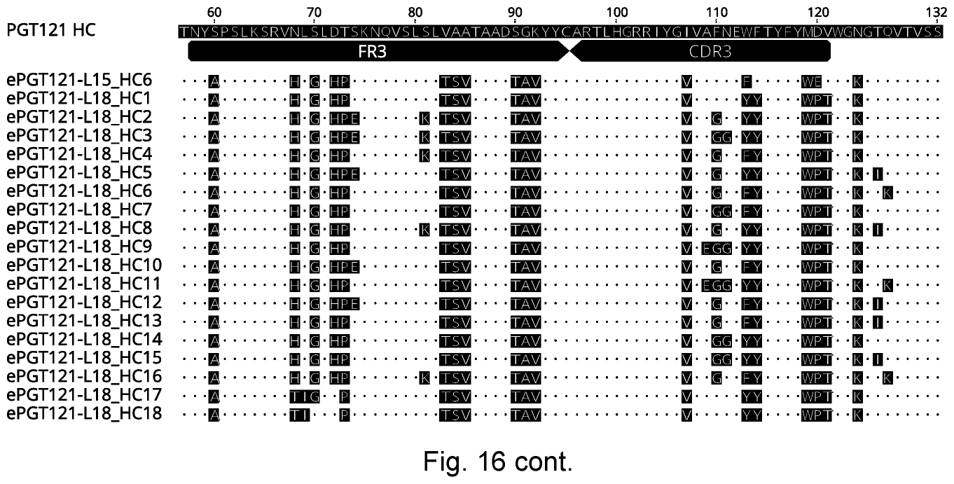

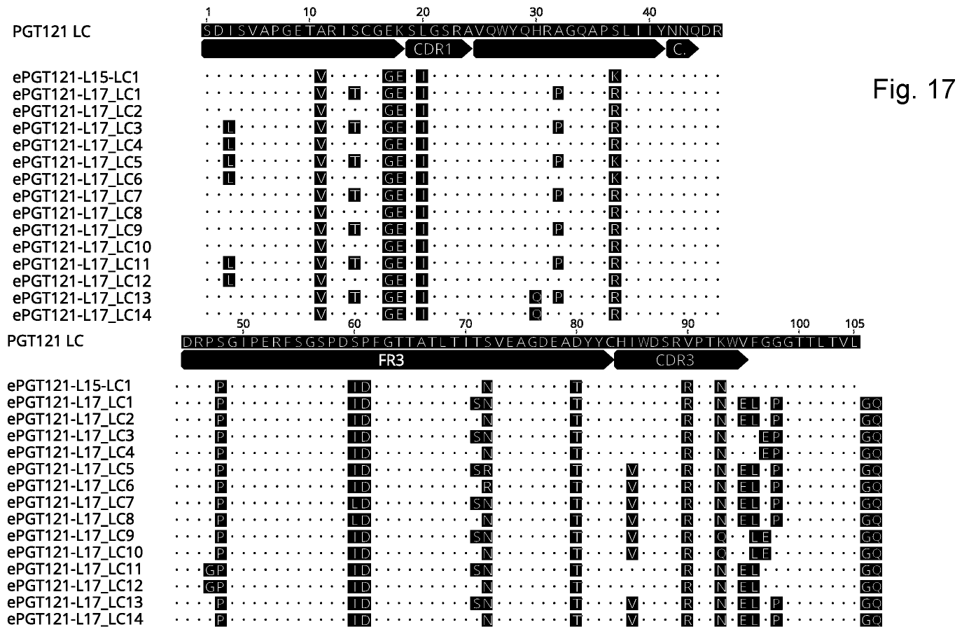

The amino acid sequence of additional ePGT121 variant VH and VL domains tested are shown in FIGS. 16 and 17.

Antibody production and purification: Antibody plasmids containing heavy chain and light chain genes were co-transfected (1:2.5 ratio) in either HEK 293T, 293F or expi293 cells using X-tremeGENE (Roche) or 293fectin (Invitrogen) as transfection reagents. Antibody containing supernatants were harvested 7 days after transfection and 0.22 μm sterile filtered. Antibodies produced in 293T cells were quantified by anti-Fc ELISA and used directly in neutralization assays for screening purposes. Antibody supernatants produced in 293F cells were purified over Protein A Sepharose 4 Fast Flow (GE healthcare) columns as described previously. Sok, D. et al., Proc. Natl. Acad. Sci. U.S.A. 111, 17624-17629 (2014)

HEp-2 cell staining assay: The HEp-2 cell-staining assay was performed using kits purchased from Aesku Diagnostics (Oakland, Calif.) according to manufacturer's instructions. These Aesku slides use optimally fixed human epithelial (HEp-2) cells (ATCC) as substrate and affinity purified, FITC-conjugated goat anti-human IgG for the detection. Briefly, 2.5 μg or 25 μl of 100 μg/ml mAb and controls were added to wells and incubated on HEp-2 slides in a moist chamber at room temperature for 30 min. Slides were then rinsed and submerged in PBS and 25 μl of FITC-conjugated goat anti-human IgG was immediately applied to each well. Slides were allowed to incubate at room temperature in a moist chamber for another 30 min. Slides were then washed in the same manner as above and then mounted on coverslips using the provided mounting medium. Slides were viewed at 20× magnification and photographed on an EVOS f1 fluorescence microscope at a 250 ms exposure with 100% intensity. Positive and negative control sera were provided by the vendor. Samples showing fluorescence greater than the negative control were considered positive for HEp-2 staining.

Polyspecificity reagent (PSR) binding assay: Monoclonal antibodies were screened for reactivity with preparations of solubilized membrane proteins (SMP) substantially as described in Jardine et al., PLOS Pathogens at htps://doi.org/10.1371/journal.ppat. 1005815. Briefly, SMP was extracted from CHO cells (ATCC). The protein concentration was determined using the Dc-protein assay kit (BioRad). SMP was then immobilized on ELISA plates for mAb screening. The results were established by reading the absorbance at 450 nm of the examined samples.

Single autoantigen reactivity: Single antigen ELISA assays for ssDNA, histone, insulin, hemocyanin, cardiolipin, transferrin, apotransferrin, disialoganglioside, LPS, gDNA, gp41 was performed using standard ELISA protocols. 96 wells were separately coated with the cellular and nuclear antigens for the qualitative detection of mAbs reactivity.

### Example 4. The Engineered Variant PGT-121 Antibodies have Enhanced Neutralization Breadth and Potency

The breadth and potency of the enhanced engineered PGT-121 variant antibodies' neutralization activity was determined using standard assays. Briefly, plasmids encoding various HIV Env isolates were co-transfected into HEK 293T cells (ATCC) with pSG3ΔEnv, an Env-deficient genomic backbone plasmid, in a 1:2 ratio using X-tremeGENE HP (Roche) as transfection reagent. Cell culture supernatants were harvested 3 days post transfection and sterile filtered through a 0.22 μm filter. Neutralizing activity was measured by incubating monoclonal antibodies or sera with replication incompetent pseudovirus for 1h at 37C before transferring onto TZM-bl target cells (aidsreagent.org) as described previously. Walker, L. M. et al., Nature 477, 466-470 (2011).

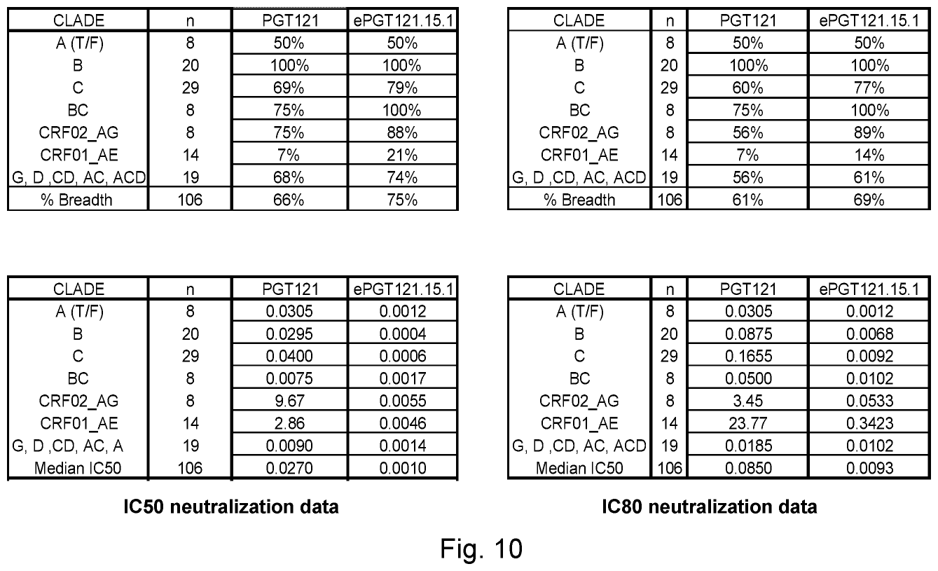

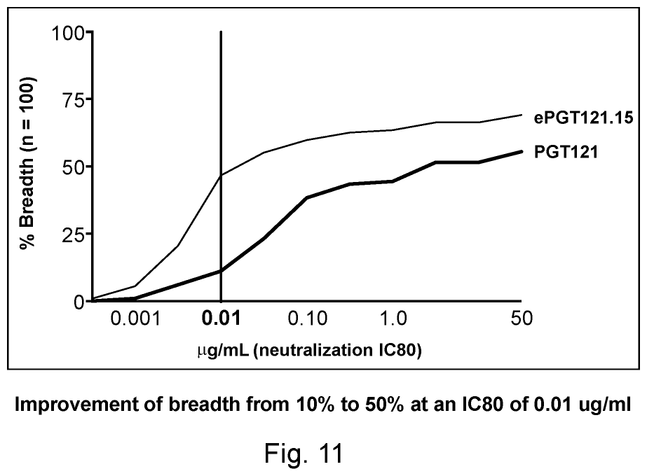

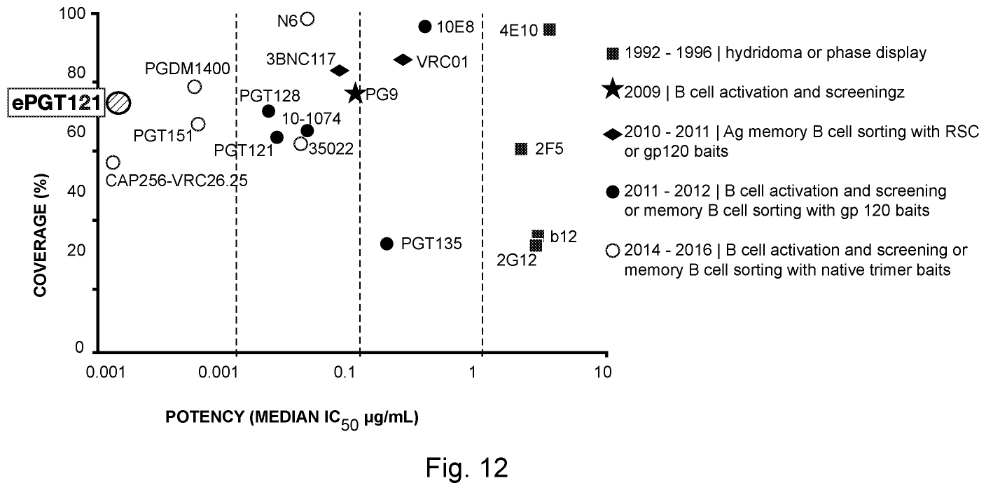

ePGT-121.15.H1.L1 was found to have 75% and 69% neutralization breadth with a potent median IC50 of 0.0010 μg/ml and IC80 of 0.0093 μg/ml, respectively, on a 106-virus panel and (FIG. 10). On the same panel, the parental PGT-121 antibody had a 66% and 61% neutralization breadth with a median IC50 of 0.0270 μg/ml and IC80 of 0.0850 μg/ml, respectively. As shown in FIG. 11, the breadth of neutralization at an IC80 of 0.01 microg/ml improved from improved from 10% (parent PGT-121) to 50% (ePGT-121.15.1). FIG. 12 further shows that the engineered PGT-121 variant antibodies have enhanced neutralization breadth and potency compared to the previously identified broadly neutralizing anti-HIV env antibodies.

Neutralization breadth and potency of the ePGT121.H1.L1 antibody comprising the PGT-121.15.H1 heavy chain (SEQ ID NO: 3) and LC1 light chain (SEQ ID NO: 10), and PGT-121 parent on a 106 cross-clade virus panel are shown in the Table below.

TABLE 5
Neutralization breadth and potency of the ePGT121.H1.L1 antibody.

| Virus | Clade | PGT121 | ePGT121.15.H1.L1 |
|---|---|---|---|
| 6535.3 | B | 0.002 | 0.0003 |
| QH0692.42 | B | 0.302 | 0.0008 |
| SC422661.8 | B | 0.038 | 0.0005 |
| PVO.4 | B | 0.098 | 0.0004 |
| TRO.11 | B | 0.005 | 0.0001 |
| AC10.0.29 | B | 0.024 | 0.0002 |
| RHPA4259.7 | B | 0.015 | 0.0003 |
| REJO4541.67 | B | 4.77 | 3.17 |
| TRJO4551.58 | B | 1.31 | 0.0005 |
| WITO4160.33 | B | 0.334 | 0.0011 |
| CAAN5342.A2 | B | 0.007 | 0.0002 |
| WEAU_d15_410_5017 | B (T/F) | 0.026 | 0.0001 |
| 1006_11_C3_1601 | B (T/F) | 0.002 | 0.0003 |
| 1054_07_TC4_1499 | B (T/F) | 0.064 | 0.0001 |
| 1056_10_TA11_1826 | B (T/F) | 0.004 | 0.0001 |
| 1012_11_TC21_3257 | B (T/F) | 0.003 | 0.0009 |
| 6240_08_TA5_4622 | B (T/F) | 0.033 | 0.0022 |
| 6244_13_B5_4576 | B (T/F) | 0.061 | 0.0037 |
| 62357_14_D3_4589 | B (T/F) | 2.6 | 0.0040 |
| SC05_8C11_2344 | B (T/F) | 0.019 | 0.0009 |
| Du156.12 | C | 0.004 | 0.0001 |
| Du172.17 | C | 0.033 | 0.0002 |
| Du422.1 | C | 0.039 | 0.0007 |
| ZM197M.PB7 | C | >50 | >50 |
| ZM214M.PL15 | C | 0.46 | 0.0006 |
| ZM233M.PB6 | C | 3.69 | 0.0124 |
| ZM249M.PL1 | C | >50 | 0.0024 |
| ZM53M.PB12 | C | 0.001 | 0.0006 |
| ZM109F.PB4 | C | 8.64 | 10.7 |
| ZM135M.PL10a | C | 0.716 | 0.1316 |
| CAP45.2.00.G3 | C | 1.63 | 5.02 |
| CAP210.2.00.E8 | C | 26.3 | 0.0052 |
| HIV-001428-2.42 | C | 0.014 | 0.0001 |
| HIV-0013095-2.11 | C | >50 | 0.0001 |
| HIV-16055-2.3 | C | 0.153 | 0.0003 |
| HIV-16845-2.22 | C | 3.97 | 0.0072 |
| Ce0393_C3 | C (T/F) | >50 | >50 |
| Ce1176_A3 | C (T/F) | 0.013 | 0.0004 |
| Ce2010_F5 | C (T/F) | >50 | >50 |
| Ce1172_H1 | C (T/F) | 0.011 | 0.0003 |
| Ce2060_G9 | C (T/F) | >50 | >50 |
| Ce703010054_2A2 | C (T/F) | >50 | >50 |
| BF1266.431a | C (T/F) | >50 | >50 |
| 246F_C1G | C (T/F) | 0.041 | 0.0013 |
| 249M_B10 | C (T/F) | >50 | 0.0022 |
| ZM247v1(Rev−) | C (T/F) | 0.028 | 0.0003 |
| 7030102001E5(Rev−) | C (T/F) | 0.009 | 0.0002 |
| 1394C9G1(Rev−) | C (T/F) | 0.264 | 0.0004 |
| Ce704809221_1B3 | C (T/F) | 0.025 | 0.0000 |
| CNE19 | BC | 0.008 | 0.0000 |
| CNE20 | BC | 0.001 | 0.0003 |
| CNE21 | BC | 0.007 | 0.0001 |
| CNE17 | BC | 7.6 | 0.0037 |
| CNE30 | BC | >50 | 0.0024 |
| CNE52 | BC | 2.05 | 0.0203 |
| CNE53 | BC | 0.007 | 0.0010 |
| CNE58 | BC | >50 | 0.0025 |
| MS208.A1 | A | >50 | >50 |
| Q23.17 | A | 0.001 | 0.0004 |
| Q461.e2 | A | >50 | >50 |
| Q769.d22 | A | >50 | >50 |
| 0330.v4.c3 | A | 0.05 | 0.0015 |
| 0260.v5.c36 | A | 0.053 | 0.0040 |
| 191955_A11 | A (T/F) | >50 | >50 |
| 191084_B7-19 | A (T/F) | 0.011 | 0.0009 |
| T257-31 | CRF02_AG | >50 | >50 |
| 928-28 | CRF02_AG | 44.2 | 0.0178 |
| 263-8 | CRF02_AG | 0.648 | 0.0044 |
| T250-4 | CRF02_AG | 0.001 | 0.0002 |
| T251-18 | CRF02_AG | 29 | 0.0055 |
| T278-50 | CRF02_AG | >50 | 0.0124 |
| T255-34 | CRF02_AG | 18.7 | 0.0221 |
| 235-47 | CRF02_AG | 0.137 | 0.0019 |
| 620345.c01 | CRF01_AE | >50 | >50 |
| C1080.c03 | CRF01_AE | >50 | >50 |
| R2184.c04 | CRF01_AE | >50 | >50 |
| R1166.c01 | CRF01_AE | >50 | >50 |
| R3265.c06 | CRF01_AE | >50 | >50 |
| C3347.c11 | CRF01_AE | >50 | >50 |
| C4118.c09 | CRF01_AE | >50 | >50 |
| CNE8 | CRF01_AE | >50 | >50 |
| CNE5 | CRF01_AE | >50 | 15.8 |
| BJOX009000.02.4 | CRF01_AE | 2.86 | 0.0046 |
| BJOX015000.11.5 | CRF01_AE (T/F) | >50 | >50 |
| BJOX010000.06.2 | CRF01_AE (T/F) | >50 | >50 |
| BJOX025000.01.1 | CRF01_AE (T/F) | >50 | >50 |
| BJOX028000.10.3 | CRF01_AE (T/F) | >50 | 0.0021 |
| X1193_c1 | G | 0.016 | 0.0001 |
| X1254_c3 | G | 0.014 | 0.0014 |
| X2088_c9 | G | 0.003 | 0.0007 |
| X2131_C1_B5 | G | 0.004 | 0.0004 |
| P1981_C5_3 | G | 0.001 | 0.0014 |
| X1632_S2_B10 | G | >50 | >50 |
| A07412M1.vrc12 | D | 0.009 | 0.0022 |
| 231965.c01 | D | >50 | >50 |
| 3817.v2.c59 | CD | 18.9 | 0.7236 |
| 6480.v4.c25 | CD | 0.001 | 0.0012 |
| 6952.v1.c20 | CD | 0.056 | 0.0009 |
| 6811.v7.c18 | CD | 0.001 | 0.0012 |
| 89-F1_2_25 | CD | >50 | >50 |
| 3301.v1.c24 | AC | 0.008 | 0.0029 |
| 6041.v3.c23 | AC | >50 | 12.4 |
| 6540.v4.c1 | AC | >50 | >50 |
| 6545.v4.c1 | AC | >50 | >50 |
| 0815.v3.c3 | ACD | 0.025 | 0.0017 |
| 3103.v3.c10 | ACD | 0.009 | 0.0017 |
| | % Breadth | 66% | 75% |
| | Median IC50 | 0.027 | 0.001 |

Neutralization breadth and potency of the ePGT121.H1.L1, ePGT121.H2.L1, ePGT121.H6.L1, and PGT-121 parent antibodies assessed using on a 113-member cross-clade virus panel are shown in the Table below. ePGT121.H1.L1 comprises the ePGT121.15.H1 VH (SEQ ID NO: 3) and the ePGT121.15.L1 VL (SEQ ID NO: 10); ePGT121.H2.L1 comprises the ePGT121.15.H2 VH (SEQ ID NO: 4) and the ePGT121.15.L1 VL (SEQ ID NO: 10); ePGT121.H6.L1 comprises the ePGT121.15.H6 VH (SEQ ID NO: 8) and the ePGT121.15.L1 VL (SEQ ID NO: 10). Each of the ePGT121.H1.L1, ePGT121.H2.L1, ePGT121.H6.L1 antibodies displayed neutralization with improved breadth and potency compared to the parent PGT-121 antibody.

TABLE 6
Neutralization breadth and potency of the ePGT121.H1.L1, ePGT121.H2.L1,
and ePGT121.H6.L1 antibodies. NN = not neutralizing.

| | Breadth | 76% | 77% | 77% | 67% |
|---|---|---|---|---|---|
| | GeoMean | 0.0061 | 0.0040 | 0.0048 | 0.0229 |
| CLADE | VIRUS | ePGT121.15.H1.L1 | ePGT121.15.H2.L1 | ePGT121.15.H6.L1 | PGT121 |
| B | 62357 | 0.009821 | 0.010870 | 0.013240 | 0.391600 |
| B | QH0692 | 0.014870 | 0.011570 | 0.012630 | 0.175900 |
| B | REJO | 0.310800 | 0.434800 | 0.000008 | 0.286300 |
| B | TRJO | 0.003585 | 0.005827 | 0.006728 | 0.405800 |
| B | WITO | 0.003519 | 0.004276 | 0.001840 | 1.096000 |
| BC | CNE17 | 0.010650 | 0.005775 | 0.010070 | 0.224800 |
| BC | CNE52 | 0.051480 | 0.046850 | 0.078390 | 1.941000 |
| BC | CNE58 | 0.007949 | 0.004640 | 0.014480 | 1.281000 |
| C | 1394C9G | 0.004032 | 0.002030 | 0.002879 | 0.122200 |
| C | 249M B10 | 0.006388 | 0.002710 | 0.011790 | NN |
| C | CAP210 | 0.022860 | 0.020710 | 0.031070 | 0.617800 |
| C | CAP45 | 3.062000 | 0.000009 | 0.000018 | 0.071270 |
| C | Du172.17 | 0.002398 | 0.000836 | 0.002033 | 0.031600 |
| C | HIV-0013095 | 1.068000 | 0.697600 | 0.635300 | NN |
| C | HIV-16055 | 1.068000 | 0.697600 | 0.635300 | NN |
| C | HIV-16845 | 0.020860 | 0.022220 | 0.028310 | 0.249800 |
| C | ZM135 | 0.110000 | 0.122200 | 0.168200 | 1.320000 |
| C | ZM214 | 0.014580 | 0.007271 | 0.009928 | 0.118600 |
| C | ZM249 | 0.004035 | 0.008367 | 0.012690 | NN |
| CD | 3817.v2.c59 | 0.004950 | 0.003284 | 0.003610 | 0.009485 |
| AE | BJOX 9000 | 0.025540 | 0.025910 | 0.016590 | 0.243500 |
| AE | BJOX 28000 | 0.027850 | 0.005775 | 0.001715 | NN |
| AG | 928-28 | 0.086840 | 0.048800 | 0.085210 | 0.836100 |
| AG | T211-9 | 0.005317 | 0.005081 | 0.007515 | 0.890900 |
| AG | T251-18 | 0.027380 | 0.025200 | 0.027360 | 1.157000 |
| AG | T255-34 | 0.002581 | 0.003752 | 0.002138 | 0.045950 |
| AG | T263-8 | 0.007070 | 0.003507 | 0.006129 | 0.108600 |
| AG | T278-50 | 0.023170 | 0.012690 | 0.026340 | NN |
| B | 6535.3 | 0.001212 | 0.001038 | 0.001209 | 0.003248 |
| B | SC422661.8 | 0.003718 | 0.001452 | 0.002848 | 0.027160 |
| B | PVO.4 | 0.004402 | 0.003462 | 0.005175 | 0.115100 |
| B | TRO.11 | 0.001559 | 0.001153 | 0.001397 | 0.008435 |
| B | AC10.0.29 | 0.002722 | 0.001956 | 0.002783 | 0.028950 |
| B | RHPA4259.7 | 0.002332 | 0.001561 | 0.002251 | 0.009154 |
| B | WITO4160.33 | 0.004782 | 0.002412 | 0.003104 | 0.391400 |
| B | CAAN5342.A2 | 0.001918 | 0.001122 | 0.002208 | 0.008718 |
| B | WEAU_d15_410_5017 | 0.002085 | 0.001677 | 0.002049 | 0.014380 |
| C | 1006_11_C3_1601 | 0.001947 | 0.001749 | 0.002135 | 0.003816 |
| C | 1054_07_TC4_1499 | 0.001671 | 0.001138 | 0.001287 | 0.033370 |
| C | 1056_10_TA11_1826 | 0.003913 | 0.000864 | 0.001858 | 0.016180 |
| C | 1012_11_TC21_3257 | 0.002624 | 0.001722 | 0.001943 | 0.009171 |
| C | 6240_08_TA5_4622 | 0.005201 | 0.003999 | 0.004648 | 0.042700 |
| C | 6244_13_B5_4576 | 0.006970 | 0.006892 | 0.005500 | 0.140300 |
| C | SC05_8C11_2344 | 0.007623 | 0.006639 | 0.004726 | 0.027820 |
| C | Du156.12 | 0.001240 | 0.000443 | 0.000917 | 0.005341 |
| C | ZM197M.PB7 | NN | NN | NN | NN |
| C | ZM233M.PB6 | 0.007912 | 0.004800 | 0.009905 | 0.126200 |
| C | ZM233, 4C | 0.012830 | 0.006722 | 0.006930 | 0.236800 |
| C | ZM109F.PB4 | 2.146000 | 0.873400 | 0.741600 | 0.558200 |
| C | HIV-001428-2.42 | 0.001534 | 0.001356 | 0.001814 | 0.011090 |
| C | Ce0393_c3 | NN | NN | NN | NN |
| C | Ce1176_A3 | 0.002825 | 0.002059 | 0.003017 | 0.008821 |
| C | Ce2010_F5 | NN | NN | NN | NN |
| C | Ce1172_H1 | 0.000750 | 0.000582 | 0.000952 | 0.006934 |
| C | Ce2060_G9 | 0.010230 | 0.013200 | 0.036930 | NN |
| C | Ce703010054_2A2 | NN | NN | NN | NN |
| C | BF1266.431a | NN | NN | NN | NN |
| C | 246F C1G | 0.004285 | 0.002399 | 0.002780 | 0.021480 |
| C | ZM247v1(Rev−) | 0.001575 | 0.000909 | 0.001908 | 0.011360 |
| C | 7030102001E5(Rev−) | 0.002611 | 0.001969 | 0.002409 | 0.006787 |
| C | Ce704809221_1B3 | 0.001618 | 0.001426 | 0.002306 | 0.027990 |
| BC | CNE19 | 0.001028 | 0.000602 | 0.000806 | 0.003148 |
| BC | CNE20 | 0.000933 | 0.000538 | 0.001333 | 0.001467 |
| BC | CNE21 | 0.000929 | 0.000874 | 0.001236 | 0.005248 |
| BC | CNE53 | 0.002883 | 0.001625 | 0.003063 | 0.007009 |
| A | MS208.A1 | NN | NN | NN | NN |
| A | Q23.17 | 0.001255 | 0.001057 | 0.001464 | 0.004119 |
| A | Q769.d22 | NN | NN | NN | NN |
| A | 0330.v4.c3 | 0.006213 | 0.005303 | 0.007644 | 0.051300 |
| A | 191955_A11 | NN | NN | NN | NN |
| A | 191084 B7-19 | 0.002788 | 0.001960 | 0.003116 | 0.024740 |
| AG | T263-8 | 0.007777 | 0.004879 | 0.004646 | 0.124200 |
| AG | T255_34 at4C | 0.004548 | 0.002445 | 0.002263 | 0.013700 |
| AG | T235-47 | 0.006098 | 0.004662 | 0.003628 | 0.045700 |
| AE | 620345.c01 | NN | NN | NN | NN |
| AE | C1080.c03 | NN | NN | NN | NN |
| AE | R2184.c04 | NN | NN | NN | NN |
| D | A0742M1.vrc12 | 0.000964 | 0.000944 | 0.002045 | 0.009487 |
| AE | R1166.c01 | NN | NN | NN | NN |
| AE | R3265.c06 | NN | NN | NN | NN |
| AE | C3347.c11 | NN | NN | NN | NN |
| AE | C4118.c09 | NN | NN | NN | NN |
| G | X1254 C3 | 0.002990 | 0.002346 | 0.002724 | 0.020590 |
| G | X2088_C9 | 0.001474 | 0.000985 | 0.001082 | 0.002144 |
| G | P1981_C5_3 | 0.000690 | 0.000449 | 0.000543 | 0.001456 |
| G | X1632_S2_B10 | NN | NN | NN | NN |
| D | 231965.c01 | 0.680100 | 0.285400 | 1.270000 | NN |
| CD | 6811.v7.c18 | 0.001085 | 0.001940 | 0.001139 | 0.001250 |
| CD | 89-F1_2_25 | NN | NN | NN | NN |
| AC | 3301.v1.c24 | 0.001658 | 0.001059 | 0.001343 | 0.008691 |
| AC | 6041.v3.c23 | NN | NN | NN | NN |
| AC | 6540.v4.c1 | 0.248600 | 0.652300 | 0.612100 | NN |
| AC | 6545.v4.c1 | NN | NN | NN | NN |
| ACD | 0815.v3.c3 | 0.001668 | 0.000394 | 0.000220 | 0.000079 |
| ACD | 3103.v3.c10 | 0.005087 | 0.004364 | 0.005126 | 0.020640 |
| C | Du422.1 | 0.002433 | 0.003255 | 0.002638 | 0.001716 |
| C | ZM53M.PB12 | 0.002063 | 0.001773 | 0.002036 | 0.000380 |
| BC | CNE30 | 0.002995 | 0.003121 | 0.002886 | 0.003126 |
| A | Q461.c2 | NN | NN | NN | NN |
| A | 0260.v5.c36 | 0.002466 | 0.001991 | 0.002166 | 0.001438 |
| AG | T257-31 | NN | NN | NN | NN |
| AG | T250-4 | 0.000854 | 0.000840 | 0.000789 | 0.000210 |
| AE | CNE8 | NN | NN | NN | NN |
| AE | CNE5 | NN | 0.742900 | 2.717000 | NN |
| AE | BJOX 15000 | NN | NN | NN | NN |
| AE | BJOX 10000 | NN | NN | NN | NN |
| AE | BJOX 25000 | NN | NN | NN | NN |
| G | X1193_C1 | 0.000256 | 0.000174 | 0.000518 | 0.000417 |
| G | P0402_C2_11 | 0.002678 | 0.001926 | 0.001500 | 0.000423 |
| D | 3016.v5.c45 | NN | NN | NN | NN |
| CD | 6480.v4.c25 | 0.000305 | 0.000045 | 0.000826 | 0.000306 |
| CD | 6952.v1.c20 | 0.000810 | 0.000842 | 0.000793 | 0.000292 |
| AC | 6041.v3.c23 | 0.396400 | 0.275100 | 0.416700 | NN |

As demonstrated by the data presented in the tables above, the engineered PGT-121 variant antibodies provided herein have significantly improved neutralization breadth and potency compared to the PGT-121 parental antibody. In particular, the engineered PGT-121 variant antibodies provided herein are suitable for neutralizing PGT-121 resistant isolates.

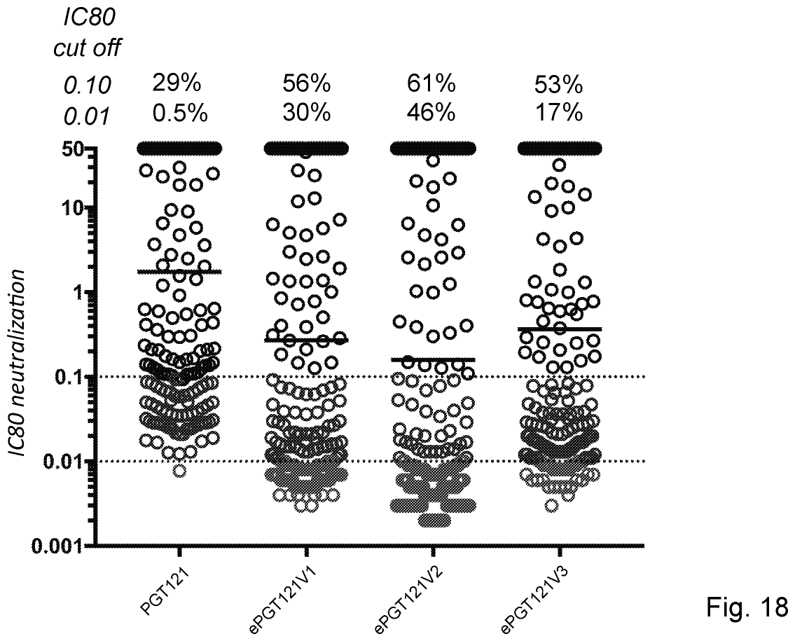

Neutralization breadth and potency of the PGT121, ePGT121.15, ePGT121.18, and ePGT121.18.17 antibodies assessed on a 208-member cross-clade virus panel are shown in Table 7 and 8 below and in FIG. 18. IC50 and IC80 values in microg/ml are shown in Tables 7 and 8, respectively. ePGT121.15 comprises ePGT121.15.H6 VH and ePGT121.15.L1 VL, ePGT121.18 comprises ePGT121.18.H1 VH and ePGT121.17.L1 VL, and ePGT121.18.17 comprises ePGT121.18.H17 VH and ePGT121.17.L1 VL. Each of ePGT121.15, ePGT121.18, and ePGT121.18.17 displayed neutralization with improved breadth and potency compared to the parent PGT121 antibody.

TABLE 7
Neutralization breadth and potency of the PGT121, ePGT121.15, ePGT121.18,
and ePGT121.18.17 antibodies assessed on a 208-member panel. Values
represent IC50 in microg/ml. Median and Geometric Mean titers are
calculated only for samples with IC50 < 50 ug/ml

| Virus ID | Clade | PGT121 | ePGT121.15 | ePGT121.18 | ePGT121.18.17 |
|---|---|---|---|---|---|
| 0260.v5.c36 | A | 0.058667 | 0.004 | 0.002 | 0.009 |
| 0330.v4.c3 | A | 0.059667 | 0.006 | 0.003 | 0.012 |
| 0439.v5.c1 | A | >50 | 4.72 | 5.67 | >50 |
| 3365.v2.c20 | A | 0.112333 | 0.006 | 0.004 | 0.007 |
| 3415.v1.c1 | A | >50 | 0.017 | 0.006 | 0.062 |
| 3718.v3.c11 | A | 0.559 | 0.489 | 0.267 | 0.957 |
| 398-F1.F6.20 | A | 0.022333 | 0.004 | 0.003 | 0.005 |
| BB201.B42 | A | 0.009333 | 0.003 | 0.002 | 0.003 |
| BB539.2B13 | A | >50 | >50 | >50 | >50 |
| BG505.W6M.C2 | A | 0.051333 | 0.066 | 0.014 | 0.047 |
| BI369.9A | A | 0.028333 | 0.002 | 0.001 | 0.004 |
| BS208.B1 | A | >50 | >50 | >50 | >50 |
| KER2008.12 | A | 0.553 | 0.01 | 0.003 | 0.01 |
| KER2018.11 | A | >50 | >50 | >50 | >50 |
| KNH1209.18 | A | 0.011667 | 0.003 | 0.001 | 0.003 |
| MB201.A1 | A | 0.009333 | 0.004 | 0.001 | 0.004 |
| MB539.2B7 | A | >50 | >50 | >50 | >50 |
| MI369.A5 | A | 0.050667 | 0.004 | 0.002 | 0.008 |
| MS208.A1 | A | >50 | >50 | >50 | >50 |
| Q23.17 | A | 0.010667 | 0.003 | 0.002 | 0.006 |
| Q259.17 | A | >50 | >50 | >50 | >50 |
| Q769.d22 | A | >50 | >50 | >50 | >50 |
| Q769.h5 | A | >50 | >50 | >50 | >50 |
| Q842.d12 | A | 0.021667 | 0.003 | 0.002 | 0.007 |
| QH209.14M.A2 | A | >50 | >50 | >50 | >50 |
| RW020.2 | A | 0.01 | 0.003 | 0.002 | 0.003 |
| UG037.8 | A | 0.092 | 0.004 | 0.002 | 0.009 |
| 246-F3.C10.2 | AC | >50 | 11.6 | 4.8 | 18.5 |
| 3301.v1.c24 | AC | 0.021667 | 0.006 | 0.003 | 0.009 |
| 3589.v1.c4 | AC | >50 | 0.229 | 0.252 | 2.73 |
| 6540.v4.c1 | AC | >50 | >50 | >50 | >50 |
| 6545.v4.c1 | AC | >50 | >50 | >50 | >50 |
| 0815.v3.c3 | ACD | 0.032333 | 0.002 | 0.001 | 0.005 |
| 6095.v1.c10 | ACD | 1.98 | 0.019 | 0.006 | 0.045 |
| 3468.v1.c12 | AD | 0.076333 | 0.002 | 0.001 | 0.004 |
| Q168.a2 | AD | >50 | 5.07 | 2.93 | 1.52 |
| Q461.e2 | AD | >50 | >50 | >50 | >50 |
| 620345.c1 | AE | >50 | >50 | >50 | >50 |
| BJOX009000.02.4 | AE | 0.750667 | 0.183 | 0.042 | 0.156 |
| BJOX010000.06.2 | AE | >50 | >50 | >50 | >50 |
| BJOX025000.01.1 | AE | >50 | >50 | >50 | >50 |
| BJOX028000.10.3 | AE | >50 | 0.363 | 0.054 | 0.338 |
| C1080.c3 | AE | >50 | >50 | >50 | >50 |
| C2101.c1 | AE | >50 | >50 | >50 | >50 |
| C3347.c11 | AE | >50 | >50 | >50 | >50 |
| C4118.09 | AE | >50 | >50 | >50 | >50 |
| CM244.ec1 | AE | >50 | 7.02 | 0.46 | 7.23 |
| CNE3 | AE | >50 | >50 | >50 | >50 |
| CNE5 | AE | >50 | >50 | 7.18 | 47 |
| CNE55 | AE | >50 | >50 | >50 | >50 |
| CNE56 | AE | >50 | >50 | >50 | >50 |
| CNE59 | AE | >50 | >50 | >50 | >50 |
| CNE8 | AE | >50 | >50 | >50 | >50 |
| M02138 | AE | >50 | >50 | >50 | >50 |
| R1166.C1 | AE | >50 | >50 | >50 | >50 |
| R2184.c4 | AE | >50 | >50 | >50 | >50 |
| R3265.c6 | AE | >50 | >50 | >50 | >50 |
| TH023.6 | AE | >50 | >50 | >50 | >50 |
| TH966.8 | AE | >50 | >50 | >50 | >50 |
| TH976.17 | AE | >50 | >50 | >50 | >50 |
| 235-47 | AG | 0.131667 | 0.009 | 0.006 | 0.022 |
| 242-14 | AG | >50 | 42.8 | 17.3 | >50 |
| 263-8 | AG | 0.483667 | 0.014 | 0.004 | 0.02 |
| 269-12 | AG | 0.128333 | 0.011 | 0.003 | 0.01 |
| 271-11 | AG | >50 | >50 | >50 | >50 |
| 928-28 | AG | 5.76 | 0.161 | 0.076 | 0.462 |
| DJ263.8 | AG | 0.057667 | 0.013 | 0.002 | 0.014 |
| T250-4 | AG | 0.004333 | 0.002 | 0.0008 | 0.002 |
| T251-18 | AG | 3.05 | 0.056 | 0.019 | 0.041 |
| T253-11 | AG | >50 | 0.009 | 0.004 | 0.025 |
| T255-34 | AG | 2.042667 | 0.045 | 0.016 | 0.032 |
| T257-31 | AG | >50 | >50 | >50 | >50 |
| T266-60 | AG | 0.143333 | 0.006 | 0.003 | 0.014 |
| T278-50 | AG | >50 | 4.87 | 1.17 | 1.2 |
| T280-5 | AG | 0.016 | 0.004 | 0.002 | 0.006 |
| T33-7 | AG | >50 | >50 | >50 | >50 |
| 3988.25 | B | 0.012667 | 0.004 | 0.003 | 0.004 |
| 5768.04 | B | 0.118333 | 0.028 | 0.004 | 0.029 |
| 6101.10 | B | 0.012333 | 0.002 | 0.001 | 0.004 |
| 6535.3 | B | 0.006333 | 0.0007 | 0.001 | 0.002 |
| 7165.18 | B | 0.036333 | 0.003 | 0.001 | 0.006 |
| 45_01dG5 | B | 0.011 | 0.003 | 0.001 | 0.003 |
| 89.6.DG | B | 0.033667 | 0.002 | 0.001 | 0.004 |
| AC10.29 | B | 0.067 | 0.002 | 0.001 | 0.004 |
| ADA.DG | B | 0.033333 | 0.005 | 0.002 | 0.005 |
| Bal.01 | B | 0.011333 | 0.002 | 0.002 | 0.003 |
| BaL.26 | B | 0.022667 | 0.003 | 0.002 | 0.004 |
| BG1168.01 | B | >50 | >50 | >50 | >50 |
| BL01.DG | B | 19.55 | 13.1 | 6.91 | 1.51 |
| BR07.DG | B | 0.132 | 0.009 | 0.005 | 0.008 |
| BX08.16 | B | 0.009333 | 0.004 | 0.003 | 0.004 |
| CAAN.A2 | B | 0.019333 | 0.005 | 0.002 | 0.004 |
| CNE10 | B | 0.019333 | 0.004 | 0.002 | 0.004 |
| CNE12 | B | 0.015333 | 0.003 | 0.002 | 0.004 |
| CNE14 | B | 0.010333 | 0.002 | 0.001 | 0.003 |
| CNE4 | B | 1.903333 | 0.004 | 0.002 | 0.008 |
| CNE57 | B | 0.028333 | 0.004 | 0.002 | 0.006 |
| HO86.8 | B | >50 | >50 | >50 | >50 |
| HT593.1 | B | >50 | 0.008 | 0.003 | 0.014 |
| HXB2.DG | B | >50 | 0.011 | 0.003 | 0.016 |
| JRCSF.JB | B | 0.056333 | 0.008 | 0.003 | 0.007 |
| JRFL.JB | B | 0.039667 | 0.014 | 0.006 | 0.011 |
| MN.3 | B | >50 | 0.004 | 0.002 | 0.004 |
| PVO.04 | B | 0.189 | 0.006 | 0.003 | 0.009 |
| QH0515.01 | B | 2.83 | 0.014 | 0.007 | 0.035 |
| QH0692.42 | B | 0.972 | 0.015 | 0.006 | 0.035 |
| REJO.67 | B | 1.956667 | 1.46 | 0.731 | 1.43 |
| RHPA.7 | B | 0.035667 | 0.003 | 0.002 | 0.004 |
| SC422.8 | B | 0.114667 | 0.011 | 0.005 | 0.016 |
| SF162.LS | B | 0.007333 | 0.003 | 0.002 | 0.003 |
| SS1196.01 | B | 0.012333 | 0.003 | 0.002 | 0.004 |
| THRO.18 | B | >50 | >50 | >50 | >50 |
| TRJO.58 | B | 1.633333 | 0.007 | 0.003 | 0.023 |
| TRO.11 | B | 0.020333 | 0.003 | 0.002 | 0.005 |
| WITO.33 | B | 0.479 | 0.008 | 0.004 | 0.015 |
| X2278.C2.B6 | B | 0.026 | 0.002 | 0.001 | 0.003 |
| YU2.DG | B | 0.039667 | 0.005 | 0.003 | 0.011 |
| BJOX002000.03.2 | BC | 0.026 | 0.003 | 0.002 | 0.004 |
| CH038.12 | BC | 0.011333 | 0.001 | 0.0008 | 0.002 |
| CH070.1 | BC | 0.013667 | 0.001 | 0.0008 | 0.003 |
| CH117.4 | BC | >50 | >50 | >50 | >50 |
| CH119.10 | BC | 0.031333 | 0.005 | 0.004 | 0.013 |
| CH181.12 | BC | 0.031333 | 0.003 | 0.001 | 0.004 |
| CNE15 | BC | 6.645 | 3.52 | 5.69 | 14 |
| CNE19 | BC | 0.009333 | 0.004 | 0.001 | 0.003 |
| CNE20 | BC | 0.005 | 0.003 | 0.001 | 0.002 |
| CNE21 | BC | 0.014333 | 0.002 | 0.0009 | 0.002 |
| CNE40 | BC | 0.281 | 0.028 | 0.015 | 0.036 |
| CNE7 | BC | 0.047667 | 0.005 | 0.003 | 0.007 |
| 286.36 | C | 0.012 | 0.002 | 0.001 | 0.003 |
| 288.38 | C | 0.016 | 0.001 | 0.0008 | 0.002 |
| 0013095-2.11 | C | >50 | 3.01 | 0.537 | 1.68 |
| 001428-2.42 | C | 0.033667 | 0.002 | 0.001 | 0.004 |
| 0077.v1.c16 | C | >50 | >50 | >50 | >50 |
| 00836-2.5 | C | >50 | 2.25 | 14.4 | 48.9 |
| 0921.v2.c14 | C | >50 | 0.454 | 0.091 | 0.189 |
| 16055-2.3 | C | 0.312 | 0.029 | 0.011 | 0.036 |
| 16845-2.22 | C | 2.723333 | 0.077 | 0.036 | 0.232 |
| 16936-2.21 | C | 0.011 | 0.003 | 0.002 | 0.004 |
| 25710-2.43 | C | 0.022667 | 0.006 | 0.004 | 0.008 |
| 25711-2.4 | C | 0.017333 | 0.004 | 0.002 | 0.006 |
| 25925-2.22 | C | 0.045 | 0.007 | 0.003 | 0.011 |
| 26191-2.48 | C | 0.085 | 0.005 | 0.003 | 0.011 |
| 3168.v4.c10 | C | 0.446333 | 0.026 | 0.012 | 0.053 |
| 3637.v5.c3 | C | >50 | >50 | >50 | >50 |
| 3873.v1.c24 | C | 0.045 | 0.004 | 0.003 | 0.011 |
| 426c | C | >50 | 0.385 | 0.065 | 0.26 |
| 6322.v4.c1 | C | >50 | >50 | >50 | >50 |
| 6471.v1.c16 | C | >50 | 1.04 | 0.378 | 1.09 |
| 6631.v3.c10 | C | >50 | 0.015 | 0.01 | 0.063 |
| 6644.v2.c33 | C | 0.037 | 0.003 | 0.002 | 0.005 |
| 6785.v5.c14 | C | 0.050333 | 0.004 | 0.002 | 0.005 |
| 6838.v1.c35 | C | 0.061333 | 0.234 | 0.386 | 0.576 |
| 96ZM651.02 | C | 0.021333 | 0.001 | 0.0007 | 0.003 |
| BR025.9 | C | 0.009667 | 0.002 | 0.001 | 0.003 |
| CAP210.E8 | C | 4.276667 | 0.052 | 0.02 | 0.124 |
| CAP244.D3 | C | >50 | >50 | 21.2 | >50 |
| CAP256.206.C9 | C | 0.02 | 0.003 | 0.001 | 0.004 |
| CAP45.G3 | C | 0.866 | 2.25 | 1.74 | 8.91 |
| Ce1176.A3 | C | 0.024333 | 0.003 | 0.003 | 0.008 |
| CE703010217.B6 | C | 0.007 | 0.004 | 0.002 | 0.005 |
| CNE30 | C | 0.114 | 0.008 | 0.005 | 0.018 |
| CNE31 | C | 0.653667 | 0.042 | 0.015 | 0.089 |
| CNE53 | C | 0.026333 | 0.007 | 0.003 | 0.006 |
| CNE58 | C | 9.34 | 0.023 | 0.016 | 0.053 |
| DU123.06 | C | 0.047667 | 0.003 | 0.002 | 0.004 |
| DU151.02 | C | 0.011667 | 0.002 | 0.001 | 0.003 |
| DU156.12 | C | 0.014667 | 0.002 | 0.001 | 0.003 |
| DU172.17 | C | 0.065 | 0.003 | 0.003 | 0.007 |
| DU422.01 | C | 0.062 | 0.01 | 0.006 | 0.013 |
| MW965.26 | C | 0.018 | 0.004 | 0.003 | 0.004 |
| SO18.18 | C | 0.005333 | 0.004 | 0.003 | 0.004 |
| TV1.29 | C | 0.145667 | 0.004 | 0.002 | 0.011 |
| TZA125.17 | C | 1.45 | 0.07 | 0.028 | 0.18 |
| TZBD.02 | C | 0.025667 | 0.003 | 0.002 | 0.003 |
| ZA012.29 | C | 0.011667 | 0.004 | 0.002 | 0.005 |
| ZM106.9 | C | 0.012667 | 0.002 | 0.001 | 0.004 |
| ZM109.4 | C | 1.956667 | 3.37 | 7.26 | 8.44 |
| ZM135.10a | C | 0.748 | 0.119 | 0.026 | 0.145 |
| ZM176.66 | C | 4.1 | 0.073 | 0.017 | 0.122 |
| ZM197.7 | C | >50 | >50 | >50 | >50 |
| ZM214.15 | C | 0.392333 | 0.028 | 0.018 | 0.08 |
| ZM215.8 | C | 0.030667 | 0.017 | 0.006 | 0.016 |
| ZM233.6 | C | 0.731333 | 0.016 | 0.006 | 0.017 |
| ZM249.1 | C | >50 | 0.249 | 0.134 | 0.332 |
| ZM53.12 | C | 0.002667 | 0.008 | 0.003 | 0.015 |
| ZM55.28a | C | 0.080667 | 0.009 | 0.005 | 0.012 |
| 3326.v4.c3 | CD | >50 | 0.362 | 0.306 | 0.633 |
| 3337.v2.c6 | CD | 1.389667 | 0.589 | 0.232 | 0.363 |
| 3817.v2.c59 | CD | 22.2 | 0.984 | 0.546 | 4.75 |
| 191821.E6.1 | D | >50 | 0.249 | 0.015 | 0.104 |
| 231965.c01 | D | >50 | 12.5 | 9.17 | >50 |
| 247-23 | D | >50 | >50 | >50 | >50 |
| 3016.v5.c45 | D | >50 | >50 | >50 | >50 |
| 57128.vrc15 | D | 1.266 | 0.007 | 0.004 | 0.012 |
| 6405.v4.c34 | D | 0.042 | 0.003 | 0.001 | 0.004 |
| A03349M1.vrc4a | D | 0.074667 | 0.006 | 0.002 | 0.009 |
| A07412M1.vrc12 | D | 0.042 | 0.005 | 0.003 | 0.004 |
| NKU3006.ec1 | D | >50 | >50 | >50 | >50 |
| UG021.16 | D | 0.916333 | 0.006 | 0.006 | 0.013 |
| UG024.2 | D | >50 | 0.516 | 2.36 | 0.429 |
| P0402.c2.11 | G | 0.012333 | 0.004 | 0.002 | 0.004 |
| P1981.C5.3 | G | 0.007 | 0.002 | 0.001 | 0.002 |
| X1193.c1 | G | 0.045667 | 0.003 | 0.002 | 0.005 |
| X1254.c3 | G | 0.034667 | 0.004 | 0.002 | 0.006 |
| X1632.S2.B10 | G | >50 | >50 | >50 | >50 |
| X2088.c9 | G | 0.006333 | 0.003 | 0.002 | 0.006 |
| X2131.C1.B5 | G | 0.013 | 0.002 | 0.0009 | 0.002 |
| SIVmac251.30.SG3 | NA | >50 | >50 | >50 | >50 |
| SVA.MLV | NA | >50 | >50 | >50 | >50 |

| | PGT121 | ePGT121.15 | ePGT121.18 | ePGT121.18.17 |
|---|---|---|---|---|
| # Viruses | 208 | 208 | 208 | 208 |
| Total VS Neutralized | | | | |
| IC50 < 50 ug/ml | 136 | 160 | 162 | 158 |
| IC50 < 10 ug/ml | 134 | 156 | 159 | 154 |
| IC50 < 1.0 ug/ml | 117 | 145 | 148 | 143 |
| IC50 < 0.1 ug/ml | 92 | 130 | 137 | 126 |
| IC50 < 0.01 ug/ml | 14 | 101 | 116 | 83 |
| % vs Neutralized | | | | |
| IC50 < 50 ug/ml | 65 | 77 | 78 | 76 |
| IC50 < 10 ug/ml | 64 | 75 | 76 | 74 |
| IC50 < 1.0 ug/ml | 56 | 70 | 71 | 69 |
| IC50 < 0.1 ug/ml | 44 | 63 | 66 | 61 |
| IC50 < 0.01 ug/ml | 7 | 49 | 56 | 40 |
| Median IC50 | 0.0397 | 0.0055 | 0.0030 | 0.0090 |
| Geometric Mean | 0.0716 | 0.0148 | 0.0081 | 0.0202 |

TABLE 8
Neutralization breadth and potency of the PGT121, ePGT121.15, ePGT121.18, and ePGT121.18.17
antibodies assessed on a 208-member panel. Two separate measurements (M1 and M2)
are shown for ePGT.121.18. Values represent IC80 in microg/ml. Median and Geometric
Mean titers are calculated only for samples with IC80 < 50 ug/ml

| Virus ID | Clade | PGT121 | ePGT121.15 | ePGT121.18 M1 | ePGT121.18 M2 | ePGT121.18.17 |
|---|---|---|---|---|---|---|
| 0260.v5.c36 | A | 0.159333 | 0.009 | 0.005 | 0.006 | 0.018 |
| 0330.v4.c3 | A | 0.211333 | 0.021 | 0.008 | 0.014 | 0.028 |
| 0439.v5.c1 | A | >50 | >50 | >50 | >50 | >50 |
| 3365.v2.c20 | A | 0.639333 | 0.021 | 0.01 | 0.011 | 0.037 |
| 3415.v1.c1 | A | >50 | 0.092 | 0.029 | 0.039 | 0.375 |
| 3718.v3.c11 | A | 4.756667 | 2.47 | 4.75 | 11.1 | 19.4 |
| 398-F1.F6.20 | A | 0.052667 | 0.009 | 0.005 | 0.007 | 0.016 |
| BB201.B42 | A | 0.024333 | 0.008 | 0.005 | 0.005 | 0.008 |
| BB539.2B13 | A | >50 | >50 | >50 | >50 | >50 |
| BG505.W6M.C2 | A | 0.165333 | 0.78 | 0.109 | 0.081 | 0.763 |
| BI369.9A | A | 0.085 | 0.006 | 0.003 | 0.004 | 0.009 |
| BS208.B1 | A | >50 | >50 | >50 | >50 | >50 |
| KER2008.12 | A | 6.555 | 0.062 | 0.014 | 0.015 | 0.073 |
| KER2018.11 | A | >50 | >50 | >50 | >50 | >50 |
| KNH1209.18 | A | 0.028667 | 0.007 | 0.004 | 0.004 | 0.006 |
| MB201.A1 | A | 0.028333 | 0.015 | 0.007 | 0.007 | 0.036 |
| MB539.2B7 | A | >50 | >50 | >50 | >50 | >50 |
| MI369.A5 | A | 0.128 | 0.009 | 0.005 | 0.005 | 0.02 |
| MS208.A1 | A | >50 | >50 | >50 | >50 | >50 |
| Q23.17 | A | 0.026667 | 0.009 | 0.005 | 0.005 | 0.013 |
| Q259.17 | A | >50 | >50 | >50 | >50 | >50 |
| Q769.d22 | A | >50 | >50 | >50 | >50 | >50 |
| Q769.h5 | A | >50 | >50 | >50 | >50 | >50 |
| Q842.d12 | A | 0.059667 | 0.01 | 0.005 | 0.007 | 0.02 |
| QH209.14M.A2 | A | >50 | >50 | >50 | >50 | >50 |
| RW020.2 | A | 0.017667 | 0.006 | 0.003 | 0.005 | 0.008 |
| UG037.8 | A | 0.307333 | 0.014 | 0.008 | 0.009 | 0.027 |
| 246-F3.C10.2 | AC | >50 | >50 | >50 | >50 | >50 |
| 3301.v1.c24 | AC | 0.05 | 0.016 | 0.008 | 0.008 | 0.017 |
| 3589.v1.c4 | AC | >50 | 1.92 | 6.5 | 34 | >50 |
| 6540.v4.c1 | AC | >50 | >50 | >50 | >50 | >50 |
| 6545.v4.c1 | AC | >50 | >50 | >50 | >50 | >50 |
| 0815.v3.c3 | ACD | 0.111667 | 0.006 | 0.003 | 0.005 | 0.011 |
| 6095.v1.c10 | ACD | >50 | 0.388 | 0.149 | 0.14 | 0.596 |
| 3468.v1.c12 | AD | 0.413 | 0.005 | 0.003 | 0.004 | 0.009 |
| Q168.a2 | AD | >50 | >50 | >50 | >50 | >50 |
| Q461.e2 | AD | >50 | >50 | >50 | >50 | >50 |
| 620345.c1 | AE | >50 | >50 | >50 | >50 | >50 |
| BJOX009000.02.4 | AE | 9.016667 | 0.853 | 0.389 | 0.157 | 1.07 |
| BJOX010000.06.2 | AE | >50 | >50 | >50 | >50 | >50 |
| BJOX025000.01.1 | AE | >50 | >50 | >50 | >50 | >50 |
| BJOX028000.10.3 | AE | >50 | 1.45 | 0.45 | 0.315 | 1.85 |
| C1080.c3 | AE | >50 | >50 | >50 | >50 | >50 |
| C2101.c1 | AE | >50 | >50 | >50 | >50 | >50 |
| C3347.c11 | AE | >50 | >50 | >50 | >50 | >50 |
| C4118.09 | AE | >50 | >50 | >50 | >50 | >50 |
| CM244.ec1 | AE | >50 | >50 | 6.26 | 14 | >50 |
| CNE3 | AE | >50 | >50 | >50 | >50 | >50 |
| CNE5 | AE | >50 | >50 | >50 | >50 | >50 |
| CNE55 | AE | >50 | >50 | >50 | >50 | >50 |
| CNE56 | AE | >50 | >50 | >50 | >50 | >50 |
| CNE59 | AE | >50 | >50 | >50 | >50 | >50 |
| CNE8 | AE | >50 | >50 | >50 | >50 | >50 |
| M02138 | AE | >50 | >50 | >50 | >50 | >50 |
| R1166.c1 | AE | >50 | >50 | >50 | >50 | >50 |
| R2184.c4 | AE | >50 | >50 | >50 | >50 | >50 |
| R3265.c6 | AE | >50 | >50 | >50 | >50 | >50 |
| TH023.6 | AE | >50 | >50 | >50 | >50 | >50 |
| TH966.8 | AE | >50 | >50 | >50 | >50 | >50 |
| TH976.17 | AE | >50 | >50 | >50 | >50 | >50 |
| 235-47 | AG | 0.614 | 0.027 | 0.017 | 0.024 | 0.068 |
| 242-14 | AG | >50 | >50 | >50 | >50 | >50 |
| 263-8 | AG | 2.496667 | 0.046 | 0.011 | 0.019 | 0.066 |
| 269-12 | AG | 0.627 | 0.036 | 0.009 | 0.01 | 0.027 |
| 271-11 | AG | >50 | >50 | >50 | >50 | >50 |
| 928-28 | AG | 29.85 | 0.719 | 0.332 | 0.377 | 1.31 |
| DJ263.8 | AG | 0.202 | 0.017 | 0.013 | 0.012 | 0.036 |
| T250-4 | AG | 0.012333 | 0.003 | 0.002 | 0.002 | 0.004 |
| T251-18 | AG | 23.25 | 0.184 | 0.069 | 0.056 | 0.175 |
| T253-11 | AG | >50 | 0.069 | 0.017 | 0.018 | 0.155 |
| T255-34 | AG | >50 | 1.35 | 0.402 | 0.664 | 0.803 |
| T257-31 | AG | >50 | >50 | >50 | >50 | >50 |
| T266-60 | AG | 0.548333 | 0.019 | 0.007 | 0.009 | 0.039 |
| T278-50 | AG | >50 | 27.7 | 10.7 | 2.51 | 9.19 |
| T280-5 | AG | 0.038 | 0.012 | 0.005 | 0.008 | 0.018 |
| T33-7 | AG | >50 | >50 | >50 | >50 | >50 |
| 3988.25 | B | 0.028667 | 0.008 | 0.004 | 0.008 | 0.008 |
| 5768.04 | B | 0.916667 | 0.286 | 0.018 | 0.023 | 0.255 |
| 6101.10 | B | 0.035333 | 0.005 | 0.003 | 0.005 | 0.01 |
| 6535.3 | B | 0.021667 | 0.003 | 0.002 | 0.003 | 0.005 |
| 7165.18 | B | 0.103333 | 0.007 | 0.005 | 0.006 | 0.014 |
| 45_01dG5 | B | 0.025667 | 0.006 | 0.003 | 0.004 | 0.01 |
| 89.6.DG | B | 0.078333 | 0.006 | 0.004 | 0.006 | 0.011 |
| AC10.29 | B | 0.148667 | 0.006 | 0.003 | 0.004 | 0.01 |
| ADA.DG | B | 0.109333 | 0.017 | 0.007 | 0.008 | 0.021 |
| Bal.01 | B | 0.027667 | 0.005 | 0.004 | 0.003 | 0.009 |
| BaL.26 | B | 0.06 | 0.008 | 0.005 | 0.006 | 0.014 |
| BG1168.01 | B | >50 | >50 | >50 | >50 | >50 |
| BL01.DG | B | >50 | 45.6 | 22.2 | 9.32 | 14.3 |
| BR07.DG | B | 0.596333 | 0.038 | 0.014 | 0.013 | 0.032 |
| BX08.16 | B | 0.021 | 0.01 | 0.006 | 0.008 | 0.013 |
| CAAN.A2 | B | 0.050333 | 0.011 | 0.005 | 0.006 | 0.011 |
| CNE10 | B | 0.048 | 0.01 | 0.005 | 0.004 | 0.01 |
| CNE12 | B | 0.034667 | 0.005 | 0.004 | 0.005 | 0.008 |
| CNE14 | B | 0.024333 | 0.005 | 0.003 | 0.005 | 0.007 |
| CNE4 | B | 25.3 | 0.014 | 0.005 | 0.007 | 0.025 |
| CNE57 | B | 0.077667 | 0.011 | 0.006 | 0.005 | 0.013 |
| HO86.8 | B | >50 | >50 | >50 | >50 | >50 |
| HT593.1 | B | >50 | 0.02 | 0.009 | 0.01 | 0.052 |
| HXB2.DG | B | >50 | 0.146 | 0.013 | 0.012 | 0.08 |
| JRCSF.JB | B | 0.162333 | 0.016 | 0.009 | 0.011 | 0.022 |
| JRFL.JB | B | 0.107 | 0.039 | 0.016 | 0.014 | 0.03 |
| MN.3 | B | >50 | 0.015 | 0.004 | 0.005 | 0.012 |
| PVO.04 | B | 0.498 | 0.015 | 0.007 | 0.008 | 0.023 |
| QH0515.01 | B | >50 | 0.052 | 0.021 | 0.028 | 0.13 |
| QH0692.42 | B | 5.803333 | 0.038 | 0.016 | 0.017 | 0.083 |
| REJO.67 | B | >50 | 12.9 | 17.5 | 23.1 | >50 |
| RHPA.7 | B | 0.093 | 0.008 | 0.005 | 0.006 | 0.013 |
| SC422.8 | B | 0.3 | 0.022 | 0.011 | 0.014 | 0.038 |
| SF162.LS | B | 0.021 | 0.006 | 0.004 | 0.006 | 0.008 |
| SS1196.01 | B | 0.030667 | 0.007 | 0.005 | 0.005 | 0.009 |
| THRO.18 | B | >50 | >50 | >50 | >50 | >50 |
| TRJO.58 | B | 18.63333 | 0.022 | 0.009 | 0.014 | 0.054 |
| TRO.11 | B | 0.058333 | 0.007 | 0.004 | 0.005 | 0.012 |
| WITO.33 | B | 2.023333 | 0.03 | 0.01 | 0.013 | 0.043 |
| X2278.C2.B6 | B | 0.072 | 0.006 | 0.003 | 0.004 | 0.009 |
| YU2.DG | B | 0.125667 | 0.014 | 0.008 | 0.01 | 0.027 |
| BJOX002000.03.2 | BC | 0.068 | 0.008 | 0.005 | 0.006 | 0.01 |
| CH038.12 | BC | 0.029 | 0.004 | 0.002 | 0.003 | 0.006 |
| CH070.1 | BC | 0.042333 | 0.004 | 0.002 | 0.002 | 0.007 |
| CH117.4 | BC | >50 | >50 | >50 | >50 | >50 |
| CH119.10 | BC | 0.093667 | 0.016 | 0.01 | 0.014 | 0.028 |
| CH181.12 | BC | 0.083 | 0.008 | 0.003 | 0.004 | 0.011 |
| CNE15 | BC | >50 | >50 | 36.4 | >50 | >50 |
| CNE19 | BC | 0.041 | 0.03 | 0.006 | 0.004 | 0.014 |
| CNE20 | BC | 0.012667 | 0.005 | 0.003 | 0.004 | 0.005 |
| CNE21 | BC | 0.038333 | 0.004 | 0.002 | 0.002 | 0.005 |
| CNE40 | BC | 1.21 | 0.082 | 0.047 | 0.047 | 0.13 |
| CNE7 | BC | 0.121333 | 0.013 | 0.008 | 0.007 | 0.017 |
| 286.36 | C | 0.027 | 0.006 | 0.003 | 0.004 | 0.006 |
| 288.38 | C | 0.044333 | 0.005 | 0.002 | 0.002 | 0.003 |
| 0013095-2.11 | C | >50 | 11.9 | 4.21 | 2.16 | 13.4 |
| 001428-2.42 | C | 0.107667 | 0.005 | 0.003 | 0.004 | 0.008 |
| 0077.v1.c16 | C | >50 | >50 | >50 | >50 | >50 |
| 00836-2.5 | C | >50 | >50 | >50 | >50 | >50 |
| 0921.v2.c14 | C | >50 | 5.71 | 1.03 | 1.49 | 3.48 |
| 16055-2.3 | C | 2.776667 | 0.211 | 0.089 | 0.059 | 0.207 |
| 16845-2.22 | C | 18.6 | 0.264 | 0.127 | 0.105 | 0.629 |
| 16936-2.21 | C | 0.024 | 0.007 | 0.003 | 0.005 | 0.011 |
| 25710-2.43 | C | 0.066 | 0.021 | 0.01 | 0.013 | 0.026 |
| 25711-2.4 | C | 0.049667 | 0.008 | 0.005 | 0.006 | 0.017 |
| 25925-2.22 | C | 0.086667 | 0.013 | 0.008 | 0.01 | 0.021 |
| 26191-2.48 | C | 0.235667 | 0.015 | 0.008 | 0.013 | 0.025 |
| 3168.v4.c10 | C | 1.43 | 0.075 | 0.034 | 0.045 | 0.171 |
| 3637.v5.c3 | C | >50 | >50 | >50 | >50 | >50 |
| 3873.v1.c24 | C | 0.113333 | 0.011 | 0.006 | 0.011 | 0.02 |
| 426c | C | >50 | 1.38 | 0.302 | 0.337 | 1 |
| 6322.v4.c1 | C | >50 | >50 | >50 | >50 | >50 |
| 6471.v1.c16 | C | >50 | 6.35 | 2.94 | 2.62 | 10 |
| 6631.v3.c10 | C | >50 | 0.065 | 0.04 | 0.044 | 0.269 |
| 6644.v2.c33 | C | 0.139667 | 0.008 | 0.005 | 0.006 | 0.015 |
| 6785.v5.c14 | C | 0.108 | 0.01 | 0.006 | 0.006 | 0.015 |
| 6838.v1.c35 | C | 0.21 | 1.34 | 2.59 | 13.4 | 4.34 |
| 96ZM651.02 | C | 0.061333 | 0.004 | 0.002 | 0.003 | 0.006 |
| BR025.9 | C | 0.025333 | 0.006 | 0.004 | 0.004 | 0.008 |
| CAP210.E8 | C | >50 | 0.268 | 0.078 | 0.106 | 0.645 |
| CAP244.D3 | C | >50 | >50 | >50 | >50 | >50 |
| CAP256.206.C9 | C | 0.046 | 0.006 | 0.003 | 0.004 | 0.009 |
| CAP45.G3 | C | >50 | 24.1 | 20.6 | 47.5 | >50 |
| Ce1176.A3 | C | 0.066 | 0.01 | 0.006 | 0.006 | 0.017 |
| CE703010217.B6 | C | 0.017333 | 0.012 | 0.008 | 0.008 | 0.016 |
| CNE30 | C | 0.296333 | 0.027 | 0.013 | 0.016 | 0.047 |
| CNE31 | C | 2.08 | 0.147 | 0.049 | 0.078 | 0.25 |
| CNE53 | C | 0.072333 | 0.015 | 0.006 | 0.009 | 0.019 |
| CNE58 | C | >50 | 0.075 | 0.039 | 0.033 | 0.195 |
| DU123.06 | C | 0.135333 | 0.008 | 0.006 | 0.005 | 0.013 |
| DU151.02 | C | 0.028333 | 0.004 | 0.003 | 0.003 | 0.005 |
| DU156.12 | C | 0.036667 | 0.005 | 0.003 | 0.003 | 0.006 |
| DU172.17 | C | 0.410333 | 0.007 | 0.005 | 0.005 | 0.018 |
| DU422.01 | C | 0.175667 | 0.022 | 0.013 | 0.013 | 0.036 |
| MW965.26 | C | 0.093 | 0.009 | 0.006 | 0.004 | 0.011 |
| SO18.18 | C | 0.016667 | 0.009 | 0.005 | 0.007 | 0.011 |
| TV1.29 | C | 0.437667 | 0.01 | 0.005 | 0.008 | 0.031 |
| TZA125.17 | C | 9.426667 | 0.403 | 0.137 | 0.186 | 0.549 |
| TZBD.02 | C | 0.137333 | 0.007 | 0.004 | 0.006 | 0.013 |
| ZA012.29 | C | 0.034667 | 0.012 | 0.006 | 0.007 | 0.014 |
| ZM106.9 | C | 0.032 | 0.006 | 0.003 | 0.004 | 0.008 |
| ZM109.4 | C | >50 | >50 | >50 | >50 | >50 |
| ZM135.10a | C | 3.686667 | 0.508 | 0.14 | 0.188 | 0.78 |
| ZM176.66 | C | 27.6 | 0.31 | 0.091 | 0.106 | 0.454 |
| ZM197.7 | C | >50 | >50 | >50 | >50 | >50 |
| ZM214.15 | C | 1.57 | 0.126 | 0.053 | 0.067 | 0.294 |
| ZM215.8 | C | 0.084 | 0.047 | 0.023 | 0.026 | 0.048 |
| ZM233.6 | C | 3.596667 | 0.073 | 0.024 | 0.024 | 0.065 |
| ZM249.1 | C | >50 | 1.01 | 0.992 | 0.874 | 4.25 |
| ZM53.12 | C | 0.007667 | 0.062 | 0.02 | 0.032 | 0.079 |
| ZM55.28a | C | 0.216 | 0.029 | 0.014 | 0.014 | 0.029 |
| 3326.v4.c3 | CD | >50 | 5.05 | 2.15 | 6.86 | 32 |
| 3337.v2.c6 | CD | >50 | 2.63 | 1.25 | 1.43 | 1.34 |
| 3817.v2.c59 | CD | >50 | 7.22 | 2.58 | 6.79 | 17.9 |
| 191821.E6.1 | D | >50 | 3 | 0.095 | 0.105 | 0.727 |
| 231965.c01 | D | >50 | >50 | >50 | >50 | >50 |
| 247-23 | D | >50 | >50 | >50 | >50 | >50 |
| 3016.v5.c45 | D | >50 | >50 | >50 | >50 | >50 |
| 57128.vrc15 | D | >50 | 0.026 | 0.009 | 0.01 | 0.038 |
| 6405.v4.c34 | D | 0.136 | 0.009 | 0.004 | 0.005 | 0.012 |
| A03349M1.vrc4a | D | 0.359333 | 0.019 | 0.007 | 0.011 | 0.02 |
| A07412M1.vrc12 | D | 0.207667 | 0.012 | 0.007 | 0.006 | 0.011 |
| NKU3006.ec1 | D | >50 | >50 | >50 | >50 | >50 |
| UG021.16 | D | >50 | 0.022 | 0.02 | 0.019 | 0.078 |
| UG024.2 | D | >50 | 4.72 | >50 | >50 | >50 |
| P0402.c2.11 | G | 0.029667 | 0.007 | 0.004 | 0.005 | 0.009 |
| P1981.C5.3 | G | 0.013 | 0.005 | 0.003 | 0.003 | 0.005 |
| X1193.c1 | G | 0.146667 | 0.007 | 0.004 | 0.005 | 0.013 |
| X1254.c3 | G | 0.106333 | 0.009 | 0.005 | 0.006 | 0.015 |
| X1632.S2.B10 | G | >50 | >50 | >50 | >50 | >50 |
| X2088.c9 | G | 0.019 | 0.01 | 0.005 | 0.009 | 0.017 |
| X2131.C1.B5 | G | 0.036 | 0.004 | 0.002 | 0.003 | 0.007 |
| SIVmac251.30.SG3 | NA | >50 | >50 | >50 | >50 | >50 |
| SVA.MLV | NA | >50 | >50 | >50 | >50 | >50 |

| | PGT121 | ePGT121.15 | ePGT121.18 M1 | ePGT121.18 M2 | ePGT121.18.17 |
|---|---|---|---|---|---|
| # Viruses | 208 | 208 | 208 | 208 | 208 |
| Total VS Neutralized | | | | | |
| IC80 < 50 ug/ml | 122 | 151 | 152 | 151 | 147 |
| IC80 < 10 ug/ml | 116 | 146 | 147 | 145 | 141 |
| IC80 < 1.0 ug/ml | 102 | 132 | 137 | 137 | 132 |
| IC80 < 0.1 ug/ml | 61 | 117 | 126 | 124 | 111 |
| IC80 < 0.01 ug/ml | 1 | 56 | 91 | 80 | 30 |
| % vs Neutralized | | | | | |
| IC80 < 50 ug/ml | 58.65385 | 72.59615 | 73.07692 | 72.59615 | 70.67308 |
| IC80 < 10 ug/ml | 55.76923 | 70.19231 | 70.67308 | 69.71154 | 67.78846 |
| IC80 < 1.0 ug/ml | 49.03846 | 63.46154 | 65.86538 | 65.86538 | 63.46154 |
| IC80 < 0.1 ug/ml | 29.32692 | 56.25 | 60.57692 | 59.61538 | 53.36538 |
| IC80 < 0.01 ug/ml | 0.480769 | 26.92308 | 43.75 | 38.46154 | 14.42308 |
| Median IC80 | 0.0985 | 0.015 | 0.007 | 0.008 | 0.02 |
| Geometric Mean | 0.154347 | 0.035076 | 0.01772 | 0.020206 | 0.04427 |

### Example 5. The Engineered Variant PGT-121 Antibodies have Increased Binding Affinity to Gp120 at Low pH

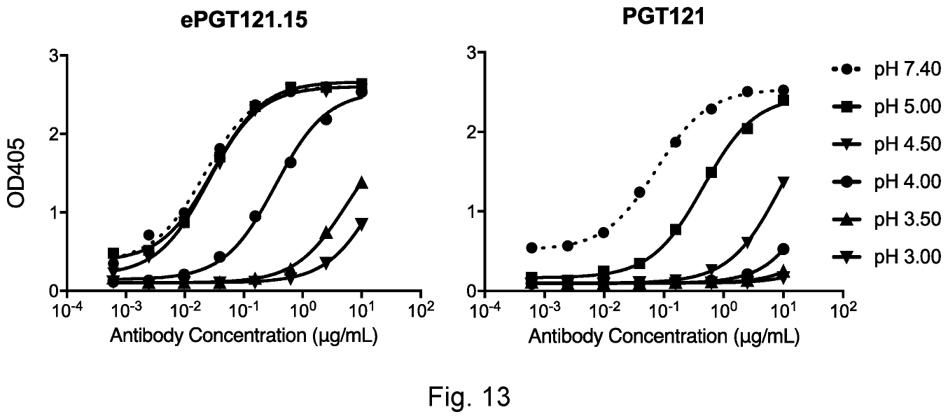

Binding affinity of ePGT-121.15.1 to gp120 (isolate: 92BR020) was measured at various pH levels using an ELISA assay as described in Example 1. gp120 binding by PGT-121.15.1 (comprising the ePGT121.15.H1 VH and ePGT121.15.L1 VL) is much less sensitive to low pH than binding by the parental PGT-121 antibody. In particular, ePGT-121.15.1 displayed significantly higher binding affinity at pH 4.50, 4.00, 3.50, and 3.00 than the parental PGT-121 antibody. FIG. 13. Thus, the engineered PGT-121 variant antibodies provided herein are suitable for intravaginal application.

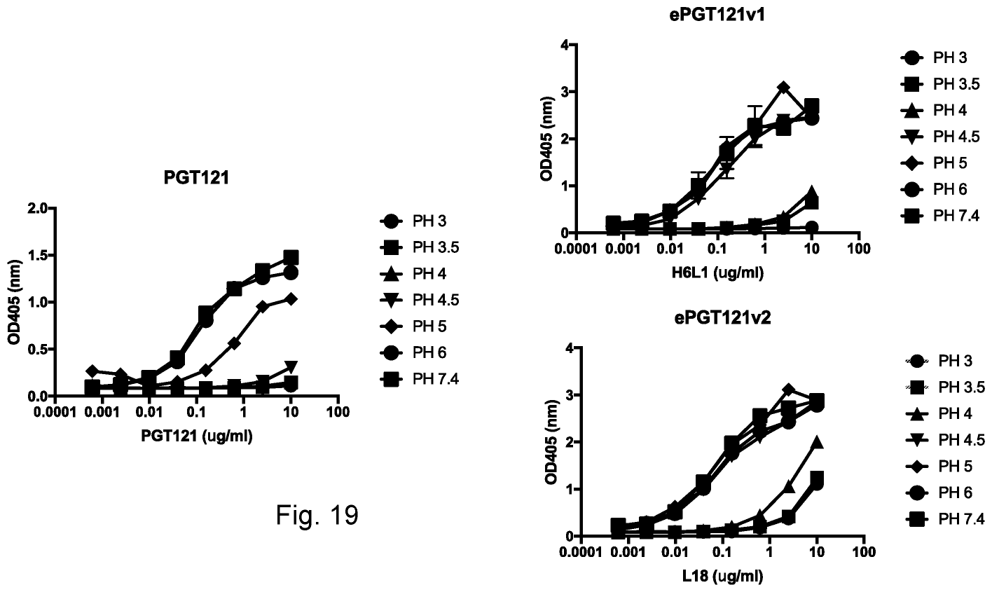

Binding affinity of PGT121. ePGT121v1 and ePGT121v2 to gp120 was measured at various pH levels using an ELISA assay as described in Example 1. ePGT121v1 comprises ePGT121.L15.H6 VH and ePGT121.L15.L1 VL, and ePGT121v2 comprises ePGT121.L18.H1 VH and ePGT121.L17.L1 VL. gp120 binding by ePGT121v1 and ePGT121v2 is much less sensitive to low pH than binding by the parental PGT-121 antibody. Both ePGT121v1 and ePGT121v2 displayed significantly higher binding affinity at pH 4.50, 4.00, 3.50, and 3.00 than the parental PGT-121 antibody. FIG. 19.

### Example 6. The Engineered Variant PGT-121 Antibodies Target the Same Epitope as PGT121

Epitope specificity of ePGT121 antibodies was assessed by comparing their neutralizing activity on a panel of mutant virus isolates. A complete loss of neutralization was observed for PGT121 and for two ePGT121 antibodies (comprising (a) ePGT121.L15.H6 VH and ePGT121.L15.L1 VL, or (2) ePGT121.L18.H1 VH and ePGT121.L17.L1 VL) when the glycan sequon at position N332 on HIV Env is removed. Furthermore, PGT121 and the two ePGT121 antibodies displayed reduced of neutralization potency when residue Asp325 is removed on HIV Env using either the D325A or R327A mutation.

### Example 7. The Engineered Variant PGT-121 Antibodies are Suitable for Pharmaceutical Development

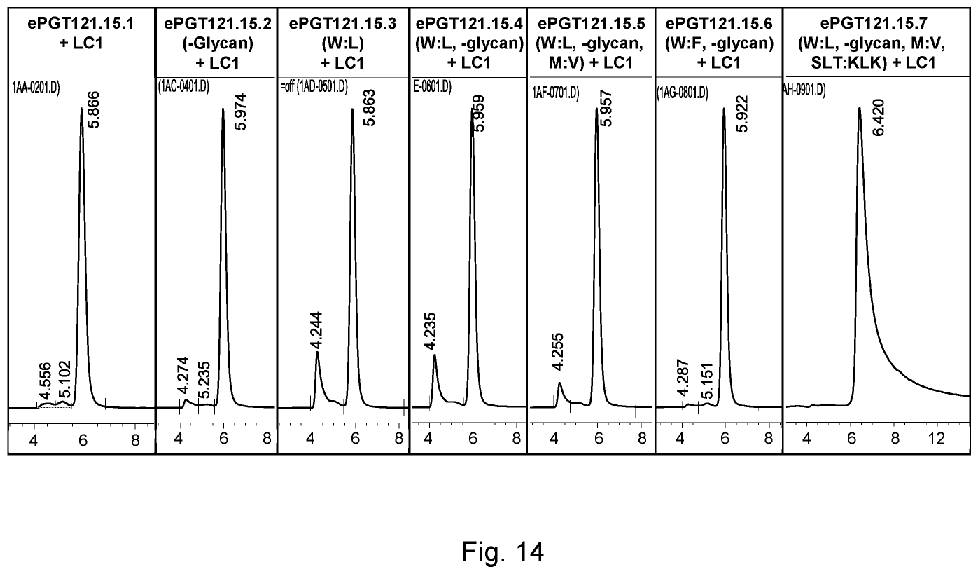

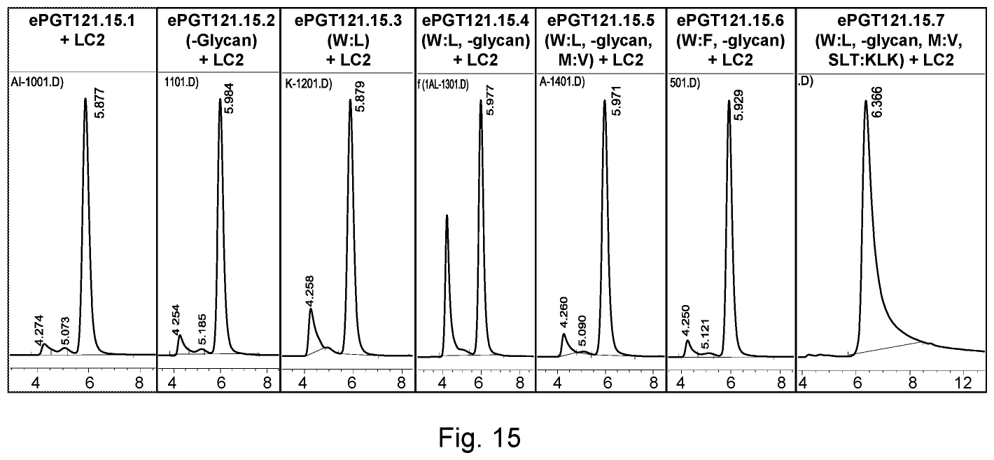

Recombinantly produced engineered PGT-121 variant antibody preparations were tested by SEC. FIGS. 14 and 15. The overwhelming majority of the product for all antibodies tested appeared in a single peak. Thus, the engineered PGT-121 variant antibodies provided herein are suitable for large scale production and pharmaceutical development.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

1. An antibody or antigen binding fragment thereof capable of binding HIV gp120, wherein the antibody or antigen binding fragment thereof comprises a VH and a VL, wherein the VH comprises a VH CDR1, VH CDR2, and VH CDR3, and the VL comprises a VL CDR1, VL CDR2, and VL CDR3, wherein
(1)
a1) the VH CDR1 comprises the sequence of X₁SYWS, wherein X₁ is D or S (SEQ ID NO: 26);
b1) the VH CDR2 comprises the sequence of YX₁HX₂SGDTNYX₃PSLKS wherein X₁ is V or T, X₂ is K or H, and X₃ is S or A (SEQ ID NO: 27);
c1) the VH CDR3 comprises the sequence of
TLHGRRX₁YGX₂VAFX₃EX₄X₅TYFYX₆X₇X₈ wherein X₁ is I or C, X₂ is I or V, X₃ is N or G, X₄ is W, C, F, L or Y, X₅ is F or Y, X₆ is M or W, X₇ is D, P or E, and X₈ is V or T (SEQ ID NO: 71) or
TLHGRRIYGX₁VAFX₂EX₃X₄TYFYX₅X₆X₇ wherein X₁ is I or V, X₂ is N or G, X₃ is W, F, L or Y, X₄ is F or Y, X₅ is M or W, X₆ is D, P or E, and X₇ is V or T (SEQ ID NO: 72);
d1) the VL CDR1 comprises the sequence of GX₁X₂SX₃GSRAVQ wherein X₁ is E or G, X₂ is K or E, and X₃ is L or I (SEQ ID NO: 29);
e1) the VL CDR2 comprises the sequence of NNQDRX₁X₂ wherein X₁ is P or G, and X₂ is S or P (SEQ ID NO: 168); and
f1) the VL CDR3 comprises the sequence of HX₁WDSRX₂PTX₃WX₄ wherein X₁ is I or V, X₂ is V or R, X₃ is K, Q or N, and X₄ is V or E (SEQ ID NO: 73); or
(2)
a2) the VH CDR1 comprises the sequence of X₁SYWS, wherein X₁ is D or S (SEQ ID NO: 26);
b2) the VH CDR2 comprises the sequence of YX₁HX₂SGDTNYX₃PSLKS wherein X₁ is V or T, X2 is K or H, and X3 is S or A (SEQ ID NO: 27);
c2) the VH CDR3 comprises the sequence of
TLHGRRX₁YGX₂VAFX₃EX₄FTYFYXSX₆V wherein X₁ is I or C, X₂ is I or V, X₃ is N or G, X₄ is W, C, F or L, X₅ is M or W, and X₆ is D or E (SEQ ID NO: 28) or TLHGRRIYGX₁VAFX₂EX₃FTYFYX₄X₅V wherein X₁ is I or V, X₂ is N or G, X₃ is W, F or L, X₄ is M or W, and X₅ is D or E (SEQ ID NO: 64);
d2) the VL CDR1 comprises the sequence of GX₁X₂SX₃GSRAVQ wherein X₁ is E or G, X₂ is K or E, and X₃ is L or I (SEQ ID NO: 29);
e2) the VL CDR2 comprises the sequence of NNQDRPX₁ wherein X₁ is S or P (SEQ ID NO: 30); and
f2) the VL CDR3 comprises the sequence of HIWDSRX1PTX2WV wherein X1 is V or R, and X2 is K or N (SEQ ID NO: 31);
wherein at least one of the VH CDR1, VH CDR2, VH CDR3, VL CDR1, VL CDR2, and VL CDR3 comprises a different amino acid sequence than the corresponding CDR of the PGT-121 antibody.

2. (canceled)

3. The antibody or antigen binding fragment thereof of claim 1, wherein the VH CDR1 comprises SEQ ID NO: 12 or 18; the VH CDR2 comprises the sequence of SEQ ID NO: 13, 19, or 61; the VH CDR3 comprises the sequence of 14, 20, 21, 22, 59, 60, 62, 67, or 140-144; the VL CDR1 comprises the sequence of SEQ ID NO: 15, 23, or 63; the VL CDR2 comprises the sequence of SEQ ID NO: 16, 24, or 145; and/or the VL CDR3 comprises the sequence of SEQ ID NO: 17, 25, 68, or 146, or 147.

4. The antibody or antigen binding fragment thereof of claim 1, wherein
a) the VH CDR1, VH CDR2, and VH CDR3 comprises the sequence of 12, 13, and 14, respectively; 18, 19, and 20, respectively; 18, 19, and 21, respectively; 18, 19, and 22, respectively; 12, 61, and 62, respectively; 18, 19, and 59, respectively; 18, 19, and 60, respectively; 18, 19, and 67, respectively; 18, 19, and 140, respectively; 18, 19, and 141, respectively; 18, 19, and 142, respectively; 18, 19, and 143, respectively; or 18, 19, and 144, respectively; and/or
b) the VL CDR1, VL CDR2 and VLCDR3 comprises the sequence of SEQ ID NO: 15, 16, 17, respectively; 23, 24, and 25, respectively; 63, 15, 25, respectively; 23, 24, 68, respectively; 23, 24, 146, respectively; 23, 24, 147, respectively; or 23, 145, 68, respectively.

5-11. (canceled)

12. The antibody or antigen binding fragment thereof of claim 1, wherein the VH framework regions comprise one or more of a V at H2, G at H15, L at H37, E at H39, T at H40, L or I at H45, H or T at H68, I at H69, G at H70, H at H72, P at H73, E at H74, K at H81, K or T at H82A, S at H82B, V at H82C, T at H87, A at H88, V at H89, and N or K at H105, I at H107, K at H108, wherein the VH framework residues are numbered according to Kabat.

13-17. (canceled)

18. The antibody or antigen binding fragment thereof of claim 1, wherein the VH has at least about 70%, at least about 75%, at least about 80%, at least about 90%, or at least about 95% sequence identity to SEQ ID NO: 3-9, 45, 47, 49-54, 65, 69, 70, or 112-126.

19. The antibody or antigen binding fragment thereof of claim 1, wherein the VL framework regions comprise one or more of a S at L2, Y at L3, Vat L4, L at L5, Tat L6, Q at L7, P at L8, P at L9, S at L10, Vat L11, Vat L19, Tat L22, Q at L38, P at L40, K or R at L45, I at L68, D at L69, S at L79, N or R at L80, T at L88, L at L101, E at 102L, and P at L103, wherein the VL framework residues are numbered according to Kabat.

20-24. (canceled)

25. The antibody or antigen binding fragment thereof of claim 1, wherein the VL has at least about 70%, at least about 75%, at least about 80%, at least about 90%, or at least about 95% sequence identity to SEQ ID NO: 10, 11, 46, 48, 55, 66, or 127-139.

26-59. (canceled)

60. An antibody or antigen binding fragment of claim 1, wherein the antibody comprises
a. a VH comprising the amino acid sequence of SEQ ID NO: 3 and a VL comprising the amino acid sequence of SEQ ID NO: 10;
b. a VH comprising the amino acid sequence of SEQ ID NO: 3 and a VL comprising the amino acid sequence of SEQ ID NO: 11;
c. a VH comprising the amino acid sequence of SEQ ID NO: 4 and a VL comprising the amino acid sequence of SEQ ID NO: 10;
d. a VH comprising the amino acid sequence of SEQ ID NO: 4 and a VL comprising the amino acid sequence of SEQ ID NO: 11;
e. a VH comprising the amino acid sequence of SEQ ID NO: 5 and a VL comprising the amino acid sequence of SEQ ID NO: 10;
f. a VH comprising the amino acid sequence of SEQ ID NO: 5 and a VL comprising the amino acid sequence of SEQ ID NO: 11;
g. a VH comprising the amino acid sequence of SEQ ID NO: 6 and a VL comprising the amino acid sequence of SEQ ID NO: 10;
h. a VH comprising the amino acid sequence of SEQ ID NO: 6 and a VL comprising the amino acid sequence of SEQ ID NO: 11;
i. a VH comprising the amino acid sequence of SEQ ID NO: 7 and a VL comprising the amino acid sequence of SEQ ID NO: 10;
j. a VH comprising the amino acid sequence of SEQ ID NO: 7 and a VL comprising the amino acid sequence of SEQ ID NO: 11;
k. a VH comprising the amino acid sequence of SEQ ID NO: 8 and a VL comprising the amino acid sequence of SEQ ID NO: 10;
l. a VH comprising the amino acid sequence of SEQ ID NO: 8 and a VL comprising the amino acid sequence of SEQ ID NO: 11;
m. a VH comprising the amino acid sequence of SEQ ID NO: 9 and a VL comprising the amino acid sequence of SEQ ID NO: 10;
n. a VH comprising the amino acid sequence of SEQ ID NO: 9 and a VL comprising the amino acid sequence of SEQ ID NO: 11;
o. a VH comprising the amino acid sequence of SEQ ID NO: 65 and a VL comprising the amino acid sequence of SEQ ID NO: 55;
p. a VH comprising the amino acid sequence of SEQ ID NO: 69 and a VL comprising the amino acid sequence of SEQ ID NO: 55;
q. a VH comprising the amino acid sequence of SEQ ID NO: 70 and a VL comprising the amino acid sequence of SEQ ID NO: 55;
r. a VH comprising the amino acid sequence of SEQ ID NO: 65 and a VL comprising the amino acid sequence of SEQ ID NO: 66;
s. a VH comprising the amino acid sequence of SEQ ID NO: 69 and a VL comprising the amino acid sequence of SEQ ID NO: 66;
t. a VH comprising the amino acid sequence of SEQ ID NO: 70 and a VL comprising the amino acid sequence of SEQ ID NO: 66; or
u. a VH comprising the amino acid sequence of SEQ ID NO: 112-126 and a VL comprising the amino acid sequence of SEQ ID NO: 127-139.

61. The antibody or antigen binding fragment thereof of claim 1, which is capable of binding HIV gp120 at pH 4.0.

62-75. (canceled)

76. The antibody or antigen binding fragment thereof of claim 1, which is capable of neutralizing at least two cross-clade isolates of HIV.

77-81. (canceled)

82. The antibody or antigen binding fragment thereof of claim 1, wherein
a) the antibody is a recombinant antibody, a chimeric antibody, a bispecific antibody, or a trispecific antibody;
b) the antibody fragment comprises a single-chain Fv (scFv), F(ab) fragment, F(ab′)2 fragment, or an isolated VH domain;
c) the antibody comprises a human heavy and light chain constant region; and/or
d) the antibody comprises a human heavy and light chain constant region, wherein the heavy chain constant region comprises a variant amino acid sequence.

83-88. (canceled)

89. A pharmaceutical composition comprising the antibody or antigen binding fragment thereof of claim 1 and a pharmaceutically acceptable excipient.

90. An isolated polynucleotide encoding the antibody or antigen binding fragment thereof of claim 1.

91-93. (canceled)

94. An isolated vector, a recombinant virus or a host cell comprising the polynucleotide of claim 90.

95-99. (canceled)

100. A method of producing the antibody or antigen binding fragment thereof of claim 1 comprising culturing the host cell comprising a nucleotide encoding the antibody or antigen binding fragment thereof so that the antibody or antigen binding fragment thereof is expressed and the antibody or antigen binding fragment thereof is produced.

101. A method of neutralizing an HIV virus comprising contacting the virus with a sufficient amount of the antibody or antigen binding fragment thereof of claim 1.

102. A method of reducing the likelihood of HIV infection in a subject exposed to HIV comprising administering to the subject a therapeutically sufficient amount of the antibody or antigen binding fragment thereof of claim 1.

103. A method of treating HIV/AIDS comprising administering to a subject in need thereof a therapeutically sufficient amount of the antibody or antigen binding fragment thereof of claim 1.

104. A method of reducing viral load comprising administering to a subject in need thereof a therapeutically sufficient amount of the antibody or antigen binding fragment thereof of claim 1.

105. (canceled)

106. The method of claim 103, further comprising administering at least one additional therapeutic agent, wherein the additional therapeutic agent is an antiretroviral agent, a reservoir activator, or a second antibody.

107-111. (canceled)

112. A method of producing an engineered variant of PGT121 comprising
a. substituting one or more amino acid residues of the PGT121 VH to a V at H2, G at H15, S at H31, L at H37, Eat H39, Tat H40, L or I at H45, Tat H51, H at H53, A at H60, H or T at H68, I at H69, G at H70, H at H72, P at H73, E at H74, K at H81, K or T at H82A, S at H82B, V at H82C, T at H87, A at H88, V at H89, C at H100A, V at H100B, V at H100D, E at H100F, G at H100G, G at H100H, L, F, Y or C at H100J, Y at H100K, W at H100P, E or P at H101, T at H102, and N or K at H105, I at H107, K at H108, wherein the VH residues are numbered according to Kabat; and/or
b. substituting one or more amino acid residues of the PGT121 VL to a S at L2, Y at L3, V at L4, L at L5, Tat L6, Q at L7, P at L8, P at L9, S at L10, Vat L11, Vat L19, Tat L22, G at L25, E at L26, I at L28, Q at L38, P at L40, K or R at L45, G at L55, P at L56, I at L68, D or A at L69, S at L79, N or R at L80, T at L88, V at L93, R at L95C, N or Q at L95F, E at L97, L at L101, E at 102L, and P at L103, wherein the VL residues are numbered according to Kabat.
